(19) United States
(12) Patent Application Publication
(10) Pub. No.: US 2022/0380801 A1
(43) Pub. Date: Dec. 1, 2022

(54) TRIPARTITE SYSTEMS FOR PROTEIN DIMERIZATION AND METHODS OF USE

(71) Applicant: MEDIMMUNE LIMITED, Cambridge (GB)

(72) Inventors: Lisa BAMBER, Cambridge (GB); Roger Bradley DODD, Cambridge (GB); Sandrine LEGG, Cambridge (GB); Thomas Vincent MURRAY, Cambridge (GB); David Gareth REES, Cambridge (GB); Anna Gudny SIGURDARDOTTIR, Cambridge (GB); Natalie Jo TIGUE, Cambridge (GB); Lisa Marie Kitching VINALL, Cambridge (GB); Christina SCHINDLER, Cambridge (GB); Bruck TADDESE, Cambridge (GB)

(21) Appl. No.: 17/627,210

(22) PCT Filed: Jul. 15, 2020
(86) PCT No.: PCT/IB2020/056657

§ 371 Date: Jan. 14, 2022

Related U.S. Application Data
(60) Provisional application No. 62/874,025, filed on Jul. 15, 2019.

(51) Int. Cl. C12N 15/86; C07K 14/005; A61K 31/4709; C07K 14/81

(52) U.S. Cl. CPC C12N 15/86; C07K 14/005; A61K 31/4709; C07K 14/81; A61K 2039/5156

(57) ABSTRACT

The disclosure provides compositions and methods that make use of a target protein that is capable of binding to a small molecule in order to form a complex, and a binding member that specifically binds to the complex, wherein the target protein is derived from a non-human protein and the small molecule is an inhibitor of the non-human protein. The non-human protein may be derived from a viral, bacterial, fungal or protozoal protein. These compositions and methods permit the controlled interaction of polypeptides that are individually fused to the target protein and binding member, respectively, and can be used to control the activity of dimerization-inducible proteins such as split transcription factors and split chimeric antigen receptors through the addition of the small molecule. The disclosure provides expression vectors, binding members, dimerization-inducible proteins, nucleic acids, cells, viral particles, kits, systems and methods that involve these components.

### CROSS-REFERENCE TO RELATED APPLICATIONS

This application is a U.S. National Stage application of International Application No. PCT/IB2020/056657, filed on Jul. 15, 2020, said International Application No. PCT/IB2020/056657 claims benefit under 35 U.S.C. § 119(e) of the U.S. Provisional Application No. 62/874,025, filed Jul. 15, 2019. Each of the above listed applications is incorporated by reference herein in its entirety for all purposes.

### FIELD

The present disclosure relates to compositions and methods that permit the controlled interaction of polypeptides to which a target protein and binding members are fused to. The compositions and methods make use of a target protein that binds to a small molecule to form a complex and a binding member that specifically binds the complex, wherein the target protein is derived from a non-human protein and the small molecule is an inhibitor of the non-human protein. The non-human protein may be derived from a bacterial, viral, fungal or protozoal protein. The non-human protein may be derived from a viral protease and the small molecule is a viral protease inhibitor. The present disclosure also relates to dimerization-inducible proteins, such as split transcription factors and split chimeric antigen receptors, that contain the target protein and binding member. The methods and compositions described herein find application, for example, in cell and gene therapy methods that involve the controlled expression and/or activation of proteins.

### BACKGROUND

Protein-protein interactions (PPIs) represent a universal regulatory mechanism that controls multiple biological functions. For example, gene transcription, protein folding, protein localisation, protein degradation, and signal transduction all rely on the interaction or proximity of one protein to another, or indeed several others. By temporally controlling protein-protein interactions, researchers can readily monitor the functional consequences of a PPI, enabling the dissection of complex biological mechanisms. Furthermore, the ability to control biological functions are being utilised in cell and gene therapy to control therapeutic activity, enabling safer and more personalised therapies.

A commonly used technique for controlling protein-protein interactions is to use so-called chemical inducers of dimerization (CID), small molecules that bring together two proteins that do not interact in the absence of the CID, to form a tripartite ternary complex (Stanton, Chory, and Crabtree 2018). The most widely used CID is rapamycin (an immunosuppressive drug derived from Streptomyces hygroscopicus) and analogues thereof, that forms a heterodimeric complex with the proteins FKBP12 (12-kDa FK506-binding protein) and FRB (a domain from mTOR (mammalian target of rapamycin)) (Sabers et al. 1995).

An attractive feature of rapamycin, along with other naturally-occurring CIDs, such as the plant hormones S-(+)-abscisic acid (ABA) and gibberellin (GA3-AM), is its co-operative binding mechanism whereby protein 2 can only bind to the protein 1:CID complex ((Banaszynski, Liu, and Wandless 2005). De novo CIDs have also been generated through the chemical linkage of two small molecules that bind the same, or different proteins, with these proteins constituting the dimerization protein pair (Belshaw, Ho, et al. 1996; Belshaw, Spencer, et al. 1996). In these systems however, at high concentrations of the bi-functional CID, non-productive complexes between one protein partner and the CID out-compete the production of tripartite complexes, meaning that a linear dose-response cannot be achieved.

As such, there is a growing urgency for new co-operative binding CID systems that can be used to regulate cellular function and to expand the number of orthogonal systems that can be used in complex genetic circuits. Furthermore, there are very few CIDs that have been approved for chronic human use. Recently, a method to generate de novo CID systems (AbCIDs) using antibody-based phage display selection methods was described (Hill et al. 2018). The CID used in that study was ABT-737, a Bcl-2 and Bcl-xL inhibitor, and Bcl-xL itself was employed as one of the protein partners. The second protein was then selected from a phage display library of single chain Fab (scFab) molecules to be selective for the Bcl-xL:ABT-737 complex over Bcl-xL alone.

The approach described in Hill et al. 2018 and WO 2018/213848 A1 of identifying complex-specific molecules by utilising existing small molecules and their targets is an attractive one, however, the overexpression of certain human proteins (e.g. the anti-apoptotic Bcl-xL protein) and use of small molecules that bind to human targets within the body is not without its risks. For example, overexpression of a functional human protein will have consequences for the cells in which it is expressed, which could impact cell health and viability. Additionally, the use of small molecules whose targets are expressed in the body, can result in an increased dose requirement due to the competition of binding of the small molecule to the endogenous target and the overexpressed target. Moreover, the binding of the small molecule to the endogenous target will affect the function of that protein that may be detrimental to the cells in which the target is expressed.

### SUMMARY

Disclosed herein is an approach aimed to overcome the limitations of the AbCID system as described by Hill et al. Firstly, the small molecules described herein are those that have already been approved for human use, to facilitate a smoother path to regulatory approval. Secondly, and importantly, rather than identifying small molecules with human targets, the inventors recognised that there were advantages associated with selecting small molecules that bind to non-human proteins, in particular viral proteins. For example, the use of a small molecule that does not have a human target is expected to improve safety when used in humans. It was also reasoned that the use of viral, bacterial, fungal or protozoal target proteins would remove the risk of an endogenous small molecule “sink” when used in a human, where the small molecule binds to endogenous targets in the human in addition to binding to the target protein. Furthermore, the expression of a viral, bacterial, fungal or protozoal protein within human cells is less likely to impact the cellular physiology of the cell than a human protein, that has endogenous function, would.

Antivirals have been approved that bind to and inhibit various viral proteins including viral polymerases, integrases, transcriptases and proteases. The present inventors recognised that target proteins derived from viral proteases in particular would be beneficial as these proteases are cytoplasmically located, are smaller, and consist of discrete domains.

Thus, the present disclosure provides one or more expression vectors comprising:
i) a first expression cassette encoding a target protein, wherein the target protein is capable of binding to a small molecule in order to form a complex between the target protein and small molecule (T-SM complex); and
ii) a second expression cassette encoding a binding member, wherein the binding member binds to the T-SM complex with a higher affinity than the binding members binds to both the target protein alone and the small molecule alone,
   wherein the target protein is derived from a non-human protein and the small molecule is an inhibitor of the non-human protein. In one embodiment, the non-human protein is derived from a viral protein and the small molecule is an inhibitor of the viral protein. In one embodiment, the non-human protein is derived from a viral protease and the small molecule is a viral protease inhibitor. In one embodiment, the non-human protein is derived from a bacterial protein and the small molecule is an inhibitor of the bacterial protein. In one embodiment, the non-human protein is derived from a fungal protein and the small molecule is an inhibitor of the fungal protein. In one embodiment, the non-human protein is derived from a protozoal protein and the small molecule is an inhibitor of the protozoal protein.

As demonstrated herein, binding of the binding member to the T-SM complex forms a tripartite complex made up of the binding member, target protein and small molecule and the formation of this tripartite complex can be controlled by the presence of the small molecule. The controlled formation of the tripartite complex is useful as, for example, it permits the controlled interaction of polypeptides to which the target protein and binding member are fused to.

The present disclosure also provides a system comprising:
i) a target protein, wherein the target protein is capable of binding to a small molecule in order to form a complex between the target protein and small molecule (T-SM complex); and
ii) a binding member, wherein the binding member specifically binds to the T-SM complex such that the binding member binds the T-SM complex at a higher affinity than it binds to both the target protein alone and the small molecule alone,
wherein the target protein is derived from a non-human protein and the small molecule is an inhibitor of the non-human protein. In one embodiment, the non-human protein is derived from a viral protein and the small molecule is an inhibitor of the viral protein. In one embodiment, the non-human protein is derived from a viral protease and the small molecule is a viral protease inhibitor. In one embodiment, the non-human protein is derived from a bacterial protein and the small molecule is an inhibitor of the bacterial protein. In one embodiment, the non-human protein is derived from a fungal protein and the small molecule is an inhibitor of the fungal protein. In one embodiment, the non-human protein is derived from a protozoal protein and the small molecule is an inhibitor of the protozoal protein.

In some embodiments, the viral protease is an HCV NS3/4A protease or HIV protease. These proteases are known to be targeted by several approved small molecules that are known to be generally well tolerated in humans and suitable for chronic dosing and therefore represent suitable target proteins for use herein.

In some embodiments, the viral protease is an HCV NS3/4A protease such as the protease having the amino acid sequence of SEQ ID NO: 1. The HCV NS3/4A protease is a small, monomeric protein that can be expressed cytoplasmically and has a limited number of endogenous human targets, therefore making it an ideal target protein.

In some embodiments, the small molecule is selected from the group consisting of simeprevir, asunaprevir, vaniprevir, boceprevir, narlaprevir, and telaprevir. All these small molecules are approved for treatment in humans. In some embodiments, the small molecule is selected from the group consisting of simeprevir, boceprevir, and telaprevir. These small molecules are approved for treatment in humans and are generally well tolerated in humans.

In some embodiments, the small molecule is simeprevir. Simeprevir (Olysio®) is a small molecule that is administered orally, is cell-permeable, and has a pharmacokinetics (PK) profile that supports once-daily dosing. It has been used chronically (up to 39 months) to treat HCV infection in combination with ribavirin and pegylated interferon, and is on the WHO essential medicines list, indicative of a well-tolerated and widely administered drug.

The inventors made the realisation that any potential off-target activity caused by overexpression of the viral protease could be mitigated by using target proteins that have attenuated viral activity compared to the viral protease from which it is derived. Thus, in some embodiments the target protein has attenuated viral activity compared to the viral protease from which it is derived.

For example, the target protein may contain one or more amino acid mutations compared to the viral protease from which it is derived. In particular embodiments where the viral protease is an HCV NS3/4A protease, the target protein may have an amino acid mutation at one or more amino acids selected from positions 72, 96, 112, 114, 154, 160 and 164, wherein the amino acid numbering corresponds to SEQ ID NO: 1. For example, the target protein may have an amino acid mutation at position 154, such as a mutation to alanine, wherein the amino acid numbering corresponds to SEQ ID NO: 1. As described below, positions 72, 96, 112, 114, 154, 160 and 164 of SEQ ID NO: 1 correspond to positions 57, 81, 97, 99, 139, 145 and 149, respectively, of the full length NS3 protein set forth in SEQ ID NO: 199. The examples refer to amino acid positions according to the amino acid numbering of the full length NS3 protein. For example, reference to a ‘S139A’ mutation in the examples corresponds to a ‘S154A’ mutation where the amino acid numbering corresponds to SEQ ID NO:1.

In some cases, it may be desirable that a competing small molecule is able to bind the target protein in the T-SM complex such that the competing small molecule is capable of displacing the small molecule in the T-SM complex, where the second small molecule is different to the small molecule in the T-SM complex. In this way, the second small molecule can decrease the half-life of the tripartite complex formed between the binding member, the target protein and the small molecule. This may be desirable, for example, in situations where it is considered useful to use the second small molecule to speed up dissociation of the tripartite complex, e.g. in order to rapidly inhibit activity of a dimerization-inducible protein activated by formation of the tripartite complex.

As demonstrated herein, simeprevir binds the target protein HCV NS3/4A protease (S139A) (SEQ ID NO: 2) with a very high affinity such that other small molecules that bind the target protein are unable to displace simeprevir from the T-SM complex. The inventors determined that certain affinity reducing mutations could be introduced in the target protein that reduce the affinity of simeprevir for the HCV NS3/4A protease and allow other small molecules to “compete” with simeprevir and disrupt the tripartite complex formed. Thus, in some embodiments where the viral protease is an HCV NS3/4A protease and the small molecule is simeprevir, the target protein may comprise an affinity reducing amino acid substitution at one or more amino acids selected from positions 151 and 183, wherein the amino acid numbering corresponds to SEQ ID NO: 1. In some embodiments, the affinity reducing amino acid mutation at position 151 is a mutation to aspartic acid, asparagine or histidine (e.g. aspartic acid or asparagine) and the affinity reducing mutation at position 183 is to glutamic acid, glutamine or alanine (e.g. glutamic acid). The target protein may comprise the affinity reducing amino acid mutation in addition to other mutations described herein, such as the amino acid mutation at one or more amino acids selected from positions 72, 96, 112, 114, 154, 160 and 164.

In some embodiments the binding member is an antibody molecule, such as a single-chain variable fragment (scFv), or an antibody mimetic, such as a Tn3 protein. In particular embodiments, the binding member is a Tn3 protein or an scFv, such as the Tn3 proteins and scFvs defined herein. Compared to the single chain Fabs (scFabs) used in the system described by Hill et al., both Tn3 proteins and scFvs are smaller in size. This may be advantageous, for example where the expression cassettes are being delivered by expression vectors that are limited in coding capacity such as viral vectors. Described herein is the development and use of particular Tn3 proteins and scFvs that bind to a complex between HCV NS3/4A protease and simeprevir, which are demonstrated to function as binding members in the context of the present disclosure. These Tn3 proteins and scFvs are termed HCV NS3/4A PR:simeprevir complex-specific binding (PRSIM) molecules.

It was realised that the approach described herein could be used where the target protein and binding member are individually fused to polypeptides (termed “component polypeptides”). In particular, it was realised that the approach could be implemented to control the activity of proteins that require dimerization or clustering to drive their activity. Such proteins are termed herein as “dimerization-inducible proteins” and include “split proteins”, “dimerization-deficient proteins” and “split complexes”. Split proteins comprise single proteins that can be segregated or split into two or more domains, rendering the component parts non-functional or minimally active; function or activity can be initiated or restored, however, when the separated component polypeptides are brought into close proximity. Examples include split fluorescent proteins (e.g. split GFP), split luciferases (e.g. NanoBiT) and split kinases. A further example describes a split transcription factor, whereby the distinct DNA binding (DBD) and activation domains (AD) are separated such that the individual transcription factor domains alone cannot initiate transcription. Only when the two domains are brought into close proximity are they able to reconstitute the transcriptional activation of relevant genes (i.e. they form a functional “transcription factor”). Dimerization-deficient proteins are proteins that require dimerization for activity, but their endogenous dimerization capacity has been disabled e.g. via mutation or removal of the dimerization domain(s). One such example is the iCasp9 molecule, a caspase 9 protein that has had its dimerization (CARD) domain removed. Split complexes denote either single proteins or 2 or more different proteins that are not optimally functional or function differently, until they are brought into close proximity or “clustered”. Once such example is the split chimeric antigen receptor (CAR). Here, specific intracellular domains of the CAR that are responsible for the activation of cell signalling are physically separated such that full cellular activation is prevented. Once the domains are brought into close proximity, cell signalling is activated (i.e. they form a fully functional CAR).

Thus, in some embodiments the target protein is fused to a first component polypeptide and the binding member is fused to a second component polypeptide. In preferred embodiments, the one or more expression vectors encode a dimerization-inducible protein, such as a split transcription factor or a split CAR.

In one embodiment: (1) the first component polypeptide comprises a DNA binding domain and is fused to the target protein to form a DBD-T (DBD-target protein) fusion protein; and the second component polypeptide comprises a transcriptional regulatory domain and is fused to the binding member to form a TRD-BM (transcriptional regulatory domain-binding molecule) fusion protein, or (2) the first component polypeptide comprises a transcriptional regulatory domain and is fused to the target protein to form a TRD-T fusion protein; and the second component polypeptide comprises a DNA binding domain and is fused to the binding member to form a DBD-BM fusion protein, wherein the first and second component polypeptide form a transcription factor upon dimerization.

In another embodiment, the first component polypeptide comprises a first co-stimulatory domain and is fused to the target protein; and the second component polypeptide comprises an intracellular signalling domain is fused to the binding member. The first component polypeptide may further comprise an antigen-specific recognition domain and a transmembrane domain; and the second component polypeptide further comprises a transmembrane domain and a second co-stimulatory domain, wherein the first and second component polypeptide form a chimeric antigen receptor (CAR) upon dimerization.

Alternatively, the first component polypeptide comprises an intracellular signalling domain and is fused to the target protein and the second component polypeptide comprises a first co-stimulatory domain and is fused to the binding member. The first component polypeptide further comprises a transmembrane domain and a second co-stimulatory domain; and the second component polypeptide further comprises an antigen-specific recognition domain and a transmembrane domain, and wherein the first and second component polypeptide form a chimeric antigen receptor (CAR) upon dimerization.

In another embodiment, the first component polypeptide comprises a first caspase component; and the second component polypeptide comprises a second caspase component, and the first and second component polypeptides form a caspase upon dimerization.

In some embodiments the one or more expression vector is a viral vector, such as an AAV vector.

The present disclosure also provides an in vitro method of making viral particles comprising transfecting host cells with the viral vector(s) defined herein and expressing viral proteins necessary for viral particle formation in the host cells; culturing the transfected cells in a culture medium, such that the cells produce viral particles.

The present disclosure also provides one or more viral particles comprising
i) a first expression cassette encoding a target protein, wherein the target protein is capable of binding to a small molecule in order to form a complex between the target protein and the small molecule (T-SM complex); and
ii) a second expression cassette encoding a binding member, wherein the binding member specifically binds to the T-SM complex such that the binding member binds the T-SM complex at a higher affinity than it binds both the target protein alone and the small molecule alone,
   wherein the target protein is derived from a non-human protein and the small molecule is an inhibitor of the non-human protein, and wherein the first and second expression cassettes form part of a viral genome in the one or more viral particles. In one embodiment, the non-human protein is derived from a viral protein and the small molecule is an inhibitor of the viral protein. In one embodiment, the non-human protein is derived from a viral protease and the small molecule is a viral protease inhibitor. In another embodiment, the non-human protein is derived from a bacterial, fungal or protozoal protein.

The expression cassettes, target protein, small molecule, binding member in the one or more viral particles may be as further described herein. The target protein and binding member may be fused to a first and second component polypeptide, respectively, (e.g. for encoding a dimerization-inducible protein) as further described herein.

The viral particle may be an AAV particle.

In one aspect the present disclosure provides a binding member that specifically binds to a complex between i) a target protein derived from a non-human protein and ii) a small molecule that is an inhibitor of the non-human protein, wherein the binding member binds the complex at a higher affinity than it binds both the target protein alone and the small molecule alone. In one embodiment, the non-human protein is derived from a viral protein and the small molecule is an inhibitor of the viral protein. In one embodiment, the non-human protein is derived from a viral protease and the small molecule is a viral protease inhibitor. In another embodiment, the non-human protein is derived from a bacterial, fungal or protozoal protein. As described herein, such complex-specific binding members are useful as a way of controlling formation of a tripartite complex between the binding member, target protein and small molecule in a manner that overcomes the drawbacks of the binding molecules described by Hill et al.

In another aspect, the present disclosure provides dimerization-inducible proteins comprising the target proteins and binding members, as defined herein. The dimerization-inducible proteins may be a split transcription factor, a split CAR or a split caspase protein, for example.

In one aspect, the present disclosure provides cells, e.g. allogeneic or autologous cells, including stem cells, induced pluripotent stem (iPS) cells or immune cells, comprising one or more of the expression cassettes, expression vectors, binding members, target proteins or dimerization inducible proteins defined herein. The cells may express the binding member, target protein or dimerization-inducible protein described herein. The present disclosure also provides methods of genetically modifying a cell to produce cells expressing the binding member or dimerization inducible protein described herein, the method comprising administering expression vectors to the cell. This method may be carried out in vitro or ex vivo.

It was additionally recognised that the approach described herein where the target protein and binding member are fused to component polypeptides of a split transcription factor could have uses in gene therapy methods that involve regulating the expression of a desired expression product (e.g. a desired polypeptide) in a cell.

Thus, in one aspect the present disclosure provides a method of regulating the expression of a desired expression product in a cell, comprising:
i) expressing the dimerization-inducible protein defined herein in the cell, wherein the first and second component polypeptides form a transcription factor upon dimerization, and wherein the DNA binding domain binds to a target sequence in the cell such that the transcription factor is capable of regulating expression of the desired expression product in the cell; and
ii) administering the small molecule to the cell in order to regulate expression of the desired expression product.

In some embodiments of the method, the DNA binding domain target sequence is located in a promoter that is operably linked to a coding sequence for the desired expression product.

The method may involve delivery of the expression cassettes encoding the dimerization-inducible protein to control expression of a desired expression product that is also delivered exogenously to the cell.

Thus, in some embodiments, the method comprises administering a third expression cassette to a cell, wherein the third expression cassette encodes the desired expression product, and wherein the third expression cassette comprises the target sequence of the DNA binding domain.

Alternatively, the method may involve delivery of the expression cassettes encoding the dimerization-inducible protein to control expression of a desired expression product that is already present as part of the genome of the cell (i.e. an endogenous desired expression product).

Thus, in other embodiments of the method, the target sequence is located in the genome of the cell.

Furthermore, it was recognised that the approach described herein could have use in methods of cellular therapy. Such methods typically involve taking cells from an individual (autologous cells), modifying the cells ex vivo to express a particular protein, e.g. a dimerization-inducible protein, and administered back into the individual.

Thus, in another aspect the present disclosure provides a method of treatment, the method comprising:
i) administering the cell comprising the expression cassettes encoding the dimerization-inducible protein as defined herein to an individual in need thereof; and
ii) administering the small molecule to the individual.

In one aspect, the present disclosure provides nucleic acids encoding the binding members, target proteins and dimerization-inducible proteins as defined herein.

In one aspect the present disclosure provides kits, as defined herein.

It was additionally recognised that it would be possible to make use of an additional small molecule (termed herein as a “competing small molecule”) to induce disassembly of a tripartite complex formed between the binding member, target protein and small molecule. This may be useful, for example, where it is desirable to rapidly inactivate a chemical inducer of dimerization (CID) disclosed herein, such as in order to turn off transgene expression or therapeutic activity association with activity of a dimerization-inducible protein.

This, in another aspect the present disclosure provides a method of inducing disassembly of a tripartite complex, the method comprising administering a competing small molecule to a cell comprising the tripartite complex,

wherein the tripartite complex is formed between a binding member and a complex formed of a target protein and a small molecule (T-SM complex), wherein the binding member binds the T-SM complex at a higher affinity than it binds both the target protein alone and the small molecule alone, and
wherein the competing small molecule is capable of binding the target protein in the T-SM complex and displacing the small molecule from the T-SM complex.

Methods of determining whether the competing small molecule is capable of binding to the target protein in the T-SM complex and displacing the small molecule from the T-SM complex include assays where a pre-formed tripartiate complex is generated and the ability of the binding member to bind the T-SM complex is measured (e.g. by a homogeneous time-resolved florescence (HTFR) binding assay) as increasing concentrations of the competing small molecule are added. A competing small molecule may be capable to displaying the small molecule from the T-SM complex if it is capable of inhibiting binding of inhibiting the binding member from binding the T-SM complex by at least 50%, by at least 75%, by at least 80%, by at least 85%, by at least 90%, or by at least 95% when measured using the HTFR binding assay. In some embodiments, the competing small molecule is asunaprevir, paritaprevir, vaniprevir, grazoprevir, danoprevir or glecaprevir. The binding member, target protein and small molecule using in the method may be as further defined herein in relation to other aspects of the disclosure.

In particular embodiments, the target protein may be derived from an HCV NS3/4A protease and the small molecule in the T-SM complex may be simeprevir and, optionally, the binding member may be PRSIM_23. For example, the target protein may have an amino acid sequence having at least 90% identity to SEQ ID NO: 1. As demonstrated herein, simeprevir binds the target protein HCV NS3/4A protease (S139A) (SEQ ID NO: 2) with a very high affinity such that other small molecules that bind the target protein are unable to displace simeprevir from the T-SM complex. As further demonstrated herein, it is possible to introduce mutations in the HCV NS3/4A protease that reduce affinity for simeprevir to the HCV NS3/4A protease and allow for a competing small molecule to disrupt the tripartite complex formed between the HCV NS3/4A protease, simeprevir and the binding member PRSIM_23

Accordingly, in embodiments where target protein is derived from an HCV NS3/4A protease and the small molecule is simeprevir, the target protein may have an affinity reducing amino acid mutation (e.g. substitution) at one or more amino acids selected from positions 151 and 183, wherein the amino acid numbering corresponds to SEQ ID NO: 1. In some embodiments, the affinity reducing amino acid mutation at position 151 is a mutation to aspartic acid, asparagine or histidine, and the affinity reducing mutation at position 183 is to glutamic acid, glutamine or alanine. In some embodiments, the affinity reducing amino acid mutation at position 151 is a mutation to aspartic acid or asparagine and the affinity reducing mutation at position 183 is to glutamic acid. The target protein may comprise the affinity reducing amino acid mutation in addition to another amino acid mutation described herein (e.g. in addition to the amino acid mutation at position 154, such as to an alanine).

The present disclosure includes the combination of the aspects and preferred features described except where such a combination is clearly impermissible or expressly avoided.

### SUMMARY OF THE FIGURES

Embodiments and experiments illustrating the principles of the present disclosure will now be discussed with reference to the accompanying figures in which:

FIG. 1 shows a schematic of the three components of the exemplary PRSIM-based chemical inducer of dimerization (CID). A represents the target protein (e.g. the exemplified HCV NS3/4A PR (S139A) mutant), B represents the small molecule (e.g. the exemplified simeprevir), and C represents the binding member (e.g. an scFv or Tn3 that is specific for the complex of simeprevir and HCV NS3/4A PR (S139A)).

FIG. 2 depicts the three-dimensional structure of simeprevir in complex with HCV NS3/4A PR (PDB code: 3KEE; 2.4 A) and illustrates the shallow binding site of HCV NS3/4A PR and large surface-exposed area of simeprevir.

FIG. 3A shows an SDS-PAGE gel of recombinant WT and S139A HCV NS3/4A PR. The S139A HCV NS3/4A PR comprises a serine to alanine mutation at a position that corresponds to amino acid position 139 of the full length NS3 protein (SEQ ID NO: 199). The position of this serine to alanine mutation corresponds to position 154 of the HCV NS3/4A protease provided here as SEQ ID NO: 1.

FIG. 3B illustrates the minimal activity of the S139A mutant of HCV NS3/4A PR, compared to its WT counterpart in a peptide cleavage assay.

FIG. 3C shows isothermal calorimetry data that demonstrates an equivalent affinity of simeprevir for the WT and S139A versions of HCV NS3/4A PR.

FIG. 4A shows the selection strategy that was adopted to isolate HCV NS3/4A PR (S139A):simeprevir-selective binding molecules (PRSIMs).

FIG. 4B shows the outputs from different rounds of selection for three different libraries as represented by the fold-change in ELISA signal in the presence of simeprevir, compared to the binding signal obtained in the presence of HCV NS3/4A PR (S139A) alone.

FIG. 5 shows a schematic of the homogeneous time-resolved fluorescence (HTRF) assay employed to measure the binding of PRSIM molecules to HCV NS3/4A PR (S139A) alone or in complex with simeprevir.

FIG. 6 shows the HTRF data obtained with a panel of PRSIM molecules that demonstrate HCV NS3/4A PR (S139A):simeprevir-selective binding. The top row is in the presence of simeprevir and the bottom row is in the absence of simeprevir.

FIGS. 7A-B show BIAcore-derived affinity data for HCV NS3/4A PR (S139A) binding to FIG. 7A: PRSIM_57 and FIG. 7B: PRSIM_23 in the presence of simeprevir (left) and no significant binding in the absence of simeprevir (middle). BSA in the presence of simeprevir was used as a control (right). Grey curves represent measured data points and dashed black lines represent the global-fit lines used for analysis.

FIG. 7C shows a titration curve for the induction of HCV NS3/4A PR (S139A)/PRSIM_57 (left; EC50=4.57 nM) or HCV NS3/4A PR (S139A)/PRSIM_23 (right; EC50=4.03 nM) heterodimerisation by simeprevir. ⋄=40 nM HCV NS3/4A PR (S139A)+0 nM simeprevir.

FIG. 8 shows a schematic (left) of the nanoBiT system (Promega) that was used to identify PRSIM molecules capable of reconstituting the function of nanoLuc by bringing the LgBiT and SmBiT domains into close proximity. The different orientations of LgBiT- and SmBiT-fusion proteins generated and tested are also depicted (right).

FIG. 9 shows the data obtained from the nanoBiT screen where the fold-change luminescence signal in the presence of simeprevir over the signal in the absence of simeprevir is depicted and demonstrates that several of the PRSIM binding molecules are capable of reconstituting nanoLuc activity.

FIG. 10 depicts the components of the two plasmids used in transient transfections to measure the ability of simeprevir to reconstitute a split transcription factor, and activate transcription of a luciferase reporter gene, when the component parts are fused to HCV NS3/4A PR (S139A) and different PRSIM molecules.

FIGS. 11A-B show the dose-response data obtained from the split transcription factor assay for Tn3-based PRSIM molecules (FIG. 11A), and scFv-based PRSIM molecules (FIG. 11B). Several of the PRSIM molecules tested enable dose-dependent activation of transcription of the luciferase reporter gene.

FIG. 12A show the dose-response data obtained from the split transcription factor assay for PRSIM_23 and PRSIM_57 compared to the rapamycin-inducible FRB:FKBP12 positive control, whereby superior fold-change and EC50 values were obtained.

FIG. 12B show the data obtained from the split transcription factor assay for PRSIM_23 and PRSIM_57 compared to the rapamycin-inducible FRB:FKBP12 positive control, in the absence of simeprevir or rapamycin, respectively, indicating that the PRSIM-based CIDs have lower basal expression levels, and are therefore more tightly regulated.

FIG. 13 depicts the anticipated increase in reporter gene expression when three copies of the molecule to which the DBD is fused is used, compared to a single copy, through recruitment of more AD domains, and associated regulatory molecules.

FIG. 14A shows the data obtained from plasmids encoding a single versus three copies of PRSIM_23 or FKBP12 fused to the DBD, indicating that an increase in copy number has a synergistic effect on the fold-change of expression.

FIG. 14B shows the data obtained from plasmids encoding varying copies of PRSIM_23 and a null Tn3 fused to the DBD, indicating that an increase in copy number has a synergistic effect on the fold-change of expression.

FIG. 15A depicts the plasmid used to express a PRSIM-based split chimeric antigen receptor, and the proteins expressed from this plasmid.

FIG. 15B demonstrates the effect of addition of simeprevir on the association of the PRSIM-based split CAR components, and the resultant cell activation achieved.

FIG. 16 shows the dose-dependent increase in IL-2 release, as a marker of T cell activation, from cells expressing a PRSIM-based split CAR in the presence of simeprevir, compared to an equivalent FRB:FKBP12-based CAR.

FIG. 17 shows the dose-response of simeprevir in inducing the expression of MED18852 via reconstitution of a split transcription factor assay using a PRSIM_23-containing CID.

FIG. 18A depicts the vectors used to generate separate AAV particles encoding either the inducible luciferase transgene or the PRSIM_23/HCV NS3/4A PR (S139A)-based split transcription factor components. Also depicted are the proteins expressed after transduction with both AAV particles, and luciferase expression after treatment with simeprevir.

FIG. 18B shows that the PRSIM_23 switch can activate dose-dependent expression of luciferase in the presence of simeprevir when the PRSIM_23 switch and the inducible luciferase transgene are delivered to cells in separate AAV particles.

FIG. 18C depicts the vector used to generate AAV particles encoding both the inducible IL-2 transgene and the PRSIM_23/HCV NS3/4A PR (S139A)-based split transcription factor components. Also depicted are the proteins expressed after transduction with these AAV particles, and IL-2 expression after treatment with simeprevir.

FIG. 18D shows that the PRSIM_23 switch can activate dose-dependent expression of IL-2 in the presence of simeprevir when the PRSIM_23 switch and the inducible IL-2 transgene are delivered to cells in the same AAV particle.

FIG. 18E shows that the level of IL-2 expression induced by the PRSIM_23 switch when the PRSIM_23 switch and the inducible IL-2 transgene are delivered to cells in the same AAV particle is similar to the level of IL-2 expression achieved by AAV delivery of IL-2 constitutively expressed from a CAG promoter.

FIG. 19A depicts the components of both the PRSIM-based activation plasmid and the IL-2 targeting gRNA plasmid, used to determine the ability of simeprevir to regulate endogenous gene expression within a CRISPRa approach.

FIG. 19B shows the induction of IL-2 expression from cells expressing both a PRSIM-based activation plasmid and an IL-2 targeting gRNA plasmid, only in the presence of Simeprevir.

FIG. 20 shows the dose-dependent induction of complex formation with a panel of small molecule HCV protease inhibitors.

FIG. 21 illustrates two-dimensional interactions diagram of simeprevir binding site of HCV NS3/NS4A.

FIG. 22 shows the ability of a panel of mutant HCV proteases to form a complex with PRSIM_23 and simeprevir.

FIG. 23 shows Octet-derived affinity data for simeprevir binding to HCV NS3/NS4A (S139A) PR (FIG. 23A), HCV NS3/NS4A K136D PR (FIG. 23B), HCV NS3/NS4A K136N PR (FIG. 23C) and HCV NS3/NS4A D168E PR (FIG. 23D). Data is representative of 2-3 independent experiments.

FIG. 24A shows a titration curve for the induction of mutant HCV NS3/4A PR/PRSIM_23 binding molecule heterodimerisation by simeprevir; HCV NS3/4A PR ‘WT’ (S139A) (•), HCV PR NS3/4A K136D (▪), HCV PR NS3/4A K136N (▴) and HCV PR NS3/4A D168E (⋄).

FIGS. 24B-E show BIAcore-derived affinity data for HCV NS3/4A PR ‘WT’ (S139A) (FIG. 24B), HCV PR NS3/4A K136D (FIG. 24C), HCV PR NS3/4A K136N (FIG. 24D) and HCV PR NS3/4A D168E (FIG. 24E) binding to PRSIM_23 in the presence of simeprevir (20, 800, 40 and 20 nM simeprevir, respectively) (left) and no significant binding in the absence of simeprevir (right). Grey curves represent measured data points and dashed black lines represent the global-fit lines used for analysis. Data is representative of 3 independent experiments.

FIG. 25A compares addition of small molecule inhibitors of HCV NS3/4A PR to inhibit formation of the switch complex with and without simeprevir/HCV NS3/4A PR pre-incubation.

FIG. 25B Small molecule inhibitors of HCV NS3/4A PR can disrupt the switch complex by competing with simeprevir for binding to HCV NS3/4A PR variants with an amino acid mutation at position 168 or 136.

FIG. 26A show the data obtained from the split transcription factor assay for PRSIM_23 HCV NS3/4A PR mutants compared to wild-type.

FIG. 26B depicts the vectors used to generate monoclonal cell lines expressing GFP-PEST under control of PRSIM_23 HCV NS3/4 PR WT and mutants achieved by AAVS1 transgene knockin via CRISPR. Also depicted are the proteins expressed and the effect of simeprevir addition resulting in the cell activation.

FIG. 26C shows representative histograms that demonstrate GFP fluorescence intensity as measured by flow cytometry in cell lines expressing GFP-PEST under control of split transcription factor PRSIM_23 HCV NS3/4 PR WT and mutants. Monoclonal cell lines were induced with simeprevir for 24 hr.

FIG. 26D show the data obtained for GFP fluorescence in cell lines expressing GFP-PEST under control of the split transcription factor PRSIM_23 HCV NS3/4A PR wt or mutants. Cells were treated with Simeprevir to induce expression. Simeprevir was removed and GFP fluorescence was determined at various timepoints after removal using flow cytometry.

FIG. 27A shows the overall structure of the HCV NS3/4A (S193A) PR:PRSIM_57: simeprevir ternary complex. Upper image: The HCV NS3/4A (S193A) PR (light grey) and PRSIM_57 (dark grey) are shown in a surface representation, with the simeprevir molecule shown in ball-and-stick format (black) sandwiched in the interface of the two proteins. Lower image: The HCV NS3/4A (S193A) PR (light grey) and PRSIM_57 (dark grey) are shown in cartoon format. The simeprevir is shown in ball-and-stick format (black) with the 2mFo-DFc electron density contoured at 2σ.

FIG. 27B shows details of the molecular interactions between HCV NS3/4A (S193A) PR, PRSIM_57 and simeprevir. Upper panel: Details of the interactions made with simeprevir by HCV NS3/4A (S193A) PR and PRSIM_57. HCV NS3/4A (S193A) PR residues interacting with simeprevir (ball-and-stick, black) are as previously determined (PDB 3KEE) and are shown with side chains in ball-and-stick format (carbon—light grey, oxygen/nitrogen—black). Hydrophobic residues in PRSIM_57 forming the hydrophobic cavity (Phe77, Ile74, Ile125 and Trp249) around simeprevir are shown in ball-and-stick format (carbon—dark grey, oxygen/nitrogen—black). A direct interaction occurs between the side chain of Phe77 and the simeprevir quinoline. Lower panel: Details of interactions between HCV NS3/4A (S193A) PR and PRSIM_57 coloured as in left panel. Interacting residues are shown in ball-and-stick format.

FIGS. 28A-C show design of kill switch. FIG. 28A: homodimerization of Caspase 9 (Casp9) via its CARD dimerization domain is crucial for induction of cell death via apoptosis. FIG. 28B: Replacement of CARD domain with PRSIM switch components. FIG. 28C: Addition of simeprevir induces formation of the PRSIM23-HCV PR heterodimer resulting in dimerisation of Casp9 active domains and subsequent induction of apoptosis.

FIGS. 29A-E show functionality of kill switch upon addition of simeprevir. FIG. 29A: Phase contrast images of HEK293 cells stably transduced with the wt kill switch showing rapid cell death upon treatment with simeprevir. FIG. 29B: Phase contrast images of human tumour cell lines HCT116 and HT29 stably transduced with the wt kill switch showing rapid cell death upon treatment with simeprevir. FIG. 29C: Schematic outlining Caspase 3 assay. FIG. 29D: Caspase 3 activity in wt kill switch-transduced HEK293+/−10 nM Simeprevir relative to treated untransduced HEK293 cells. FIG. 29E: Caspase 3 activity in three single cell clones for kill switch transduced HCT116 and HT29 relative to non-transduced HCT116 and HT29 in the presence of 10 nM simeprevir. **** p<0.0001; ns=not significant.

FIG. 30 shows the confluency over time of a non-transduced ES cell line Sa121, and the same cell line transduced with the simeprevir-inducible wt kill switch, upon addition of increasing concentrations of simeprevir.

FIGS. 31A-C B2M locus-targeted knock-in of the kill switch in induced pluripotency stem cells (iPSCs) facilitates simeprevir-induced cell killing. FIG. 31A: Schematic of the knock-in strategy of the kill switch. The Kill switch (iCasp9) was knocked in to the B2M gene locus of iPSCs. Adeno-associated viral (AAV) vector was used to deliver the donor template containing the iCasp9 expression cassette flanked by the B2M homologous arms. The light symbol indicated the CRISPR targeting site. LHA, left homologous arm; RHA, right homologous arm; EF1a promt, EF-1alpha promoter; P2A, porcine teschovirus-1 derived 2A self-cleaving peptides; Puro, puromycin-resistant gene; blast, blasticidin-resistant gene; bGH pA; bovine growth hormone polyadenylation signal; PrimerF, forward primer for genotyping; PrimerR, reverse primer for genotyping. FIG. 31B: Genotyping of single-cell clones of the kill switch-containing iPSCs. Five single-cell iPSC clones (1B7, 1D6, 1D12, 1G8 and 2D8) were isolated after gene knock-in. Genome DNA from these clones were extracted. Primers indicated in A) were used to amplify the targeted gene locus. Amplicons were loaded in 1.2% agarose gel for electrophoresis. Genotyping data indicated that single cell clones 1B7, 1D12, 1G8 and 2D8 have bi-allelic B2M-targeted kill switch knockin, while clone 1D6 has a mono-allelic kill switch knockin. iPSC-WT, wild type (unmodified) iPSCs; KI, amplicons of knock-in allele; WT, amplicons of wild type allele. FIG. 31C: Cell proliferation index quantified by xCELLigence Real-Time Cell Analysis (RTCA) assay. iPSC single-cell clones were cultured for 1 day prior to the simeprevir induction. Cell index were monitored for 3 days before and after the induction.

FIGS. 32A-B show functionality of kill switch S196A mutant upon addition of simeprevir. FIG. 32A: Phase contrast images of HEK293 cells stably transduced with the kill switch S196A mutant showing rapid cell death upon treatment with simeprevir. FIG. 32B: Caspase 3 activity in wt and S196A mutant kill switch-transduced HEK293+1-10 nM Simeprevir relative to treated untransduced HEK293 cells. *** p<0.0005; ns=not significant.

### DETAILED DESCRIPTION

Aspects and embodiments of the present disclosure will now be discussed with reference to the accompanying figures. Further aspects and embodiments will be apparent to those skilled in the art. All documents mentioned in this text are incorporated herein by reference.

### Expression Vectors and Expression Cassettes

An “expression vector” as used herein is a DNA molecule used for expression of foreign genetic material in a cell. Any suitable vectors known in the art may be used. Suitable vectors include DNA plasmids, binary vectors, viral vectors and artificial chromosomes (e.g. yeast artificial chromosomes). In certain embodiments, the expression vector is a viral vector as described in more detail below. In certain embodiments, the expression vector is a DNA plasmid.

An “expression cassette” as used herein is a polynucleotide sequence that is capable of effecting transcription of an expression product, which may be a protein. A “coding sequence” is intended to mean a portion of a gene's polynucleotide sequence that encodes the expression product. Where the expression product is a protein, this sequence may be referred to as a “protein coding sequence”. The protein coding sequence typically begins at the 5′ end by a start codon and ends at the 3′ end with a stop codon. The expression cassette may be part of an expression vector, or part of a viral genome in a viral particle, as described in more detail below.

Typically, the expression cassette comprises a promoter operably linked to a protein coding sequence. The term “operably linked” includes the situation where a selected coding sequence and promoter are covalently linked in such a way as to place the expression of the protein coding sequence under the influence or control of the promoter. Thus, a promoter is operably linked to the protein coding sequence if the promoter is capable of effecting transcription of the protein coding sequence. Where appropriate, the resulting transcript may then be translated into a desired protein.

Any suitable promoter known in the art may be used in the expression cassette providing it functions in the cell type being used. For example, where the cell is a mammalian cell, the promoter may be a cytomegalovirus (CMV) promoter. Where multiple expression cassettes are used, each coding sequence may be independently operably linked to its own promoter. Alternatively, the coding sequence for one or more of the expression cassettes may be operably linked to the same promoter.

Where multiple expression cassettes are described, e.g. a first and second expression cassette, they may be part of the same or different expression vectors. Thus, in some embodiments, the first and second expression cassettes may be located on the same expression vector. In other embodiments, the first expression cassette is located on a first expression vector and the second expression cassette is located on a second expression vector.

Where multiple expression cassettes are located on the same expression vector, the individual expression cassettes (e.g. first and second expression cassettes) may be separated by an Internal Ribosome Entry Site (IRES) or 2A element. The use of IRES or 2A elements allows multiple expression products to be expressed using the same promoter. In other words, when first and second expression cassettes are separated by an IRES or 2A element, both the first and second expression cassettes can be operably linked to the same promoter.

### Target Proteins and Small Molecules

Aspects and embodiments of the present disclosure are directed to target proteins that are derived from a non-human protein, i.e. a protein that is not endogenous to a human. In one embodiment, the non-human protein is derived from a viral, bacterial, fungal or protozoal protein. In one embodiment, the non-human protein is derived from a viral protein and the small molecule is an inhibitor of the viral protein. In one embodiment, the non-human protein is derived from a bacterial protein and the small molecule is an inhibitor of the bacterial protein. In one embodiment, the non-human protein is derived from a fungal protein and the small molecule is an inhibitor of the fungal protein. In one embodiment, the non-human protein is derived from a protozoal protein and the small molecule is an inhibitor of the protozoal protein. In one embodiment, the non-human protein is derived from a viral protease and the small molecule is an inhibitor of the viral protease.

The term “derived from” in the context of target proteins is intended to mean that the target protein has a similar, but not necessarily identical, amino acid sequence to the protein from which it is derived and the target protein is still capable of binding to the small molecule. A target protein that is derived from a protein may have an amino acid sequence that is at least 90%, 91%, 92%, 93%, 94%, 95%, 96%, 97%, 98%, or 99% identical to the protein from which it is derived. A target protein that is derived from a protein may contain less than 50, less than 40, less than 30, less than 20, less than 10, less than 9, less than 8, less than 7, less than 6, less than 5, less than 4, less than 3, or less than 2 sequence alterations compared to the protein from which it is derived. For example, a target protein having the amino acid sequence set forth in SEQ ID NO: 2 is derived from the viral protease having the sequence set forth in SEQ ID NO: 1. Additionally, the target protein may have fewer amino acids (i.e. it is a shorter protein) than the protein from which it is derived.

Viral proteases are enzymes encoded by the genetic material of viral pathogens. The normal function of these enzymes is to catalyse the cleavage of specific peptide bonds in viral polyprotein precursors or in cellular proteins. Examples of viral proteases include those encoded by hepatitis C virus (HCV), human immunodeficiency virus (HIV), herpesvirus, retrovirus and human rhinovirus (HRV) families. Certain viral proteases, along with examples of small molecule inhibitors of these proteases, are described for example in Patick and Potts. 1998.

A small molecule is an organic compound that typically has a molecular weight of 2000 daltons or less. The small molecule may be synthetic or naturally occurring.

The choice of viral protease inhibitor as small molecule is not particularly limited provided it a) is able to bind the target protein and b) has been evaluated for clinical purposes in humans. Viral protease inhibitors that have been evaluated for clinical purposes in humans include those that have been approved by a regulatory agency for clinical use in humans, for example, inhibitors approved for treatment by the Food and Drug Administration (FDA) and/or by the European Medicines Agency (EMA). Viral protease inhibitors that have been evaluated for clinical purposes also include those that are being/have been tested in clinical trials involving humans and have preferably have proceeded past phase I clinical trials. Preferably the viral protease inhibitor is approved for clinical use in humans. Preferably the viral protease inhibitor is suitable for chronic dosing (daily for six months or greater), cell permeable, orally dosed and/or not used as a first line therapy.

The viral protease used may be monomeric or multimeric (e.g. dimeric, trimeric, tetrameric, etc.). The use of a monomeric viral protease may be preferred, for example where a strict 1:1 ratio of the target protein fusion protein and binding member fusion protein elicit the desired functional activity. There may be alternative situations where a multimeric viral protease is preferred, for example when the target protein is fused to a transcriptional regulatory domain in a split transcription factor and the use of a multimeric viral protease could increase the number of transcriptional regulatory domains that are recruited to a target gene.

In some embodiments the viral protease is an HCV NS3/4A protease or a HIV protease. Both these proteases are known to be targeted by several approved small molecule inhibitors that are known to be generally well tolerated in humans and suitable for chronic dosing. Examples of small molecule inhibitors that target HCV NS3/4A protease are described in De Clercq. 2014. Examples of small molecule inhibitors that target HIV protease are described in Lv et al. 2015.

In some embodiments the viral protease is an HCV NS3/4A protease. HCV NS3/4A PR is monomeric, relatively small in size (21 kDa), can be expressed cytoplasmically, and is not found associated with DNA, making it an ideal candidate as a viral protease for use in the disclosure. The HCV NS3/4A protease may have the amino acid sequence of amino acid positions 1030-1206 of the amino acid sequence set forth in UniProt accession number A8DG50-1 (version 2 of the sequence; sequence update 29 Apr. 2008). In some embodiments the HCV NS3/4A protease may have the amino acid sequence set forth in SEQ ID NO: 1. A target protein that is derived from a HCV NS3/4A protease may have an amino acid sequence that is at least 90%, 91%, 92%, 93%, 94%, 95%, 96%, 97%, 98%, or 99% identical to the amino acid sequence set forth in SEQ ID NO: 1.

There are several small molecule inhibitors that are known to bind the HCV NS3/4A protease and have been approved for human use. Some of these are set forth in the following table:

| | | | Small |
|---|---|---|---|
| | | Structure | molecule |
| | | accession | accession |
| | Small | number | number |
| Target protein | molecule | in PDB | in PDB |
| HCV NS3/4A protease | asunaprevir | 4WF8 | 2R9 |
| HCV NS3/4A protease | vaniprevir | 3SU6 | SU3 |
| HCV NS3/4A protease | boceprevir | 3LOX | MCX |
| HCV NS3/4A protease | narlaprevir | 3LON | NNA |
| HCV NS3/4A protease | simeprevir | 3KEE | 30B |
| HCV NS3/4A protease | telaprevir | 3SV6 | SV6 |
| HCV NS3/4A protease | grazoprevir | 6CVY | FHD |
| HCV NS3/4A protease | danoprevir | 6C2N | TSV |

The structures of the target proteins in complex with the respective small molecule are provided as PDB accession numbers, which correspond to the crystal structures available from the Protein Data Bank (PDB). The small molecule structures and chemical names are also provided as PDB accession numbers.

The small molecule may be a peptide mimetic. The terms “peptide mimetic”, “peptidomimetic” and “peptide analogue” are used interchangeably and refer to a chemical compound that is not composed of amino acids but has substantially the same characteristics as a peptidic compound that is entirely composed of amino acids.

Other small molecule inhibitors that are being/have been tested in clinical trials involving humans include faldaprevir, sovaprevir, vedroprevir.

In some embodiments, the small molecule is selected from the group consisting of simeprevir, boceprevir, telaprevir, asunaprevir, vaniprevir, voxilaprevir, glecaprevir, paritaprevir, narlaprevir, danoprevir, faldaprevir, grazoprevir, sovaprevir, vedroprevir, or a pharmacologically acceptable analog or derivative thereof. All these small molecules have been approved for human use and/or have been tested in clinical trials involving humans. In some embodiments, the small molecule is selected from the group consisting of simeprevir, boceprevir, telaprevir, asunaprevir, vaniprevir, voxilaprevir, glecaprevir, paritaprevir, grazoprevir, danoprevir and narlaprevir, or a pharmacologically acceptable analog or derivative thereof. These small molecules have been approved for human use.

In particular embodiments, the small molecule is selected from the group consisting of simeprevir, boceprevir and telaprevir, or a pharmacologically acceptable analog or derivative thereof. These small molecules (simeprevir, boceprevir and telaprevir) are well tolerated in humans and have been approved for chronic human use. In particular embodiments, the small molecule may be simeprevir or a pharmacologically acceptable analog or derivative thereof. Simeprevir (Olysio®) is a small molecule that is administered orally, is cell-permeable, and has a pharmacokinetics (PK) profile that supports once-daily dosing. It has been used chronically (up to 39 months) to treat HCV infection in combination with ribavirin and pegylated interferon, and is on the WHO essential medicines list, indicative of a well-tolerated and widely administered drug.

Pharmacologically acceptable analogs and derivatives of the small molecules include compounds that differ from the “parent” small molecule but contain a similar antiviral activity as the parent small molecule and include tautomers, regioisomers, geometric isomers, and where applicable, stereoisomers, including optical isomers (enantiomers) and other steroisomers (diastereomers) thereof, as well as pharmaceutically acceptable salts and derivatives (including prodrug forms) thereof where applicable, in context. For example, analogs of simeprevir include those compounds encompassed by formula (I) defined in WO 2007014926 A1.

Simeprevir may have the following chemical structure:

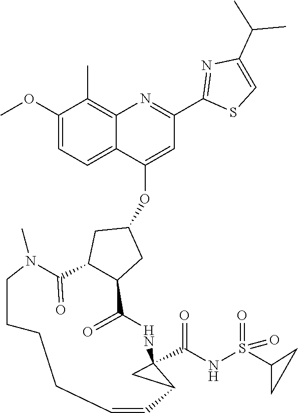 [SMILES: COc1ccc2c(O[C@@H]3C[C@H]4C(=O)N[C@]5(C(=O)NS(=O)(=O)C6CC6)C[C@H]5/C=C\CCCCN(C)C(=O)[C@@H]4C3)cc(-c3nc(C(C)C)cs3)nc2c1C]

In some embodiments the viral protease is a HIV protease. HIV protease exists as a 22 kDa homodimer, with each subunit made up of 99 amino acids. The HIV protease may have the amino acid sequence of amino acid positions 501-599 of the amino acid sequence set forth in UniProt accession number P03366-1 (version 3 of the sequence; sequence update 23 Jan. 2007). A target protein that is derived from a HIV protease may have an amino acid sequence that is at least 90%, 91%, 92%, 93%, 94%, 95%, 96%, 97%, 98%, or 99% identical to the amino acid sequence of amino acid positions 501-599 of the amino acid sequence set forth in UniProt accession number P03366-1. A target protein that is derived from a HIV protease may be a monomeric protein. For example, the target protein may contain one or more amino acid mutations that reduce the likelihood of the formation of a homodimeric protein.

There are several small molecule inhibitors that are known to bind the HIV protease and have been approved for human use. Some of these are set forth in the following table:

| | | | Small molecule |
|---|---|---|---|
| | Small | Structure accession | accession |
| Target protein | molecule | number in PDB | number in PDB |
| HIV protease | amprenavir | 3NU3 | 478 |
| HIV protease | atazanavir | 3EKY | DR7 |
| HIV protease | darunavir | 2HS1 | 017 |
| HIV protease | fosamprenavir | Not available | Not available |
| HIV protease | indinavir | 2AVO/3WSJ | MK1 |
| HIV protease | lopinavir/ritonavir | 2Q5K | AB1 |
| HIV protease | nelfinavir | 1OHR | 1UN |
| HIV protease | ritonavir | 4EYR | RIT |
| HIV protease | saquinavir | 2NMZ | ROC |
| HIV protease | tipranavir | 3SPK | TPV |

Fosamprenavir is a prodrug form of amprenavir that has better solubility and bioavailability than amprenavir.

In some embodiments, the small molecule is selected from the group consisting of atazanavir, darunavir and fosamprenavir, amprenavir, indinavir, lopinavir/ritonavir, nelfinavir, ritonavir, saquinavir and tipranavir, or a pharmacologically acceptable analog or derivative thereof.

In particular embodiments, the small molecule is selected from the group consisting of atazanavir, darunavir and fosamprenavir, or a pharmacologically acceptable analog or derivative thereof. These small molecules are well tolerated in humans and have good bioavailability. Furthermore, HIV protease inhibitors are typically used in patients for long periods of time and it is expected that these small molecule inhibitors would be tolerated for use over a long period of time.

In some embodiments, the target protein has attenuated viral activity compared to the viral protease from which it is derived. Attenuated viral activity in this context is intended to mean that the target protein has a lower enzymatic activity, e.g. lower protease activity, compared to the viral protease from which it is derived. Enzymatic activity can be tested, for example, using a fluorogenic peptide cleavage assay as described in the examples or described in Sabariegos et al. 2009. Briefly, the fluorgenic peptide cleavage assay involves using incubating the target protein/viral protease with a fluorogenic protease FRET substrate containing a donor-quencher pair such that cleavage of the peptide separates the donor from the quencher, emitting energy that can be detected at a certain wavelength, e.g. 490 nm.

In some embodiments, the target protein is considered to have attenuated viral activity compared to the viral protease from which it is derived if the target protein has an activity that is less than 10% of the activity of the viral protease as measured in an enzymatic activity assay, such as a fluorogenic peptide cleavage assay. In some embodiments, the target protein does not display any detectable viral activity when measured in an enzymatic activity assay, such as a fluorogenic peptide cleavage assay, when the target protein is at a concentration less than 1 nM, less than 10 nM, less than 100 nM, or less than 1 μM.

The target protein may comprise one or more amino acid mutations (e.g. substitutions/insertions/deletions) compared to the viral protease from which it is derived (e.g. compared to SEQ ID NO: 1). The target protein comprising the one or more amino acid mutations should retain its ability to form a tripartite complex with the small molecule and binding member, which can be determined, e.g. using a homogeneous time-resolved fluorescence (HTRF) assay as described in the examples.

In some embodiments, the target protein comprises one or more amino acid mutations compared to the viral protease from which it is derived, wherein the one or more amino acid mutations attenuate the viral activity of the target protein. The one or more amino acid mutations may be in the active site of the viral protease.

For example, the HCV NS3/4A protease contains a catalytic triad involving the amino acid residues H57, D81 and S139 of the HCV NS3/4A protease. See, e.g. Grakoui et al. 1993; Eckart et al. 1993; and Bartenschlager et al. 1993. These amino acid residues correspond to positions H72, D96 and S154 of the amino acid sequence of SEQ ID NO: 1. Thus, the target protein may contain an amino acid mutation at one or more amino acids selected from positions 72, 96 and 154 of the HCV NS3/4A protease, wherein amino acid numbering corresponds to SEQ ID NO: 1. Other residues of the HCV NS3/4A protease that are known to be involved in viral activity include C97, C99, C145 and H149 of the HCV NS3/4A protease (corresponding to positions C112, C114, C160 and H164 of SEQ ID NO: 1). See, e.g. Hikikata et al. 1993; and Stempniak et al. 1997. In some embodiments, the target protein contains an amino acid mutation (e.g. substitution) at one or more amino acids selected from positions 72, 96, 112, 114, 154, 160 and 164 of the HCV NS3/4A protease, wherein amino acid numbering corresponds to SEQ ID NO: 1.

In particular embodiments, the target protein comprises an amino acid mutation at position 154 of the HCV NS3/4A protease, wherein amino acid numbering corresponds to SEQ ID NO: 1, such as a mutation to alanine. In certain embodiments, the target protein has an amino acid sequence of SEQ ID NO: 2.

The full-length sequence of the NS3 protein is provided in SEQ ID NO: 199. The amino acid mutation described here at position 154 of SEQ ID NO: 1 corresponds to the position 139 of SEQ ID NO: 199.

A table identifying the potential amino acid mutations described above numbered according to the full length NS3 protein (SEQ ID NO: 199) and their corresponding positions in the NS3/4A protease amino acid sequence set forth in SEQ ID NO: 1 is set out as follows:

| Location of potential mutation | Corresponding position |
|---|---|
| in full length NS3 protein | wherein number is according |
| provided as SEQ ID NO: 199 | to SEQ ID NO: 1 |
| H57 | H72 |
| D81 | D96 |
| S139 | S154 |
| C97 | C112 |
| C99 | C114 |
| C145 | C160 |
| H149 | H164 |

As a further example, the HIV protease contains a catalytic triad involving the amino acid residues D25, T26 and G27, wherein amino acid numbering is according to the HIV protease having the amino acid sequence of amino acid positions 501-599 of the amino acid sequence set forth in UniProt accession number P03366-1 (version 3 of the sequence; sequence update 23 Jan. 2007). Thus, the target protein may contain an amino acid mutation at one or more amino acids selected from positions 25, 26 and 27 of the HIV protease, wherein amino acid numbering is according to the HIV protease having the amino acid sequence of amino acid positions 501-599 of the amino acid sequence set forth in UniProt accession number P03366-1 (version 3 of the sequence; sequence update 23 Jan. 2007).

The target protein and small molecule interact to form a complex between the target protein and small molecule referred to herein as a T-SM complex. The interaction may be a covalent interaction or a non-covalent interaction. In some embodiments the small molecule binds to the target protein with a kD that is lower than 1 mM, preferably lower than 500 nM, more preferably lower than 200 nM, even more preferably lower than 100 nM, or yet more preferably lower than 50 nM, when measured for example using surface plasmon resonance or bio-layer interferometry. In some embodiments, the small molecule binds to the target protein with a kD between 25 nM and 200 nM, between 25 nM and 100 nM, or between 25 and 75 nM, when measured for example using surface plasmon resonance or bio-layer interferometry.

It may be desirable to introduce amino acid mutations (e.g. substitutions) in the target protein in order to reduce the affinity of the small molecule for the target protein and allow a second small molecule to displace the small molecule in the T-SM complex. For example, as demonstrated herein, simeprevir binds the target protein HCV NS3/4A protease (S139A) (SEQ ID NO: 2) with a very high affinity such that other small molecules that bind the target protein are unable to displace simeprevir from the T-SM complex. Reducing the binding affinity of simeprevir to HCV NS3/4A protease by introducing amino acid modification(s) in the target protein allows for the use of different small molecules inhibitors of the HCV NS3/4A protease to disrupt the tripartite complex formed between HCV NS3/4A protease (S139A), simeprevir and PRSIM_23. Thus, in some embodiments the target protein comprises one or more affinity reducing amino acid mutations (e.g. substitutions) compared the viral protease from which it is derived (e.g. SEQ ID NO: 1), such that the small molecule binds the target molecule with a lower affinity than the small molecule binds a parent target protein. The ‘parent target protein’ in this context lacks the one or more affinity reducing amino acid mutations but is otherwise identical to the target protein. The parent target protein may be the viral protease from which the target protein is derived from (e.g. the parent target protein may have the amino acid sequence set forth in SEQ ID NO: 1), or the parent target protein may itself be derived from a viral protease (e.g. the parent target protein may have the amino acid sequence set forth in SEQ ID NO: 2).

The one or more affinity reducing amino acid mutations may result in the small molecule binding the target protein with at least a 1.5-fold lower affinity than the small molecule binds the parent target protein. The one or more affinity reducing amino acid mutations may result in the small molecule binding the target protein with an affinity that is between 1.5-fold and 10-fold lower than the small molecule binds the parent target protein, or between 1.5-fold and 5-fold lower than the small molecule binds the parent target protein. The one or more affinity reducing amino acid mutations may result in the small molecule binding the target protein with a KD between 25 nM and 200 nM, between 25 and 100 nM, or between 25 and 75 nM, optionally where affinity is measured using bio-layer interferometry, such as using an Octet RED384.

As demonstrated herein, amino acid substitutions at positions 151 and 183 of a HCV NS3/4A protease, wherein numbering amino acid numbering corresponds to SEQ ID NO: 1, were found to reduce the affinity of simeprevir to the HCV NS3/4A protease and allow a second small molecule that disrupt the tripartite complex formed between the HCV NS3/4A protease, simeprevir and the binding member PRSIM_23. Further, target proteins comprising these affinity reducing mutations were also demonstrated to retain functionality in dimerization-inducible proteins such as in split transcription factors. Amino acid positions 151 and 183 of SEQ ID NO: 1 correspond to amino acid positions 136 and 168, respectively, of the full length NS3 protein set forth in SEQ ID NO: 99.

Thus, in some embodiments where the target protein is derived from a viral protease that is an HCV NS3/4A protease, the target protein may have an affinity reducing amino acid mutation (e.g. substitution) at one or more amino acids selected from positions 151 and 183, wherein the amino acid numbering corresponds to SEQ ID NO: 1. In some embodiments, the affinity reducing amino acid mutation at position 151 is a mutation to aspartic acid, asparagine or histidine, and the affinity reducing mutation at position 183 is to glutamic acid, glutamine or alanine. In some embodiments, the affinity reducing amino acid mutation at position 151 is a mutation to aspartic acid or asparagine and the affinity reducing mutation at position 183 is to glutamic acid. The target protein may comprise the affinity reducing amino acid mutation in addition to another amino acid mutation described herein (e.g. in addition to the amino acid mutation at position 154, such as to an alanine).

In certain embodiments, the target protein has an amino acid sequence having at least 90%, 91%, 92%, 93%, 94%, 95%, 96%, 97%, 98%, 99% identity to SEQ ID NO: 1 and comprises alanine at position 154 and aspartic acid, asparagine or histidine (e.g. aspartic acid or asparagine) at position 151, wherein the amino acid numbering corresponds to SEQ ID NO: 1. In certain embodiments, the target protein is derived from a viral protease having the amino acid sequence set forth in SEQ ID NO: 1, wherein the target protein differs from the viral protease in that it comprises alanine at position 154 and aspartic acid, asparagine or histidine (e.g. aspartic acid or asparagine) at position 151, and optionally 1, 2, 3, 4, 5, 6, 7, 8, 9, 10, 11, 12, 13, 14, 15, 16, 17, 18, 19 or 20 additional sequence alterations (e.g. functionally conservative substitutions), wherein the amino acid numbering corresponds to SEQ ID NO: 1. In certain embodiments, the target protein comprises an amino acid sequence having at least 90%, 91%, 92%, 93%, 94%, 95%, 96%, 97%, 98%, 99% or 100% sequence identity to any one of the sequences set forth in SEQ ID NOs: 211 and 215.

In certain embodiments, the target protein has an amino acid sequence having at least 90%, 91%, 92%, 93%, 94%, 95%, 96%, 97%, 98%, 99% identity to SEQ ID NO: 1 and comprises alanine at position 154 and glutamic acid, glutamine or alanine (e.g. glutamic acid) at position 183, wherein the amino acid numbering corresponds to SEQ ID NO: 1. In certain embodiments, the target protein is derived from a viral protease having the amino acid sequence set forth in SEQ ID NO: 1, wherein the target protein differs from the viral protease in that it comprises alanine at position 154 and aspartic acid, asparagine or histidine (e.g. aspartic acid) at position 151, and optionally 1, 2, 3, 4, 5, 6, 7, 8, 9, 10, 11, 12, 13, 14, 15, 16, 17, 18, 19 or 20 additional sequence alterations (e.g. functionally conservative substitutions), wherein the amino acid numbering corresponds to SEQ ID NO: 1. In certain embodiments, the target protein comprises an amino acid sequence having at least 90%, 91%, 92%, 93%, 94%, 95%, 96%, 97%, 98%, 99% or 100% sequence identity to the sequences set forth in SEQ ID NOs: 213.

### Binding Members

As used herein “binding member” refers to a polypeptide or protein that specifically binds to the T-SM complex. The term “specific” may refer to the situation in which the binding member will not show any significant binding to molecules other than the T-SM complex. Such molecules are referred to as “non-target molecules” and include the target protein alone and the small molecule alone, i.e. the target protein or small molecule when not part of the T-SM complex.

In some embodiments, the binding member is considered to not show any significant binding to a non-target molecule if the extent of binding to a non-target molecule is less than about 10% of the binding of the binding member to the T-SM as measured, e.g., by isothermal calorimetry, ELISA, surface plasmon resonance (SPR), Bio-Layer Interferometry (BLI), homogeneous time-resolved fluorescence (HTRF), MicroScale Thermophoresis (MST), or by a radioimmunoassay (RIA). In some embodiments, the extent of binding to a non-target molecule is less than about 5% or less than about 1% of the binding of the binding member to the T-SM. Methods used to determine the extent of binding involving SPR (Biacore) and HTRF are described in the Examples. In some embodiments, where the extent of binding is measured by HTFR, the binding member described herein binds to the T-SM complex with an affinity that is at least 2-fold greater than the affinity towards another, non-target molecule, e.g. the target protein alone or small molecule alone. In some embodiments, the binding member binds to its target molecule with an affinity that is one of at least 3-, 5-, 10-, 20-fold greater than the affinity towards another, non-target molecule. Alternatively, the binding specificity may be reflected in terms of binding affinity, where the binding member described herein binds to the T-SM complex with an affinity that is at least 10-fold greater than the affinity towards another, non-target molecule, e.g. the target protein alone or small molecule alone. Binding affinity may be measured by surface plasmon resonance, e.g. Biacore. In some embodiments, the binding member binds to its target molecule with an affinity that is one of at least 50-, 100-, 1000-, 10000-fold greater than the affinity towards another, non-target molecule.

Binding affinity is typically measured by Kd (the equilibrium dissociation constant between the binding member and its target). As is well understood, the lower the Kd value, the higher the binding affinity of the binding member. For example, a binding member that binds to the T-SM complex with a Kd of 1 nM would be considered to be binding the T-SM complex with an affinity that is greater than a binding member that binds to a non-target molecule with a Kd of 100 nM.

The binding member may bind to the T-SM complex with an affinity having a Kd equal to or lower than 50 nM, 25 nM, 20 nM, 15 nM or 10 nM. The binding member may bind to the target protein alone or small molecule alone with an affinity having a Kd equal to or higher than 500 nM, 1 μM, 10 μM, 100 μM, or 1 mM. Binding affinity may be measured by SPR, e.g. by Biacore. The binding member may show minimal or no binding to the target protein alone and/or to the small molecule alone when measured by SPR.

In some embodiments, the binding member specifically binds the T-SM complex at an epitope that is only present on the T-SM complex and not on the target protein alone or small molecule alone. For example, the binding member may bind to a site of the T-SM complex comprising at least a portion of the small molecule and a portion of the target protein. Alternatively, the formation of a T-SM complex may induce a conformational change in the target protein that results in the formation of a new epitope that is specifically bound by the binding member. Methods of determining whether the binding member binds to a specific epitope include X-ray crystallography, peptide scanning, site-directed mutagenesis mapping and mass spectrometry.

In embodiments where the T-SM complex comprises a target protein derived from a HCV NS3/4A protease (e.g. SEQ ID NO: 2) and the small molecule simeprevir, the binding member may specifically bind the T-SM by forming interactions with at least one of the following residues of the target protein: Tyr71, Gly75, Thr76, Va193, Asp94, where the amino acid numbering corresponds to SEQ ID NO: 1. The binding member may form interactions with 1, 2, 3, 4, or most preferably all 5 of these residues. The binding member may additionally specifically bind the T-SM complex by forming interactions with the quinoline moiety of simeprevir. At least some of these interactions may by hydrophobic interactions and/or water-mediated interactions. Interactions can be determined using X-ray crystallography, for example as described in the examples.

The binding member may be an antibody molecule, such as a single chain variable fragment, or an antibody mimetic, such as a Tn3 protein.

Antibody Molecules

Aspects and embodiments of the present disclosure are directed to binding members that are antibody molecules, such as single chain variable fragments (scFv).

The term “antibody molecule” describes an immunoglobulin whether natural or partly or wholly synthetically produced. The antibody molecule may be human or humanised. The antibody molecule may be a monoclonal antibody molecule. Examples of antibodies are the immunoglobulin isotypes, such as immunoglobulin G (IgG), and their isotypic subclasses, such as IgG1, IgG2, IgG3 and IgG4, as well as fragments thereof.

An antibody molecule generally comprises six complementarity-determining regions (CDRs); three in the variable heavy (VH) region: HCDR1, HCDR2 and HCDR3, and three in the variable light (VL) region: LCDR1, LCDR2, and LCDR3. The six CDRs together define the paratope of the antibody molecule, which is the part of the antibody molecule which binds to the T-SM complex. The VH region and VL region comprise framework regions (FRs) either side of each CDR, which provide a scaffold for the CDRs. From N-terminus to C-terminus, VH regions comprise the following structure: N term-[HFR1]-[HCDR1]-[HFR2]-[HCDR2]-[HFR3]-[HCDR3]-[HFR4]-C term; and VL regions comprise the following structure: N term-[LFR1]-[LCDR1]-[LFR2]-[LCDR2]-[LFR3]-[LCDR3]-[LFR4]-C term.

There are several different conventions for defining antibody CDRs and FRs, such as those described in Kabat et al., Sequences of Proteins of Immunological Interest, 5th Ed. Public Health Service, National Institutes of Health, Bethesda, Md. (1991), Chothia et al., J. Mol. Biol. 196:901-917 (1987), IMGT numbering as described in LeFranc et al., Nucleic Acids Res. (2015) 43 (Database issue):D413-22, and VBASE2, as described in Retter et al., Nucl. Acids Res. (2005) 33 (suppl 1): D671-D674. The CDRs and FRs of the VH regions and VL regions of the antibody molecules described herein were defined according to Kabat (Kabat, E. A et al (1991).

The term “antibody molecule”, as used herein, includes antibody fragments, provided they display binding to the relevant target molecule(s). Examples of antibody fragments include Fv, scFv, Fab, scFab, F(ab′)₂, Fab₂, diabodies, triabodies, scFv-Fc, minibodies and single domain antibodies (e.g. VhH), etc.). Unless the context requires otherwise, the term “antibody molecule”, as used herein, is thus equivalent to “antibody molecule or antigen-binding fragment thereof”. In particular exemplified embodiments, the antibody molecule is a single chain variable fragment (scFv).

Antibody molecules and methods for their construction and use are well-known in the art and are described in, for example, Holliger & Hudson, Nature Biotechnology 23(9):1126-1136 (2005). It is possible to take monoclonal and other antibody molecules and use techniques of recombinant DNA technology to produce other antibody or chimeric molecules which retain the specificity of the original antibody. Such techniques may involve introducing CDRs or variable regions of one antibody molecule into a different antibody molecule (EP-A-184187, GB 2188638A and EP-A-239400).

In view of today's techniques in relation to monoclonal antibody technology, antibody molecules can be prepared to most antigens. The antigen-binding domain may be a part of an antibody (for example a Fab fragment) or a synthetic antibody fragment (for example an scFv). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al (1988, 8th International Biotechnology Symposium Part 2, 792-799).

The sequence identifiers (SEQ ID NOs) for HCDR1, HCDR2, HCDR3, LCDR1, LCDR2, LCDR3, variable heavy (VH) chain, variable light (VL) chain and scFv amino acid sequences for PRSIM_57, PRSIM_01, PRSIM_04, PRSIM_67, PRSIM_72 and PRSIM_75 are as set forth in the following table:

| PRSIM | | | | | | | | | |
|---|---|---|---|---|---|---|---|---|---|
| clone | HCDR1 | HCDR2 | HCDR3 | LCDR1 | LCDR2 | LCDR3 | VH chain | VL chain | scFv |
| PRSIM_57 | 151 | 152 | 153 | 154 | 155 | 156 | 186 | 187 | 12 |
| PRSIM_01 | 151 | 152 | 198 | 154 | 155 | 156 | 188 | 189 | 10 |
| PRSIM_04 | 151 | 152 | 163 | 154 | 155 | 164 | 190 | 191 | 11 |
| PRSIM_67 | 165 | 166 | 167 | 168 | 169 | 170 | 192 | 193 | 13 |
| PRSIM_72 | 171 | 172 | 173 | 174 | 175 | 176 | 194 | 195 | 14 |
| PRSIM_75 | 177 | 178 | 179 | 180 | 181 | 182 | 196 | 197 | 15 |

In some embodiments, the antibody molecule comprises heavy chain complementarity determining regions (HCDRs) 1 to 3 and/or light chain complementarity determining regions (LCDRs) of:
i) PRSIM_57 set forth in SEQ ID NOs: 151, 152, 153, 154, 155, and 156, respectively;
ii) PRSIM_01 set forth in SEQ ID NOs 151, 152, 198, 154, 155, and 156, respectively;
iii) PRSIM_04 set forth in SEQ ID NOs: 151, 152, 163, 154, 155, and 164, respectively;
iv) PRSIM_67 set forth in SEQ ID NOs: 165, 166, 167, 168, 169, and 170, respectively;
v) PRSIM_72 set forth in SEQ ID NOs: 171, 172, 173, 174, 175, and 176, respectively; or
vi) PRSIM_75 set forth in SEQ ID NOs: 177, 178, 179, 180, 181, and 182, respectively,
   wherein the CDR sequences are defined according to the Kabat numbering scheme.

In some embodiments, the binding member comprises a number of sequence alterations, e.g. one, two, three, four, or five sequence alterations, in any one or more of the CDRs defined above.

In some embodiments, the antibody molecule comprises a variable heavy (VH) chain and/or variable light (VL) chain of:
i) PRSIM_57 set forth in SEQ ID NOs: 186 and 187, respectively;
ii) PRSIM_01 set forth in SEQ ID NOs 188 and 189, respectively;
iii) PRSIM_04 set forth in SEQ ID NOs: 190 and 191, respectively;
iv) PRSIM_67 set forth in SEQ ID NOs: 192 and 193, respectively;
v) PRSIM_72 set forth in SEQ ID NOs: 194 and 195, respectively; or
vi) PRSIM_75 set forth in SEQ ID NOs: 196 and 197, respectively.

In particular embodiments, the antibody molecule is a single-chain variable fragment (scFv). Typically, an scFV comprises a VH chain and a VL chain separated by a peptide linker. The peptide linker may be as defined herein. In some embodiments, the peptide linker separating the VH and VL chain may comprise the amino acid sequence of SEQ ID NO: 204.

In some embodiments, the scFv comprises an amino acid sequence having at least 90%, 91%, 92%, 93%, 94%, 95%, 96%, 97%, 98% or 99% identity with the amino acid sequence of:
i) PRSIM_57 set forth in SEQ ID NO: 12;
ii) PRSIM_01 set forth in SEQ ID NO: 10;
iii) PRSIM_04 set forth in SEQ ID NO: 11;
iv) PRSIM_67 set forth in SEQ ID NO: 13;
v) PRSIM_72 set forth in SEQ ID NO: 14; or
vi) PRSIM_75 set forth in SEQ ID NOs: 15.

In particular embodiments, the scFv comprises an amino acid sequence of:
i) PRSIM_57 set forth in SEQ ID NO: 12;
ii) PRSIM_01 set forth in SEQ ID NO: 10;
iii) PRSIM_04 set forth in SEQ ID NO: 11;
iv) PRSIM_67 set forth in SEQ ID NO: 13;
v) PRSIM_72 set forth in SEQ ID NO: 14; or
vi) PRSIM_75 set forth in SEQ ID NOs: 15.

Antibody Mimetics

The binding member may be an antibody mimetic. Antibody mimetics are organic compounds that are able to specifically bind antigens but are structurally different to antibody molecules. Examples of antibody mimetics include scaffold proteins such as Tn3 proteins, affibodies, affilins, affimers, affitins, alphabodies, anticalins, avimers, DARPins, flynomers, Kunitz domain peptides, monobodies and nanoCLAMPs.

In particular aspects and embodiments, the binding member is a Tn3 protein.

Tn3 proteins are based on the structure of a type III fibronectin module (FnIII) and are derived from the third FnIII domain of human tenascin C. The generation and use of Tn3 proteins is described for example in WO 2009/058379, WO 2011/130324, WO2011130328 and Gilbreth et al. 2014.

The Tn3 proteins and the native FnIII domain from tenascin C are characterized by the same tridimensional structure, namely a beta-sandwich structure with three beta strands (A, B, and E) on one side and four beta strands (C, D, F, and G) on the other side, connected by six loop regions. These loop regions are designated according to the beta-strands connected to the N- and C-terminus of each loop. Accordingly, the AB loop is located between beta strands A and B, the BC loop is located between strands B and C, the CD loop is located between beta strands C and D, the DE loop is located between beta strands D and E, the EF loop is located between beta strands E and F, and the FG loop is located between beta strands F and G. FnIII domains possess solvent exposed loops tolerant of randomization, which facilitates the generation of diverse pools of protein scaffolds capable of binding specific targets with high affinity.

A wild-type Tn3 protein may comprise the sequence SEQ ID NO: 134. In the wild-type Tn3 protein, the BC, DE and FG loops are located at positions 23 to 31, 51 to 56 and 75 to 80, wherein the amino acid numbering corresponds to SEQ ID NO: 134. The Tn3 protein may contain one, preferably two, more preferably three, even more preferably four of the stabilising mutations selected from the list consisting of 132F, D49K, E861 and T89K, wherein the amino acid numbering corresponds to SEQ ID NO: 134. The amino acid sequence of a wild-type Tn3 protein comprising all four stabilising mutations is set forth in SEQ ID NO: 135. The Tn3 protein may additionally contain one or more of the stabilising mutations described in Gilbreth et al. 2014 (see, in particular, Table 1 of Gilbreth et al. 2014).

Tn3 proteins can be subjected to directed evolution designed to randomize one or more of the loops which are analogous to the complementarity-determining regions (CDRs) of an antibody variable region. Such a directed evolution approach results in the production of antibody-like binding members with high affinities for targets of interest, e.g., the T-SM complexes described herein.

Thus, the Tn3 protein that specifically binds to the T-SM complex described herein may comprise the BC, DE and FG loops of PRSIM_23, PRSIM_32, PRSIM_33, PRSIM_36, or PRSIM_47. For example, the Tn3 protein may comprise the sequence of SEQ ID NO: 134 or SEQ ID NO: 135, where the BC, DE and FG loops located at positions 23 to 31, 51 to 56, and 75 to 80, respectively, are substituted for the BC, DE and FG loops of PRSIM_23, PRSIM_32, PRSIM_33, PRSIM_36, or PRSIM_47, wherein the amino acid numbering corresponds to SEQ ID NO: 134.

A person skilled in the art would be readily able to determine the amino acid sequences of the BC, DE and FG loops of the PRSIM clones described herein. For example, the amino acid sequences of the PRSIM clones could be compared to the amino acid sequences of the wild-type Tn3 protein, e.g. those amino acid sequences set forth in SEQ ID NO: 134 or 135.

The Tn3 sequence, amino acid positions and sequences of the BC, DE and FG loops of PRSIM_23, PRSIM_32, PRSIM_33, PRSIM_36, or PRSIM_47 are as set forth in the following table:

| | | BC loop | | DEloop | | FGloop | |
|---|---|---|---|---|---|---|---|
| | | location | | location | | location | |
| PRSIM | Tn3 | Tn3 | BC loop | in Tn3 | DEloop | in Tn3 | FGloop |
| clone | sequence | sequence | sequence | sequence | sequence | sequence | sequence |
| PRSIM_23 | SEQ ID | 23 to 32 | VDPRYDDIWW | 52 to 57 | YLNDPY | 76to 85 | YTGDSYSRSGSNPA |
| | NO: 5 | | (SEQ ID | | (SEQ ID | | (SEQ ID NO: 138) |
| | | | NO: 136) | | NO: 137) | | |
| PRSIM_32 | SEQ ID | 23 to 34 | WSPRYYYASI | 54 to 59 | DYASND | 78 to 87 | WNYGDWRYSSSNPA |
| | NO: 6 | | SG | | (SEQ ID | | (SEQ ID NO: 141) |
| | | | (SEQ ID | | NO: 140) | | |
| | | | NO: 139) | | | | |
| PRSIM_33 | SEQ ID | 23 to 34 | YPPGRWYDDI | 54 to 59 | ARGDDV | 78 to 87 | WGPDRGDRAGSNPA |
| | NO: 7 | | WY | | (SEQ ID | | (SEQ ID NO: 44) |
| | | | (SEQ ID | | NO: 143) | | |
| | | | NO: 142) | | | | |
| PRSIM_36 | SEQ ID | 23 to 34 | SWPRDDDYDI | 54 to 59 | LNYASP | 78 to 87 | VVPDTYGRGTSNPA |
| | NO: 8 | | WY | | (SEQ ID | | (SEQ ID NO: 147) |
| | | | (SEQ ID | | NO: 146) | | |
| | | | NO: 145) | | | | |
| PRSIM_47 | SEQ ID | 23 to 31 | SRPGVSIWY | 51 to 56 | DYRSYY | 75 to 84 | GSYGLVGVRASNPA |
| | NO: 9 | | (SEQ ID | | (SEQ ID | | (SEQ ID NO: 150) |
| | | | NO: 148) | | NO: 149) | | |

In some embodiments, the Tn3 protein comprises the BC, DE and FG loops of:
i) PRSIM_23, set forth in SEQ ID NOs: 136, 137, and 138, respectively;
ii) PRSIM_32, set forth in SEQ ID NOs: 139, 140, and 141, respectively;
iii) PRSIM_33, set forth in SEQ ID NOs: 142, 143, and 144, respectively;
iv) PRSIM_36, set forth in SEQ ID NOs: 145, 146, and 147, respectively; or
v) PRSIM_47, set forth in SEQ ID NOs: 148, 149, and 150, respectively,

In some embodiments, the Tn3 protein comprises the BC, DE and FG loops of:
i) PRSIM_23, wherein the BC loop comprises amino acids at positions 23 to 32 of SEQ ID NO: 5; the DE loop comprises amino acids at position 52 to 57 of SEQ ID NO: 5; and the FG loop comprises amino acids at positions 76 to 85 of SEQ ID NO: 5;
ii) PRSIM_32, wherein the BC loop comprises amino acids at positions 23 to 34 of SEQ ID NO: 6; the DE loop comprises amino acids at position 54 to 59 of SEQ ID NO: 6; and the FG loop comprises amino acids at positions 78 to 87 of SEQ ID NO: 6;
iii) PRSIM_33, wherein the BC loop comprises amino acids at positions 23 to 34 of SEQ ID NO: 7; the DE loop comprises amino acids at position 54 to 59 of SEQ ID NO: 7; and the FG loop comprises amino acids at positions 78 to 87 of SEQ ID NO: 7;
iv) PRSIM_36, wherein the BC loop comprises amino acids at positions 23 to 34 of SEQ ID NO: 8; the DE loop comprises amino acids at position 54 to 59 of SEQ ID NO: 8; and the FG loop comprises amino acids at positions 78 to 87 of SEQ ID NO: 8; or
v) PRSIM_47, wherein the BC loop comprises amino acids at positions 23 to 31 of SEQ ID NO: 9; the DE loop comprises amino acids at position 51 to 56 of SEQ ID NO: 9; and the FG loop comprises amino acids at positions 75 to 84 of SEQ ID NO: 9.

In some embodiments, the Tn3 protein comprises a number of sequence alterations, e.g. one, two, three, four, or five sequence alterations, in any one or more of the BC, DE and EF loops defined above. In some embodiments, the Tn3 protein comprises a number of sequence alterations, e.g. one, two, three, four, or five sequence alterations, outside the BC, DE and EF loops defined above.

In some embodiments, the Tn3 protein comprises an amino acid sequence having at least 90%, 91%, 92%, 93%, 94%, 95%, 96%, 97%, 98% or 99% identity with the amino acid sequence of:
i) PRSIM_23 set forth in SEQ ID NO: 5;
ii) PRSIM_32 set forth in SEQ ID NO: 6;
iii) PRSIM_33 set forth in SEQ ID NO: 7;
iv) PRSIM_36 set forth in SEQ ID NO: 8; or
v) PRSIM_47 set forth in SEQ ID NOs: 9.

In particular embodiments, the Tn3 protein comprises an amino acid sequence of:
i) PRSIM_23 set forth in SEQ ID NO: 5;
ii) PRSIM_32 set forth in SEQ ID NO: 6;
iii) PRSIM_33 set forth in SEQ ID NO: 7;
iv) PRSIM_36 set forth in SEQ ID NO: 8; or
v) PRSIM_47 set forth in SEQ ID NOs: 9.

### Dimerization-Inducible Proteins

In some embodiments the target protein is fused to a first component polypeptide and the binding member is fused to a second component polypeptide. In particular embodiments the first and second component polypeptides form part of a dimerization-inducible protein.

As used herein “dimerization-inducible protein” refers to a protein or complex comprising a first and second component polypeptide, wherein the first and second polypeptide form a functional protein upon dimerization. The term “dimerization-inducible proteins” includes “split proteins”, “dimerization-deficient proteins” and “split complexes”. The term “component polypeptide” is intended to encompass both single-chain and multi-chain polypeptides. The first and second component polypeptides in the dimerization-inducible protein typically do not have activity or have less activity when separated, but upon dimerization are brought into close proximity and as such become active or have increased activity. As described in the examples, the combination of particular binding members, target proteins and small molecules described herein are able to regulate dimerization of the dimerization-inducible protein such that a significant increase in activity is observed when the binding member is bound to the T-SM complex compared to the separate components of the dimerization-inducible protein alone.

Examples of dimerization-inducible proteins include split chimeric antigen receptor (split CAR; e.g. as described in Wu et al. 2015), split kinases (e.g. as described in Camacho-Soto et al. 2014), split transcription factors (e.g. as described in Taylor et al. 2010), split apoptotic proteins (e.g. split caspases as described in Chelur et al. 2007), split reporter systems (e.g. as described in Dixon et al. 2016).

The dimerization-inducible protein will have increased activity when the binding member is bound to the T-SM complex. Increased activity can be compared to the activity observed when the binding member is not bound to the T-SM complex (e.g. because one or more of the target protein, small molecule or binding member is not present). In some embodiments, the increased activity observed when the binding member is bound to the T-SM complex is at least a 1.5-fold, 2-fold, 3-fold, 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 105-fold, 110-fold, 115-fold, or 120-fold increase in activity as compared to activity observed when the binding member is not bound to the T-SM complex.

Methods of measuring the activity of the dimerization-inducible protein will depend upon the particular dimerization-inducible protein being studied. Where the first and second component polypeptide form a chimeric antigen receptor (CAR) upon dimerization, CAR activity can be determined by measuring the immune cell activation and/or proliferation. As described in the examples, CAR activity can be measured by interleukin-2 (IL-2) production, e.g. by ELISA, after stimulation of the CAR by an antigen. Where the first and second component polypeptide form a kinase upon dimerization, activity of the kinase can be measured by incorporation of phosphate, e.g. radioactive ³²P, into a peptide substrate as described in Camacho-Soto et al. 2014. Where the first and second component polypeptides form a transcription factor upon dimerization, transcriptional activity can be determined by measuring expression of a downstream desired expression cassette modulated by the split transcription factor as described in the examples. Where the first and second component polypeptide form a therapeutic protein upon dimerization, activity can be measured by using suitable assays for determining functional activity of the protein. Where the first and second component polypeptides form a caspase upon dimerization, caspase activity can be measured using a caspase activity assay or by measuring apoptotic cell death. Where the first and second component polypeptides form a reporter system upon dimerization, reporter activity can be determined by measuring expression of the reporter, e.g. a luciferase.

The first component polypeptide may be fused to the C-terminus or the N-terminus of the target protein or binding member. The second component polypeptide may be fused to the C-terminus or the N-terminus of the target protein or binding member. The component polypeptides may be fused to the target protein or binding member via a peptide linker. Suitable peptide linkers include those represented by [G]n, [S]n, [A]n, [GS]n, [GGS]n, [GGGS]n (SEQ ID NO.: 239), [GGGGS)n (SEQ ID NO.: 240), [GGSG]n (SEQ ID NO.: 241), [GSGG]n (SEQ ID NO.: 242), [SGGG]n (SEQ ID NO.: 243), [SSGG]n (SEQ ID NO.: 244), [SSSG]n (SEQ ID NO.: 245), [GG]n, [GGG]n, [SA]n, [TGGGGSGGGGS]n (SEQ ID NO.: 185), and combinations thereof, wherein n is an integer between 1 and 30. For example, n may be 1, 2, 3, 4, 5, 6, 7, 8, 9, 10, or any number up to 30. The component polypeptide may be fused to the target protein or binding member directly, e.g. in the format-first component polypeptide-peptide linker-target protein. Alternatively, the component polypeptide may be fused to the target protein or binding member indirectly with one or more additional polypeptides separating the first component polypeptide from the target protein or binding member, e.g. first component polypeptide-additional polypeptide-peptide linker-target protein.

In some embodiments, the first component polypeptide is fused to more than one target protein or binding member. In some embodiments, the second component polypeptide is fused to more than one target protein or binding member or a combination of both. For example, the first or second component polypeptide may be fused to 1, 2, 3, 4, 5, 6, 7, 8, 9, or 10 binding members. In some embodiments, the first or second component polypeptide is fused to between 2 and 10, or between 2 and 5 binding members. In particular embodiments, the first or second component polypeptide is fused to 3 binding members. For example, the first or second component polypeptide may be fused to 1, 2, 3, 4, 5, 6, 7, 8, 9, or 10 target proteins. In some embodiments, the first or second component polypeptide is fused to between 2 and 10, or between 2 and 5 target proteins. In particular embodiments, the first or second component polypeptide is fused to 3 target proteins. Where multiple binding members or target proteins are present, they may be fused to each other by peptide linkers, e.g. those peptide linkers described above.

Split Transcription Factor

The dimerization-inducible protein may be a split transcription factor. In some embodiments, the first component polypeptide comprises a DNA binding domain; and the second component polypeptide comprises a transcriptional regulatory domain, and wherein the first component polypeptide and second component polypeptide form a transcription factor upon dimerization. By “form a transcription factor” it is meant that the first and second component polypeptides are brought into close enough proximity that they are able to reconstitute the transcriptional regulatory activity of desired expression products. The dimerization-inducible protein will have increased transcriptional regulatory activity when the binding member is bound to the T-SM complex, wherein the transcriptional regulatory activity is increased compared to the transcriptional regulatory activity observed when the binding member is not bound to the T-SM complex.

The transcriptional regulatory domain may be a transcriptional activation domain that is capable of upregulating transcription of a gene that the split transcription factor binds to. Suitable transcriptional activation domains include the p65 subunit of nuclear factor kappa B (Bitko & Barik, J. Virol. 72:5610-5618 (1998) and Doyle & Hunt, Neuroreport 8:2937-2942 (1997)); Liu et al., Cancer Gene Ther. 5:3-28 (1998)); the replication and transcription activator (RTA; Lukac et al., J Virol. 73, 9348-61 (1999)), a the HSV VP16 activation domain (see, e.g., Hagmann et al., J. Virol. 71, 5952-5962 (1997)) nuclear hormone receptors (see, e.g., Torchia et al., Curr. Opin. Cell. Biol. 10:373-383 (1998)); or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, Oct 1, Oct-2A, Sp1, AP-2, and CTF1 (Seipel et al., EMBO J. 11, 4961-4968 (1992) as well as p300, CBP, PCAF, SRC1 PvALF, AtHD2A and ERF-2. See, for example, Robyr et al. (2000) Mol. Endocrinol. 14:329-347; Collingwood et al. (1999) J. Mol. Endocrinol. 23:255-275; Leo et al. (2000) Gene 245:1-11; Manteuffel-Cymborowska (1999) Acta Biochim. Pol. 46:77-89; McKenna et al. (1999) J. Steroid Biochem. Mol. Biol. 69:3-12; Malik et al. (2000) Trends Biochem. Sci. 25:277-283; and Lemon et al. (1999) Curr. Opin. Genet. Dev. 9:499-504. Additional exemplary activation domains include, but are not limited to, OsGAI, HALF-1, C1, AP1, ARF-5,-6,-7, and -8, CPRF1, CPRF4, MYC-RP/GP, and TRAB1 and a modified Cas9 transactivator protein. See, for example, Ogawa et al. (2000) Gene 245:21-29; Okanami et al. (1996) Genes Cells 1:87-99; Goff et al. (1991) Genes Dev. 5:298-309; Cho et al. (1999) Plant Mol. Biol. 40:419-429; Ulmason et al. (1999) Proc. Natl. Acad. Sci. USA 96:5844-5849; Sprenger-Haussels et al. (2000) Plant J. 22:1-8; Gong et al. (1999) Plant Mol. Biol. 41:33-44; Hobo et al. (1999) Proc. Natl. Acad. Sci. USA 96:15,348-15,353; and Perez-Pinera et al. (2013) Nature Methods 10:973-976). The transcriptional activation domain may comprise any combination of the above exemplary activation domains. In some embodiments multiple transcriptional activation domains may be used, e.g. tandem reports of the same domains or fusions of different domains. In some embodiments the transcriptional activation domain is VPR, a tripartite activate made up of the VP64, p65 and Rta domains. An example of a TRD-T fusion protein comprising VPR is set forth in SEQ ID NO: 225 (NS4A/3 PR S139A-VPR). Generation and use of VPR as a transcriptional activator is described for example in Chavez et al. 2015. In some embodiments the transcriptional activation domain is HSF-1, optionally in combination with p65.

Alternatively, the transcriptional regulatory domain may be a transcriptional repression domain that is capable of downregulating transcription of a gene that the split transcription factor binds to. Transcriptional repression domains include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B), Rb, and MeCP2. See, for example, Bird et al. (1999) Cell 99:451-454; Tyler et al. (1999) Cell 99:443-446; Knoepfler et al. (1999) Cell 99:447-450; and Robertson et al. (2000) Nature Genet. 25:338-342. Additional exemplary repression domains include, but are not limited to, ROM2 and AtHD2A. See, for example, Chem et al. (1996) Plant Cell 8:305-321; and Wu et al. (2000) Plant J. 22:19-27.

The DNA binding domain may be any protein that binds to a target sequence in a sequence specific manner. For example, the DNA binding domain may be or may contain a transcription factor that binds to a target sequence in a sequence specific manner, or a DNA-binding fragment thereof. It is expected that any transcription factor, or DNA-binding fragment thereof, that is capable of binding to a target sequence in a specific manner can be used with the split transcription factors disclosed herein. The DNA-binding domain may be or comprise a naturally occurring DNA-binding domain such as a binding domain from a human transcription factor. For example, the DNA-binding protein may be any of the human transcription factors described in Vaquerizas et al. (2009) (e.g. any of those listed in Supplementary information S3), or a DNA-binding fragment thereof. For example, the DNA-binding protein may be a member of the C2H2 zinc-finger family, the homeodomain family or the helix-loop-helix family or a DNA-binding fragment thereof. In particular embodiments the DNA binding domain may be zinc finger homeodomain transcription factor 1 (ZFHD1). ZFHD1 contains zinc fingers 1 and 2 from the Zif268 transcription factor and the Oct-1 homeodomain. The design and construction of ZFHD1 is described for example in Pomerantz et al. 1995.

The DNA binding domain may be or comprise a DNA-binding domain such as a zinc finger DNA binding domain, a TALE DNA binding domain, a DNA binding domain from a meganuclease (e.g. based on Iscel) or a DNA binding domain from a CRISPR/Cas system. These binding domains can be engineered to bind a target sequence of choice, e.g. a target sequence in a target gene that is naturally present (endogenous) in a cell or a target sequence that has been provided in trans (e.g. as part of a third expression cassette). The engineering of zinc finger DNA binding domains to bind particular target sequences is described for example in U.S. Pat. No. 6,453,242B1. In one embodiment, the DNA-binding domain is a TALE DNA binding domain. The engineering of TALE DNA binding domain domains to bind particular target sequences is described for example in WO2010079430A1. In one embodiment, the DNA binding domain is an engineered DNA binding domain from a meganuclease. The engineering of meganucleases to bind particular target sequence is described for example in WO2007047859A1. A meganuclease may be engineered such that they no longer cleave DNA. In one embodiment, the DNA binding domain is an engineered DNA binding domain from a CRISPR/Cas system. The engineering of DNA binding domains from CRISPR/Cas systems to bind particular sequences is described for example in WO2013176772A1. CRISPR/Cas systems generally involve an RNA-guided endonuclease (e.g. Cas9) that is directed to a specific DNA sequence through complementarity between the associated guide RNA (gRNA) and its target sequence. Thus, the engineered DNA binding domain from a CRISPR/Cas system typically comprises a complex of a RNA-guided endonuclease (e.g. Cas9 or a variant thereof) and a guide RNA. Variants of Cas9 have been generated that lack the endonucleolytic activity but retain the capacity to interact with DNA. See for example Chavez et al. 2015 which describes the use of nuclease-null (dCas9) variants in a method of transcriptional regulation. Thus, the DNA-binding domain may include a nuclease null Cas9 variant which, upon addition of a particular gRNA specific for a target sequence, binds to the target sequence. An example of a DBD-BM fusion protein comprising dCas9 as a DNA-binding domain is set forth in SEQ ID NO: 227 (spdCas9-PRSIM_23×3). An example of a guide RNA that targets the DBD-BM to human IL-2 is set forth in SEQ ID NO: SEQ ID NO: 229. The use of a dCas9 variant as part of a split transcription factor is described in Hill et al. 2018 and WO 2018/213848 A1.

The binding member may be fused to the transcriptional regulatory domain or to the DNA binding domain.

In some embodiments:
(1) the first component polypeptide comprises a DNA binding domain and is fused to a target protein to form a DBD-T fusion protein; and
   the second component polypeptide comprises a transcriptional regulatory domain and is fused to a binding member to form a TRD-BM fusion protein, or
(2) the first component polypeptide comprises a transcriptional regulatory domain and is fused to a target protein to form a TRD-T fusion protein; and
the second component polypeptide comprises a DNA binding domain and is fused to a binding member to form a DBD-BM fusion protein,
wherein the DNA binding domain, target protein, transcriptional regulatory domain and binding member are as further defined herein.

In certain embodiments:
(1) the first component polypeptide comprises a DNA binding domain and is fused to a target protein to form a DBD-T fusion protein, wherein the target protein comprises an amino acid sequence having at least 90% identity to the amino acid sequence set forth in SEQ ID NO: 1, and
   the second component polypeptide comprises a transcriptional regulatory domain and is fused to a binding member to form a TRD-BM fusion protein, or
(2) the first component polypeptide comprises a transcriptional regulatory domain and is fused to a target protein to form a TRD-T fusion protein, wherein the target protein has an amino acid sequence having at least 90% identity to SEQ ID NO: 1, and
   the second component polypeptide comprises a DNA binding domain and is fused to a binding member to form a DBD-BM fusion protein,
wherein in either (1) or (2):
a) the binding member comprises the BC, DE and FG loops, or Tn3 sequence, of PRSIM_23;
b) the binding member comprises the BC, DE and FG loops, or Tn3 sequence, of PRSIM_32;
c) the binding member comprises the BC, DE and FG loops, or Tn3 sequence, of PRSIM_33;
d) the binding member comprises the BC, DE and FG loops, or Tn3 sequence, of PRSIM_36;
e) the binding member comprises the BC, DE and FG loops, or Tn3 sequence, of PRSIM_47;
the binding member comprises the HCDRs and/or LCDRs, or VH and/or VL sequence, of PRSIM_57;
g) the binding member comprises the HCDRs and/or LCDRs, or VH and/or VL sequence, of PRSIM_01;
h) the binding member comprises the HCDRs and/or LCDRs, or VH and/or VL sequence, of PRSIM_04;
i) the binding member comprises the HCDRs and/or LCDRs, or VH and/or VL sequence, of PRSIM_67;
j) the binding member comprises the HCDRs and/or LCDRs, or VH and/or VL sequence, of PRSIM_72; or
k) the binding member comprises the HCDRs and/or LCDRs, or VH and/or VL sequence, of PRSIM_75.

The DBD-T fusion protein may comprise an amino acid sequence having at least 90% identity to the amino acid sequence set forth in SEQ ID NO: 45. In particular embodiments TRD-BM fusion protein defined in (1) above may comprise an amino acid sequence having at least 90% sequence identity to the amino acid sequence set forth in any one of SEQ ID NOs: 57-67.

The TRD-T fusion protein may comprise an amino acid sequence having at least 90% identity to the amino acid sequence set forth in SEQ ID NO: 44. In particular embodiments, the DBD-BM fusion protein defined in (2) above may comprise an amino acid sequence having at least 90% sequence identity to the amino acid sequence set forth in any one of SEQ ID NOs: 46-56.

As described in the examples, some of the exemplified binding members showed a preference for fusion to either the DNA binding domain or the transcriptional regulatory domain, whereby increased transcriptional regulatory activity was observed depending on if the particular binding member was fused to the DNA binding domain or transcriptional regulatory domain. Thus, in some embodiments:
(1) the first component polypeptide comprises a DNA binding domain and is fused to a target protein to form a DBD-T fusion protein, wherein the target protein comprises an amino acid sequence having at least 90% identity to the amino acid sequence set forth in SEQ ID NO: 1, and
   the second component polypeptide comprises a transcriptional regulatory domain and is fused to a binding member to form a TRD-BM fusion protein,
   wherein:
   a) the binding member in the TRD-BM fusion protein comprises the BC, DE and FG loops, or Tn3 sequence, of PRSIM_23;
   b) the binding member in the TRD-BM fusion protein comprises the BC, DE and FG loops, or Tn3 sequence, of PRSIM_47, or
   c) the binding member in the TRD-BM fusion protein comprises the HCDRs and/or LCDRs, or VH and/or VL sequence, of PRSIM_04;
   d) the binding member in the TRD-BM fusion protein comprises the HCDRs and/or LCDRs, or VH and/or VL sequence, of PRSIM_72;
   e) the binding member in the TRD-BM fusion protein comprises the HCDRs and/or LCDRs, or VH and/or VL sequence, of PRSIM_67; or
   f) the binding member in the TRD-BM fusion protein comprises the HCDRs and/or LCDRs, or VH and/or VL sequence, of PRSIM_75, or
(2) the first component polypeptide comprises a transcriptional regulatory domain and is fused to a target protein to form a TRD-T fusion protein, wherein the target protein has an amino acid sequence having at least 90% identity to SEQ ID NO: 1, and
   the second component polypeptide comprises a DNA binding domain and is fused to a binding member to form a DBD-BM fusion protein,
   wherein:
   g) the binding member in the DBD-BM fusion protein comprises the BC, DE and FG loops, or Tn3 sequence, of PRSIM_23;
   h) the binding member in the DBD-BM fusion protein comprises the HCDRs and/or LCDRs, or VH and/or VL sequence, of PRSIM_01;
   i) the binding member in the DBD-BM fusion protein comprises the HCDRs and/or LCDRs, or VH and/or VL sequence, of PRSIM_57;
   j) the binding member in the DBD-BM fusion protein comprises and the BC, DE and FG loops, or Tn3 sequence, of PRSIM_32;
   k) the binding member in the DBD-BM fusion protein comprises the BC, DE and FG loops, or Tn3 sequence, of PRSIM_33; or
   l) the binding member in the DBD-BM fusion protein comprises the BC, DE and FG loops, or Tn3 sequence, of PRSIM_36.

In some embodiments, the binding member or target protein is fused to the C-terminus of the DNA binding domain. In other embodiments, the binding member or target protein is fused to the N-terminus of the transcriptional regulatory domain. The binding member or target protein may be fused to the DNA binding domain or transcriptional regulatory domain via a peptide linker, for example via one or more of the peptide linkers set out above. In particular embodiments the linkers have the amino acid sequence TGGGGSGGGGS (SEQ ID NO: 185) or SA.

As described in the examples, PRSIM_23 was found to provide strong gene expression regulation in both orientations. Thus, in some embodiments:
(1) the first component polypeptide comprises a DNA binding domain and is fused to a target protein to form a DBD-T fusion protein, wherein the target protein comprises an amino acid sequence having at least 90% identity to the amino acid sequence set forth in SEQ ID NO: 1; and:
   the second component polypeptide comprises a transcriptional regulatory domain and is fused to a binding member to form a TRD-BM fusion protein, or
(2) the first component polypeptide comprises a transcriptional regulatory domain and is fused to a target protein to form a TRD-T fusion protein, wherein the target protein has an amino acid sequence having at least 90% identity to SEQ ID NO: 1; and
   the second component polypeptide comprises a DNA binding domain and is fused to a binding member to form a DBD-BM fusion protein,
wherein in either (1) or (2), the binding member comprises the BC, DE and FG loops, or Tn3 sequence, of PRSIM_23.

In particular embodiments:
(1) the DBD-T fusion protein comprises an amino acid sequence having at least 90% identity to SEQ ID NO: 45; and the TRD-BM fusion protein has an amino acid sequence having at least 90% identity to the amino acid sequence set forth in SEQ ID NO: 57, or
(2) the DBD-BM fusion protein comprises an amino acid sequence having at least 90% identity to the amino acid sequence set forth in SEQ ID NO: 46; and the TRD-T fusion protein comprises an amino acid sequence having at least 90% identity to the amino acid sequence set forth in SEQ ID NO: 44.

As also demonstrated in the examples, the PRSIM-based CIDs can also be applied to an activating CRISPR (CRISPRa) system. This can be used, for example, to facilitate endogenous gene regulation.

Thus, in some embodiments the DBD-BM fusion protein comprises an amino acid sequence having at least 90% identity to the amino acid sequence set forth in SEQ ID NO: 227; and the TRD-T fusion protein comprises an amino acid sequence having at least 90% identity to the amino acid sequence set forth in SEQ ID NO: 225. The DBD-BM fusion protein can be guided to a target sequence through the use of particular guide RNAs that are specific for said target sequence.

As demonstrated in the examples, split transcription factors comprising a DNA binding domain fused to multiple copies of the target protein or binding member exhibited increased expression relative to a split transcription factor comprising a DNA binding domain fused to a single copy of the target protein or binding member.

Thus, in some embodiments,
the DBD-T fusion protein comprises the DNA binding domain fused to multiple copies of the target protein (e.g. two, three, four, five or more target proteins); or
the DBD-BM fusion protein comprises the DNA binding domain fused to multiple copies of the target protein (e.g. two, three, four, five or more binding members).

The multiple binding members or multiple target proteins may be separated by a linker, for example by one or more peptide linkers as set out above. In particular exemplified embodiments the DBD-T fusion protein comprises a DNA binding domain fused to three target proteins, or the DBD-BM fusion protein comprises a DNA binding domain fused to three binding members.

The first and/or second component polypeptide may additionally comprise nuclear localization signals (such as, for example, that from the SV40 medium T-antigen).

A split transcription factor may also be provided with a third expression cassette, wherein the third expression cassette encodes a desired expression product, wherein the DNA binding domain of the split transcription factor binds to a target sequence in the third expression cassette such that the transcription factor is capable of regulating expression of the desired expression product. By “capable of regulating expression” it is intended to mean that the DNA binding domain is able to bind the target sequence and upon forming a transcription factor with the transcriptional regulatory domain (i.e. upon dimerization of the dimerization-inducible protein), has transcriptional regulatory activity that regulates (increases or decreases) expression of the desired expression product. The desired expression product can be RNA or peptidic (peptide, polypeptide or protein). Preferably the desired expression product is peptidic. The desired expression product may be a therapeutic protein, i.e. a protein that exerts a therapeutic effect in the subject.

The target sequence may be located in or in close proximity to a promoter that is operably linked to a coding sequence for the desired expression product. By “close proximity” it is meant that the target sequence is within 500 bp, within 250 bp, within 100 bp, within 50 bp, or within 25 bp of the sequence corresponding to the promoter.

Split Chimeric Antigen Receptor

The dimerization-inducible protein may be a split chimeric antigen receptor (split CAR).

CARs combine both antibody-like recognition with T-cell-activating function. They are typically composed of an antigen-specific recognition domain, e.g. derived from an antibody, a transmembrane domain to anchor the CAR to the T cell, a co-stimulatory domain and one or more intracellular signalling domains that induce persistence, trafficking and effector functions in transduced T cells. The design and use of CARs is well known in the art and is described, for example in Sadelain et al. 2013.

Split CARs have been designed that require an exogenous, user-provided signal to activate the CAR, for example as described in Wu et al. 2015. In these split receptors, antigen binding and intracellular signalling components only assemble in the presence of a heterodimerizing small molecule, allowing the user to precisely control the timing, location and dosage of T-cell activity. Such split CARs are expected to mitigate toxicity for example by inducing less off-target effects.

In one embodiment the dimerization-inducible protein comprises:
a first component polypeptide comprising a co-stimulatory domain and is fused to the target protein as defined herein; and
a second component polypeptide comprising an intracellular signalling domain and is fused to the binding member as defined herein.

The first component polypeptide set out above may further comprise an antigen-specific recognition domain and a transmembrane domain and the second component polypeptide further comprises a transmembrane domain and a second co-stimulatory domain, and wherein the first and second component polypeptide form a chimeric antigen receptor (CAR) upon dimerization. By “form a CAR” it is meant that the first and second component polypeptides are brought into close enough proximity that they are able to reconstitute a fully functional CAR.

In another embodiment the dimerization-inducible protein comprises:
a first component polypeptide comprising an intracellular signalling domain and is fused to the target protein as defined herein; and
a second component polypeptide comprising a first co-stimulatory domain and is fused to the binding member as defined herein.

The first component polypeptide set out above may further comprise a transmembrane domain and a second co-stimulatory domain and the second component polypeptide further comprises an antigen-specific recognition domain and a transmembrane domain, wherein the first and second component polypeptide form a chimeric antigen receptor (CAR) upon dimerization,

The split CAR will have increased activity when the binding member is bound to the T-SM complex, wherein the activity is increased compared to the activity observed when the binding member is not bound to the T-SM complex.

In one embodiment the first component polypeptide comprises, from N-terminal to C-terminal:
i) an antigen-specific recognition domain;
ii) a transmembrane domain; and
ii) a first co-stimulatory domain;
and the second component polypeptide comprises, from N-terminal to C-terminal:
i) a transmembrane domain;
ii) a second co-stimulatory domain; and
iii) an intracellular signalling domain,
   wherein the first component polypeptide and second component polypeptide form a CAR upon dimerization.

In some embodiments the target protein and binding member are fused at a location that is C-terminal to the respective transmembrane domains in the first and second component polypeptides. For example, the target protein or binding member may be fused to the N-terminus or C-terminus of the respective co-stimulatory domains in the first and second component polypeptides. In a particular embodiment, one of the target protein and binding member is fused to the C-terminus of the first co-stimulatory domain and the other is fused to the C-terminus of the second co-stimulatory domain.

For example, in one embodiment the first component polypeptide comprises from N-terminal to C-terminal:
i) an antigen-specific recognition domain;
ii) a transmembrane domain
iii) a first co-stimulatory domain;
   and the second component polypeptide comprises from N-terminal to C-terminal:
i) a transmembrane domain;
ii) a second co-stimulatory domain; and
iii) an intracellular signalling domain,
   wherein, the target protein is fused to the C-terminus of the first co-stimulatory domain and the binding member is fused to the C-terminus of the second co-stimulatory domain.

For example, in another embodiment the first component polypeptide comprises from N-terminal to C-terminal:
i) an antigen-specific recognition domain;
ii) a transmembrane domain
iii) a first co-stimulatory domain;
   and the second component polypeptide comprises from N-terminal to C-terminal:
i) a transmembrane domain;
ii) a second co-stimulatory domain; and
iii) an intracellular signalling domain,
   wherein, the binding member is fused to the C-terminus of the first co-stimulatory domain and the target protein is fused to the C-terminus of the second co-stimulatory domain.

The target protein and/or binding member may be fused directed to the respective co-stimulatory domains. More preferably, the target protein and binding member are separated from their respective co-stimulatory domains by peptide linkers. The peptide linkers may be as further defined herein. In some embodiments, the target protein and binding member are separated from their respective co-stimulatory domains by a linker comprising the amino acid sequence set forth in SEQ ID NO: 204. Similarly, peptide linkers may separate the various domains in the first and second component polypeptides. For example, the transmembrane domain may be separated from the second co-stimulatory domain by a peptide linker, e.g. a peptide linker comprising the amino acid sequence GS, and/or the second co-stimulatory domain may be separated from the intracellular signalling domain by a peptide linker, e.g. a peptide linker comprising the amino acid sequence set forth in SEQ ID NO: 204.

Non-limiting examples of suitable co-stimulatory domains include, but are not limited to, activation domains from 4-1BB (CD137), CD28, ICOS, OX-40, BTLA, CD27, CD30, GITR, and HVEM. In one embodiment the first and second co-stimulatory domain is a 4-1 BB activation domain.

Non-limiting examples of suitable intracellular signalling domains include, but are not limited to, cytoplasmic sequences of the T cell receptor (TCR) and co-receptors that act in concert to initiate signal transduction following antigen receptor engagement, as well as any derivative or variant of these sequences and any synthetic sequence that has the same functional capability. Particular intracellular signalling domains are those that include signaling motifs which are known as immunoreceptor tyrosine-based activation motifs or ITAMs. Examples of ITAM containing signaling domains include those derived from TCR zeta, FcR gamma, FcR beta, CD3 gamma, CD3 delta, CD3 epsilon, CD3 zeta, CD5, CD22, CD79a, CD79b, and CD66d. In particular embodiments the intracellular signalling domain is derived from CD3 zeta.

The transmembrane domain may be derived either from a natural or from a synthetic source. Where the source is natural, the domain may be derived from any membrane-bound or transmembrane protein. Transmembrane regions may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, or from an immunoglobulin such as IgG4. Alternatively, the transmembrane domain may be synthetic, in which case it will comprise predominantly hydrophobic residues such as leucine and valine. A triplet of phenylalanine, tryptophan and valine may be found at each end of a synthetic transmembrane domain. Optionally, a short oligo- or polypeptide linker, preferably between 2 and 10 amino acids in length may form the linkage between the transmembrane domain and the intracellular signalling domain of the CAR. A glycine-serine doublet provides a particularly suitable linker. In particular embodiments, the transmembrane domain is derived from CD28.

The first and second polypeptides may additionally include a hinge domain, such as an IgG4 or CD8a hinge domain, N-terminal to the transmembrane domains in the first and/or second polypeptides. Examples of hinge domains are described in, for example, Qin et al. 2017. In particular embodiments, the hinge domain is a human IgG4 hinge domain.

An antigen-specific recognition domain suitable for use in a dimerization-inducible protein of the present disclosure can be any antigen-binding polypeptide, a wide variety of which are known in the art. In some instances, the antigen-binding domain is a single chain Fv (scFv). Other antibody-based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use. In some instances, T-cell receptor (TCR) based recognition domains such as single chain TCR (scTv, single chain two-domain TCR containing v vβ) are also suitable for use.

In particular embodiments, the antigen-specific recognition domain is a single chain Fv (scFv). As described elsewhere, an scFv typically comprises a VH chain separated from a VL chain by a peptide linker, e.g. a peptide linker comprising the amino acid sequence set forth in SEQ ID NO: 204.

An antigen-specific recognition domain suitable for use in a dimerization-inducible protein of the present disclosure can have a variety of antigen-binding specificities. In some cases, the antigen-binding domain is specific for an epitope present in an antigen that is expressed by (synthesized by) a cancer cell, i.e., a cancer cell associated antigen. The cancer cell associated antigen can be an antigen associated with, e.g., a breast cancer cell, a B cell lymphoma, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a glioblastoma, a medulloblastoma, a colorectal cancer cell, etc. A cancer cell associated antigen may also be expressed by a non-cancerous cell.

In particular exemplary embodiments, the target protein used in the split-CAR is derived from an HCV NS3/4A protease, the small molecule is simeprevir and the binding member is based on PRSIM_23 (e.g. comprises the BC, DE and FG loops or Tn3 sequence of PRSIM_23, optionally with the sequence identity and/or alterations described herein).

In some embodiments the first component polypeptide comprises from N-terminal to C-terminal:
i) an antigen-specific recognition domain;
ii) a transmembrane domain
iii) a first co-stimulatory domain;
   and the second component polypeptide comprises from N-terminal to C-terminal:
i) a transmembrane domain;
ii) a second co-stimulatory domain; and
iii) an intracellular signalling domain,
   wherein the target protein is fused to the C-terminus of the first co-stimulatory domain and the binding member is fused to the C-terminus of the second co-stimulatory domain, wherein the first component polypeptide fused to the target protein comprises an amino acid sequence having at least 90% identity to the amino acid sequence set forth in SEQ ID NO: 70; and wherein the second component polypeptide fused to the binding member comprises an amino acid sequence having at least 90% identity to the amino acid sequence set forth in SEQ ID NO: 200, optionally wherein the antigen-specific recognition domain (e.g. scFv) is located N-terminal to the amino acid sequence having at least 90% identity to the amino acid sequence set forth in SEQ ID NO: 70.

In some embodiments, the first component polypeptide comprises a first signal peptide located N-terminal to the antigen-specific recognition domain. The first signal peptide may comprise the amino acid sequence set forth in SEQ ID NO: 201 or SEQ ID NO: 202. In exemplified embodiments, the first signal peptide comprises the amino acid sequence set forth in SEQ ID NO: 201.

In some embodiments, the second component polypeptide comprises a second signal peptide located N-terminal to the transmembrane domain. The second signal peptide may comprise the amino acid sequence set forth in SEQ ID NO: 201 or SEQ ID NO: 202. In exemplified embodiments, the second signal peptide comprises the amino acid sequence set forth in SEQ ID NO: 202. In one embodiment, the second component polypeptide comprises an amino acid sequence having at least 90% identity to the amino acid sequence set forth in SEQ ID NO: 203.

Also provided is an engineered immune cell comprising the split CAR disclosed herein. In one embodiment the immune cell is a T-cell. Also provided is a method of genetically modifying an immune cell to express a split CAR disclosed herein. The method may be carried out ex vivo. The method may comprise administering the one or more expression vectors described herein to the immune cell such that the split CAR is expressed on the surface of the immune cell.

Split Reporter System

The dimerization-inducible protein may be a split reporter system. The split reporter system may be an enzyme or fluorescent protein that provides an observable phenotype when the first and second component polypeptides dimerise. The observable phenotype may be a colorimetric signal, a luminescent signal or a fluorescent signal. Particular examples of split reporter systems are provided in Dixon et al. 2017.

In some embodiments, the first component polypeptide comprises a first reporter component; and the second component polypeptide comprises a second reporter component, and wherein the first component polypeptide and second component polypeptide form a reporter system upon dimerization, optionally wherein the reporter system provides an increased colorimetric, luminescent, or a fluorescent signal when the binding member is bound to the T-SM complex.

Split Apoptotic Protein

The dimerization-inducible protein may be a split apoptotic protein. A split apoptotic protein is any protein that is capable of inducing apoptosis when the first and second component polypeptides of the split apoptotic protein dimerise. An example of a split apoptotic protein is a split caspase (e.g. split caspase 9 or split caspase 3), that is capable to inducing apoptosis upon dimerization and as such can be used to kill specific cells that contain the split apoptotic protein (e.g. diseased cells, or therapeutic cells that have been administered for cell therapy purposes). Examples of split caspases are provided in Chelur et al. 2007. The use of an inducible caspase 9 suicide gene system is described, for example, in Gargett et al. 2014.

In some embodiments, the first component polypeptide comprises a first caspase component; and the second component polypeptide comprises a second caspase component, wherein the first component polypeptide and second component polypeptide form a caspase upon dimerization. The split caspase may be capable of inducing cell death when the binding member is bound to the T-SM complex.

In certain embodiments, the first and second caspase components are identical, for example both caspase components comprise caspase 9 activation domains. An exemplary caspase 9 activation domain is provided as amino acids residues 152-414 of the human caspase 9 amino acid sequence provided as NCBI accession number AAO21133.1 (version 1; last updated 1 Dec. 2009). In cases where the first and second caspase components are identical, the first and second caspase components may be encoded from the same expression cassette. For example, a split apoptotic protein may be encoded from one or more expression cassettes encoding the target protein, the binding member and the caspase 9 activation domain, where both the target protein and the binding member are fused to a caspase 9 activation domain. Upon expression, a plurality of proteins comprising the target protein, binding member and caspase 9 activation domain are produced and dimerization of the caspase 9 activation domains (i.e. at least a first and a second caspase 9 activation domain) can be regulated through the addition of the small molecule.

In certain exemplary embodiments, the split apoptotic protein comprises an amino acid sequence having at least 90%, 91%, 92%, 93%, 94%, 95%, 96%, 97%, 98%, 99% or 100% sequence identity to SEQ ID NO: 223.

Other Dimerization-Inducible Proteins

Other dimerization proteins contemplated for use with the present disclosure include split therapeutic proteins, split TEV proteases and split Cas9. A split therapeutic protein is any protein that is capable of exerting a therapeutic effect when the first and second component polypeptides of the split therapeutic protein dimerize.

### Viral Vectors and Viral Particles

In one embodiment the expression vector is a viral vector. Suitable viral vectors for use include adeno-associated virus vectors, adenovirus vectors, herpes simplex virus vectors, retrovirus vectors, lentivirus vectors, alphavirus vectors, flavivirus vectors, rhabdovirus vectors, measles virus vectors, Newcastle disease virus vectors, poxvirus vectors and picornavirus vectors.

As used herein a viral vector means a DNA expression vector which comprises the first and second expression cassettes such that the expression cassettes are converted into a viral genome that is packaged in the viral particle when expressed in a cell alongside the necessary components for the assembly of the viral particle. Additionally, in one embodiment, the viral vector comprises a third expression cassette encoding a desired expression product.

In a particular embodiment the expression vector is an adeno-associated virus (AAV) vector. AAVs are one of the most actively investigated gene therapy vehicles and are characterized by excellent safety profile and high efficiency of transduction in a broad range of target tissues. The use of AAVs as a vector for gene therapy is described in for example Naso et al. 2017 and Colella et al. 2018.

Various AAV serotypes, including AAV1, AAV3, AAV4, AAV5, AAV6, AAV6.2, AAV6.2FF, AAV8, AAV 8.2, AAV9, and AAV rh10 and pseudotyped AAV such as AAV2/8, AAV2/5 and AAV2/6 can also be used in accordance with the present disclosure. Further examples of serotypes and their isolation are described in Srivastava, 2006.

The AAV particle is a small (25-nm) virus from the Parvoviridae family, and it is composed of a non-enveloped icosahedral capsid (protein shell) that contains a linear single-stranded DNA genome of around 4.8 kb. The AAV genome encodes for several protein products, namely, four non-structural Rep proteins, three capsid proteins (VP1-3), and the assembly-activating protein (AAP). The AAV genes are flanked by two AAV-specific palindromic inverted terminal repeats (ITRs).

Thus, where the expression vector is an AAV vector, this may mean that the first and second expression cassettes are flanked by ITRs (e.g. ITR-first expression cassette-second expression cassette-ITR), such that the expression cassettes are converted into a single-stranded genome that is packaged in an AAV particle when expressed in a cell alongside the necessary components for the assembly of the AAV particle.

The AAV vector may be engineered, for example in order to improve their function. Examples of AAVs that have been engineered for clinical gene therapy are described in Kotterman and Schaffer, 2014.

AAV vectors have a packaging capacity of less than 5 kb, which can limits the size of the genetic material (e.g. expression cassettes) that can be introduced in the viral genome. As demonstrated herein, the use of components that have a relatively small size, such as Tn3 proteins and scFvs as the binding members, allow for the expression cassette(s) encoding the tripartite complex (e.g. as part of a dimerization-inducible protein such as a split transcription factor) to fit within a single AAV vector. As additionally demonstrated herein, the small size of the expression cassette(s) encoding the tripartite complex allowed for a transgene (e.g. as part of a third expression cassette) to be introduced into the same AAV vector as the components of the split transcription factor, allowing the split transcription factor to be delivered “in cis” with the transgene.

The disclosure also includes in vitro methods of making viral particles. In one embodiment, a method of making viral particles involves transfecting host cells such as mammalian cells with a viral vector as described herein and expressing viral proteins necessary for particle formation in the cells and culturing the transfecting cells in a culture medium, such that the cells produce viral particles. The viral particles may be released into the culture medium, or the method may additionally involve lysing and isolating particles from the cell lysates. An example of a suitable mammalian cell is a human embryonic kidney (HEK) 293 cell.

Typically, multiple plasmid expression vectors are utilised to generate the various protein components that generate the viral particles. It is also possible to make use of cell lines that constitutively express components for viral packaging, enabling the use of few plasmids.

For example, construction of an AAV particle requires the Rep and Cap proteins and additional genes from adenovirus to mediate AAV replication. Making AAV particles is described for example in Robert et al. 2017

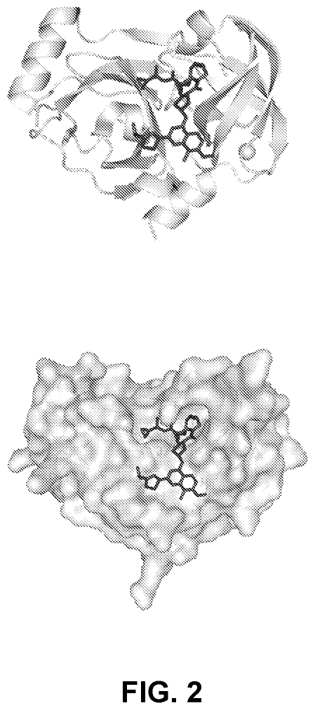

An exemplary method of producing AAV particles is described in Robert et al. 2017. Briefly, this involves transfection of a mammalian cell line, such as HEK293 cells, with three plasmids. One vector encodes the rep and cap genes of AAV (pRepCap) using their endogenous promoters; one vector (pHelper) encodes three additional adenoviral helper genes (E4, E2A and VA RNAs) not present in HEK293 cells and; one vector (the viral vector) (pAAV-GOI) contains the one or more expression cassettes flanked by two ITRs. See FIG. 2 of Robert et al.

Following release of viral particles, the culture medium comprising the viral particles may be collected and, optionally the viral particles may be separated from the cell lysate. Optionally, the viral particles may be concentrated.

Following production and optional concentration, the viral particles may be stored, for example by freezing at −80° C. ready for use by administering to a cell and/or use in therapy.

The disclosure also provides viral particles, such as AAV particles, for example those produced by the methods described herein. As used herein, a viral particle comprises a viral genome packaged within the viral envelope that is capable of infecting a cell, e.g. a mammalian cell.

Disclosed herein are one or more viral particles comprising a viral genome encoding:
i) a target protein, wherein the target protein is capable of binding to a small molecule in order to form a complex between the target protein and small molecule (T-SM complex); and
ii) a binding member, wherein the binding member specifically binds to the T-SM complex such that the binding member binds the T-SM complex at a higher affinity than it binds both the target protein alone and the small molecule alone,
wherein the target protein is derived from a viral protease and the small molecule is a viral protease inhibitor. In one embodiment, the target protein is fused to a first component polypeptide and the binding member is fused to a second component polypeptide.

Also disclosed herein are one or more viral particles comprising:
i) a first expression cassette encoding a target protein, wherein the target protein is capable of binding to a small molecule in order to form a complex between the target protein and the small molecule (T-SM complex); and
ii) a second expression cassette encoding a binding member, wherein the binding member specifically binds to the T-SM complex such that the binding member binds the T-SM complex at a higher affinity than it binds both the target protein alone and the small molecule alone,
wherein the target protein is derived from a non-human protein and the small molecule is an inhibitor of the non-human target protein, and wherein the first and second expression cassettes form part of a viral genome in the one or more viral particles. In one embodiment, the non-human protein is derived from a viral protease and the small molecule is a viral protease inhibitor. In one embodiment, the target protein is fused to a first component polypeptide and the binding member is fused to a second component polypeptide.

In some embodiments, the first and second expression cassettes form part of the same viral genome of a viral particle. In other embodiments, the first expression cassette is located in a first viral genome of a first viral particle and the second expression cassette is located in a second viral genome of a second viral particle.

The expression cassette, target protein, binding member, small molecule and first and second component polypeptides may be as further defined above. Depending on the viral particle used, the viral genome may be a single stranded or double stranded nucleic acid and may be RNA or DNA. For example, when the viral particle is an AAV particle, the viral genome is a single stranded DNA viral genome. The viral genome may encode the split proteins as defined above.

### Gene Therapy

The agents (i.e. the one or more expression vectors, expression products or viral particles, plus small molecule) may be administered to a patient as part of a method of treatment or a method of prophylaxis of a disease. Following binding of the binding member to the T-SM complex the recipient individual may experience a reduction in symptoms of the disease or disorder being treated. This may have a beneficial effect on the disease condition in the individual.

The term “treatment,” as used herein in the context of treating a condition, pertains generally to treatment and therapy of a human, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

“Prophylaxis” in the context of the present specification should not be understood to circumscribe complete success i.e. complete protection or complete prevention. Rather prophylaxis in the present context refers to a measure which is administered in advance of detection of a symptomatic condition with the aim of preserving health by helping to delay, mitigate or avoid that particular condition.

The method of treatment may involve expressing one or more dimerization-inducible proteins as defined further herein in a cell. The dimerization-inducible protein may, for example, comprise a first component polypeptide and a second component polypeptide that form a therapeutic polypeptide upon dimerization. In this way, addition of the small molecule can result in the therapeutic protein having increased activity and can be used, for example, in a method of treatment of a disease where the therapeutic protein is deficient.

Disclosed herein is a method of regulating the expression of a desired expression product in a cell, comprising i) expressing a dimerization-inducible protein described herein in the cell, wherein the first and second component polypeptides form a transcription factor upon dimerization, and wherein the DNA binding domain binds to a target sequence in the cell such that the transcription factor is capable of regulating (i.e. increasing or decreasing) expression of the desired expression product in the cell, and ii) administering the small molecule to the cell in order to regulate expression of the desired expression product.

Additionally disclosed herein is a dimerization-inducible protein for use in a method of regulating the expression of a desired expression product in a cell in a human or animal subject, the method comprising expressing the dimerization-inducible protein described herein in the cell, wherein the first and second component polypeptides form a transcription factor upon dimerization, and administering the small molecule to the cell in order to regulate (e.g. increase or decrease) expression of the desired expression product. Also disclosed herein is a small molecule for use in a method of regulating the expression of a desired expression product in a cell in a human or animal subject, the method comprising expressing the dimerization-inducible protein described herein in the cell, wherein the first and second component polypeptides form a transcription factor upon dimerization, and administering the small molecule to the cell in order to regulate (e.g. increase or decrease) expression of the desired expression product.

The method may comprise administering one or more expression vectors or viral particles as described herein in order to express the dimerization-inducible protein in the cell. In other embodiments the method may comprise administering an expression product produced from the one or more expression vectors, e.g. mRNA encoding the dimerization-inducible protein, to the cell. The particular administration would be at the discretion of the physician who would also select dosages using his/her common general knowledge and dosing regimens known to a skilled practitioner.

The desired expression product can be RNA or a peptidic (peptide, polypeptide or protein). Preferably the desired expression product is peptidic. The desired expression product may be a therapeutic protein, i.e. a protein that exerts a therapeutic effect in the subject.

The desired expression product may be part of an endogenous gene present in the genome of the target cell. For example, where the method is carried out in a human cell, the desired expression product may be part of a human gene. Alternatively, the desired expression product may be part of a transgene delivered to the target cell, e.g. a therapeutic transgene. Regulating expression of the gene may be used in a method of treatment or a method of prophylaxis of a disease. Following expression of the split transcription factor and administration of the small molecule, the recipient individual may exhibit reduction in symptoms of the disease or disorder being treated. This may have a beneficial effect on the disease condition in the individual.

Where the target sequence is part of a transgene delivered to the cell, the method may further comprise administering a third expression cassette to the cell, wherein the third expression cassette encodes the desired expression product and wherein the third expression cassette comprises the target sequence. The transgene may comprise a promoter that is operably linked to a coding sequence for the desired expression product, which may be a therapeutic protein, e.g. a therapeutic antibody. An example of a therapeutic antibody is MEDI8852, having the heavy chain amino acid sequence set forth as SEQ ID NO: 205 and the light chain amino acid sequence set forth as SEQ ID NO: 206. The third expression cassette may be part of the same expression vector or viral particle as one or both of the first and second expression cassettes. In other words, the transgene may be delivered “in cis” with the split transcription factor to the cell, such within the same viral (e.g. AAV) particle. Alternatively, the third expression cassette may be part of a different expression vector or viral particle as one or both of the first and second expression cassettes. In other words, the transgene may be delivered “in trans” with the split transcription factor to the cell, such as within separate viral (e.g. AAV) particles. As demonstrated herein, the split transcription factors of the disclosure are suitable for both “in cis” and “in trans” delivery with the transgene.

The target sequence may be located in or in close proximity to a promoter that is operably linked to a coding sequence for the desired expression product. By “close proximity” it is meant that the target sequence is within 500 bp, within 250 bp, within 100 bp, within 50 bp, or within 25 bp of the sequence corresponding to the promoter.

Administration to the cell may occur by any suitable means. For example, the expression cassettes may be delivered by viral, e.g. as part of a viral particle described herein, or by non-viral means. Non-viral means of delivery include electroporation, lipofection, microinjection, biolistics, virosomes, liposomes, immunoliposomes, polycation or lipid:nucleic acid conjugates, naked DNA, naked RNA, artificial virions, and agent-enhanced uptake of DNA. In one embodiment, the expression cassettes are delivered as mRNA. In one embodiment, the expression cassettes are delivered as DNA plasmids.

In any of the in vivo methods disclosed herein, the small molecule may be orally administered to a human subject, for example in an acceptable dosage form such as a capsule, tablet, aqueous suspension or solution. The amount used will depend on the host treated and the particular mode of administration. The small molecule may be administered as a single dose, multiple doses or over an established period of time.

Where the method involves administering a viral particle to a cell, the unit dose may be calculated in terms of the dose of viral particles being administered. Viral doses include a particular number of virus particles or plaque forming units (pfu) or viral genome copies (vgc). For embodiments involving AAV, particular unit doses include 10³, 10⁴, 10⁵, 10⁶, 10⁷, 10⁸, 10⁹, 10¹⁰, 10¹¹, 10¹², 10¹³, 10¹⁴, 10¹⁵, 10¹⁶ viral genome copies (vgc) per kg of body weight. Particle doses may be somewhat higher (10 to 100-fold) due to the presence of infection-defective particles.

Without wishing to be bound by theory, infection and transduction of cells by viral particles (e.g. AAV particles) is believed to occur by a series of sequential events as follows: interaction of the viral capsid with receptors on the surface of the target cell, internalization by endocytosis, intracellular trafficking through the endocytic/proteasomal compartment, endosomal escape, nuclear import, virion uncoating, and viral DNA double-strand conversion that leads to the transcription and expression of proteins encoded by the viral genome in the viral particle.

While it is possible for the one or more expression vectors, expression products, viral particles, and small molecules to be used (e.g., administered) alone, it is often preferable to present the individual components as a composition or formulation e.g. with a pharmaceutically acceptable carrier or diluent.

For example, the one or more viral particles may be administered as a pharmaceutical composition comprising the one or more viral particles and a pharmaceutically acceptable carrier or diluent. As another example, the small molecules may be administered as a pharmaceutical composition comprising the small molecule and a pharmaceutically acceptable carrier or diluent.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

The agents (i.e. the one or more expression vectors, DNA plasmids or viral particles, plus small molecule) may be administered simultaneously or sequentially and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the agent(s) being administered. In one embodiment, the small molecule is administered after administration of the one or more expression vectors, DNA plasmids or viral particles.

### Cellular Therapy

Also provided are methods of cellular therapy. Cellular therapy involves administering cells that have been genetically modified to express an expression product, such as a dimerization-inducible protein, to a patient.

Cells such as stem cells may be used methods of cellular therapy. One potential advantage associated with using stem cells is that they can be differentiated into other cell types in vitro, and can be introduced into a mammal (such as the donor of the cells) where they will engraft in the bone marrow. Suitable stem cells include embryonic stem cells, induced pluripotent stem cells, hematopoietic stem cells, mesenchymal stem cells, neuronal stem cells, cardiac stem cells and mesenchymal stem cells.

For example, the cellular therapy may involve administering the one or more expression vectors described herein to a cell (e.g. a stem cell) in an ex vivo method such that a dimerization-inducible protein is expressed by the cell and administering the cell to a patient. Following administration of the cell expressing the dimerization-inducible protein, a small molecule may be administered to the individual in order to induce dimerization of the first and second component polypeptides in order to reconstitute their function upon dimerization. For example, the first and second component polypeptides may form a transcription factor upon dimerization, or the first and second component polypeptides may form a CAR upon dimerization.

Disclosed herein is a method of treatment comprising administering a cell expressing a dimerization-inducible protein defined herein to a patient, the method comprising:
i) administering the cell to an individual; and
ii) administering the small molecule to the individual.

The dimerization-inducible protein may be for example a split transcription factor, a split CAR, a split apoptotic protein or a split therapeutic protein. The method of treatment may be a method of treating cancer.

Cellular therapy may involve isolating cells from a patient, transfecting the cells with one or more expression vectors ex vivo and the cells are administered to the patient. Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

For example, the cellular therapy may involve isolating a cell from a patient, administering the one or more expression vectors described herein to the cell in an ex vivo method such that a dimerization-inducible protein is expressed by the cell, and administering the cell back to the patient. Following administration of the cell expressing the dimerization-inducible protein, a small molecule may be administered to the individual in order to induce dimerization of the first and second component polypeptides as described herein.

In one embodiment, the cell is an immune cell (such as a T-cell) and the dimerization-inducible protein expressed by the cell is a split CAR. Methods of treatment involving CAR T-cell therapy are known in the art and are described for example in Miliotou and Papadopoulou, 2018.

Disclosed herein is a method of treatment comprising administering a cell expressing the dimerization-inducible protein defined herein to a patient thereof, wherein the first and second component polypeptide form a CAR upon dimerization, the method comprising:
i) administering the cell to an individual; and
ii) administering the small molecule to the individual.

The method of treatment may be a method of treating cancer.

### Nucleic Acids

The disclosure also provides a nucleic acid molecule or molecules encoding a binding member or dimerization-inducible protein defined herein. The nucleic acid molecule or molecules may be isolated nucleic acid molecule or molecules. The nucleic acids encoding the binding members and dimerization-inducible proteins may have the requisite features and sequence identity as described herein in relation to the expression vectors. The skilled person would have no difficulty in preparing such nucleic acid molecules using methods well-known in the art.

In some embodiments the nucleic acid molecule or molecules encode the VH and/or VL domain(s) of PRSIM_57, PRSIM_01, PRSIM_04, PRSIM_67, PRSIM_72, or PRSIM_75. The amino acid sequences for those VH or VL domains are defined herein.

In some embodiments, the nucleic acid molecule or molecules encode the binding member of PRSIM_23, PRSIM_32, PRSIM_33, PRSIM_36, PRSIM_47, PRSIM_57, PRSIM_01, PRSIM_04, PRSIM_67, PRSIM_72, or PRSIM_75. The amino acid sequences for those binding members are defined herein.

In some embodiments, the nucleic acid molecule or molecules comprise a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the exemplary nucleic acid sequences set forth for PRSIM_23, PRSIM_32, PRSIM_33, PRSIM_36, PRSIM_47, PRSIM_57, PRSIM_01, PRSIM_04, PRSIM_67, PRSIM_72, or PRSIM_75. In some embodiments, the nucleic acid molecule or molecules comprise a nucleic acid sequence of PRSIM_23, PRSIM_32, PRSIM_33, PRSIM_36, PRSIM_47, PRSIM_57, PRSIM_01, PRSIM_04, PRSIM_67, PRSIM_72, or PRSIM_75. The nucleic acid sequences for those exemplary binding members are set forth in the following table:

| Binding member | Nucleic acid sequence provided as: |
|---|---|
| PRSIM_23 | SEQ ID NO: 73 |
| PRSIM_32 | SEQ ID NO: 74 |
| PRSIM_33 | SEQ ID NO: 75 |
| PRSIM_36 | SEQ ID NO: 76 |
| PRSIM_47 | SEQ ID NO: 77 |
| PRSIM_57 | SEQ ID NO: 80 |
| PRSIM_01 | SEQ ID NO: 78 |
| PRSIM_04 | SEQ ID NO: 79 |
| PRSIM_67 | SEQ ID NO: 81 |
| PRSIM_72 | SEQ ID NO: 82 |
| PRSIM_75 | SEQ ID NO: 83 |

In some embodiments, the nucleic acid molecule or molecules encodes the first component polypeptide and/or second component polypeptides fused to the target protein or binding member as described above. The amino acid sequences for those component polypeptides are defined herein.

In some embodiments, the nucleic acid molecule or molecules encodes one or more of the DBD-T fusion protein, TRD-BM fusion protein, DBD-BM fusion protein, and TRD-T fusion protein as described above. The amino acid sequences for those fusion proteins are defined herein.

In some embodiments, the nucleic acid molecule or molecules encoding a TRD-T fusion protein has a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid sequence set forth in SEQ ID NO: 108. In some embodiments, the nucleic acid molecule or molecules encoding a TRD-T fusion protein has the nucleic acid sequence of SEQ ID NO: 108.

In some embodiments, the nucleic acid molecule or molecules encoding a DBD-T fusion protein has a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid sequence set forth in SEQ ID NO: 109. In some embodiments, the nucleic acid molecule or molecules encoding a DBD-T fusion protein has the nucleic acid sequence of SEQ ID NO: 109.

In some embodiments, the nucleic acid molecule or molecules encoding a DBD-BM fusion protein has a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with any one of the nucleic acid sequences set forth in SEQ ID NOs: 110-120. In some embodiments, the nucleic acid molecule or molecules encoding a DBD-BM fusion protein has the nucleic acid sequence of any one of SEQ ID NOs: 110-120.

In some embodiments, the nucleic acid molecule or molecules encoding a TRD-BM fusion protein has a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with any one of the nucleic acid sequences set forth in SEQ ID NO: 121-131. In some embodiments, the nucleic acid molecule or molecules encoding a TRD-BM fusion protein has the nucleic acid sequence of any one of SEQ ID NOs: 121-131.

In some embodiments the nucleic acid molecule or molecules encode a split CAR as defined herein. In some embodiments the nucleic acid molecule or molecules encoding a split CAR has a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid sequence set forth in SEQ ID NO: 133 and a nucleic acid sequence encoding the antigen-specific recognition domain. In some embodiments, the nucleic acid molecule or molecules encoding a split CAR has the nucleic acid sequence of SEQ ID NO: 133 and a nucleic acid sequence encoding the antigen-specific recognition domain. In some embodiments, the nucleic acid molecule or molecules encoding a split CAR comprises a nucleic acid sequence encoding an antigen-specific recognition domain (e.g. an scFv) located between positions 66 and 67, wherein the nucleotide numbering corresponds to SEQ ID NO: 133.

An isolated nucleic acid molecule may be used to express a binding member or dimerization-inducible protein disclosed herein. The nucleic acid will generally be provided in the form of one or more expression vectors, for example having the features of the expression vectors described herein.

### Kits

The disclosure also provides kits that comprise one or more expression vectors, one or more viral particles, cells, or one or more nucleic acids, all as defined herein, with a small molecule, also as defined herein. In some embodiments, the small molecule is simeprevir. Where the one or more expression vector or nucleic acid encodes a polypeptide containing a DNA binding domain that is from a CRISPR/Cas system, the kit may additionally include a guide RNA specific for the target sequence, or a nucleic acid encoding the guide RNA specific for the target sequence.

### Sequence Identity and Alterations

Sequence identity is commonly defined with reference to the algorithm GAP (Wisconsin GCG package, Accelerys Inc, San Diego USA). GAP uses the Needleman and Wunsch algorithm to align two complete sequences, maximising the number of matches and minimising the number of gaps. Generally, default parameters are used, with a gap creation penalty equaling 12 and a gap extension penalty equaling 4. Use of GAP may be preferred but other algorithms may be used, e.g. BLAST (which uses the method of Altschul et al. (1990)), FASTA (which uses the method of Pearson and Lipman (1988)), or the Smith-Waterman algorithm (Smith and Waterman (1981)), or the TBLASTN program, of Altschul et al. (1990) supra, generally employing default parameters. In particular, the psi-Blast algorithm may be used.

Where the disclosure makes reference to a particular amino acid sequence having at least 90% sequence identity to a reference amino acid sequence, this includes the amino acid sequence having 90%, 91%, 92%, 93%, 94%, 95%, 96%, 97%, 98%, 99% and 100% sequence identity to the reference amino acid sequence.

The term “sequence alterations” as used herein is intended to encompass the substitution, deletion and/or insertion of an amino acid residue. Thus, a protein containing one or more amino acid sequence alterations compared to a reference sequence contains one or more substitutions, one or more deletions and/or one or more insertions of an amino acid residues as compared to the reference sequence. The term “amino acid mutation” is also herein used interchangeably with “sequence alteration”, unless the context clearly identifies otherwise.

In some embodiments in which one or more amino acids are substituted with another amino acid, the substitutions may be conservative substitutions, for example according to the following Table. In some embodiments, amino acids in the same block in the middle column are substituted, i.e. a non-polar amino acid is substituted for another non-polar amino acid for example. In some embodiments, amino acids in the same line in the rightmost column are substituted, i.e. G is substituted for A or P for example.

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F WY |

In some embodiments, substitution(s) may be functionally conservative. That is, in some embodiments the substitution may not affect (or may not substantially affect) one or more functional properties (e.g. binding affinity) of the protein comprising the substitution as compared to the equivalent unsubstituted protein.

The binding member may also comprise a variant of a BC, DE or FG loop, Tn3, CDR, VH domain, VL domain, and/or scFv sequence as disclosed herein. Suitable variants can be obtained by means of methods of sequence alteration, or mutation, and screening. In a preferred embodiment, a binding member comprising one or more variant sequences retains one or more of the functional characteristics of the parent binding member, such as binding specificity and/or binding affinity for the T-SM complex. For example, a binding member comprising one or more variant sequences preferably binds to T-SM complex with the same affinity as, or a higher affinity than, the (parent) binding member. The parent binding member is a binding member which does not comprise the amino acid substitution(s), deletion(s), and/or insertion(s) which has (have) been incorporated into the variant binding member.

For example, a binding member may comprise a BC, DE or FG loops, Tn3, CDR, VH domain, VL domain, or scFv sequence which has at least 70%, at least 75%, at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to a BC, DE or FG loops, Tn3, CDR, VH domain, VL domain, or scFv sequence disclosed herein.

A binding member may comprise a BC, DE or FG loops, Tn3, CDR, VH domain, VL domain, or scFv sequence which has one or more amino acid sequence alterations (addition, deletion, substitution and/or insertion of an amino acid residue), preferably 20 alterations or fewer, 15 alterations or fewer, 10 alterations or fewer, 5 alterations or fewer, 4 alterations or fewer, 3 alterations or fewer, 2 alterations or fewer, or 1 alteration compared with a BC, DE or FG loops, Tn3, CDR, VH domain, VL domain, or scFv sequence disclosed herein.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the present disclosure in diverse forms thereof.

While the present disclosure has been described in conjunction with the exemplary embodiments described above, many equivalent modifications and variations will be apparent to those skilled in the art when given this disclosure. Accordingly, the exemplary embodiments of the present disclosure set forth above are considered to be illustrative and not limiting. Various changes to the described embodiments may be made without departing from the spirit and scope of the present disclosure.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

### EXAMPLES

### Example 1—Materials and Methods

### Solvent-Accessible Surface Area Calculations

The Visual Molecular Dynamics (VMD) software (University of Illinois at Urbana-Champaign) built-in measure sasa command was used to calculate the solvent accessible surface area (SASA) of simeprevir from the three-dimensional structure of HCV NS3/4A PR:simeprevir complex available from the Protein Data Bank (PDB; http://www.rcsb.org/); PDB code 3KEE. The—restrict option, and a radius of 1.4 Å was used to calculate the surface of simeprevir not bound to HCV NS3/4A PR, in other words, the solvent accessible surface area.

### Generation of Biotinylated HCV NS3/4A Protease

The sequence used in the design of HCV NS3/4A PR constructs is derived from Uniprot entry A8DG50 (Hepatitis C virus subtype 1a genome polyprotein) and incorporates additional modifications from US patent U.S. Pat. No. 6,800,456. The protease domain corresponds to residues 1030-1206 of the polyprotein. A single chain consisting of an 11-residue peptide derived from the viral NS4A protein fused to the N-terminus of NS3 protease (SEQ ID 1) was used to create a fully folded and activated polypeptide. This sequence with N-terminal hexahistidine (6His) and AviTag (SEQ ID 3) (to enable affinity purification and biotinylation, respectively) was purchased as a linear DNA string (GeneArt). In parallel, a DNA string encoding an equivalent sequence with the active site mutation S139A (SEQ ID 4) was ordered. The DNA strings were cloned into the pET-28a vector (for bacterial expression) using Gibson assembly. A second set of DNA strings were ordered encoding human codon-optimised versions of the His and Avitag tagged WT and S139A protease and these were cloned into a mammalian expression vector with a CMV promoter. The sequences of the final constructs were verified via Sanger sequencing of the entire coding sequences.

For bacterial expression, the pET-28a plasmids were transformed into BL21 (DE3) E. coli cells and selected on plates containing kanamycin (50 μg/ml). For each expression, a single colony was used to inoculate a 5 ml 2×TY+50 μg/ml kanamycin culture that was grown at 37° C. overnight. This culture was used to inoculate 500 ml TB Autoinduction medium (Formedium, supplemented with 10 ml/L glycerol and 100 μg/ml kanamycin) at 1:500 dilution. The culture was grown at 37° C. to an OD600 of 1.3-1.5 and then transferred to 20° C. for 20 hours for expression to be induced. Cells were harvested by centrifugation and the pellets were stored at −80° C.

For mammalian expression, plasmid DNA was prepared with the Qiagen Plasmid Plus Gigaprep kit. Gigaprep DNA was transfected into Expi293F cells (ThermoFisher) cultured in FreeStyle293 medium (ThermoFisher) using PEI-mediated delivery with cells at a density of 2.5×10⁶ cells/ml at the point of transfection. Cells were cultured at 37° C., 5% CO₂, 140 rpm, 70% humidity for 6 days. Cells were harvested at 4,000 g and pellets stored at −80° C.

For protein purification, each bacterial pellet from 500 ml culture was thawed and re-suspended in 50 ml lysis buffer (2×DPBS, 200 mM NaCl, pH 7.4). The cells were lysed using a probe sonicator and the lysate was clarified by centrifugation at 50,000 g for 40 min at 4° C. Mammalian cell pellets were lysed via resuspension in lysis buffer containing detergent (2×DPBS, 200 mM NaCl, 1 mM TCEP, cOmplete, EDTA-free Protease Inhibitor and 25 U/ml Turbonuclease, 1% Triton X-100, pH 7.4) and rotation at 10 rpm, 4° C. for 2 hours. The mammalian lysed sample was centrifuged at 50,000 g, 30 min, 4° C. All samples were filtered with 0.22 μm bottle-top filtration devices prior to column chromatography. The filtered supernatant was loaded on a 5 ml HisTrap HP column (GE Healthcare) at 5 ml/min flow rate. The column was washed with 100 ml wash buffer (2×DPBS, 200 mM additional NaCl, 20 mM Imidazole, pH 7.4) and eluted with an imidazole gradient over 5 column volumes from 20-400 mM imidazole. Fractions were analysed by SDS-PAGE and those that were enriched for the correct protein were pooled and buffer exchanged with a HiPrep 26/10 Desalting column (GE Healthcare) into lysis buffer (2×DPBS, 200 mM NaCl, pH 7.4). Desalted protein fractions were pooled, concentrated with a centrifugal concentration device and were purified on a HiLoad Superdex 75 26/600 μg column (GE Healthcare) equilibrated in 2×DPBS, 2 mM DTT, 10 μM ZnCl₂. Fractions were analysed by SDS-PAGE and those that were >95% pure were pooled, had their concentration determined via UV absorbance, and were snap frozen in liquid nitrogen prior to storage at −70° C. Final sample purity was verified with RP-HPLC on an XBridge BEH300, C4 (Waters).

The purified protein was biotinylated on its AviTag using an MBP-tagged BirA enzyme incubated with sample for 2.5 hours at 22° C. in the presence of ATP and biotin. Biotinylated protein was purified via size exclusion chromatography on a HiLoad Superdex 75 16/600 μg column (GE Healthcare) in 2×DPBS, 2 mM DTT, 1 μM ZnCl₂. Fractions were analysed by SDS-PAGE and those containing the protease were pooled and the extent of biotinylation was confirmed by intact mass spectrometry on a Xevo G2-CS MS (Waters). Biotinylated protein was split into aliquots, snap frozen in liquid nitrogen and stored at −70° C.

For production of His- and Avitag tagged NS3/4A S139A protease with the introduction of additional mutations either to reduce affinity for simeprevir, the pET-28a derived plasmid encoding the protease was used as a template for site-directed mutagenesis with the Quikchange Lightning site-directed mutagenesis kit. Mutant forms of the protease construct were verified via Sanger sequencing of the entire coding sequences prior to expression. Mutant proteins were transformed into a BL21(DE3) E. coli derivative bearing a plasmid for IPTG-inducible overexpression of BirA biotin protein ligase to enable biotinylation during bacterial expression. An overnight culture was used to inoculate 50 ml 2×TY+50 μg/ml kanamycin at a 1:20 dilution. The culture was grown at 37° C. to an OD600 of 0.6, and then supplemented with 50 μM biotin and induced with 1 mM IPTG. The induced culture was transferred to 25° C. for 20 hours for expression. Cells were harvested by centrifugation and the pellets were stored at −20° C. For purification, each pellet was resuspended in 20 ml lysis buffer (50 mM HEPES, 500 mM NaCl, 1 mM TCEP, cOmplete, EDTA-free Protease Inhibitor) and lysed by passage through a cell disruptor (Constant Systems) at 40,000 kpsi. Protein was purified in an automated 2-step procedure of IMAC followed by buffer exchange with a desalting column. Once loaded on an IMAC resin, sample was washed with lysis buffer supplemented with 20 mM imidazole and eluted with buffer containing 400 mM imidazole. Eluate was automatically captured and loaded on a desalting column equilibrated in 50 mM HEPES, 300 mM NaCl, 0.5 mM TCEP, pH 7.5. Final protein samples were split into aliquots, snap frozen in liquid nitrogen and stored at −70° C.

### HCV NS3/4A PR Protease Activity Assay

To assess enzyme activity, cleavage of a fluorogenic HCV protease FRET substrate with an EDANS-DABCYL donor-quencher pair by purified HCV NS3/4A PR and the S139A mutant (RET 51, AnaSpec) was measured. When in close proximity (10-100 Å), as would be the case for the intact peptide, EDANS is excited at 340 nm, and the energy emitted from EDANS (at 490 nm) is quenched by DABCYL. Cleavage of the peptide by the HCV NS3/4A PR separates DABCYL from EDANS, allowing detection of fluorescence at 490 nm.

Serial dilutions of HCV NS3/4A PR and the active site mutant S139A in assay buffer (HEPES pH 7.8, 5 mM DTT, 100 mM NaCl, 10% glycerol, 0.01% CHAPS) were incubated with the fluorogenic substrate at room temperature. Fluorescence was measured after 3 hours using a PerkinElmer Envision plate reader (excitation 340 nm, emission 490 nm).

### Isothermal Calorimetry

Isothermal calorimetry (ITC) was carried out using the Auto-ITC 200 (Malvern), with a preliminary injection of 0.4 μl followed by 19 injections of 2 μl each, at 120 second intervals. Rotation of the solution was set to 750 rpm and temperature 37° C. Simeprevir (125 μM) was titrated into HCV NS3/4A PR (WT 8 μM and S139A mutant 8.2 μM) or protein buffer (control); the protein buffer was enriched with 2.5% DMSO to equal the amount present in the simeprevir solution. The WT was run once; the S139A mutant was run in duplicate. The data were analysed with the ITC-PEAQ software (Malvern) using a one-site binding model and reference subtraction point-by-point.

### Phage Display Selections

scFv and Tn3 sequences were isolated from phage display selections using three phage display libraries as follows (i) Library 1, a Tn3 library developed as an FnIII alternative scaffold based on the third such module in human tenascin C ((Leahy et al. 1992), (Oganesyan et al. 2013), (Gilbreth et al. 2014)), (ii) Library 2, a restricted framework scFv library and (iii) Library 3 a naïve scFv library.

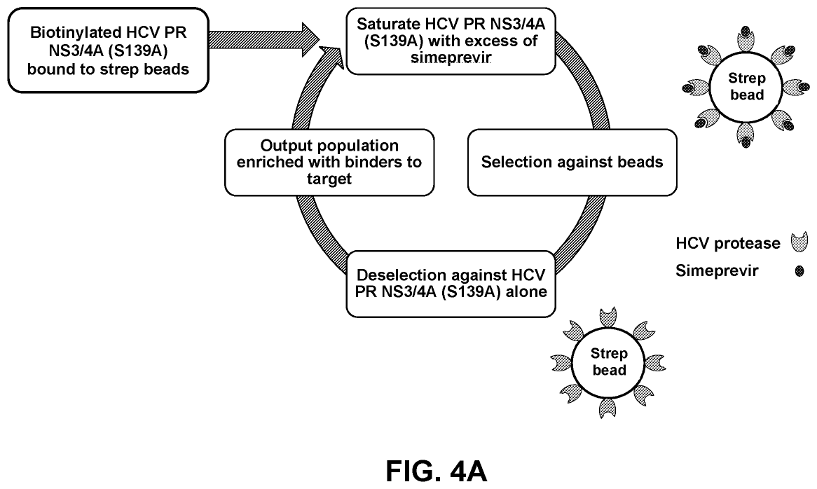

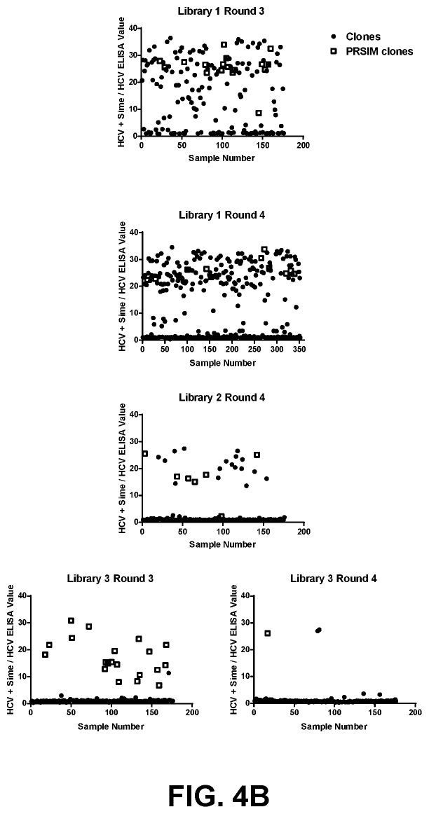

All phage selections were performed according to previously established protocols ((Vaughan et al. 1996), (Swers et al. 2013)). Phage display selections were performed using biotinylated HCV NS3/4A PR (S139A) captured on streptavidin coated magnetic beads (Promega). In total, 4 rounds of phage display selection were performed for each phage library, using decreasing concentrations of biotinylated HCV NS3/4A PR and simeprevir (FIG. 4A and FIG. 4B).

The biotinylated HCV NS3/4A PR (S139A) antigen was pre-incubated with a 50-fold molar excess of simeprevir prior to selections commencing, to ensure saturation of the protease. Prior to each selection, the phage pool was incubated with streptavidin beads alone to deplete the library of any binders to the streptavidin beads. For phage display selections rounds 1 and 2, no deselection step on biotinylated HCV NS3/4A PR (S139A) in the absence of simeprevir was performed. However, for rounds 3 and 4, selections were performed in parallel, with one arm having no deselection step on biotinylated HCV NS3/4A PR (S139A), and the other arm having a deselection step where the phage particles were pre-incubated with 250 nM biotinylated HCV NS3/4A PR (S139A) for 15 minutes at room temperature prior to removing the protease using streptavidin coated beads. Following this the resulting phage were then added to the biotinylated HCV NS3/4A PR (S139A) coated on streptavidin beads in the presence of simeprevir for the selection protocol.

Phage display selections were performed using the following concentrations of biotinylated HCV NS3/4A PR (S139A) at each round:

Round 1: 250 nM biotinylated HCV NS3/4A PR (S139A)+12.5 μM simeprevir

Round 2: 100 nM biotinylated HCV NS3/4A PR (S139A)+5 μM simeprevir

Round 3: 25 nM biotinylated HCV NS3/4A PR (S139A)+1.25 μM simeprevir

Round 4: 25 nM biotinylated HCV NS3/4A PR (S139A)+1.25 μM simeprevir

Following incubation with the biotinylated HCV NS3/4A PR (S139A) in the presence of simeprevir, the phage bound to the complex were washed three times with D-PBS (Sigma) followed by elution with trypsin. Eluted phage were used to infect mid-log phage cultures of E. coli TG1 cells and plated on agar plates (containing 100 μg/ml ampicillin and 2% (w/v) glucose).

Individual phage clones from round 3 and round 4 were picked for DNA sequencing and screening for antigen binding by phage ELISA. DNA sequence information is shown in Table 1.

### Phage Rescue

Specific binding to HCV NS3/4A PR (S139A) was assessed by phage ELISA using single phagemid scFv or Tn3 clones induced for expression as described ((Osbourn et al. 1996)). Briefly, individual TG1 colonies encoding phage clones from round 3 and round 4 selection outputs, and negative control clones, were grown in 96 well plates at 3TC shaking at 280 rpm to log phase in media containing 100 μg/ml ampicillin and 2% (w/v) glucose. Helper phage was then added to each well and the plates incubated at 3TC for 1 hour, shaking at 150 rpm. Plates were then centrifuged at 4500 rpm for 10 minutes at room temperature and the media was removed and replaced with media containing 100 μg/ml ampicillin and 50 μg/ml kanamycin. Plates were then incubated overnight at 25° C., shaking at 280 rpm. The following day, phage preparations were blocked by adding an equal volume of 2×PBS containing 6% (w/v) skimmed milk powder (Marvel) to each well of the plate.

### Phage ELISA

Biotinylated HCV NS3/4A PR (S139A) was used to coat 96 well streptavidin-coated plates at 5 μg/ml (1.875 μM) in the presence and absence of a 3-fold excess of simeprevir (5.6 μM). Coated plates were washed with PBS and blocked with PBS containing 3% (w/v) skimmed milk powder (Marvel) for one hour. Following this blocking step, the plate wells were washed three times with PBS, prior to adding the blocked phage preps (produced as described in the phage rescue section). Phage preps were incubated with the antigens for 1 hour at room temperature prior to washing three times with PBS/Tween 20 (0.1% v/v). Phage that bound specifically to the antigen coated plate were detected using an anti-M13 phage-HRP tagged antibody (GE Healthcare), followed by detection using 3,3′, 5,5′-Tetramethylbenzidine (TMB; Sigma). The detection reaction was stopped using 0.5 M H₂S0₄ and plates were read using a fluorescent plate reader at 450 nm. Fluorescent readings determined for each clone binding to biotinylated HCV NS3/4A PR (S139A) in the presence of simeprevir was compared to those binding in the absence of simeprevir, by dividing the signal observed in the presence of simeprevir to the signal observed in the absence of simeprevir. These data were plotted on graphs (FIG. 4B). From these data, a panel of scFv and Tn3 clones named PRSIM_xx where xx refers to the clone number were selected for further study. Clones that were selected had unique DNA sequences and demonstrated no binding to HCV NS3/4A PR (S139A) in the absence of simeprevir as determined by phage ELISA (except controls PRSIM 51, PRSIM 54, PRSIM 55 and PRSIM 85 which demonstrated binding to the HCV NS3/4A PR (S139A) in both the presence and absence of simeprevir).

Expression of scFv and Tn3 PRSIM Binding Molecules

scFv and Tn3 PRSIM binding molecules were purified from E. coli using methods previously described (Vaughan et al., 1996), using nickel-chelate chromatography, followed by size exclusion chromatography. To increase the expression level of the most promising Tn3 PRSIM binding molecules, the DNA sequences encoding them were subcloned to the pET16b vector, using the oligonucleotides Tn3_pETFwd2 (5′-CGATCATATGGACTACAAGGACGACGATGACAAGGGCAGCCGTCTGGATGCACCGAGCCAG-3′ (SEQ ID NO: 183)) and Tn3_pETRev2 (5′-ATCGGGATCCCTACAGACCGGTTTTAAGGTAATTTTTGCCGG-3′ (SEQ ID NO: 184)) and expressed cytoplasmically in BL21 (DE3) E. coli (New England Biolabs). Following lysis in BugBuster plus Benzonase (EMD Millipore), Tn3-based PRSIM binding molecules were purified to homogeneity using nickel-chelate chromatography, followed by size exclusion chromatography to provide a monomeric protein in PBS (pH 6.5).

### Homogeneous Time-Resolved Fluorescence (HTRF) Binding Screens

scFv and Tn3 PRSIM binding molecules that were selective for the HCV NS3/4A PR (S139A) were identified in homogeneous time-resolved fluorescence (HTRF®) assays run in parallel to measure binding in the presence and absence of simeprevir. HCV NS3/4A PR (S139A), and serial dilutions of purified PRSIM binding molecules, were prepared in assay buffer (PBS containing 0.4 M potassium fluoride and 0.1% BSA). Streptavidin cryptate (Cisbio) was pre-mixed with either anti-FLAG XL665 (to detect the Tn3 molecules) or anti-c-myc XL665 (to detect the scFv molecules) in assay buffer. For each assay 2.5 μl of sample titration was added to 2.5 μl HCV NS3/4A PR (S139A) and 2.5 μl of pre-mixed detection reagents. Either 2.5 μl simeprevir or 2.5 μl of a DMSO blank were also added to each well. Background was defined using wells with zero sample addition. Assay plates were incubated overnight at 4° C., prior to reading the time resolved fluorescence at 620 nm and 665 nm emission wavelengths using a PerkinElmer Envision plate reader. Data was analysed by calculating % Delta F values for each sample. Delta F was determined according to equation 1.

% Delta F=((sample 665 nm/620 nm ratio value)−(background 665 nm/620 nm ratio value)/(background 665 nm/620 nm ratio value))×100 Equation 1

Selective binding molecules are defined as those scFv and Tn3 PRSIM binding molecules that bind to HCV NS3/4A PR (S139A) in complex with simeprevir and no binding to HCV NS3/4A PR (S139A).

### Binding Kinetics Analysis

The affinity of the scFv and Tn3 PRSIM binding molecules were measured using the Biacore 8K (GE Healthcare) at 25° C. The scFv and Tn3 PRSIM binding molecules were covalently immobilised to a CM5 chip surface using standard amine coupling techniques at a concentration of 1 μg/ml in 10 mM sodium acetate pH 4.5.

The HCV NS3/4A PR (S139A), or BSA control, was diluted 1:4 (1.25-20 nM)±10 nM simeprevir in 10 mM Hepes pH 7.4, 150 mM NaCl, 0.05% Surfactant P20, 0.01% DMSO, ensuring constant simeprevir and DMSO concentration. The samples were flowed over the chip at 50 μl/min using single cycle kinetics, with 120 sec association and 600 sec dissociation. The chip surface was regenerated with two 20 sec pulses of 10 mM Glycine-HCl pH 3.0. The final sensorgrams were analysed using the Biacore 8K Evaluation Software and the affinity constant K_{D} was determined using a 1:1 binding model. The same method was used for measuring the affinity of the HCV NS3/4A PR mutants for PRSIM_23 with minor deviations. The mutants were diluted 1:4 (2.5-40 nM)±simeprevir in 10 mM Hepes pH 7.4, 150 mM NaCl, 0.05% Surfactant P20, 0.08% DMSO, ensuring constant simeprevir and DMSO concentration. The samples were flowed over the chip at 50 μl/min using single cycle kinetics, with 180 sec association and 600 sec dissociation.

The effect of simeprevir concentration on the formation of the HCV NS3/4A PR (S139A)/PRSIM binding molecule complex was also measured using the Biacore 8K. PRSIM_57 and PRSIM_23 were covalently immobilised on a CM5 chip surface, as before. Simeprevir was diluted 1:2 (0.0152-300 nM) in 10 mM Hepes pH 7.4, 150 mM NaCl, 0.05% Surfactant P20, 0.3% DMSO at a constant 40 nM HCV NS3/4A PR (S139A) concentration. The samples were flowed over the chip at 50 μl/min using multi cycle kinetics, with 240 sec association and 600 sec dissociation. Regeneration conditions were as described above. Titration curves for the induction of HCV NS3/4A PR (S139A)/PRSIM dimerization by simeprevir were generated. The response for each simeprevir concentration at 225 sec (15 sec before the end of the association) was normalized as a percentage of the response for 300 nM Simeprevir at 225 sec and plotted against the Simeprevir concentration. Each data point represents the mean of 3 independent experiments±s.e.m. The EC₅₀ reported was calculated using nonlinear regression curve fit. The same method was used for the mutant HCV NS3/4A proteases, except simeprevir was diluted 1:2 (0.0457-900 or 0.412-8,100 nM) in 10 mM Hepes pH 7.4, 150 mM NaCl, 0.05% Surfactant P20, 0.82% DMSO at a constant 40 nM HCV NS3/4A PR (S139A) concentration and the response for each simeprevir concentration was normalized against the highest simeprevir concentration.

The affinity of simeprevir was measured using the Octet RED384 (ForteBio) at 25° C. The biotinylated HCV NS3/4A PR (S139A), HCV NS3/4A K136D PR, HCV NS3/4A K136N PR and HCV NS3/4A D168E PR were loaded on High Precision Streptavidin (SAX) biosensors at a concentration of 2 μg/ml in 10 mM Hepes pH 7.4, 150 mM NaCl, 0.05% Surfactant P20, 0.3% DMSO. The simeprevir was diluted 1:1 (46.88-3,000 nM) in the same buffer and the loaded biosensors were dipped into the simeprevir samples for 180 sec to measure the association. For the dissociation the biosensors were dipped into the buffer for 600 sec. The traces were analysed using ForteBio Data Analysis software and fit globally using a 1:1 binding model.

### Split NanoLuc Reconstitution Assay

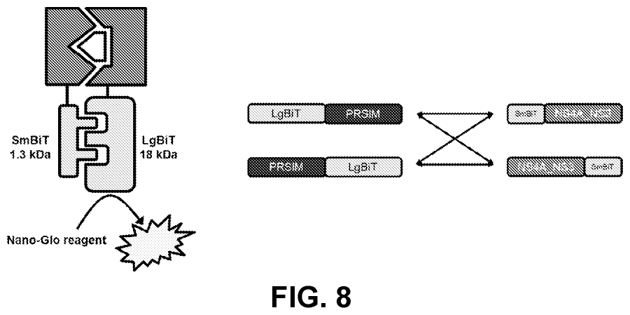

The ability of the PRSIM binding molecules to promote dimerization of two proteins to which they are fused was assessed with the NanoBiT system (Promega) that measures the reconstitution of a split Nanoluciferase (NanoLuc) and the resultant luminescence upon supply of a live cell imaging Nano-Glo NanoLuc substrate (FIG. 8). In the NanoBiT system, one interaction partner is fused via a flexible linker to an 18 kDa fragment of NanoLuc termed LgBiT (for “large bit”) (SEQ ID NO: 16) and the other is fused via an equivalent linker to a 1.3 kDa peptide SmBiT (“small bit”) (SEQ ID NO: 17). LgBit and SmBiT have a low affinity (190 μM) for each other in the absence of interacting partners and will not reconstitute to form an active luciferase enzyme. Once fused to the interacting proteins of a CID and supplied with inducer, they will reconstitute, and luminescence can be measured. The NanoBiT system supplies two sets of control proteins fused to LgBiT and SmBiT: a set of constitutively interacting proteins PRKAR2A:PRKACA; and the FRB:FKBP12 pair whose dimerization can be induced with rapamycin.

To establish the optimal orientation of the HCV NS3/4A PR (S139A) and PRSIM components, constructs whereby HCV NS3/4A PR (S139A) was fused at either the N- or C-terminus to SmBiT (SEQ ID NOs: 18 and 19, respectively) and a parallel set of constructs for each PRSIM binding module fused to either the N- or C-terminus of LgBiT (SEQ ID NOS: 20-30 and 31-41, respectively). The NanoBiT kit (Promega) supplies a set of vectors enabling these constructs to be generated. DNA strings encoding HCV NS3/4A PR (S139A) and the PRSIM molecules were purchased from GeneArt and amplified via PCR with primers with extensions containing restriction sites compatible with the NanoBiT vectors and were cloned via Gibson assembly. All constructs were verified via Sanger sequencing of the entire coding sequence.

All NanoBiT screens were performed in adherent HEK293 cells cultured in 96-well plates. Cells enzymatically dissociated from a tissue culture flask were counted and plated at 2×10⁴ cells/well in a white, opaque-bottomed 96-well plate (Costar 3917). The plates were incubated overnight at 37° C. with 5% CO₂ to allow the cells to adhere. On day 2, plasmids were co-transfected with Lipofectamine LTX (ThermoFisher) at a final concentration of 100 ng/well (50 ng/plasmid, one encoding a SmBiT fusion, the other a LgBiT fusion). On day 3, wells were treated with 100 nM of the appropriate small molecule inducer (rapamycin (FRB:FKBP12) or simeprevir (HCV NS3/4A PR:PRSIM)) or vehicle control, and luminescence was quantified with an Envision plate reader immediately following addition of Nano-Glo Live Cell Substrate (Promega).

### Transcriptional Regulation Assay

The iDimerize regulated transcription system (Takara) was used to test the ability of PRSIM-based CIDs to regulate gene expression. It is based on the reconstitution of a split transcription factor, where the DNA binding domain (DBD) and activation domain (AD) are separated such that transcription does not occur. The DBD and AD are separately fused to the two protein components of a CID such that, only in the presence of the small molecule inducer, the AD is brought into close proximity to the DBD, recruiting the transcription machinery to a promoter harbouring the DBD recognition sites. The iDimerize regulated transcription system (Takara) provides two vectors, pHet-Act1-2 and pZFHD1-Luciferase. The pHet-Act1-2 vector encodes two fusion proteins that represent a positive control: one is a fusion between FRB (T82L mutant; DmrC) and an activation domain (AD) from human p65 (SEQ ID NO: 42); the other is a fusion protein comprised of a DNA binding domain (ZFHD1) (SEQ ID NO: 43) fused to three tandem copies of FKBP12 (DmrA). These sequences are preceded by a CMV promoter and separated by an internal ribosome entry site (IRES). The ZFHD1 vector encodes luciferase preceded by an inducible promoter consisting of 12 copies of the recognition sequence of the ZFHD1 DBD upstream of a minimal IL-2 promoter. Binding of the DBD to its recognition sequence and recruitment of the transcriptional machinery by the AD initiates transcription of the luciferase reporter gene. The DNA sequence encoding HCV NS3/4A PR (S139A) was purchased as a DNA string from GeneArt and cloned into the pHet-Act1-2 vector as either an N-terminal fusion partner to the activation domain (SEQ ID NO: 44) (replacing FRB) or as a C-terminal fusion partner to the DNA binding domain (SEQ ID NO: 45) (replacing FKBP12) with flexible linkers (TGGGGSGGGGS (SEQ ID NO: 185) and SA, respectively) between the fusion partners. Subsequently, sequences encoding one copy of a panel of 12 PRSIM molecules (Table 2) were purchased as DNA strings from GeneArt and were cloned using Gibson assembly into the HCV NS3/4A PR (S139A)-containing pHetAct1-2 constructs described above, as a fusion partner to either the DBD (SEQ ID NOS: 46-56) or AD (SEQ ID NOS: 57-67), respectively. An equivalent construct was generated to replace the three copies of FKBP12 in pHet-Act1-2 with a single copy of FKBP12. The sequence of the constructs encoding both activation domain and DNA-binding domain fusion proteins was confirmed by Sanger sequencing of the entire coding region.

The DNA sequence encoding NanoLuc-PEST (Promega) (SEQ ID NO: 68) was purchased as a DNA string from GeneArt and cloned into the pZFHD1-2 vector (Takara) downstream of the ZFHD1 inducible promoter using Gibson assembly cloning. The nucleotide sequence of the final construct was confirmed by sequencing.

The DNA sequence encoding MED18852 (SEQ ID NO: 237 and SEQ ID NO: 238, separated by an internal ribosome entry site (IRES) sequence) was purchased as a as a DNA string from GeneArt and cloned into the pZFHD1-2 vector (Takara) downstream of the ZFHD1 inducible promoter using Gibson assembly cloning. The nucleotide sequence of the final construct was confirmed by sequencing.

Sequences encoding the three HCV NS3/4A PR (S139A) mutants (Table 6) were purchased as DNA strings from GeneArt and were cloned using Gibson assembly into the pHetAct1-2 HCV NS3/4A PR (S139A)-PRSIM_23 (3 tandem copy) construct described above as a fusion partner to the AD (SEQ ID NOs: 211-216).

All transcriptional regulation assays were performed in adherent HEK293 cells cultured in 384-well plates. Cells enzymatically dissociated from a tissue culture flask were counted and plated at 7.5×10³ cells/well in a 384-well plate. The plates were incubated overnight at 37° C. with 5% CO₂ to allow the cells to adhere. On day 2, the cells were co-transfected with a pHet-Act1-2 plasmid (containing the FRB:FKBP12 control fusion proteins (Clontech) or the HCV NS3/4A PR (S139A):PRSIM fusion proteins) and a pZFHD1 plasmid (encoding either luciferase (Clontech) or NanoLuc-PEST (as described above)) using Lipofectamine LTX (ThermoFisher). On day 3, wells were treated with different concentrations of either A/C heterodimeriser (for the FRB:FKBP12 control), simeprevir or with vehicle control, and 24 hours later luminescence was quantified with an Envision plate reader immediately following addition of SteadyGlo luciferase substrate (Promega) or Nano-Glo Vivazine luciferase substrate (Promega). Alternatively, reverse transfections were carried out on Day 1, addition of dimeriser on Day 2 and luminescence quantified 24 hours later on Day 3.

Luminescence readings were converted into fold-change by dividing the signal in the presence of simeprevir by that in the absence of simeprevir.

For the quantification of antibody expression (MED18852) utilising the transcriptional regulation assay, the cells were co-transfected with a pHet-Act1-2 plasmid (containing the HCV NS3/4A PR (S139A):PRSIM_23) and a pZFHD1 plasmid (encoding MED18852); 24 hours later wells were treated with different concentrations of simeprevir. Antibody concentration was determined in the supernatants 48 hours post the addition of simeprevir using MSD kit (Singleplex Human/NHP IgG Isotyping Kit (Mesoscale).

### Split Chimeric Antigen Receptor Activation Assay

A chimeric antigen receptor (CAR), a synthetic, genetically engineered version of a T-cell receptor, can direct the activation of immune cells in response to user-defined targets via target-specific recognition domains, e.g. a single chain variable antibody fragment (scFv). These multi-domain, synthetic proteins are typically constructed by fusion of the target recognition domain to a transmembrane domain, T-cell receptor co-stimulatory domain and a C-terminal CD3 zeta cytoplasmic activation domain. A split-CAR can be generated by expressing the target recognition/transmembrane/co-stimulatory domain and the CD3 zeta activation domain as two separate proteins. Addition of the appropriate heterodimerising switch components, to the respective proteins, will then allow activation of the CAR in the presence of the target protein via chemical-induced heterodimerisation.

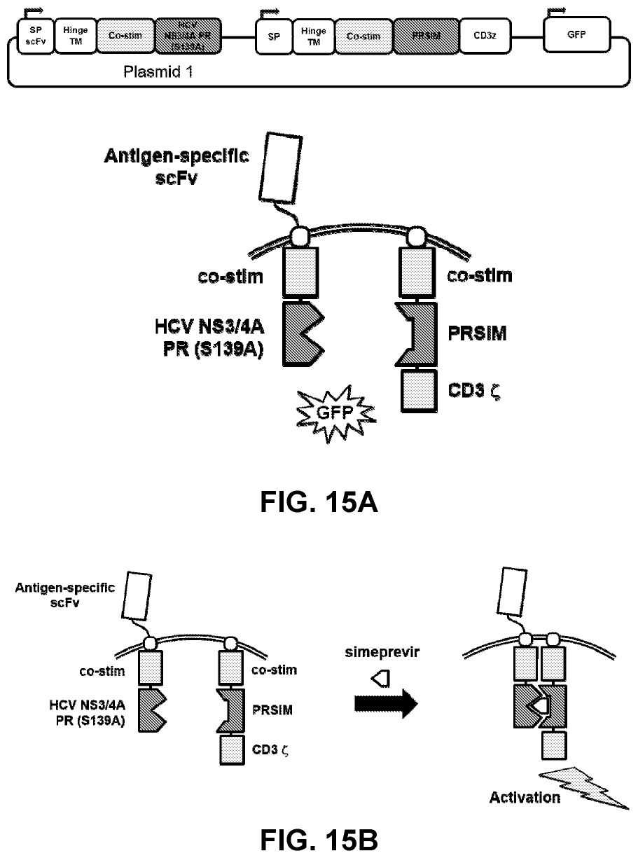

Two split CAR-encoding constructs were generated utilising either the FRB:FKBP12 or HCV NS3/4A PR (S139A):PRSIM_23 heterodimerising components. For both split CARs a tricistronic construct was generated. The three fusion proteins encoded were 1) From N-terminus to C-terminus, a signal peptide sequence, an scFv fragment that recognises the target antigen, a hinge domain from human IgG4, a transmembrane domain from CD28, the intracellular domain of co-stimulatory protein 4-1BB activation domain and either FKBP12 or HCV NS3/4A PR (S139A), 2) From N-terminus to C-terminus, a signal peptide sequence, a hinge domain from human IgG4, a transmembrane domain from CD28, the intracellular domain of co-stimulatory protein 4-1 BB activation domain, either FRB or PRSIM_23, followed by the CD3 zeta domain and 3) green fluorescent protein (GFP) which was used as a marker for transfected cells (FIG. 15A). Fusion proteins 1 and 2 were linked via a P2A self-cleaving peptide, and proteins 2 and 3 were linked via a further T2A self-cleaving peptide. The tricistronic DNA sequences encoding the FRB:FKBP12- and HCV NS3/4A PR (S129A):PRSIM_23-based split CARs were purchased from GeneArt (Life Technologies) and cloned into the pCDH expression lentivector (Systems Bioscience) and sequences were verified by Sanger sequencing. The tricistronic DNA sequences for FRB:FKBP12 split CAR (without the scFv fragment that recognises the target antigen) is provided as SEQ ID NO: 132 and the tricistonic DNA sequence for HCV NS3/4A PR (S139A):PRSIM_23 (also without the scFV fragment that recognises the target antigen) is provided as SEQ ID NO: 133. A DNA sequence encoding the scFv fragment that recognises the target antigen was inserted between nucleotide positions 66 and 67 of SEQ ID Nos: 132 and 133, respectively.

Lentiviral particles encoding each split CAR were generated using the pPACKH1 HIV lentivector packaging kit (Systems Bioscience), according to the manufacturer's protocol. Jurkat cells were transduced with lentiviral particles in the presence of 8 μg/ml polybrene for 24 hours, after which time the cells were changed into fresh growth media (RPMI-1640+10% foetal bovine serum) and allowed to grow for 5 days. Split CAR-transduced Jurkat cell pools were FACS-sorted based on GFP fluorescence to achieve equivalent expression levels for both the FKBP12:FRB and HCV NS3/4A PR (S139A):PRSIM_23 CARs before functional testing. Activation of the split-CAR-expressing Jurkat cells can be measured by interleukin-2 (IL-2) production after stimulation of the CAR (Smith-Garvin, Koretzky, and Jordan 2009). A co-culture assay was employed to facilitate CAR activation whereby CAR-expressing Jurkat cells were mixed with either HepG2 (antigen positive) or A375 (antigen negative) cells at a ratio of 1:1. Different concentrations of simeprevir or the vehicle control (DMSO) was added to the cell mixtures and incubated for 24 hours. Following incubation, the cells are pelleted by centrifugation and the supernatant was tested for IL-2 expression via a commercially-available IL-2 ELISA (R&D Systems) as per the manufacturer's protocol.

### AAV Transduction Experiments

AAV expression vectors were generated by subcloning specific promoter and transgene elements into an intermediate vector derived from pAAV-CMV (Takara) in which the CMV promoter downstream of the 5′ITR was removed and a WPRE element and SV40 polyA sequence were inserted upstream of the 3′ ITR.

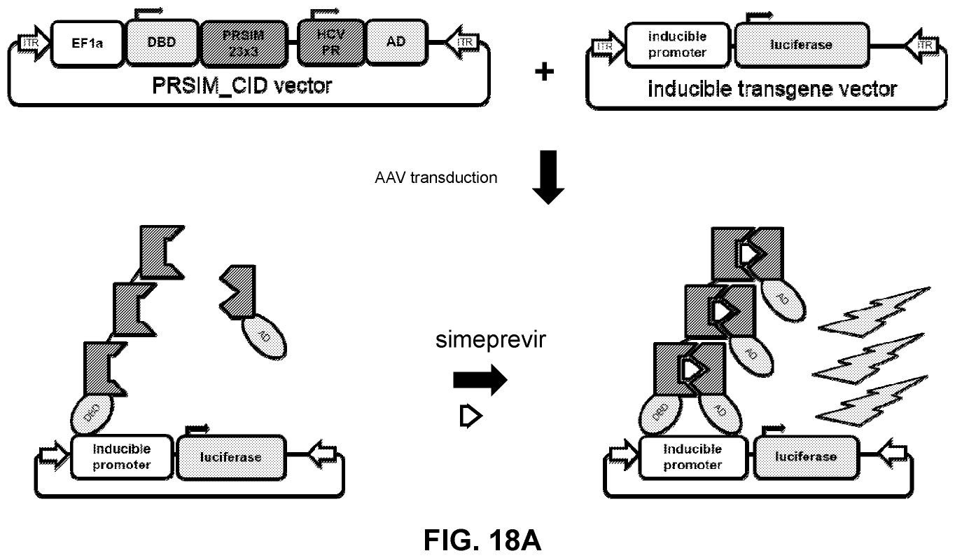

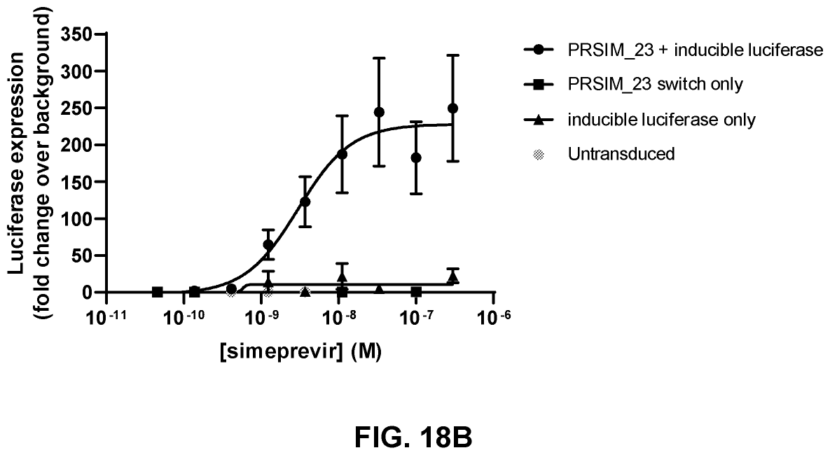

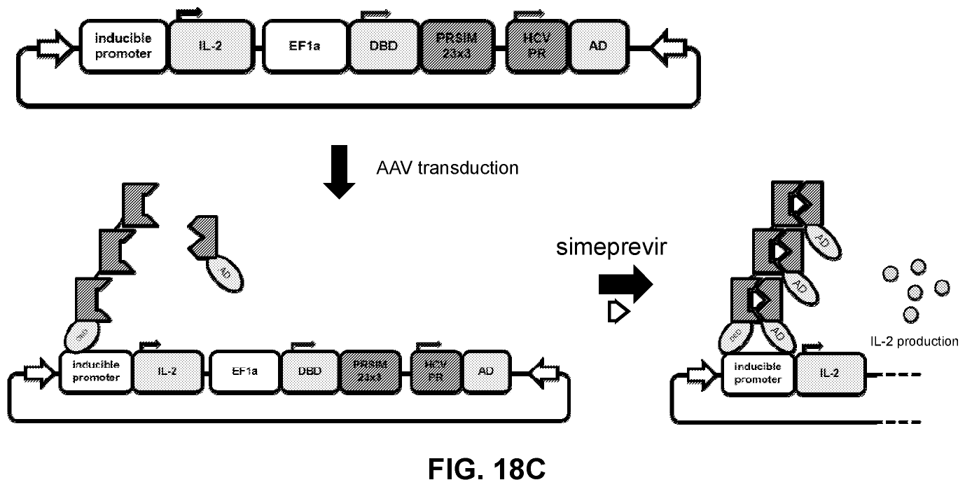

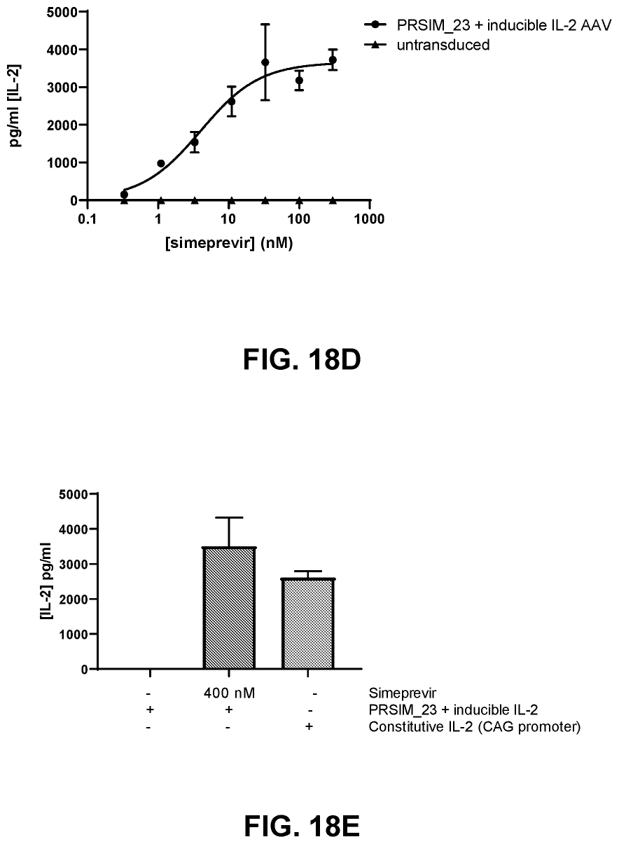

To generate AAV encoding an inducible luciferase transgene, the ZHFD1-luciferase cassette was amplified by PCR from pZFHD1-Luciferase provided in the iDimerize regulated transcription system (Takara) and subcloned into the intermediate AAV vector. To generate AAV encoding constitutively expressed huIL-2, a gene encoding human IL-2 (SEQ ID NO: 210) was subcloned downstream of a CAG promoter in the intermediate AAV vector (FIG. 18A). To generate AAV encoding the PRSIM_23 CID in the context of the split transcription factor, a cassette encoding two fusion proteins (the ZFHD1 DNA binding domain fused to 3 copies of PRSIM_23 and HCV NS3/4A PR (S139A) fused to the AD) separated by a P2A self-cleaving peptide (SEQ ID NO: 208) was subcloned downstream of a hybrid EF1 alpha-HTLV-1 promoter in the intermediate AAV vector. To generate AAV encoding an inducible IL-2 transgene in addition to the PRSIM_23 CID-split transcription factor, human IL-2 was subcloned in place of the luciferase transgene in the pZFHD1-Luciferase vector, and the ZFHD1-huIL-2 cassette was amplified by PCR and inserted immediately downstream of the 5′ ITR in the AAV vector encoding the PRSIM_23 CID-split transcription factor construct (FIG. 18C). All constructs were verified via Sanger sequencing.

Recombinant AAV (rAAV) was produced by triple-transfection of 40 T-175 cm² flasks containing HEK293 T-17 cells at 80% confluency using a standard helper-free approach. Briefly, each flask was transfected with 15 μg of a helper plasmid (a plasmid containing adenoviral E2A and E4), 7.5 μg of the AAV ITR-bearing, and transgene-encoding plasmid and 7.5 μg of the AAV capsid plasmid (containing the AAV8 capsid and the corresponding Rep genes) using 90 μg of 40 kD linear polyethylenimine (PEI). Five days after transfection, media was collected from all the flasks, treated with 2000 units of Benzonase nuclease and incubated at 37° C. for 1 hr. The media was then filtered through a 0.22 μm filter and concentrated to a volume of 80 ml using tangential flow filtration (TFF). This volume was further concentrated and buffer exchanged with PBS using an Amicon-15 ml-100 kDa filter before loading onto a stepwise iodixanol gradient (15%/25%/40%/60%) and spinning at 69000 rpm on an ultracentrifuge in a Ti70 rotor for 1.5 hrs at 18° C. Fractions were taken from the ultraclear centrifuge tubes by piercing the tube with a 19 gauge syringe in the 60% layer below the clear band representing the virus and the purity of each fraction was assessed by SDS-PAGE of each fraction and subsequent Sypro Ruby analysis. Pure fractions were combined, buffer exchanged with PBS in an Amicon-15 ml-100 kDa filter and concentrated to a final volume of 150 μl and stored at −80C in aliquots to avoid any repeated freeze/thaws. The viruses were titered using digital-droplet PCR and a TaqMan probe specific to the ITRs. Typical titres ranged from 1-3×10¹³ genome copies (GC)/ml.

All rAAV transduction assays were performed in adherent HEK293 cells cultured in 96-well plates. Cells enzymatically dissociated from a tissue culture flask were counted and plated at 2.5×10⁴ cells/well in a 96-well plate. The plates were incubated overnight at 37° C. with 5% CO₂ to allow the cells to adhere. On Day 2, the cells were transduced with 2.5-5×10⁹GC/ml (corresponding to a multiplicity of infection (MOI) of 1-2×10⁵) of the relevant rAAV. After incubation for 48-72 hours, the cells were treated with different concentrations of simeprevir or with vehicle control and incubated for a further 24 hours. For luminescence assays, SteadyGlo luciferase substrate (Promega) was added and luminescence was quantified with an Envision plate reader. Luminescence readings were converted into fold-change by dividing the signal in the presence of simeprevir by that in the absence of simeprevir. For IL-2 assays, supernatant was harvested and IL-2 quantified using a V-PLEX Human IL-2 Kit (Meso Scale Discovery) following the manufacturer's protocol.

### Endogenous Gene Regulation Assay

To demonstrate endogenous gene regulation by the PRSIM-based CID, an activating CRISPR (CRISPRa) approach was employed. CRISPRa relies on the use of a dead Cas9 enzyme (dCas9) with no endonuclease activity to bind to a target site within the promoter region of an endogenous gene via a single guide RNA. Upon recruitment of a transcriptional activator, transcription of the endogenous gene is initiated.

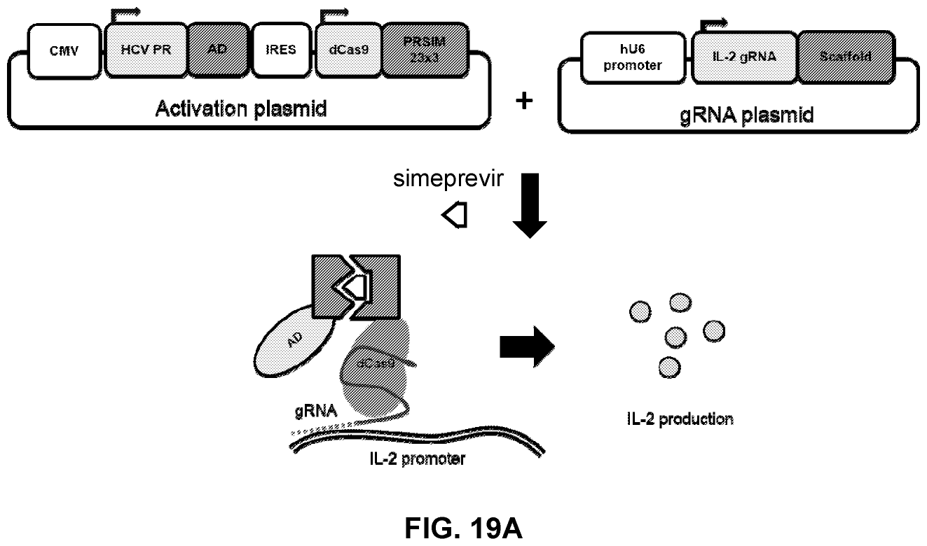

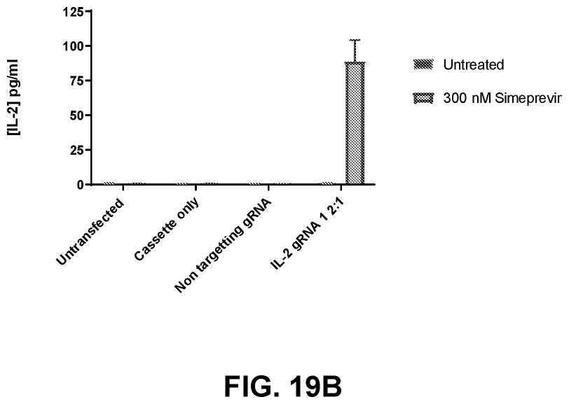

For this approach, the dCas9 and the VPR activation domain (AD) are separated such that transcription does not occur. The dCas9 and AD are separately fused to the two protein components of the CID such that, only in the presence of the small molecule inducer, the AD is brought into close proximity to dCas9, allowing recruitment of the transcription machinery to the promoter region of an endogenous gene via a single guide RNA (sgRNA). In this example, an activation plasmid was generated consisting of two functional units; an AD fused to the HCV NS3/4A PR (S139A) (SEQ ID 226) and a dCas9 fused to three tandem copies of PRSIM-23 (SEQ ID 228). The sequences are preceded by a CMV promoter and separated by an internal ribosome entry site (IRES). A gRNA plasmid was generated by golden gate assembly, utilising BsaI. The gRNA plasmid encodes the human U6 promoter, an interleukin-2 (IL-2) target sequence (GTTACATTAGCCCACACTT; SEQ ID NO: 229) and a scaffold RNA sequence to allow Cas9 binding (FIG. 19A).

Transcriptional regulation assays were performed in adherent HEK293 cells cultured in 96-well plates. Cells enzymatically dissociated from a tissue culture flask were counted and plated at 2.5×10⁴ cells/well. The plates were incubated overnight at 37° C. with 5% CO₂ to allow the cells to adhere. On day 2, the cells were co-transfected with the activation and gRNA plasmids using Lipofectamine 3000 (ThermoFisher), using a gRNA:activation plasmid DNA ratio of 2:1. On day 3, wells were incubated with 300 nM simeprevir or with vehicle control. 72 hours post-treatment (day 6), the cell supernatant was harvested and IL-2 quantified using a V-PLEX Human IL-2 Kit (Meso Scale Discovery), as per the manufacturer's protocol.

### Molecular Simulations to Identify Mutations Predicted to Reduce the Affinity of Simeprevir to Hepatitis C Virus (HCV)) to NS3/4A Protease

The co-crystal structure of HCV in complex with Simeprevir was first prepared using Protein Preparation Wizard (Sastry et al., 2013) to add hydrogen atoms, fill in missing side chains, assign the proper ionization state for both the amino acids and Simeprevir at physiological pH. The FEP+ (module) in the Schrödinger 2019-2 (Moraca et al., 2019) release with the OPLS3e force field was then used to predict the relative binding free energies upon mutations of residues H57, K136, S139 and R155 in HCV NS3/4A PR. Mutations that are predicted to reduce the affinity of HCV protease to Simeprevir are listed in Table 4.

### Generation of Stable Cell Lines Expressing GFP-PEST Under Control of Split Transcription Factor

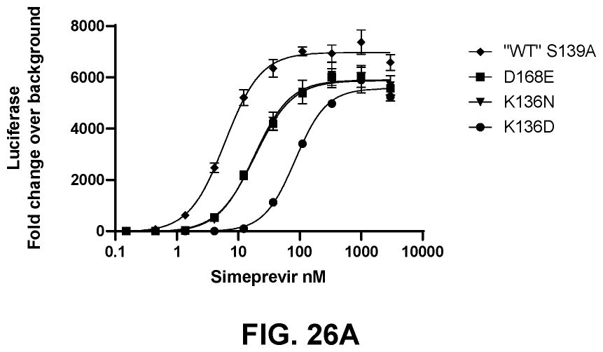

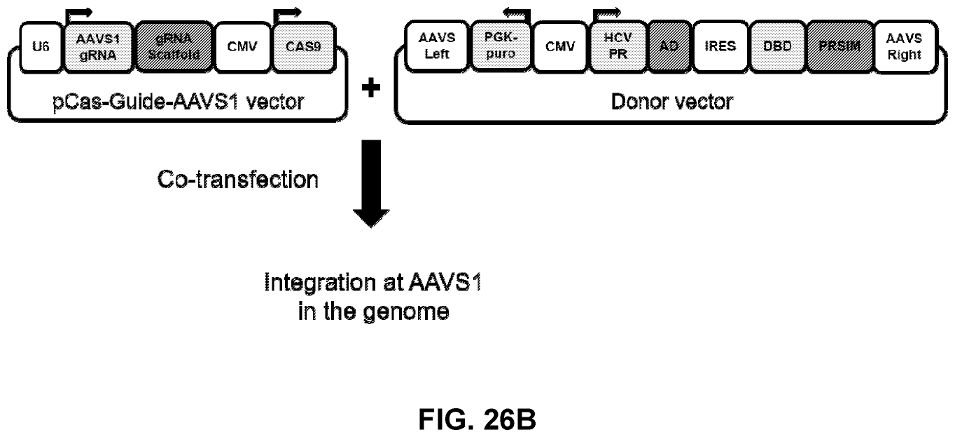

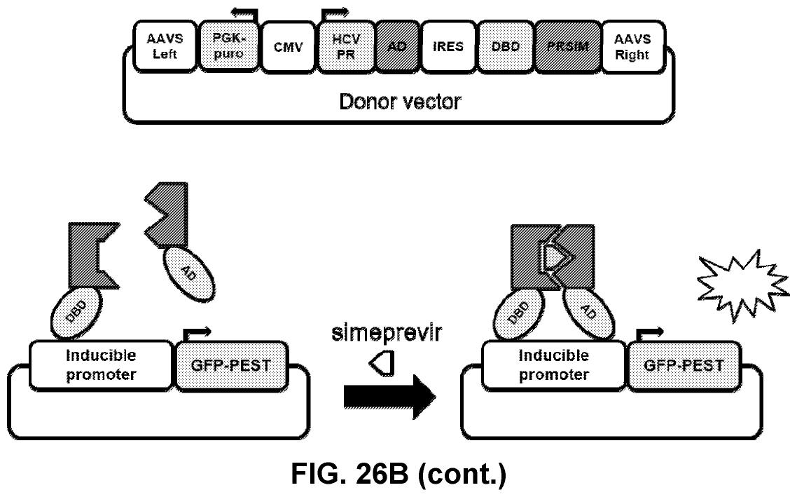

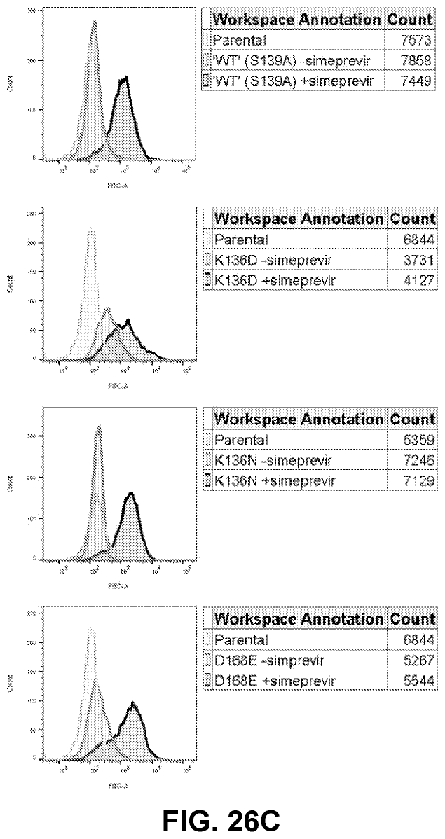

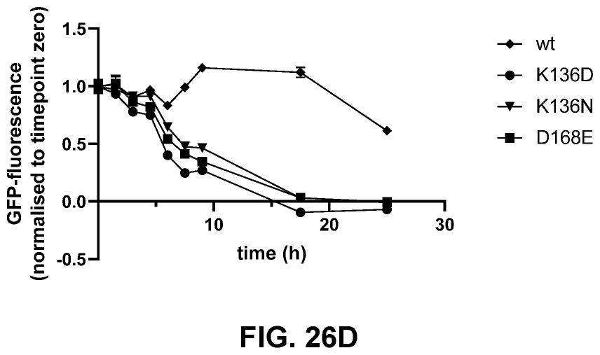

Monoclonal cell lines were generated using CRISPR-mediated knockin system for transgene integration at AAVS1 locus (ORIGENE) according to manufacturer's instructions (FIG. 26B). Initially, HEK293 cells expressing GFP-PEST (SEQ ID NO: 232, 233) under control of inducible promoter (minimal IL-2 promoter) were obtained by transient transfections with previously linearized pHet-ZFHD1-GFP-PEST plasmid. Transfected cells were selected by addition of 800 μg/ml geneticin into growth media (DMEM+10% foetal bovine serum+1% Non-essential amino acids). Subsequently, polyclonal cells were transfected with pHet-Act1-2-HCV NS3/4A PR (S139A)-PRSIM23 (3 tandem copies) plasmid and FACS sorted based on GFP fluorescence intensity in response to simeprevir treatment to isolate single cell clones. Final monoclonal cell line was used as a base for further generation of HEK293 cells expressing GFP-PEST under control of split transcription factor PRSIM_23 HCV NS3/4 PR WT and mutants.

AAVS1 safe harbor CRISPR-mediated knockin system employs two plasmids: the CRISPR all-in-one vector, pCAS-Guide-AAVS1 vector and the donor vector (pAAVS1-DNR-Puromycin) with AAVS1 homologous arms (SEQ ID NO: 234, 235). The AAVS1 targeting sequence (SEQ ID NO: 236) was previously cloned into pCAS-Guide plasmid. The donor vector was engineered by addition of SbfI and HpaI restriction enzyme sites via Gibson assembly to enable further sub-cloning of HCV NS3/4A PR (S139A) and mutants:PRSIM_23 heterodimerising components. Subsequently, pHet-Act1-2-HCV NS3/4A PR (S139A)-PRISM23 (3 tandem copies) plasmid was digested with SbfI and HpaI restriction enzymes (New England Biolabs) to obtain HCV NS3/4A PR (S139)-PRISM23 DNA which was further sub-cloned into the donor vector by Gibson Assembly. HCV NS3/4A PR variants including HCV NS3/4 PR (K136D) (SEQ ID NO: 211), HCV NS3/4 PR (D168E) (SEQ ID NO: 213) and HCV NS3/4 PR (K136N) (SEQ ID NO: 215) were sub-cloned from pHet-Act1-2-HCV NS3/4 PR (K136D/D168E or K136N)-PRISM23 into pAAVS1-HCV NS3/4A PR (S139A)-PRISM23-Puromycin plasmid by Gibson assembly using SbfI and AfeI restriction enzyme sites. The nucleotide sequences were confirmed by Sanger sequencing.

Stable cells expressing GFP-PEST under control of inducible promoter alone were co-transfected with pAAVS1-HCV NS3/4A PR (S139A; K136D; D168E; K136N)-PRISM23-Puromycin donor vector and pCAS-Guide-AAVS1 to enable targeted integration into AAVS1 locus. Transfected cells were selected by addition of 1 ug/ml puromycin into growth media (DMEM+10% foetal bovine serum+1% Non-essential amino acids+800 μg/ml Geneticin) 48 hr post-transfection. Following 14 day selection period, polyclonal cell lines were induced with 500 nM simeprevir and FACS sorted based on GFP fluorescence intensity to isolate single cell clones. Final monoclonal cell lines (FIG. 26C) were FACS characterised based on GFP signal in response to 500 nM simeprevir treatment.

Flow Cytometry to Determine the Kinetics of GFP-PEST Expression from the Simeprevir-Inducible Switch

Monoclonal cell lines expressing GFP-PEST under the control of the split transcription factor system were enzymatically dislodged from tissue culture flasks and plated into 96 well collagen-coated plates. The following day, cells were treated with 100 nM Simeprevir. 24 h post-treatment, cells were washed twice in growth medium without Simeprevir, and cells were further maintained in medium without Simeprevir. Cellular GFP-fluorescence at various timepoints after the removal of Simeprevir was determined using flow cytometry on a Fortessa Flow cytometer (BD Biosciences). For analysis, the GFP-fluorescence (in relative fluorescence units=RFU) of untreated cells was subtracted from all experimental values. RFU values were further normalised to timepoint ‘0 h’, taken at the time of Simeprevir removal.

### Structure Determination of the HCV NS3/4A PR (S139A):PRSIM 57 Complex

The single chain HCV protease construct—an 11-residue peptide derived from the viral NS4A protein fused to the N-terminus of NS3 protease with S139A mutation—was redesigned with an N-terminal hexahistidine (6His) followed by a tobacco etch virus (TEV) protease cleavage site (to enable affinity purification and removal of the tags, respectively) (SEQ ID NO: 218). A second construct was designed to express the PRSIM_57 scFv with an N-terminal pelB leader to direct periplasmic secretion and C-terminal TEV site and 6His tag (SEQ ID NO: 221). Both sequences were purchased as linear DNA strings (GeneArt) and were cloned into the pET-28a vector (for bacterial expression) using Gibson assembly. The sequences of the final constructs were verified via Sanger sequencing of the entire coding sequences.

For expression, the pET-28a plasmids were transformed into BL21(DE3) E. coli cells and selected on plates containing kanamycin (50 μg/ml). For each expression, a single colony was used to inoculate a 5 ml 2×TY+50 μg/ml kanamycin culture that was grown at 37° C. overnight. This culture was used to inoculate 500 ml TB Autoinduction medium (Formedium, supplemented with 10 ml/L glycerol and 100 μg/ml kanamycin) at 1:500 dilution. The culture was grown at 37° C. to an OD600 of 1.3-1.5 and then transferred to 25° C. (HCV NS3/4A PR (S139A)) or 30° C. (PRSIM_57) for 20 hours for expression to be induced. Cells were harvested by centrifugation and the pellets were stored at −80° C.

For protein purification of HCV NS3/4A PR (S139A), each bacterial pellet from 500 ml culture was thawed and re-suspended in 50 ml lysis buffer (50 mM HEPES, 500 mM NaCl, 1 mM TCEP, pH 8.0). The cells were lysed by passage through a cell disruptor at 30,000 kpsi and the lysate was clarified by centrifugation at 50,000 g for 30 min at 4° C. The clarified supernatant was loaded on a 5 ml HisTrap HP column (GE Healthcare) at 5 ml/min flow rate. The column was washed sequentially with wash buffers (50 mM HEPES, 500 mM NaCl, 1 mM TCEP, 20 mM Imidazole, pH 8.0 and 50 mM HEPES, 500 mM NaCl, 1 mM TCEP, 40 mM Imidazole, pH 8.0) and eluted with an imidazole gradient over 5 column volumes from 40-400 mM imidazole. Fractions were analysed by SDS-PAGE and those that were enriched for the correct protein were pooled and buffer exchanged with a HiPrep 26/10 Desalting column (GE Healthcare) into 50 mM HEPES, 200 mM NaCl, 0.3 mM TCEP, 10 μM ZnCl₂, pH 7.5 (storage buffer). Desalted protein fractions were treated with His-tagged TEV protease at 1:100 w/w overnight at 4° C. TEV protease was removed by passing the sample through a HisTrap HP column and the resulting flow-through material was polished by loading on a Superdex 75 26/600 column equilibrated in storage buffer.

The PRSIM-57 His-tagged scFv sample was released from the periplasm via osmotic shock of the cell pellets: cells were first resuspended in 300 ml 50 mM Tris, 1 mM EDTA, 20% sucrose, pH 8.0 and then pelleted and resuspended in water to exert osmotic shock and release periplasm contents. The sample was purified by loading on a HisTrap excel column and washing and eluting with the same buffers as used for the HCV NS3/4A PR (S139A) construct. The eluted protein was buffer exchanged by loading on a HiPrep 26/10 desalting column in 50 mM HEPES, 200 mM NaCl, pH 7.5 and treated with TEV protease at 1:50 w/w ratio overnight at 4° C. TEV-digested material was further purified with IMAC and size exclusion steps as for the protease and stored in 50 mM HEPES, 200 mM NaCl, pH 7.5.

To form the ternary complex of HCV NS3/4A PR (S139A), PRSIM_57 and simeprevir, HCV NS3/4A PR (S139A) at a concentration of 50 μM was mixed with a 1.1-fold excess of PRSIM_57 and to this was added simeprevir to a final concentration of 100 μM with DMSO at 3% in the final solution. The sample was incubated at room temperature for 60 min to allow equilibration and then loaded on Superdex 75 16/600 column at 0.75 ml/min in 20 mM HEPES, 200 mM NaCl, pH 7.5. Fractions containing the complex were pooled, concentrated to 12 mg/ml, split into aliquots and snap frozen in liquid nitrogen prior to storage at −70° C. An aliquot of the complex was thawed and run on an HP-SEC column to verify complex integrity and monodispersity prior to crystallisation.

The ternary complex was crystallised using sitting drop vapour diffusion method. A number of proprietary crystallisation screens were set up at 277K and 293K. Hits from these screens were optimised using sitting drop and hanging drop vapour diffusion experiments as appropriate. Final crystals were obtained at 293K from reservoir solutions comprised of 20-25% (w/v) PEG 8000, 100-300 mM magnesium chloride and HEPES buffer, pH 7.0-8.0. Crystals were exposed to cryoprotectant solution of reservoir supplemented with 20% (v/v) ethylene glycol and then frozen directly in liquid nitrogen.

Data collection was carried out at Diamond Light Source, beamline i04, at cryogenic temperatures. The CCP4 and autoBUSTER software packages were used to solve and refine the structures, and the program Coot was used for manual building of the models. The structure was solved by molecular replacement using models of HCV NS3/4A (S139A) from the Protein Data Bank.

### In Silico Prediction of Stability of HCV NS3/4A PR (S193A) Mutants

The change in stability of the HCV protein upon mutation was calculated using Schroedinger Residue Scanning tool (Schrödinger Release 2020-2: SiteMap, Schrödinger, LLC, New York, N.Y., 2020).

A Prime MM/GBSA energy function with an implicit solvent term was used for the calculations (Li et al., 2011). A cutoff of 6 Å was used for the protein refinement around the mutation. Negative values of the change in stability are linked to an increased mutant stability.

### PRISM-Based Kill Switch Cloning

The sequence encoding a kill switch fusion protein of PRSIM23, HCV NS3/4A PR and ΔCARDCaspase9 with short GGGSG between the three fragments (SEQ ID NO: 223) was purchased as a cloned gene in vector pcDNA3.1 from Geneart (Life Technologies). The fusion protein was sub-cloned into EcoRI/NotI digested lentiviral vector pCDH-EF1α-MCS-(PGK-GFP-T2A-Puro) (Systems Bioscience) using Gibson assembly cloning. To generate the Caspase 9 S196A mutation, a DNA fragment altering the equivalent Ser371 in the kill switch construct to Ala was synthesized by Geneart was cloned into ClaI/NotI cleaved kill switch vector (SEQ ID NO: 230). Gene sequences were confirmed by DNA sequencing.

### PRISM-Based Kill Switch Cell Line Generation

Lentiviral particles encoding the kill switch fusion protein (SEQ ID NO: 223) or kill switch S196A mutant fusion protein (SEQ ID NO: 230) were generated using pPACKH1 HIV lentiviral packaging kit (Systems Bioscience), according to manufacturer's instructions. HEK293 cells were transduced for 24 h in the presence of 8 μg/ml polybrene after which cells were changed into fresh growth medium (DMEM+10% foetal bovine serum+1% Non-essential amino acids). 24 h later transduced cells were selected by addition of 2 μg/ml puromycin for 5 days. Before functional testing, transduced cell pools were FACS sorted based on GFP fluorescence to isolate high expressing cell line pools and single cell clones.

HCT116 and HT29 transduced cells were generated following the same protocol with exception of using McCoy's 5A medium+10% foetal bovine serum as growth medium, supplemented with 2 μg/ml puromycin for selection of transduced cells.

The hESC line Sa121 (TakaraBio Europe) was also transduced with the lentiviral particles encoding the PRSIM-based kill switch fusion protein described above (SEQ ID 223). Cells (passage 19) were plated at 3.5×10⁵ cells/cm² in the DEF-CS culture system, and the cells were transduced 30 h later. 24 h after transduction, puromycin selection was initiated and the antibiotic selection was maintained until a stable cell pool was achieved.

### Generation of Stable iPS Cell Lines Expressing the PRSIM-Based Kill Switch

A stable induced pluripotency stem cell (iPSC) line (a single clone (B-3/1F1) derived from the fibroblast cells of a healthy human donor from Research Specimen Collection Program of Astrazeneca) stably expressing the simeprevir-inducible kill switch was generated using CRISPR/Cas9 technology, using AAV-encoded DNA as template for targeted integration into the β2 microglobulin (B2M) locus.

The donor construct (FIG. 33A) encoding the PRSIM-based kill switch (SEQ ID 223) was synthesized and purchased from GenScript, Inc. and was subcloned into an AAV shuttle plasmid backbone. The donor construct was packaged into adeno-associated viral (AAV) particles; briefly, the donor plasmid was co-transfected with two helper plasmids, pAd5Helper and pR2C6, encoding adenoviral components essential for AAV replication and AAV2 replication (rep)/AAV6 capsid (cap) proteins respectively. After 72 hours, the cells were collected and were disrupted by freeze-thawing. The cell lysate was digested with Benzonase (100 U/ml) for 1 hour at 37° C. and were centrifugated. The vector-containing supernatant was collected and applied to Iodixanol gradient followed by ultracentrifugation. After ultracentrifugation, the vector containing solution was collected and washed with 20 mL PBS in a centrifugal concentrator tube for 3 times. Finally, the solution was concentrated to 1 mL. The vector genome copies contained in the solution was titered by qPCR.

The iPSC cells seeded in Vitronectin-coated 6-well plates at 50-70% confluency (approximately 1.2×10⁶ cells) were used for transfection/transduction. Cells were maintained in 2 mL fresh StemFlex medium containing 1× RevitaCell (Life Technologies). For each well, 200 μL Opti-MEM (Life Technologies) medium containing 220 nM of CRISPR-ribonucleoprotein and 12 μL of RNAiMAX (Life Technologies) was applied. In the meantime, the AAV vectors were applied with the multiplicity of infection (MOI) of 50,000. After 24-hour incubation, the RNP/AAV-containing medium was replace by fresh StemFlex medium.

At 48-hour post transfection, the medium was replaced by fresh StemFlex medium containing 5 μg/mL Blasticidin S HCl (Life Technologies). The medium was replaced with fresh Blasticidin-containing StemFlex medium each day for another 3 to 4 days. Then, the cells were maintained in regular StemFlex medium again.

To identify cells that were B2M-negative, and thus encoding the PRSIM-based kill switch, FACS was performed. Cells were detached from the plates using TrypLE Express (Life Technologies) and resuspended in FACS buffer (HBBS containing 1% PBS and 1× RevitaCell) at a density of 1×10⁷ cells/mL containing 5% of APC-labeled anti-human B2M antibody (BioLegend, Inc.) solution. After 10-minute incubation, cells were washed using 10 times volume of FACS buffer for two times and resuspended in FACS buffer at a density of 2×10⁷ cells/mL. B2M-negative cells were collected by FACS (FACSAria; BD Biosciences) and cultured for further experiment.

Single cell clones were then isolated using single-cell printing. Cells were detached from the plates using TrypLE Express (Life Technologies) and resuspended in SCP buffer (HBBS containing 1× RevitaCell) at a density of 1.6×10⁶ cells/ml. Cell suspension was loaded to a cartridge of the Cytena CloneSelect Single-Cell Printer (Cytena). Cells were seeded at 1 cell per well in the Matrigel or Vitronectin-coated 96-well plates containing 200 μL of mL fresh mTeSR (STEMCELL Technologies) or StemFlex medium containing 1× RevitaCell (Life Technologies). The media was replaced by fresh StemFlex media on the next day of SCP.

Five single cell clones were recovered, expanded from 96-well plates to Vitronectin-coated 24-well plates, and were further expanded and maintained in Vitronectin-coated 6-well plates. For each single-cell clone, approximately 5×10⁵ cells were collected. Genome DNA was isolated using the DNeasy Blood & Tissue Kit (Qiagen). The targeted region of human B2M gene were amplified using the primers below and using the SuperFi DNA polymerase (Life Technologies). The PCR products were loaded in 1.2% agarose gel for electrophoresis. The gel were visualized to identify the gene knock-in status of the single-cell clones by the size of the amplicons (FIG. 33B). Clones 1B7, 1D12, 1G8 and 2D8 were shown to be biallelic at the B2M locus, and were used for functional analysis of the kill switch activity.

B2M_LHA_PF2
(SEQ ID NO.: 246)
GGGAGGAACTTCTTGGCACA
B2M_RHA_PR2
(SEQ ID NO.: 247)
AGGAGAGACTCACGCTGGAT

### Kill Switch Cell Viability and Caspase 3 Functional Assay

HEK293, HCT116 or HT29 cells stably expressing the PRSIM-based kill switch fusion protein (SEQ ID NO: 223) or HEK293 cells stably expressing the PRSIM kill switch S196A mutant fusion protein (SEQ ID NO: 230) were plated onto collagen-coated 96-well plates and 24 h later treated with 100 nM simeprevir. Phase contrast images were acquired at various timepoints using 10× or 20× objectives on an Incucyte Zoom (EssenBioscience).

Functional Caspase 9 activates Caspase 3, and this proteolytic activity can be determined using cleavage of non-fluorescent substrate DEVD-AMC into cleavage products DEVD and fluorescent AMC, such that AMC fluorescence signal at 430 nm is proportional to Caspase 3 activity. For the Caspase 3 assay, cells were plated in duplicate onto 6 well tissue-culture treated plates. 24 h later, one of the duplicate wells was treated with 10 nM simeprevir for 3 h. Cells lysates were analysed in triplicate using a Caspase 3 assay from BD Biosciences according to manufacturer's instruction with the modification that total protein input was normalised to 50 μg by BCA assay (LifeTechnologies). Fluorescence was determined on an Envision plate reader (PerkinElmer), Ex: 380 nm, Em: 430 nm. For quantification, RFU (raw fluorescence value) for wells that contained only the assay substrate were subtracted from all RFU derived from assay samples. Results were normalised to non-transduced, simeprevir-treated cells. Analysis was performed in Prism (GraphPad) using a One-Way-Anova followed by multiple comparisons.

### PRSIM-Based Kill Switch Activity in ESC Cells

To test the induction of the kill switch in Sa121 ES cells, the cells were plated at 3.5×10⁵/cm² two days before inducing kill switch activity by treating with simeprevir at concentrations from 10 nm to 1 uM. The cells were imaged using the Incucyte S3 (EssenBioscience) at intervals ranging from 10-20 min; kill switch efficiency was quantified by image analysis of confluency.

### Real-Time Cell Analysis (RTCA) Assay to Detect Simeprevir-Inducible Kill Switch Activity in iPS Cells

The cells for each of the single-cell clones described above were plated in the vitronectin-coated 96-well electronic microtiter plates (E-Plate® 96, ACEA Biosciences Inc.) at the density of 40,000 cells per well. The plate was set connected with the xCelligence module and incubated at 37° C. in a humidified incubator with 5% CO2 so that the cell proliferation index can be monitored without interrupting regular cell growth. The cell proliferation index was measured and recorded every 15 minutes for 24 hours. Simeprevir at different concentration was then added and the cell proliferation index measured every 5 minutes for 8 hours and then every 15 minutes for further 40 hours. All experiments were performed in triplicate wells for each clone and each condition. The average cell index were quantified by using the xCELLigence RTCA Software Pro (ACEA Biosciences Inc.)

### Example 2—Identification of Simeprevir and HCV NS3/4A PR as the Basis for a Chemical Inducer of Dimerization (CID) Module

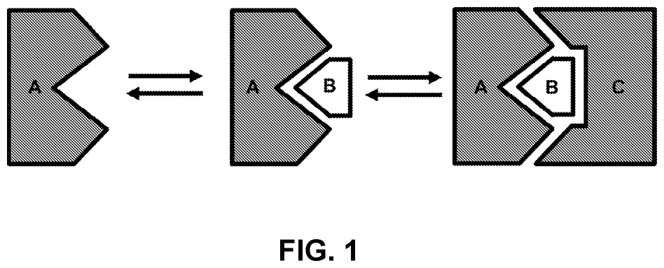

To generate a de novo chemical inducer of dimerization module, we adopted an approach whereby the small molecule inducer is a clinically approved small molecule and one of the protein components is the target of the small molecule (target protein). The second protein component (binding member) is derived from a library of binding molecules (Tn3 or scFv) and demonstrates exquisite selectivity for the target protein bound to the small molecule over the unbound target protein (FIG. 1). By focusing on approved small molecules, we reasoned that the path to regulatory approval would be significantly smoother considering that the small molecules are already considered safe for human use, at appropriate doses. Rather than use small molecules that target human proteins, we decided to focus instead on small molecules that bind to non-human proteins such as anti-viral compounds. We reasoned that the advantages of this approach are that the small molecule will not elicit any on-target pharmacology which could be detrimental and, as there is no target protein present in (uninfected) patients, there is no competition for binding to the small molecule which could impact its pharmacokinetics. To determine a preferred small molecule/target protein pair we considered the following criteria:

Ideal small molecule criteria:
Approved
Suitable for chronic dosing (daily for >6 months)
Cell permeable
Orally dosed
Not used as first line therapy as an antiviral

Ideal target protein criteria:
Monomeric
Small (≤30 kDa)
Overexpression of target protein (or domain thereof) is non-toxic OR target protein can be rendered inactive but retain SM binding
Can be expressed cytoplasmically (i.e. not integral to membrane or bound to DNA)

Small molecule:target protein complex criteria:
There is reason to believe that the bound target protein will have epitopes that are distinct from the unbound target protein

An extensive analysis was carried out, and one of the preferred small molecule/target protein pairings identified was simeprevir and its target, the NS3/4A protease from hepatitis C virus (HCV NS3/4A PR). Simeprevir (Olysio®) is a small molecule that is administered orally, is cell-permeable, and has a pharmacokinetics (PK) profile that supports once-daily dosing. It has been used chronically (up to 39 months) to treat HCV infection in combination with ribavirin and pegylated interferon, and is on the WHO essential medicines list, indicative of a well-tolerated and widely administered drug. HCV NS3/4A PR is monomeric, relatively small in size (21 kDa), can be expressed cytoplasmically, and is not found associated with DNA. Furthermore, three-dimensional X-ray crystallography of the complex (PDB code: 3KEE) reveals that simeprevir is bound in the shallow substrate-binding groove of HCV NS3/4A PR with 364 Å of exposed surface area (FIG. 2); we reasoned that this relatively large exposed area would be sufficiently different from the unbound HCV NS3/4A PR that complex-specific binding molecules could be identified.

### Example 3—a Mutant HCV NS3/4A PR (S139A) Retains Binding to Simeprevir, Despite a Significant Reduction in Activity

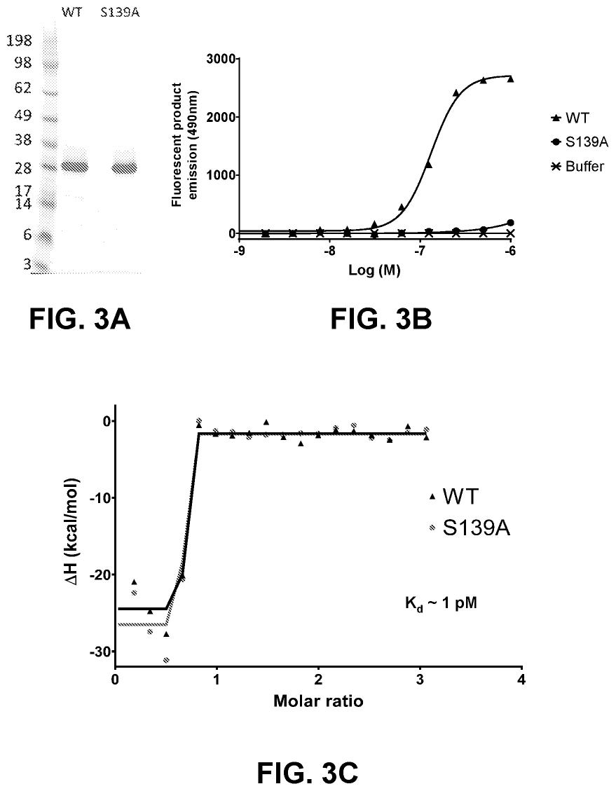

The HCV NS3/4A PR is an enzyme that cleaves at four junctions of the HCV polyprotein precursor, and it is known to cleave a limited number of endogenous human targets (Li, Sun, et al. 2005; Li, Foy, et al. 2005). To limit this activity within human cells, we reasoned that identification of a mutant form of the HCV NS3/4A PR that is enzymatically inactive but retains binding to simeprevir would be necessary. An active site mutant of HCV NS3/4A PR (S139A) had previously been shown to demonstrate significantly less activity than its wild-type counterpart (Sabariegos et al. 2009). To confirm this, and to investigate whether the mutant HCV NS3/4A PR would retain binding to simeprevir, recombinant proteins were expressed in E. coli and purified to homogeneity. HCV NS3/4A PR with an N-terminal hexahistidine and AviTag, both WT (SEQ ID NO: 3) and S139A mutant (SEQ ID NO: 4) were expressed separately in 1 litre culture of BL21(DE3) induced via autoinduction. The cultures were harvested and proteins purified using a combination of immobilised metal affinity chromatography and size exclusion chromatography. Final pooled samples were assessed via SDS-PAGE indicating a >99% level of purity (FIG. 3A). Aliquots of the purified proteins were site-specifically biotinylated at the AviTag using BirA enzyme and re-purified via size exclusion chromatography; both WT and S139A HCV NS3/4A PR had 100% biotinylation incorporation, as verified by mass spectrometry.

These recombinant HCV NS3/4A PR WT and S139A proteins were tested for enzymatic activity in a fluorogenic peptide cleavage assay, where the significantly reduced activity of the HCV NS3/4A PR S139A mutant was confirmed. No enzymatic activity could be detected at most concentrations tested, with minimal activity observed only at high nM to μM concentrations (FIG. 3B).

Isothermal calorimetry was performed to assess the binding affinity of simeprevir to the WT and S139A HCV NS3/4A PR proteins. Both proteins gave very similar results, with the same stoichiometry (˜0.6 Sim/NS3 binding sites) and ΔH values (˜22 kcal/mol) obtained (FIG. 3C). The dissociation constant calculated was very low (˜1 μM), but with a very high associated error (10 nM), suggesting the affinity is too high to be precisely measured using this technique without using a competitive ligand. Nevertheless, as the stoichiometry and ΔH values are the same, it is very likely that there is no major difference in binding affinities between the WT and S139A HCV NS3/4A PR proteins.

Based on these data we chose to proceed with the selection of HCV NS3/4A PR:simeprevir complex-specific binding (PRSIM) molecules based on the S139A mutant protein.

### Example 4—Selection of HCV NS3/4A PR (S139A):Simeprevir Complex-Specific Binding (PRSIM) Molecules

Four rounds of phage display selections were performed on biotinylated HCV NS3/4A PR (S139A) in the presence of simeprevir. From the round 3 and round 4 selection outputs, phage ELISAs were performed on biotinylated HCV NS3/4A PR (S139A) in both the presence and absence of simeprevir, and binding determined by fluorescent signal measured (FIG. 4A and FIG. 4B). The phage ELISA binding data was compared to the DNA sequence data from the same clones, and a panel of 34 scFv and 28 Tn3 clones with unique sequences that demonstrated selective binding to biotinylated HCV NS3/4A PR (S139A) in the presence of simeprevir were selected to be expressed for further biochemical studies (Table 1A and Table 1B). In addition, one scFv clone (PRSIM_51) and 3 Tn3 clones (PRSIM_54, PRSIM_55 and PRSIM_85) that demonstrated binding to biotinylated HCV N3/4A protease (S139A) in both the presence and absence of simeprevir were also selected for further biochemical study.

TABLE 1A

| | | | | Binding | |
|---|---|---|---|---|---|
| | | | | fold change | |
| | | | | [(HCV | |
| | | | | protease + | |
| | | | | Simeprevir | |
| | | | | binding)/ | HTRF |
| | | | | HCV | max. |
| Clone | | | Selection | protease | signal (% |
| Name | Format | Library | round | alone] | delta F) * |
| PRSIM_23 | Tn3 | Library 1 | 4 | 23.8 | 1573 |
| PRSIM_24 | Tn3 | Library 1 | 3 | 31.5 | 561 |
| PRSIM_25 | Tn3 | Library 1 | 4 | 24.0 | 577 |
| PRSIM_26 | Tn3 | Library 1 | 4 | 25.5 | 304 |
| PRSIM_27 | Tn3 | Library 1 | 4 | 25.3 | 422 |
| PRSIM_28 | Tn3 | Library 1 | 3 | 27.1 | 692 |
| PRSIM_29 | Tn3 | Library 1 | 3 | 26.0 | 365 |
| PRSIM_30 | Tn3 | Library 1 | 3 | 25.4 | 550 |
| PRSIM_31 | Tn3 | Library 1 | 4 | 25.0 | 351 |
| PRSIM_32 | Tn3 | Library 1 | 4 | 22.4 | 1955 |
| PRSIM_33 | Tn3 | Library 1 | 3 | 29.9 | 1704 |
| PRSIM_34 | Tn3 | Library 1 | 3 | 22.2 | 614 |
| PRSIM_35 | Tn3 | Library 1 | 3 | 24.8 | 437 |
| PRSIM_36 | Tn3 | Library 1 | 3 | 27.9 | 1440 |
| PRSIM_37 | Tn3 | Library 1 | 3 | 25.3 | 867 |
| PRSIM_38 | Tn3 | Library 1 | 3 | 23.3 | 1061 |
| PRSIM_39 | Tn3 | Library 1 | 4 | 24.9 | 1015 |
| PRSIM_40 | Tn3 | Library 1 | 3 | 26.1 | 218 |
| PRSIM_41 | Tn3 | Library 1 | 4 | 22.8 | 964 |
| PRSIM_42 | Tn3 | Library 1 | 3 | 8.8 | 1895 |
| PRSIM_43 | Tn3 | Library 1 | 3 | 28.6 | 317 |
| PRSIM_44 | Tn3 | Library 1 | 3 | 25.6 | 340 |
| PRSIM_45 | Tn3 | Library 1 | 4 | 33.3 | 842 |
| PRSIM_46 | Tn3 | Library 1 | 3 | 33.3 | 362 |
| PRSIM_47 | Tn3 | Library 1 | 3 | 25.3 | 1367 |
| PRSIM_48 | Tn3 | Library 1 | 3 | 26.6 | 1780 |
| PRSIM_49 | Tn3 | Library 1 | 4 | 30.0 | 761 |
| PRSIM_50 | Tn3 | Library 1 | 4 | 26.3 | 897 |
| PRSIM_54 | Tn3 | Library 1 | 3 | 2.1 | 703 (293) |
| PRISM_55 | Tn3 | Library 1 | 3 | 1.8 | 1655 (550) |
| PRISM_85 | Tn3 | Library 1 | 3 | 2.1 | 333 (73) |

TABLE 1B

| | | | | Binding | |
|---|---|---|---|---|---|
| | | | | fold change | |
| | | | | [(HCV | |
| | | | | protease + | |
| | | | | Simeprevir | |
| | | | | binding)/ | HTRF |
| | | | | HCV | max. |
| Clone | | | Selection | protease | signal (% |
| Name | Format | Library | round | alone] | delta F) * |
| PRSIM_04 | scFv | Library 2 | 3 | 30.3 | 1055 |
| PRSIM_06 | scFv | Library 2 | 4 | 9.0 | 234 |
| PRSIM_07 | scFv | Library 2 | 4 | 21.7 | 535 |
| PRSIM_08 | scFv | Library 2 | 4 | 39.5 | 272 |
| PRSIM_10 | scFv | Library 2 | 4 | 21.8 | 191 |
| PRSIM_56 | scFv | Library 2 | 4 | 16.3 | 829 |
| PRSIM_57 | scFv | Library 2 | 4 | 15.7 | 1076 |
| PRSIM_58 | scFv | Library 2 | 4 | 25.5 | 610 |
| PRSIM_59 | scFv | Library 2 | 4 | 17.5 | 506 |
| PRSIM_60 | scFv | Library 2 | 4 | 23.2 | 441 |
| PRSIM_61 | scFv | Library 2 | 4 | 17.8 | 450 |
| PRSIM_62 | scFv | Library 2 | 4 | 2.3 | 146 |
| PRSIM_63 | scFv | Library 2 | 4 | 23.5 | 760 |
| PRSIM_64 | scFv | Library 3 | 4 | 26.1 | 1006 |
| PRSIM_65 | scFv | Library 3 | 3 | 15.0 | 411 |
| PRSIM_66 | scFv | Library 3 | 3 | 19.6 | 559 |
| PRSIM_67 | scFv | Library 3 | 3 | 12.7 | 1708 |
| PRSIM_68 | scFv | Library 3 | 3 | 15.3 | 133 |
| PRSIM_69 | scFv | Library 3 | 3 | 8.1 | 292 |
| PRSIM_70 | scFv | Library 3 | 3 | 15.3 | 25 |
| PRSIM_71 | scFv | Library 3 | 3 | 7.9 | 83 |
| PRSIM_72 | scFv | Library 3 | 3 | 12.5 | 1107 |
| PRSIM_73 | scFv | Library 3 | 3 | 25.4 | 418 |
| PRSIM_74 | scFv | Library 3 | 3 | 10.1 | 250 |
| PRSIM_75 | scFv | Library 3 | 3 | 6.9 | 1030 |
| PRSIM_76 | scFv | Library 3 | 3 | 14.9 | 285 |
| PRSIM_77 | scFv | Library 3 | 3 | 15.2 | 288 |
| PRSIM_78 | scFv | Library 3 | 3 | 20.2 | 852 |
| PRSIM_79 | scFv | Library 3 | 3 | 22.2 | 91 |
| PRSIM_80 | scFv | Library 3 | 3 | 19.1 | 209 |
| PRSIM_81 | scFv | Library 3 | 3 | 30.8 | 111 |
| PRSIM_82 | scFv | Library 3 | 3 | 23.1 | 316 |
| PRSIM_83 | scFv | Library 3 | 3 | 21.9 | 115 |
| PRSIM_84 | scFv | Library 3 | 3 | 27.0 | 419 |
| PRSIM_51 | scFv | Library 3 | 3 | 1.0 | 878 (777) |

All data reported in the presence of simeprevir except data in parenthesis which was determined in the absence of simeprevir.

### Example 5—a Panel of PRSIM Molecules are Specific for the HCV NS3/4A PR (S139A):Simeprevir Complex

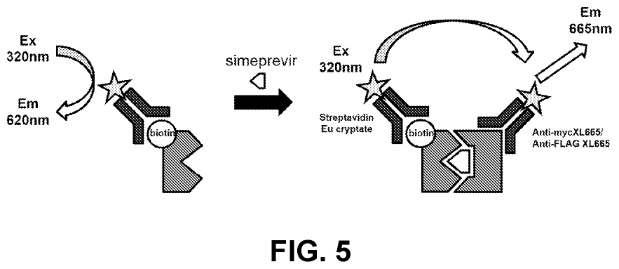

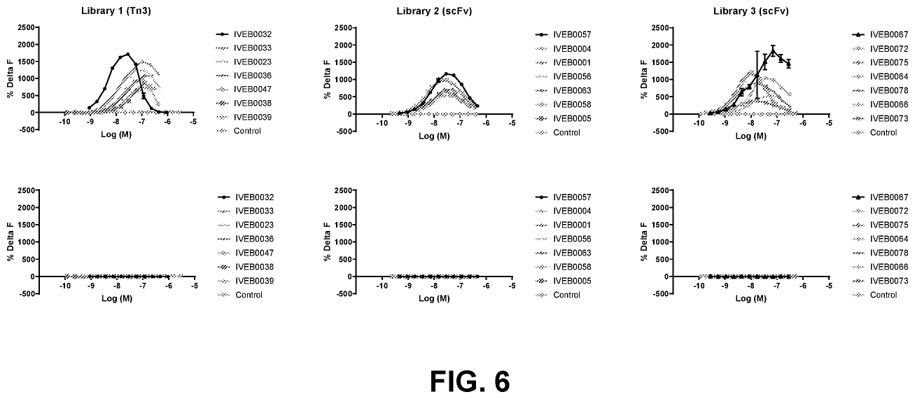

The PRSIM binding proteins identified from phage display selections as complex-specific were expressed and purified at larger scale to provide sufficient material for further analysis. A homogeneous time-resolved fluorescence (HTRF) binding screen (FIG. 5) was performed on all the HCV NS3/4A PR (S139A):simeprevir complex-specific PRSIM molecules and a panel of 8 Tn3-based and 14 scFv-based molecules were confirmed as complex-specific with no detectable binding to the HCV NS3/4A PR (S139A) protein alone (Table 1 (in bold), FIG. 6).

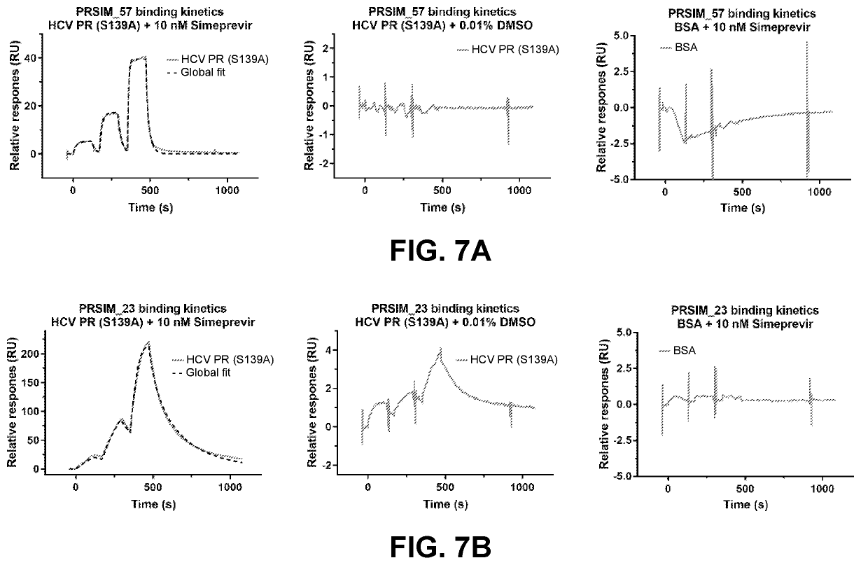

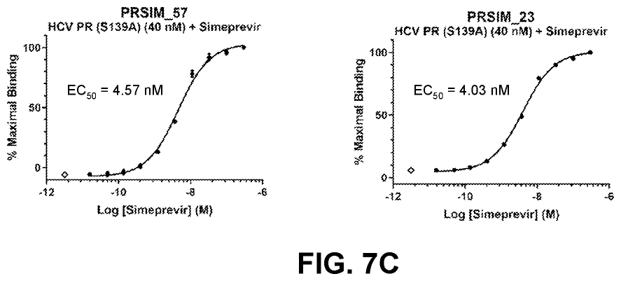

To further characterise the PRSIM binding molecules, 5 scFv molecules (PRSIM_4, PRSIM_57, PRSIM_67, PRSIM_72 and PRSIM_75) and 5 Tn3 molecules (PRSIM_23, PRSIM_32, PRSIM_33, PRSIM_36, PRSIM_47) were selected and the kinetics of HCV NS3/4A PR (S139A) protease binding in the presence or absence of simeprevir were determined using Biacore 8K (Table 2). All the PRSIM binding molecules tested showed selectivity for simeprevir-bound HCV NS3/4A PR (S139A) and only three showed minor non-specific binding to HCV NS3/4A PR (S139A) alone. PRSIM_57 (FIG. 7A) and PRSIM_23 (FIG. 7B) were selected for further characterization. HCV NS3/4A PR (S139A) had an affinity for PRSIM_57 (scFv) of 15.0 nM and for PRSIM_23 (Tn3) of 6.3 nM. The effect of simeprevir concentration on the formation of the HCV NS3/4A PR (S139A)/PRSIM_57/23 complex was also assessed (FIG. 7C). Simeprevir had an almost equivalent EC₅₀ for PRSIM_57 and PRSIM_23 in complex with HCV NS3/4A PR (S139A); 4.57 and 4.03 nM, respectively.

TABLE 2
Binding and kinetic constants measured for the binding of HCV NS3/4A PR (S139A) to PRSIM
binding molecules in the presence or absence of simeprevir. BSA in the presence of simeprevir was used
as a control.

| | | Immobilised | | HCV NS3/4APR(S139A) | | | | BSA |
|---|---|---|---|---|---|---|---|---|
| | | level | Simeprevir | kₐ | k_{d} | K_{D} | Rₘₐₓ | control |
| | ID | (RUs) | 10 nM | (M⁻¹ s⁻¹) | (s⁻¹) | (nM) | (RUs) | ($) |
| scFv | PRSIM_4 | 290 | + | 1.74E+07 | 1.95E−01 | 11.2 | 65.9 | # |
| | | | — | N.D. | N.D. | # | | |
| | PRSIM_57 | 380 | + | 1.73E+07 | 2.60E−01 | 15 | 69.1 | # |
| | | | — | N.D. | N.D. | # | | |
| | PRSIM_67 | 145 | + | 1.97E+07 | 2.26E−01 | 11.5 | 19.3 | # |
| | | | — | N.D. | N.D. | # | | |
| | PRSIM_72 | 164 | + | 1.18E+06 | 1.18E−03 | 1 | 33.4 | # |
| | | | — | N.D. | N.D. | # | | |
| | PRSIM_75 | 218 | + | 9.09E+06 | 2.67E−03 | 0.3 | 88.8 | # |
| | | | — | N.D. | N.D. | # | | |
| Tn3 | PRSIM_23 | 616 | + | 5.03E+06 | 3.18E−02 | 6.3 | 284 | # |
| | | | — | N.D. | N.D. | * | | |
| | PRSIM_32 | 532 | + | 2.51 E+09 | 4.01E+01 | 16 | 19.9 | # |
| | | | — | N.D. | N.D. | * | | |
| | PRSIM_33 | 737 | + | 3.65E+06 | 3.03E−02 | 8.3 | 171.9 | # |
| | | | — | N.D. | N.D. | # | | |
| | PRSIM_36 | 679 | + | 5.15E+06 | 4.98E−02 | 9.7 | 214.4 | # |
| | | | — | N.D. | N.D. | # | | |
| | PRSIM_47 | 674 | + | 1.32E+10 | 5.12E+01 | 3.9 | 9 | # |
| | | | — | N.D. | N.D. | # | | |

N.D. = indicates the values could not be determined due to absence of detectable binding
# = no binding
* = minimal non-specific binding
Data in italics indicates high association rate and/or lower than expected Rₘₐₓ
$ = BSA control was only measured in the presence of 10 nM Simeprevir

### Example 6—PRSIM-Based CIDs can Regulate Reconstitution of a Split Protein

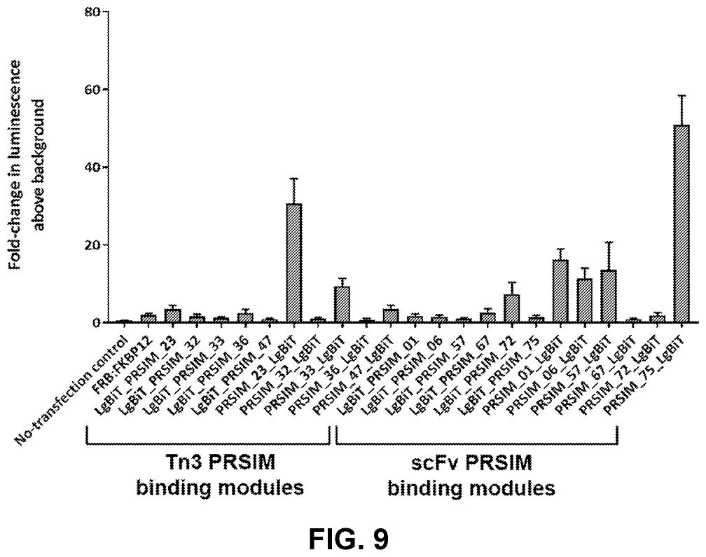

Having isolated PRSIM binding molecules that specifically bound simeprevir:HCV NS3/4A PR (S139A) complexes, we reasoned that the system could be used to regulate the reconstitution of a split protein. By providing temporal and spatial regulation of protein dimerization within a cell, the CID could be applied within a post-translational context to control a desired protein-protein interaction or activity. Numerous examples of split proteins that gain activity upon reconstitution exist, one of which is the split Nanoluciferase as provided in the NanoBiT system (Promega) (FIG. 8). We applied this system to the PRSIM-based CIDs by fusion of HCV NS3/4A PR (S139A) to SmBiT and the PRSIM binding members fused to LgBiT. A screen was performed, testing five Tn3 and six scFv PRSIM binding modules arising from the phage selection process, using both N- and C-terminal fusions to LgBiT and the equivalent N- and C-terminal fusions of HCV NS3/4A PR (S139A) fused to SmBiT. Cells were transfected with the appropriate plasmids, incubated for 24 hours and then treated with 100 nM simeprevir or vehicle control (or 100 nM rapamycin in the case of the FRB:FKBP12 control supplied with the kit). Luminescence was measured and the fold change of the signal in the presence of simeprevir over the signal obtained in the absence of simeprevir was calculated (FIG. 9). An overall trend was observed, with significant fold-change in luminescence generally only observed where LgBiT was fused to the C-terminus of a PRSIM binding module. Significant signals above background in this context were observed for the following PRSIM binding modules: PRSIM_23 (31-fold), PRSIM_33 (9-fold), PRSIM_01 (16-fold), PRSIM_06 (11-fold), PRSIM_57 (14-fold) and PRSIM_75 (S1-fold). The results indicate that in the presence of simeprevir a number of the isolated PRSIM binding modules can specifically induce the dimerization of the split NanoLuc from the NanoBiT system.

### Example 7—PRSIM-Based CIDs can Regulate Gene Expression Via Reconstitution of a Split Transcription Factor

Having demonstrated that PRSIM-based CIDs were capable of reconstituting the activity of a split protein via fusion of the HCV NS3/4A PR (S139A) and PRSIM molecules to the separate components of the split NanoLuc enzyme, we reasoned that the same CIDs could regulate expression of transgenes via fusion to the two domains of a split transcription factor. To demonstrate this, we utilised the iDimerize regulated transcription system (Takara) in which two separate vectors are provided; one vector (pHet-Act1-2) encodes FRB fused to the activation domain (AD) p65, and the DNA binding domain (DBD) ZFHD1 fused to 3 copies of FKBP12, separated by an IRES sequence and preceded by the constitutive promoter, CMV; the other vector (pZFHD1_Luciferase) encodes luciferase under the control of an inducible promoter that contains 12 copies of the ZFHD1 recognition sequence upstream of a minimal IL-2 promoter. When both plasmids are transfected into cells, the FRB-AD and DBD-FKBP12 proteins are expressed; the DBD recognises its target site on the inducible promoter, but as there is no AD in close proximity to the promoter, transcription initiation does not occur. Only when the rapalog inducer “A/C heterodimeriser” is added, is the AD recruited to the DBD bound to the promoter upstream of the luciferase gene and expression commences.

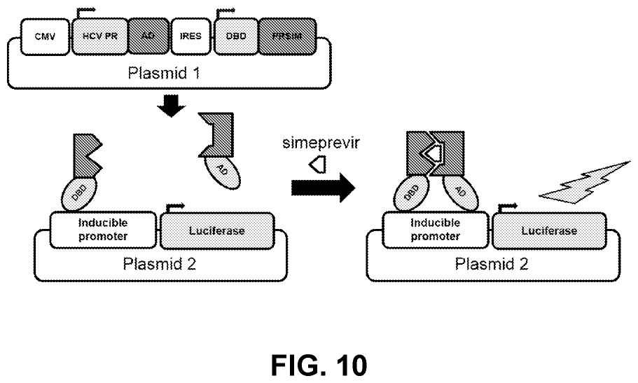

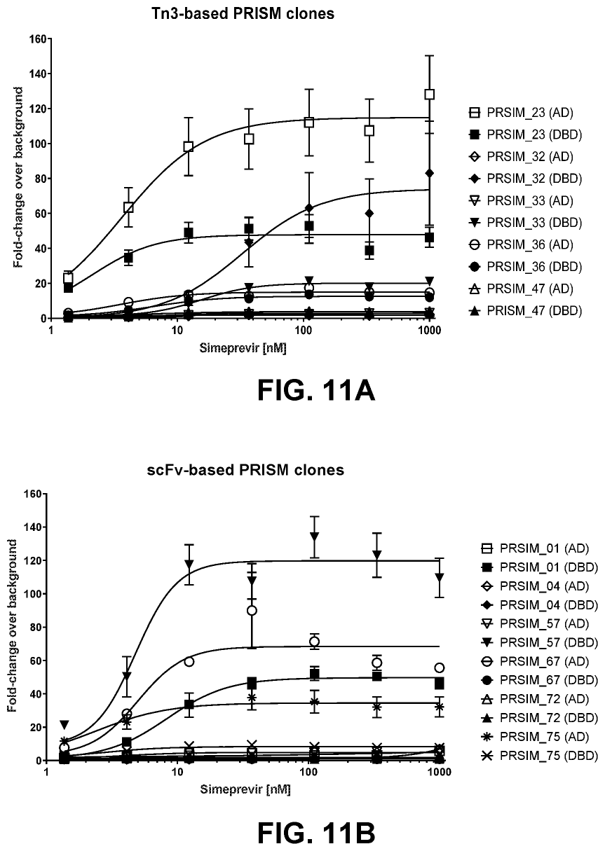

We exchanged the FRB and FKBP12 coding sequences for those encoding one copy of the HCV NS3/4A PR (S139A) and one of the 11 PRSIM molecules described below, where the PRSIM molecules were either fused to the N-terminus of the activation domain or the C-terminus of the DNA binding domain (FIG. 10). Following transfection of cells with the pHet-Act1-2 (PRSIM) and pZFHD1_Luciferase constructs, we assessed the ability of the PRSIM-based CIDs to regulate luciferase gene expression in the presence of increasing concentrations of simeprevir. The different PRSIM-based CID constructs demonstrated dose-dependent gene expression regulation ranging from 1.4- to 146-fold (FIG. 11A and FIG. 11B, Table 3) with 6 Tn3-based and 5 scFv-based PRSIM molecules demonstrating over 10-fold increases in gene expression. The highest fold-change achieved for the Tn3-based clones was 106-fold, based on PRSIM_23 fused to the activation domain. Interestingly, the majority of PRSIM clones demonstrated a preference for fusion to either the AD or the DBD; PRSIM_23 is unique in its ability to provide strong gene expression regulation in both orientations (106-fold fused to the AD and 88-fold when fused to the DBD). PRSIM_23 also demonstrated the lowest EC50 (2 nM), meaning that lower concentrations of simeprevir were required to activate transcription. The clone that demonstrated the highest fold-change upon addition of simeprevir was scFv-based PRSIM_57 fused to the DBD, which reached 146-fold induction and a low EC50 value (3 nM).

TABLE 3
EC50 and fold-change values for PRSIM-based CIDs in a split
transcription factor assay.

| | Fusion | | |
|---|---|---|---|
| PRISM clone | (AD or DBD) | EC50 [nM] | Max fold change |
| PRSIM_01 | AD | Ambiguous | 5.83 |
| PRSIM_01 | DBD | 6.99 | 86.76 |
| PRSIM_04 | AD | 158988 | 6.9 |
| PRSIM_04 | DBD | 6.55 | 1.364 |
| PRSIM_57 | AD | 3.21 | 10.73 |
| PRSIM_57 | DBD | 3.54 | 146.6 |
| PRSIM_67 | AD | 4.97 | 87.1 |
| PRSIM_67 | DBD | 2.05 | 1 |
| PRSIM_72 | AD | 4.84 | 2.2 |
| PRSIM_72 | DBD | 32.99 | 1.4 |
| PRSIM_75 | AD | 2.171 | 40.67 |
| PRSIM_75 | DBD | 2.668 | 9.4 |
| PRSIM_23 | AD | 2.82 | 106.67 |
| PRSIM_23 | DBD | 2.47 | 88.8 |
| PRSIM_32 | AD | 12.3 | 2.2 |
| PRSIM_32 | DBD | 29.3 | 65.77 |
| PRSIM_33 | AD | 82.39 | 2.9 |
| PRSIM_33 | DBD | 12.85 | 33.14 |
| PRSIM_36 | AD | 3.54 | 33.74 |
| PRSIM_36 | DBD | 5.66 | 73.3 |
| PRSIM_47 | AD | 6.15 | 3.85 |
| PRSIM_47 | DBD | 8.87 | 3 |

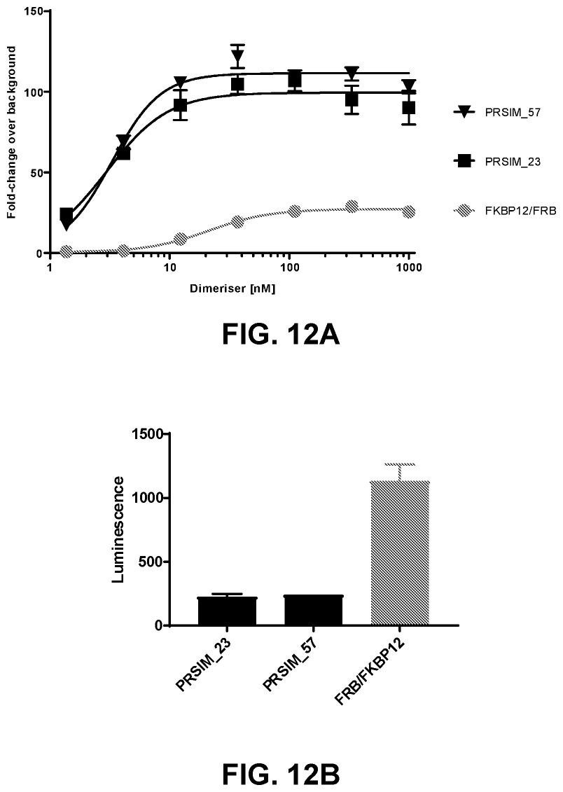

When the ability of the HCV NS3/4A PR (S139A)-AD and DBD-PRSIM_23 or DBD-PRSIM_57-based constructs to regulate the expression of luciferase in the presence of simeprevir was directly compared to the FRB:FKBP12:rapalog positive control, the PRSIM-based CIDs (100-fold increase) outperformed the FRB:FKBP12-based CID (30-fold increase) (FIG. 12A). Analysis of the luminescence values obtained in the absence of inducer (i.e. simeprevir or rapalog) revealed that the levels were higher for the FRB:FKBP12:rapalog-based CID, suggesting a level of leakiness that was improved in the PRSIM-based CID (FIG. 12B)

### Example 8—Increasing Tandem Copies of PRSIM Fused to the DBD Improves Gene Regulation

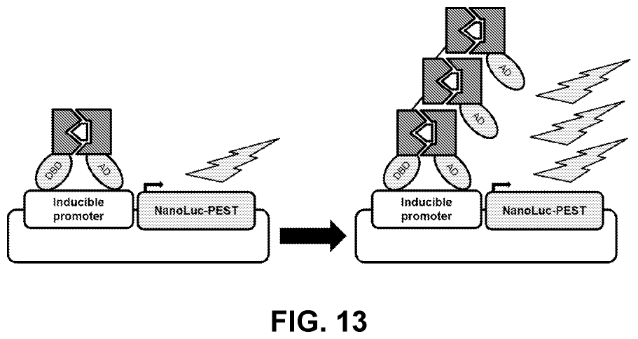

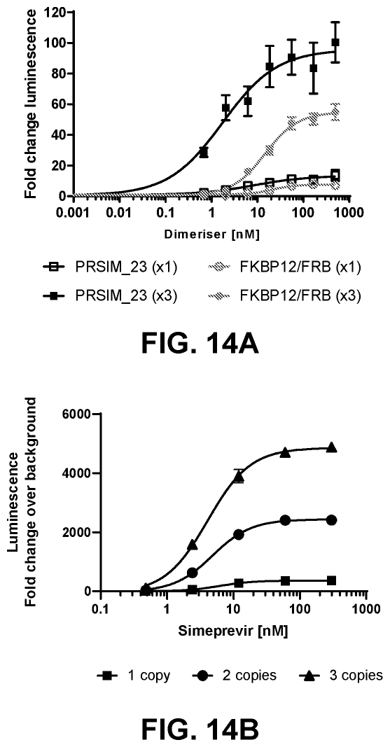

To assess the impact of copy number of the target protein fused to the DNA binding domain, we generated pHet-Act1-2-based constructs encoding FRB-AD or HCV NS3/4A PR (S139A)-AD and DBD-FKBP12 or DBD-PRSIM_23, whereby the protein fused to the DBD was included either as a single copy or as three tandem copies separated by short peptide linkers (FIG. 13). When the ability of the PRSIM_23-based CID to regulate the expression of a NanoLuc-PEST protein in the presence of simeprevir was compared to the FRB:FKBP12:rapalog positive control, we found that the PRSIM_23-based CID outperformed the FRB:FKBP12-based CIDs when either one copy (55-fold vs 13-fold) or three copies (100-fold vs 55-fold) of the DBD fusion partner were used (FIG. 14A).

Furthermore, when the impact of one, two or three tandem copies of PRSIM_23 fused to the DBD was assessed via the same split transcription factor assay, and the induction of firefly luciferase expression was measured, a graded response was observed; one copy of PRSIM_23 resulted in a max fold change of 364.5, whereas two tandem PRSIM_23 molecules resulted in max fold change of 2436 and a further increase to 4862-fold for three tandem PRSIM_23 molecule (FIG. 14B).

This data suggests that it is possible to improve the regulation of gene expression from the inducible promoter by recruiting more copies of the activation domain, and that this is a common phenomenon, independent of CID used.

### Example 9—PRSIM-Based CIDs can Regulate Activity of a Split Chimeric Antigen Receptor (CAR)

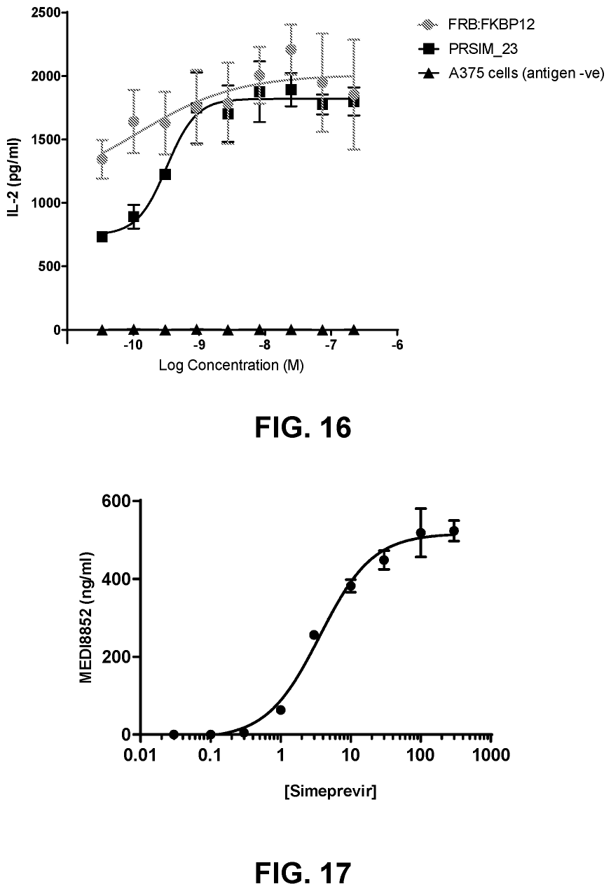

Regulation of CAR activity via chemical-induced heterodimerisation was previously shown to be an effective way to modulate CAR function (Wu et al. 2015); (Hill et al. 2018). We hypothesized that the application of the heterodimerising PRSIM components to a CAR would facilitate CAR regulation in a similar manner. The previously described FKBP12:FRB system (Wu et al. 2015) was used as a comparator to regulate CAR function. To test this, we engineered Jurkat T-cells to express PRSIM and FKBP12:FRB-regulated CARs using a lentiviral expression system (FIG. 15A). Activation of the CARs, upon antigen binding, would result in the secretion of IL-2 in the presence of either the rapamycin analog AP2196 (FKBP12:FRB dimeriser) or simeprevir (PRSIM dimeriser) in a dose-dependent manner (FIG. 15B). IL-2 expression can be rapidly quantified via an IL-2-specific ELISA (R&D Systems). The design of these systems should facilitate activation of the T-cells only in the presence of the appropriate dimeriser and upon antigen binding. In both PRSIM and FKBP12:FRB-regulated CAR systems, the addition of simeprevir or AP2196, respectively, resulted in a dose-dependent activation of the CAR-expressing Jurkats cells in the presence of antigen-positive HepG2 cells as measured by IL-2 production (FIG. 16). Importantly, no activation of either CAR was observed in the presence of antigen-negative A375 cells (FIG. 16). While both the FKBP12:FRB and PRSIM system both showed dose-dependent activation, the PRSIM system exhibited tighter control of CAR activity evidenced by lower background IL-2 levels and a larger dynamic range for CAR activation (FIG. 16). Both systems exhibited comparable maximal IL-2 expression levels. These data demonstrate that the PRSIM heterodimerising system can be used for simeprevir-mediated regulation/modulation of CAR-initiated cellular signalling pathways.

### Example 10—PRSIM-Based CIDs can Regulate Gene Expression of an Antibody (MED18852)

In addition to demonstrating gene regulation of two recombinant intracellular proteins (luciferase (Example 7) and NanoLuc-PEST (Example 8)) using a PRSIM-based CID, the regulation of gene expression of a secreted antibody (MED18852; SEQ ID NO: 205 and SEQ ID NO: 206) was also investigated. pHet-Act1-2-based constructs encoding HCV NS3/4A PR (S139A)-AD and DBD-PRSIM_23 (three tandem copies) and a construct encoding pZFHD1_MED18852) were generated. When cells were transfected with these two constructs, the expression of MED18852 was shown to be dependent on the dose of simeprevir, as measured using the Singleplex Human/NHP IgG Isotyping Kit (Mesoscale) (FIG. 17).

### Example 11—PRSIM-Based CIDs can Regulate Gene Expression of a Protein Via Adeno-Associated Virus

Recombinant adeno-associated virus (rAAV) vectors represent a well-studied platform which could be used to deliver the DNA encoding a PRSIM_23/HCV NS3/4A PR (S139A)-based CID to cells to control gene therapy. One such application is the regulation of an exogenous transgene delivered to cells either together with, or in separate AAV particles to the PRSIM_23/HCV NS3/4A PR (S139A)-based split transcription factor components described in Example 7. In the context of the system described here, the packaging capacity of AAV limits the size of the transgenes that can be delivered in the same AAV vector to ˜550 bp, or the size of transgenes that can be delivered in separate AAV particles to ˜3.6 kb.

To demonstrate delivery of the CID-encoding DNA and an inducible transgene “in trans”, two different AAV vectors were generated, one encoding the PRSIM_23/HCV NS3/4A PR (S139A)-based split transcription factor components, with expression driven by the constitutive EF1/HTLV hybrid promoter, and the second encoding the firefly luciferase gene under control of the inducible ZFHD1 promoter (FIG. 18A). AAV particles were generated from these vectors and following transduction of HEK293 cells with the two separate AAV8 preparations, we observed simeprevir-dose-dependent regulation of luciferase gene expression by the PRSIM_23/HCV NS3/4A PR (S139A)-based CID only when both AAV8 particle preps were added, with a 228-fold induction of luciferase activity (FIG. 18B).

To demonstrate that the CID and an inducible transgene can be delivered “in cis”, an AAV8 vector encoding both the PRSIM_23/HCV NS3/4A PR (S139A)-based transcription factor components and an inducible IL-2 transgene was generated (FIG. 18C). Following transduction of HEK293 cells with AAV8 particles generated using this AAV vector, we observed simeprevir-dose-dependent regulation of IL-2 gene expression by the PRSIM_23/HCV NS3/4A PR (S139A)-based, with maximal levels of ˜3500 μg/mi IL-2 observed (FIG. 18D). The level of IL-2 expression induced by the PRSIM_23/HCV NS3/4A PR (S139A)-based CID at the highest concentrations of simeprevir tested was comparable (3506+/−817 μg/mi) to that achieved from a control AAV8 vector encoding the IL-2 transgene under the control of a constitutive CAG promoter (2606+/−189 μg/mi) (FIG. 18E).

Thus, the ability of the PRSIM-based CID to control gene expression via AAV transduction was demonstrated, using either a single, or dual AAV-based system.

### Example 12—PRSIM-Based CID can Regulate the Transcription of an Endogenous Gene

Having demonstrated that PRSIM-based CIDs can regulate the expression of transgenes via fusion to the two domains of a split transcription factor, we reasoned that the PRSIM-based CID could also regulate the expression of endogenous genes. The use of chemical-induced heterodimerisation systems to regulate endogenous gene activity has previously been shown to be an effective way to modulate gene regulation (Foight et al. 2019). We therefore hypothesized that the application of the heterodimerising PRSIM components to an activating CRISPR (CRISPRa) system could facilitate endogenous gene regulation in a similar manner.

To demonstrate this, an inactive form of the Streptococcus pyogenes Cas9 enzyme (dCas9) and an activation domain (AD) consisting of a fusion of three transcriptional activators (VP64, p65 and Rta; VPR) were separately fused to the two protein components of the CID (three copies of PRSIM_23 and HCV NS3/4A PR (S139A), respectively) such that, only in the presence of the small molecule inducer, the AD is brought into close proximity to the dCas9. Co-transfection of the PRSIM-based CID and a guide RNA (gRNA) targeting the promoter of interleukin-2 (IL-2) allows dCas9 to bind to the target site on the promoter of IL-2. Upon administration of the PRSIM dimeriser (simeprevir) the AD and associated transcription machinery is subsequently recruited to the promoter region of the endogenous IL-2 gene, enabling initiation of transcription (FIG. 19A). Activation of the system can therefore by measured by IL-2 production and quantified via an IL-2 specific cytokine assay (MSD).

In HEK293 cells transiently expressing the PRSIM regulated split dcas9/AD cassette and an IL-2 targeted gRNA, the addition of simeprevir resulted in secretion of IL-2. (FIG. 19B). Importantly, no IL-2 was detected in cells expressing only part of the system (gRNA only or PRISM-dCas9 only) or in those cells expressing a non-IL-2 targeting gRNA.

This data demonstrates that the PRSIM heterodimerising system can be used for simeprevir-mediated regulation of endogenous gene expression.

### Example 13—the HCV NS3/4A PR (S139A):PRSIM 23 and HCV NS3/4A PR (S139A):PRSIM 57 Complexes are Specific for Simeprevir

Having demonstrated that formation of the active switch complex is dependent on the presence of simeprevir, we wanted to test the specificity of this interaction with respect to alternative small molecule inhibitors of HCV protease. There are several small molecule inhibitors that are known to bind the HCV NS3/4A protease and have been approved for human use. A panel of such small molecules were assessed for their ability to induce complex formation between HCV NS3/4A PR (S139A) and PRSIM_23 or PRSIM_57. These were glecaprevir, boceprevir, telaprevir, asunaprevir, paritaprevir, vaniprevir, narlaprevir, grazoprevir, and danoprevir.

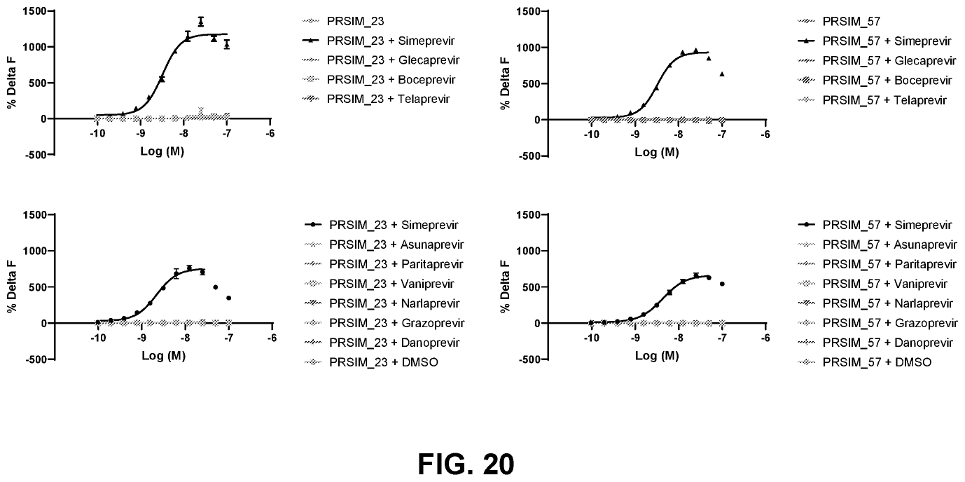

A homogeneous time-resolved fluorescence (HTRF) binding assay (FIG. 20) was performed to determine the level of HCV NS3/4A PR (S139A):PRSIM_23 and HCV NS3/4A PR (S139A):PRSIM_57 complex formed when simeprevir was substituted with in the alternative HCV PR inhibitor small molecules. We found that induction of complex formation was specific for simeprevir as none of the HCV PR inhibitors could form a complex with HCV NS3/4A PR (S139A) and PRSIM_23, nor HCV NS3/4A PR (S139A):PRSIM_57.

This data suggests that administration of other HCV NS3/4A PR inhibitor small molecules, such as in the case of a HCV-infected individual, would not be able to form an active HCV NS3/4A PR (S139A):PRSIM_23 complex, and that the HCV NS3/4A PR (S139A):PRSIM_23 complex is exquisitely specific for simeprevir.

### Example 14— Residues in HCV NS3/4A PR are Predicted to Reduce the Affinity for Simeprevir

The affinity of simeprevir for HCV NS3/4A PR is very high (Example 3; FIG. 3B), which will likely impact the rate at which the complex can dissociate once simeprevir dosing has ceased. The identification of HCV NS3/4A PR variants with a reduced affinity for simeprevir could afford some flexibility in modulating the half-life of the complex, allowing such PRSIM-based CIDs to be more rapidly inactivated where necessary e.g. if an adverse event were encountered and rapid reversal of activity were required.

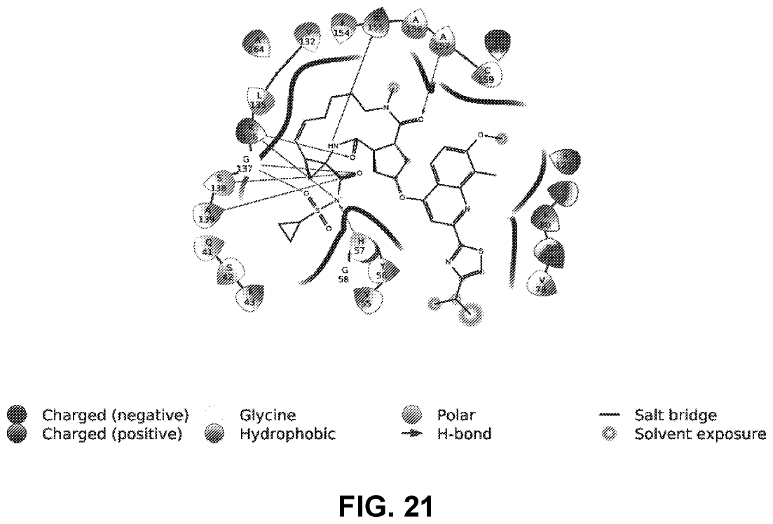

In order to identify mutations on the Hepatitis C virus (HCV) protease protein that reduce simeprevir binding, the co-crystal structure of HCV NS3/NS4A in complex with simeprevir (PDB: 3KEE, Resolution: 2.4 Å) was first analysed. The analysis showed that the HCV NS3/NS4A:simeprevir interface is made up of 25 HCV residues where 6 residues contribute towards hydrogen bond and salt bridge interactions and 12 are surface-exposed (FIG. 21). Residues were shortlisted for inclusion in a detailed mutational analysis via selection on two criteria: Firstly, residues that are solvent exposed were omitted to avoid any negative impact of mutagenesis on binding of the PRSIM molecules to the complex; secondly, those exhibiting a predicted change in free energy upon mutation to Alanine of >1 kcal/mol were included. Free energy perturbation calculations were then used to predict the relative binding free energies upon mutation of the interacting side chains of these residues (H57, K136, S139 and R155). Mutations that are predicted to reduce the affinity of HCV protease for simeprevir are listed in Table 4. Although the FEP+ alanine scanning analysis only predicted a relatively small change in predicted binding free energy for 0168, due to its published role in resistance to simeprevir we additionally evaluated 3 mutations (D168A, D168E and 0168Q) experimentally at this position.

TABLE 4
Predicted changes in binding free energies of HCV NS3/NS4A protease
for simeprevir upon mutation of key binding residues.

| | % Exposure | Predicted | Predicted |
|---|---|---|---|
| Mutation | Bound WT | ΔΔG | ΔΔG Error |
| H57D | 6% | 12.94 | 0.541 |
| H57T | | 11.35 | 0.757 |
| H57I | | 10.42 | 0.572 |
| H57K | | 6.66 | 0.449 |
| K136D | 45% | 4.85 | 0.46 |
| K136N | | 3.15 | 0.42 |
| K136H | | ~2.5 | ~0.61 |
| S139D | 0% | 17.82 | 2.23 |
| S139H | | ~10 | ~2 |
| S139N | | 11.64 | 0.75 |
| S139T | | 4.19 | 1.29 |
| R155W | 1% | 6.83 | 1.07 |
| R155F | | 4.77 | 0.28 |
| R155K | | 3.95 | 0.3 |

### Example 15—Mutations in the HCV NS3/4A PR Affect Formation of the HCV NS3/4A PR (S139A): Simeprevir: PRSIM 23 Complex

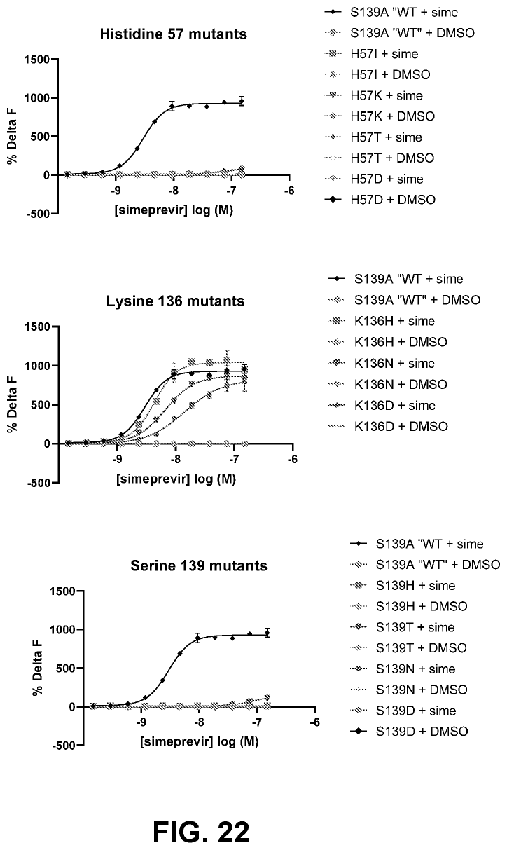

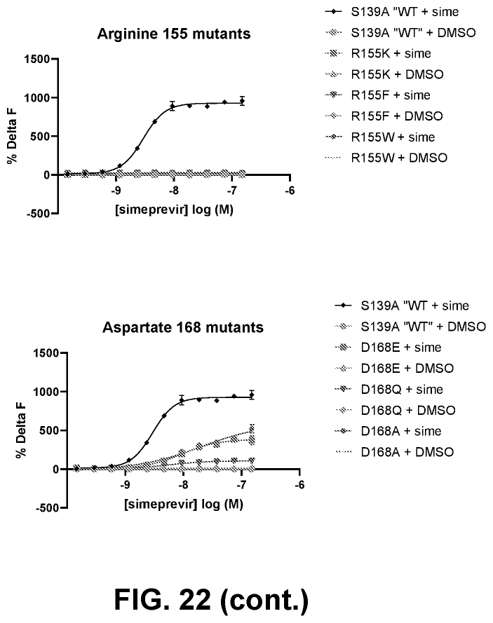

Having identified a panel of mutants predicted to reduce the affinity of HCV NS3/4A PR for simeprevir, we reasoned that if the mutations affected the affinity of HCV NS3/4A PR for simeprevir as predicted, this would the influence formation of the HCV NS3/4A PR (S139A): simeprevir: PRSIM_23 complex. To assess the impact these mutations have on the formation of the HCV NS3/4A PR (S139A): simeprevir: PRSIM_23 complex we measured the amount of complex formation in a homogeneous time-resolved fluorescence (HTRF) binding assay (FIG. 22) in the presence of increasing concentrations of simeprevir.

We found that mutations made at positions R155, H57 and S139 were not tolerated and no complex was formed. Mutations made at position K136 resulted in complex-competent HCV PR variants, with the degree of complex formation reaching the same maximum as observed with HCV PR “wt”. Mutations at residue D168 are also tolerated, but with a reduction in the amount of complex formed at the equivalent HCV PR concentration. The EC50 observed for simeprevir is increased with K136N and K136D mutants, and for all mutants at position D168, indicating that different affinities exist within the complex for these mutants.

### Example 16—Some HCV NS3/4A PR Mutants Show Decreased Affinity for Simeprevir

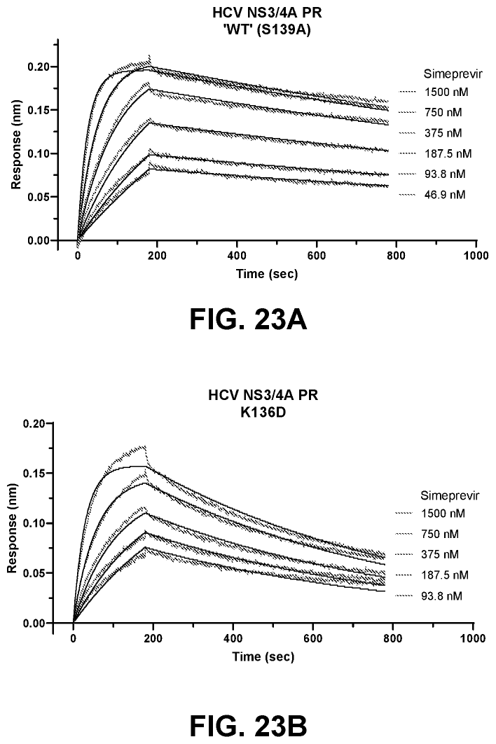

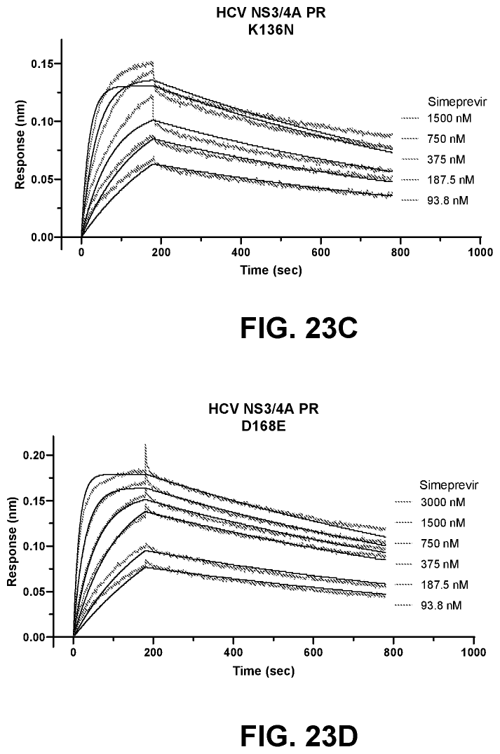

Having identified that mutations at position K136 and D168 resulted in complex competent HCV PR variants, three mutants (K136D, K136N and D168E) were selected for further characterization. To assess the impact on the affinity of simeprevir, the kinetics of simeprevir binding to HCV NS3/4A PR ‘WT’ (S139A) protease and the three mutants were determined using Octet RED384 (FIG. 23, Table 5). The K1360 mutation had the biggest effect on the simeprevir affinity, ˜3.5-fold decreased affinity compared to the HCV NS3/4A PR ‘WT’ (S139A). The K136N and 0168E had resulted in ˜2-fold decreased affinity. The changes in affinity were mainly driven by an increase in the dissociation rate (k_{off}).

TABLE 5
Binding and kinetic constants measured using Octet RED384 for the
binding of simeprevir to mutants of HCV NS3/NS4A protease.

| | Simeprevir | | | | |
|---|---|---|---|---|---|
| HCV | kₒₙ | k_{off} | K_{D} | | |
| NS3/4A PR | (M⁻¹ s⁻¹) | (s⁻¹) | (nM) | | n = |
| WT | 2.78E+04 | 4.95E−04 | 17.8 | (±1.1) | 3 |
| (S139A) | (±6.47E+03) | (±1.09E−04) | | | |
| K136D | 3.62E+04 | 2.34E−03 | 62.6 | (±10) | 3 |
| | (±2.19E+04) | (±1.67E−03) | | | |
| K136N | 3.38E+04 | 1.07E−03 | 31.5 | (±7) | 3 |
| | (±7.00E+02) | (±2.75E−04) | | | |
| D168E | 2.00E+04 | 6.65E−04 | 33.0 | (±2.8) | 2 |
| | (±4.74E+03) | (±2.10E−04) | | | |

Data is mean ± s.d.

### Example 17—the Change in Simeprevir Affinity Caused by the Mutations in the HCV NS3/4A PR (S139A) Affects Formation of the HCV NS3/4A PR (S139A): Simeprevir: PRSIM 23 Complex

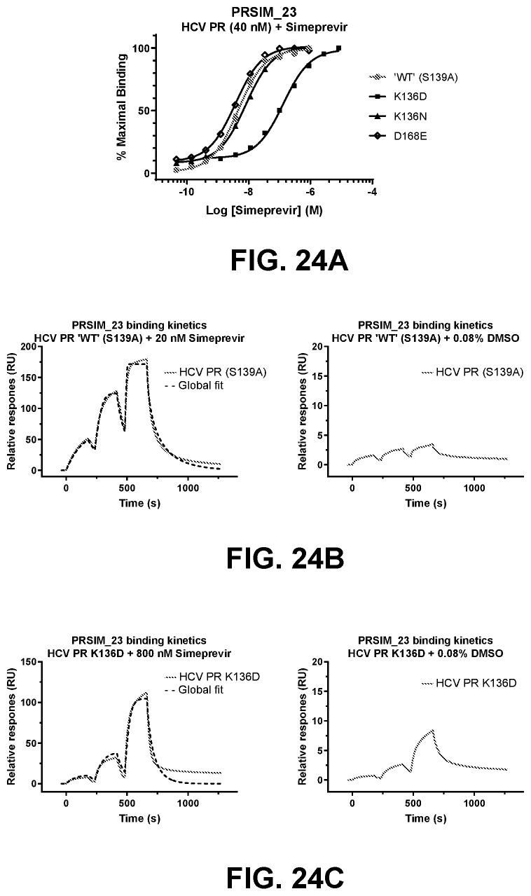

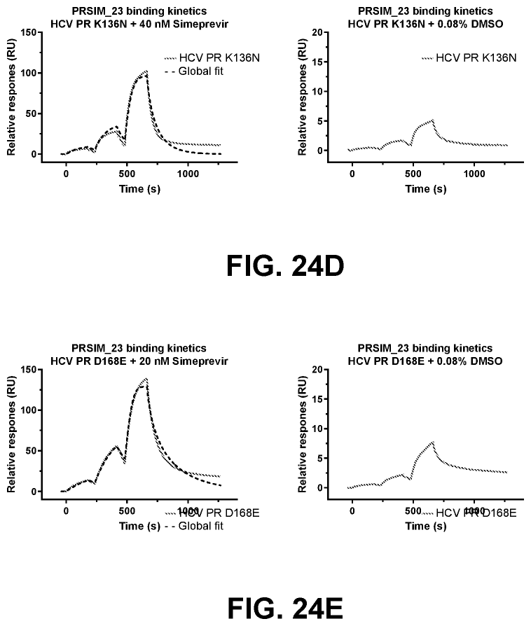

To further characterise the three mutant proteases, the effect of simeprevir concentration on the formation of the mutant HCV NS3/4A PR (S139A)/PRSIM_23 complex was also assessed using Biacore 8K (FIG. 24A, Table 6). In line with the decreased affinity of simeprevir (Table 5) the EC₅₀ of simeprevir in the HCV NS3/4A PR K136D/simeprevir/PRSIM_23 complex had increased to 131.5 nM, ˜30-fold higher than for the wt complex. The K136N mutation also resulted in a higher EC₅₀ for simeprevir compared to ‘wt’, albeit the effect was less than for the K136D mutation. The 0168E mutation however, had an almost equivalent EC₅₀ compared to the ‘wt’ complex; 3.69 and 4.53 nM, respectively.

The HCV NS3/4A PR mutants binding in the presence or absence of simeprevir to PRSIM_23 were also determined using Biacore 8K (FIG. 24B-E). All the protease mutants tested showed similar minor non-specific binding to PRSIM_23 alone, as shown previously for the HCV NS3/4A PR ‘WT’ (S139A) (Table 2, FIG. 7B). Due to the different affinities of simeprevir and the different effects the mutations had on the formation of the HCV NS3/4A PR/simeprevir/PRSIM_23 complex (FIG. 24A), a different fixed concentration of simeprevir was used for each HCV NS3/4A PR in order to form the complex on the Biacore chip. The simeprevir concentration for each mutant was determined to be 5-6× the respective EC₅₀ for simeprevir (Table 6). The complexes containing a mutant HCV NS3/4A PR all had lower affinity than the HCV NS3/4A PR ‘WT’ (S139A) complex (Table 7). HCV NS3/4A PR ‘WT’ (S139A) had an affinity for PRSIM_23 of 5.4 nM (FIG. 24B), whereas the affinity of HCV NS3/4A PR K136D (FIG. 24C) and HCV NS3/4A PR K136N (FIG. 24D) had decreased ˜6-7-fold compared to ‘Wt’ (Table 7). HCV NS3/4A PR 0168E had an affinity for PRSIM_23 of 14.7 nM (FIG. 24E), ˜3-fold lower affinity than ‘wt’ protease.

TABLE 6
Simeprevir EC₅₀ values for the induction of mutant HCV NS3/4A
PR/PRSIM_23 binding molecule heterodimerisation by simeprevir.

| | | Simeprevir | |
|---|---|---|---|
| | | EC₅₀ | 95% CI |
| | Mutation | (nM) | (nM) |
| HCV PR + | ‘WT’ (S139A) | 4.53 | 3.95-5.19 |
| PRSIM_23 | K136D | 131.5 | 116.0-149.0 |
| | K136N | 8.06 | 6.70-9.71 |
| | D168E | 3.69 | 3.40-4.01 |

TABLE 7
Binding and kinetic constants measured for the binding of mutant HCV NS3/4A PR to
PRSIM_23 binding molecule in the presence of simeprevir.

| | | Immobilised | | | | | |
|---|---|---|---|---|---|---|---|
| | | level | Simeprevir | kₐ | k_{d} | K_{D} | Rₘₐₓ |
| | Mutation | (RUs) | (nM) | (M⁻¹ s⁻¹) | (s⁻¹) | (nM) | (RUs) |
| HCV PR + | ‘WT’ | ~500 | 20 | 5.59E+09 | 3.10E+01 | 5.4 | 215 |
| PRSIM_23 | (S139A) | | | (± 4.70E+09) | (± 2.80E+01) | (± 0.3) | (± 16) |
| | K136D | ~630 | 800 | 1.69E+10 | 3.73E+02 | 33.0 | 168 |
| | | | | (± 2.72E+10) | (± 5.77E+02) | (± 16) | (± 61) |
| | K136N | ~640 | 40 | 5.31E+08 | 2.04E+01 | 39.5 | 198 |
| | | | | (± 5.27E+08) | (± 1.87E+01) | (± 5) | (± 42) |
| | D168E | ~590 | 20 | 1.47E+09 | 2.14E+01 | 14.7 | 199 |
| | | | | (± 1.44E+09) | (± 2.09E+01) | (± 0.4) | (± 20) |

Data is mean + s.d., n = 3

### Example 18—Small Molecule Inhibitors of HCV PR can Disrupt the PRSIM 23 Complex by Competing with Simeprevir for Binding to HCV PR Variants, but not to HCV PR “Wt”

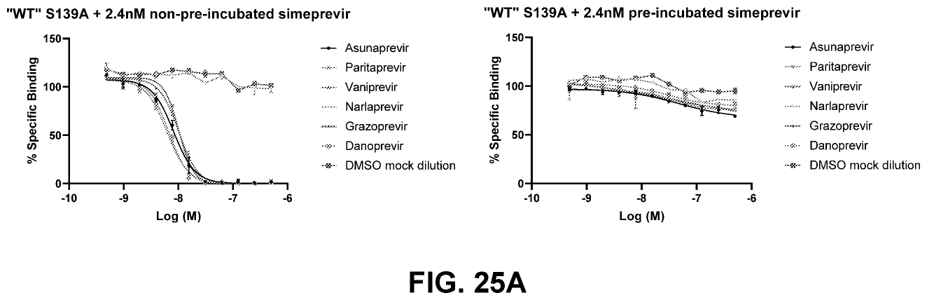

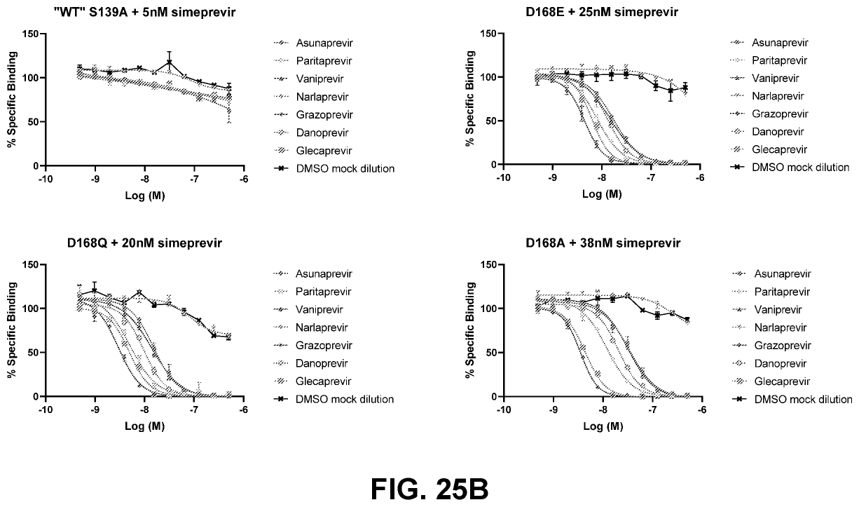

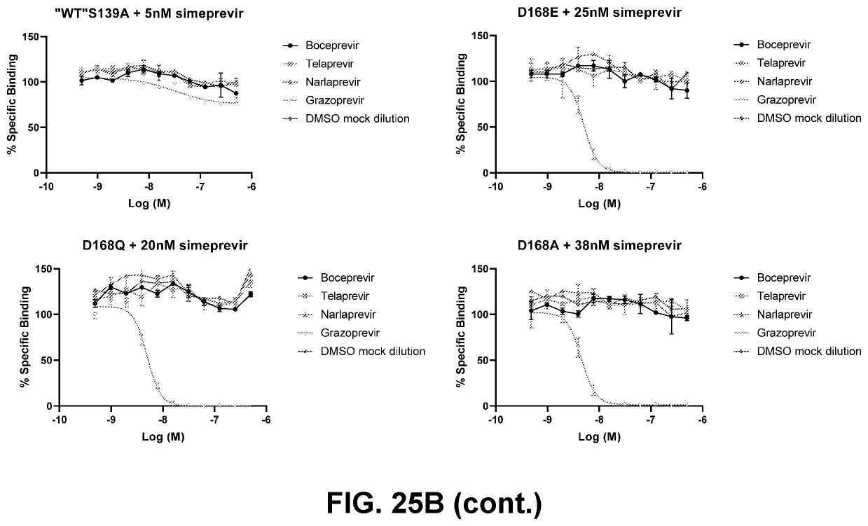

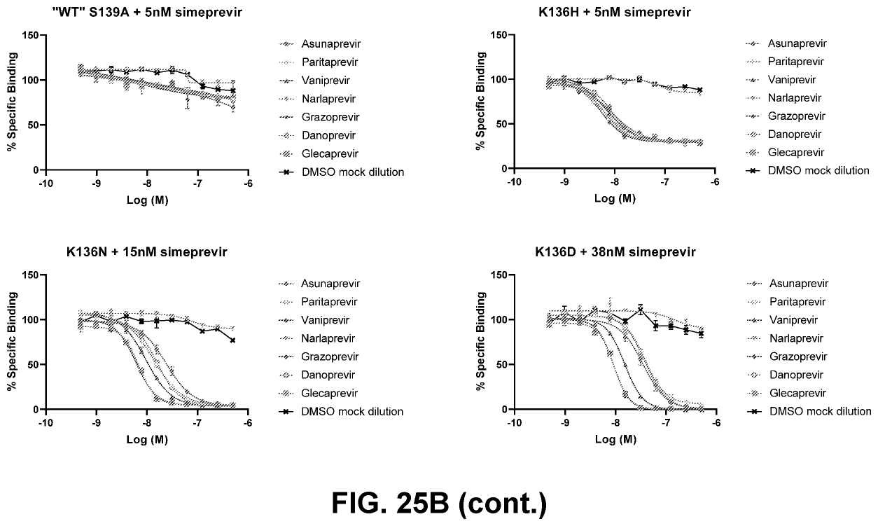

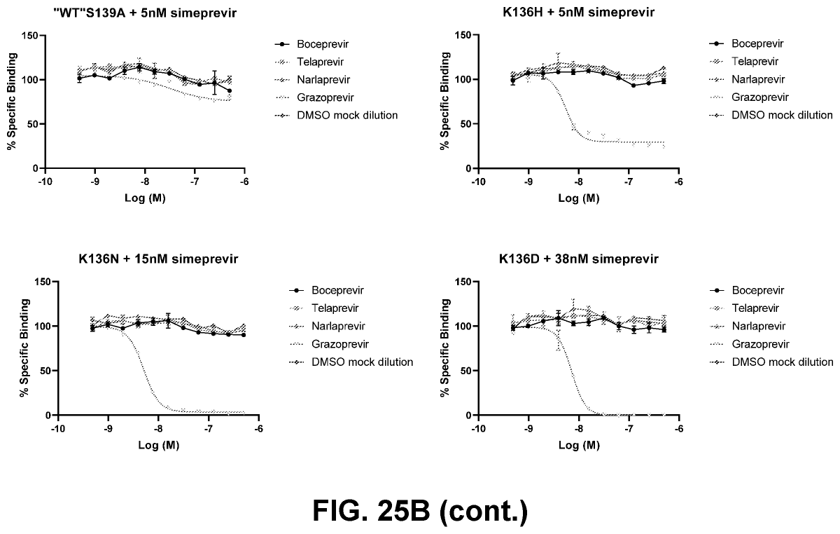

Having demonstrated that HCV NS3/4A PR (S139A):PRSIM_23 complex formation was specific for simeprevir (Example 13), we went on to investigate whether our panel of small molecule HCV PR inhibitors were able to disrupt the HCV NS3/4A PR (S139A): simeprevir: PRSIM_23 complex, by competing with simeprevir for binding to HCV PR. We found that when the small molecule inhibitors were added in a homogeneous time-resolved fluorescence (HTRF) binding assay concomitantly with simeprevir, a subset of these small molecules were able to inhibit HCV NS3/4A PR (S139A):PRSIM_23 complex formation. However, when simeprevir is pre-incubated with HCV NS3/4A PR (S139A) prior to addition of the small molecule inhibitors, no significant complex inhibition is observed (FIG. 25A).

To further characterise the mutations made to the HCV NS3/4A PR, we investigated whether the small molecules were able to disrupt pre-formed mutant HCV PR: simeprevir: PRSIM_23 complexes. Where a mutation at position 136 is made, more pronounced inhibition of the mutant HCV PR: simeprevir: PRSIM_23 complex is observed with a subset of the small molecule inhibitors (asunaprevir, paritaprevir, vaniprevir, grazoprevir, danoprevir and glecaprevir), but not with others (narlaprevir, boceprevir and telaprevir) (FIG. 25B). The degree of inhibition is dependent on the specific mutation made. Approximately 75% inhibition is observed with K136H, despite having a similar EC80 for simeprevir as HCV PR “wt”. Near complete inhibition is seen for K136N and complete inhibition is observed for K1360. Complete inhibition of the HCV NS3/4A PR (S139A): simeprevir: PRSIM_23 complex is observed for all HCV PR variants with a mutation at position 168.

The ability of other small molecule inhibitors (asunaprevir, paritaprevir, vaniprevir, grazoprevir, danoprevir and glecaprevir) to “compete” with simeprevir, and disrupt the complex between PRSIM_23 and mutant versions of HCV NS3/4A PR, provides an opportunity to rapidly inactivate any PRSIM-based CID, and turn off transgene expression or therapeutic activity. Furthermore, the inability of other inhibitors (narlaprevir, boceprevir and telaprevir) to compete with simeprevir for HCV NS3/4A PR binding provides an opportunity to develop orthogonal HCV NS3/4A PR-based molecular switches that are induced by these small molecules.

### Example 19—Mutants of HCV NS3/4A PR that are Incorporated into a Split Transcription Factor System Retain the Ability to Regulate Gene Expression

To assess the impact mutations of HCV NS3/4A PR on gene regulation we generated pHet-Act1-2-based constructs encoding HCV NS3/4A PR (S139A)-AD mutants & DBD-PRSIM_23 (three tandem copies). Following transfection of cells with these pHet-Act1-2 (HCV NS3/4A PR (S139A)-AD mutants & DBD-PRSIM_23 (three tandem copies)) constructs or ‘WT’ construct (HCV NS3/4A PR (S139A)-AD & DBD-PRSIM_23 (three tandem copies)) with the reporter construct pZFHD1_Luciferase, we assessed gene expression. The ability to regulate luciferase gene expression in the presence of increasing concentrations of simeprevir was determined. All PRSIM HCV NS3/4A PR (S139A)-AD mutants demonstrated dose-dependent gene expression of luciferase, albeit with a slight reduction of the max fold change and increase in EC50 relative to the ‘WT’ HCV NS3/4A PR (S139A)-AD (FIG. 26 and Table 8).

The combined data from examples 14-19 suggests that mutant HCV NS3/4A PR-containing PRSIM-based CIDs could provide alternatives to the HCV NS3/4A (S139A) “wt”-based CID in scenarios where rapid reversal of CID-based activity is required, through the administration of “competing” small molecule HCV PR inhibitors.

TABLE 8
EC50 and fold-change values for HCV NS3/4A PR variants in a split
transcription factor assay

| HCV NS3/4A PR | | |
|---|---|---|
| variant | EC50 (nM) | Max fold change |
| K136D | 84.66 | 5569 |
| D168E | 18.67 | 5877 |
| K136N | 18.17 | 5899 |
| “WT” S139A | 6.082 | 6973 |

To assess whether the decreased affinity/increased dissociation rate of the HCV NS3/4A PR (S139A) mutants (K1360, D168E, K136N) would impact the rate at which gene expression could be switched off upon simeprevir removal, a cell-based assay was performed using a live cell time-course assay. Monoclonal stable cell lines were generated in which the expression of a short-lived green fluorescent protein (GFP-PEST, half-live ˜2h) was placed under the control of a split transcription factor composed of HCV NS3/4A PR (S139A)-AD variants & DBD-PRSIM_23 (three tandem copies). GFP expression was induced by 24h treatment with simeprevir after which simeprevir was removed and GFP fluorescence at timepoints after removal was determined. The ‘WT’S139A retained high GFP-fluorescence over 24 h. This shows that once formed in a simeprevir-dependent fashion, the transcription factor complex containing the HCV NS3/4A PR (S139A) remains stable for a prolonged period of time to drive continued GFP-PEST-expression which does not require the continued presence of excess simeprevir in the culture medium. However, over the same period of time, all three mutants (K136D, K136N, D168E) return to a native, non-expressing state within 15-24h after removal of simeprevir demonstrating the reduced stability of the transcription factor complexes formed using HCV NS3/4A PR (S139A)-AD mutants & DBD-PRSIM_23 (three tandem copies) compared to HCV NS3/4A PR (S139A)-AD ‘WT’ & DBD-PRSIM_23 (three tandem copies).

This data suggests that, by reducing the affinity of simeprevir to mutants of HCV NS3/4A PR, it is possible to alter the kinetics of gene expression, enabling the cessation of gene expression to occur faster than when using the “wt” HCV NS3/4A PR-based CID, in the split transcription format.

### Example 20—Crystal Structure of the HCV NS3/4A PR (S139A): Simeprevir: PRSIM 57 Complex Reveals the Mechanism of Small Molecule Triggered Dimerization

Simeprevir induces the formation of a heterodimer of the HCV NS3/4A PR (S139A) and the scFv molecule PRSIM_57 by binding to a pocket on the surface of the protease and generating a new epitope that is specifically recognised by PRSIM_57. In order to understand the molecular mechanisms underlying this heterodimerisation event, a crystal structure of the complex between protease, scFv and simeprevir was determined. To derive the structure, forms of the protease and PRSIM_57 scFv with tobacco etch virus (TEV)-cleavable His-tags were both expressed separately in BL21(DE3) E. coli. The proteins were purified to homogeneity using a combination of immobilised metal affinity chromatography and size exclusion chromatography, and tags removed by treatment with TEV protease. In order to form the ternary complex, the protease was incubated with an excess of PRSIM_57 and simeprevir and the resulting complex was purified from non-complexed material using size exclusion chromatography. The fractions containing pure complex were pooled and concentrated to 12 mg/ml and set up in crystal trials. The complex was crystallised via sitting drop vapour diffusion and X-ray diffraction data were collected from crystals at a synchrotron X-ray source. The structure was solved using molecular replacement with the structure of the apo form of HCV NS3/4A PR (S139A) as the search model.

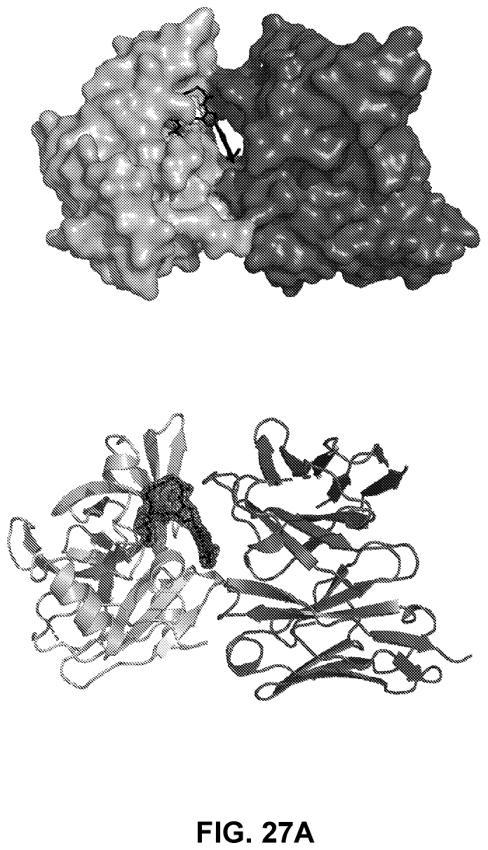

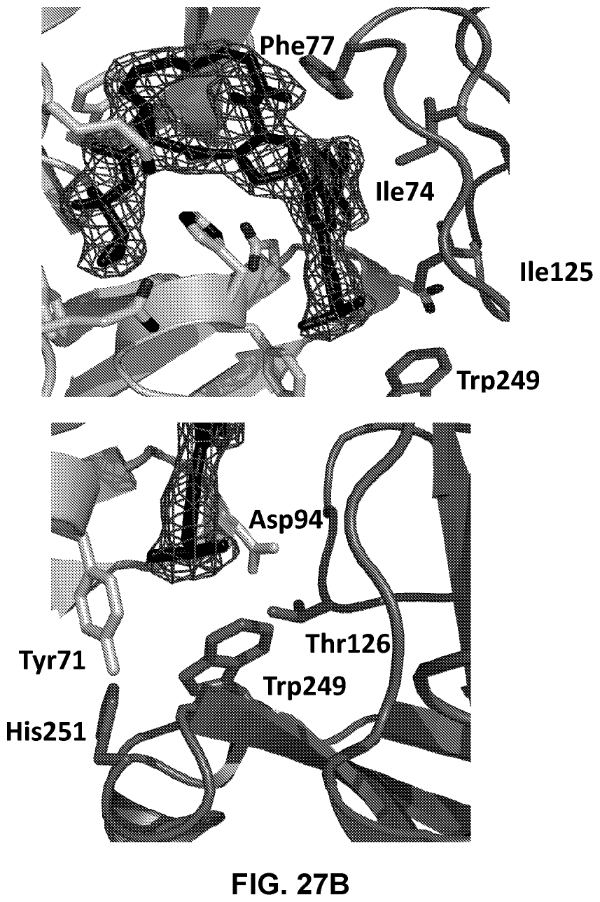

All three components of the ternary complex are clearly visible in the electron density (FIG. 27A). The simeprevir is bound to the HCV NS3/4A PR (S139A) in the same pose and via the same interactions observed previously (PDB id 3KEE). The structure reveals that the majority of the interactions made by the PRSIM_57 scFv are direct to residues in the protease, with limited contacts with simeprevir. The scFv forms a primarily hydrophobic pocket around simeprevir (including side chains of Phe77, Ile74, Ile125 and Trp249), clamping either side of it and engaging the protease. The binding is dominated by the scFv complementarity determining region (CDR) loops HCDR2, HCDR3 and LCDR3.

The following interactions can be identified between PRSIM_57 and HCV NS3/4A PR (S139A) (FIG. 27B): 1) The sidechain carboxyl of Asp94 (HCV NS3/4A PR) makes interactions with the backbone nitrogen atoms of Ile125 and Thr126 (PRSIM_57) and with the sidechain hydroxyl of Thr126. 2) The sidechain hydroxyl of Tyr71 (HCV NS3/4A PR) makes interactions with the sidechains of His251 and Trp249 (PRSIM_57). 3) A hydrophobic interaction is made between the sidechains of Va193 (HCV NS3/4A PR) and Trp249 (PRSIM_57). 4) Water-mediated interactions between Glu254 (PRSIM_57) and backbone nitrogen atoms of Gly75 and Thr76 (HCV NS3/4A PR). The major interaction between PRSIM_57 and simeprevir is an interaction of the simeprevir quinoline moiety with the side chain of Phe77 in HCDR2 (PRSIM_57).

### Example 21—PRSIM-Based CIDs can Regulate the Activity of an Apoptotic Protein to Control Cell Death

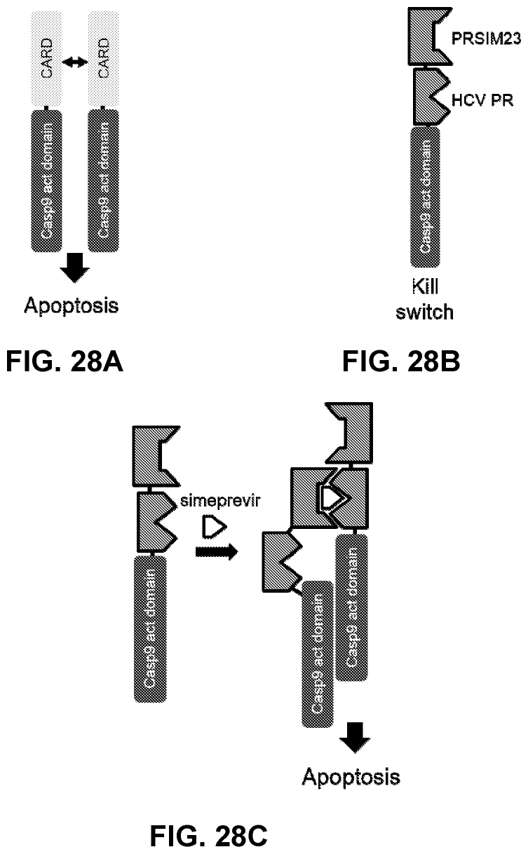

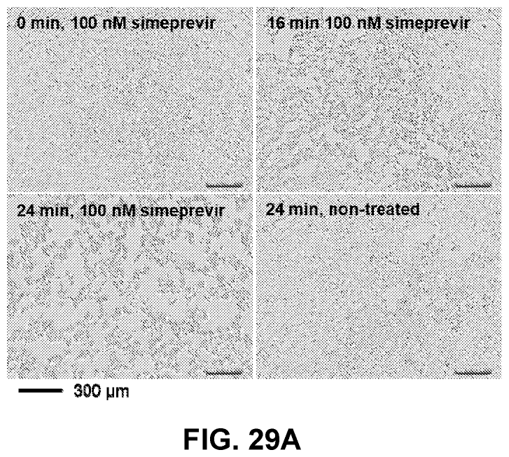

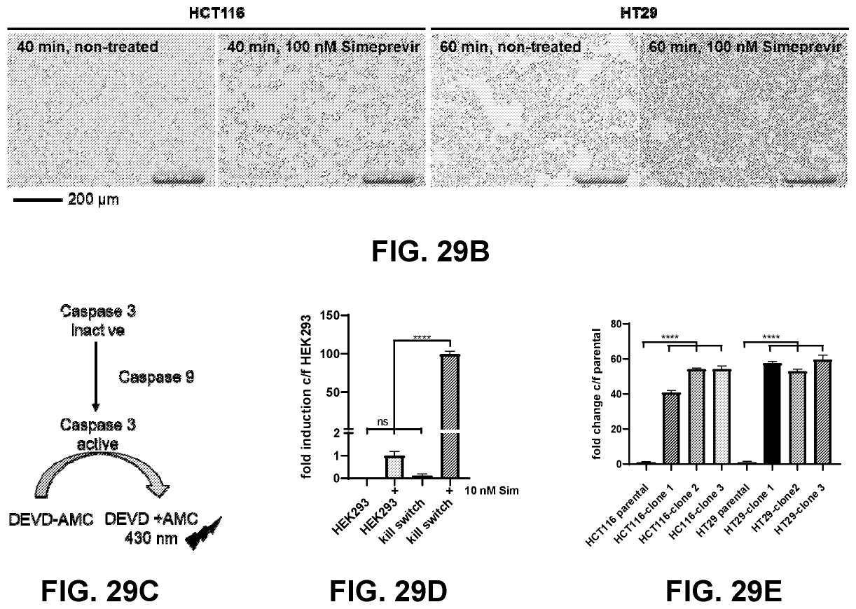

The ability to “remotely control” therapeutic cells once they have been administered, provides a safety net, in the advent of uncontrolled proliferation or adverse event. One way to control such cells is to endow them with a so called “kill switch” such that they can be removed at will once they have performed their function or pose a safety risk. As such, a PRSIM-based, simeprevir-responsive Caspase 9-based kill switch was generated and tested in vitro. The homo-dimerisation CARD domain of Caspase 9 was replaced with both the PRSIM23 and HCV NS3/4A PR (S139A) domains, separated by short linkers. An active Caspase 9 homodimer can thus only be reconstituted by addition of simeprevir (FIG. 28). Addition of simeprevir to HEK293, HCT116 and HT29 cells stably transduced with the PRSIM-based kill switch construct shows rapid cell death upon addition of 100 nM simeprevir by microscopic inspection of cells (FIG. 29A, B). Active Caspase 9 activates downstream Caspase3 by proteolytic cleavage. Caspase 3 activity is detected by cleavage of fluorogenic substrate Ac-DEVD-AMC (FIG. 29C). Caspase 3 activity is significantly (p<0.0001) up-regulated in simeprevir-treated kill-switch-transduced HEK293 cells (FIG. 29D) or kill switch transduced human tumour cell lines HCT116 and HT29 (FIG. 29E).

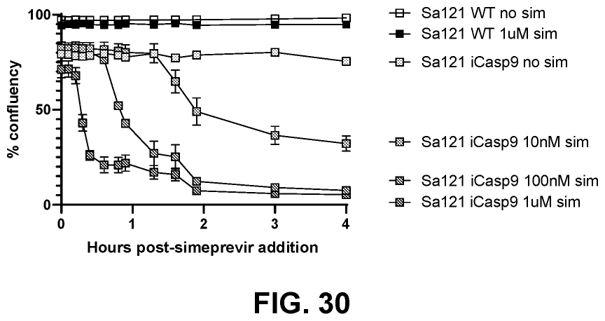

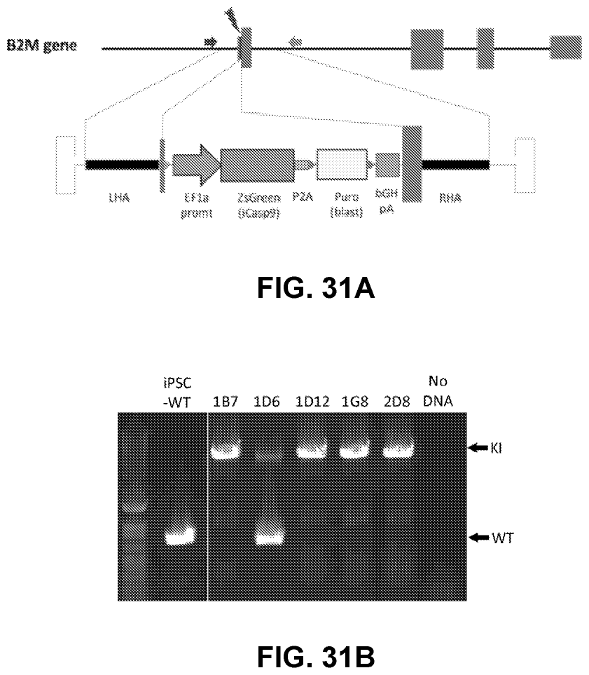

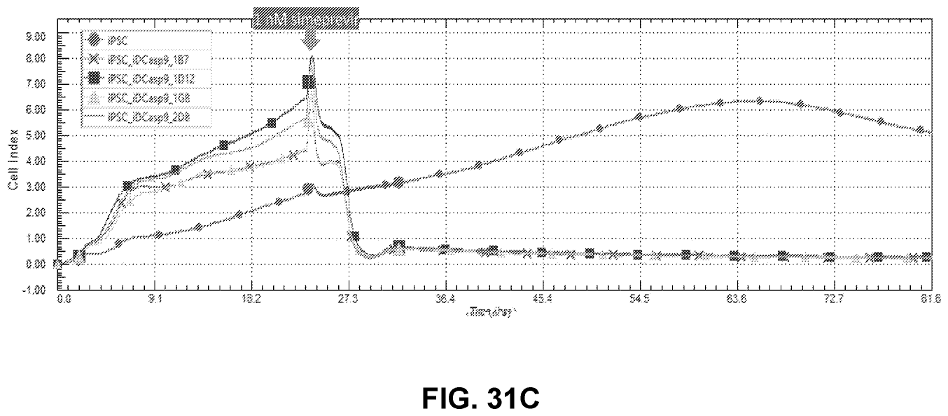

To demonstrate that the PRSIM-based kill switch can eliminate therapeutically-relevant cells, stable cell lines were made in both embryonic stem (ES) cells and induced pluripotent stem cells (iPSC). In ES cells, a dose-response to simeprevir can be observed whereby a high dose of simeprevir (1 μM) rapidly and efficiently eliminates up to 95% of cells within 4 hours, as measured by cell confluency, with an onset of ˜15 minutes (FIG. 30). Lower doses initiate cell killing with a delayed onset; 100 nM of simeprevir was able to induce ˜90% cell killing within 4 hours, whereas at 10 nM maximal cell killing was not reached within the 4 hours timeframe of the experiment. In contrast, wt Sa121 cells did not respond to treatment with simeprevir.

To demonstrate the effectiveness of the PRSIM-based kill switch in iPSC cells, four individual iPSC clones that were biallelic for the PRSIM-based kill switch at the B2M locus were generated. These cells, alongside parental iPSC cells were incubated with 1 nM simeprevir and the cell proliferation index was measured over time using the xCELLigence RTCA Software Pro (ACEA Biosciences Inc.). All cell clones that encoded the PRSIM-based kill switch showed a dramatic reduction in cell proliferation index after 5 hours which was maintained over the course of the experiment (˜60 hours, post-simeprevir addition), whereas the parental cells continued to proliferate.

These data demonstrate that a PRSIM-based kill switch can efficiently eliminate a wide range of cell types in vitro and provides a means for the rapid removal of therapeutic cells in patients.

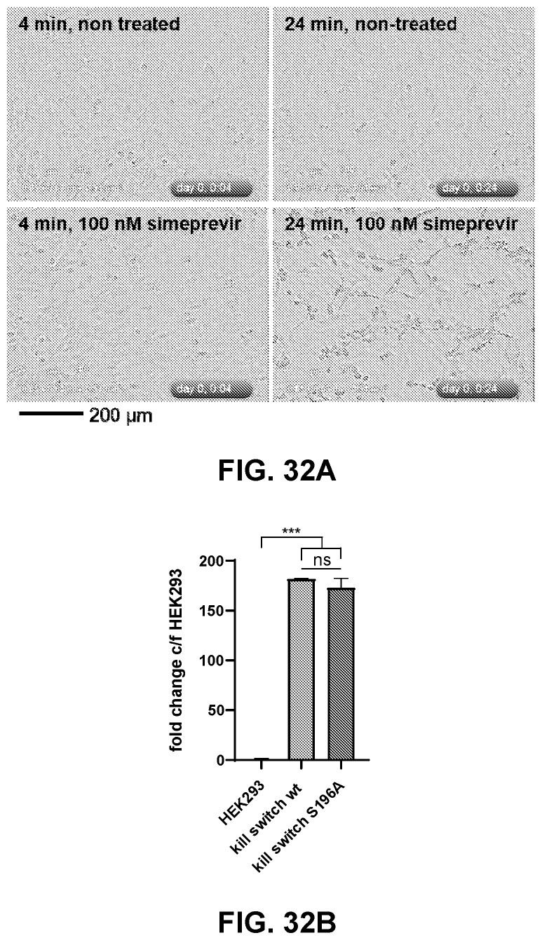

Caspase 9 can be inactivated by Aid kinase-mediated phosphorylation on Ser96. This poses a risk of “escape” from Caspase 9 mediated apoptosis by cells that have undergone phosphorylation of Ser96 on the Caspase 9 fusion protein. To mitigate this risk, a stable HEK cell line encoding the PRSIM-based kill switch fusion protein containing a Ser196 to Ala substitution was generated. Addition of 100 nM simeprevir to kill switch S196A cells showed rapid cell killing in a timeframe comparable to the wt kill switch (FIG. 32A). Activity of downstream caspase 3 was significantly (p<0.0005) upregulated in both wt and S196A mutant kill switch cells compared to non-transduced cells; in the same assay, no significant differences between wt and S196A kill switch cells were detected (FIG. 32B). This demonstrates that the S196A version of the PRSIM-based kill switch fusion protein is as active as the wild-type Caspase 9-based kill switch, and can be used as a mechanism to prevent the Akt-mediated cellular evasion mechanism.

### REFERENCES

A number of publications are cited above in order to more fully describe the present disclosure and the state of the art to which the disclosure pertains. Full citations for these references are provided below. The entirety of each of these references is incorporated herein.
Altschul S F, Gish W, Miller W, Myers E W, Lipman D J. 1990. ‘Basic local alignment search tool’. J. Mol. Biol. 215(3), 403-10.
Banaszynski, L. A., C. W. Liu, and T. J. Wandless. 2005. ‘Characterization of the FKBP.rapamycin.FRB ternary complex’, J Am Chem Soc, 127: 4715-21.
Bartenschlager R, Ahlborn-Laake L, Mous J, Jacobsen H. 1993 ‘Nonstructural protein 3 of the hepatitis C virus encodes a serine-type proteinase required for cleavage at the NS3/4 and N54/5 junctions’. J Virol.;67(7):3835-3844
Belshaw, P J, S N Ho, G R Crabtree, and S L Schreiber. 1996. ‘Controlling protein association and subcellular localization with a synthetic ligand that induces heterodimerization of proteins’, Proceedings of the National Academy of Sciences, 93: 4604-07.
Belshaw, P. J., D. M. Spencer, G. R. Crabtree, and S. L. Schreiber. 1996. ‘Controlling programmed cell death with a cyclophilin-cyclosporin-based chemical inducer of dimerization’, Chem Biol, 3: 731-8.
Chavez A, Scheiman J, Vora S, Pruitt B W, Tuttle M, P R Iyer E, Lin S, Kiani S, Guzman C D, Wiegand D J, Ter-Ovanesyan D, Braff J L, Davidsohn N, Housden B E, Perrimon N, Weiss R, Aach J, Collins J J, Church G M. Nat. Methods., 12(4): 326-8
Chelur D S, Chalfie M. 2007. ‘Targeted cell killing by reconstituted caspases.’ Proc. Natl. Acad. Sci. U.S.A., 104(7): 2283-8
Colella P, Ronzitti G, Mingozzi F. 2017. ‘Emerging Issues in AAV-Mediated In Vivo Gene Therapy.’ Mol Ther Methods Clin Dev., 8: 87-104
De Clercq E. 2014. ‘Current race in the development of DAAs (direct-acting antivirals) against HCV.’ Biochem. Pharmacol, 89(4): 441-52
Dixon A S, Schwinn M K, Hall M P, Zimmerman K, Otto P, Lubben T H, Butler B L, Binkowski B F, Machleidt T, Kirkland T A, Wood M G, Eggers C T, Encell L P, Wood K V. 2016. ‘NanoLuc Complementation Reporter Optimized for Accurate Measurement of Protein Interactions in Cells.’ ACS Chem. Biol., 11(2): 400-8
Eckart, M. R. M. Selby, F. Masiarz, C. Lee, K. Berger, K. Crawford, C. Kuo, G. Kuo, M. Houghton, Q. L. Choo. 1993 The Hepatitis C Virus Encodes a Serine Protease Involved in Processing of the Putative Nonstructural Proteins from the Viral Polyprotein Precursor, Biochemical and Biophysical Research Communications, Volume 192, Issue 2, 1993, Pages 399-406
Foight G W, Wang Z, Wei C T, et al. Multi-input chemical control of protein dimerization for programming graded cellular responses. Nat Biotechnol. 2019; 37(10):1209-1216. doi:10.1038/s41587-019-0242-8
Gargett T, Brown M P. 2014. ‘The inducible caspase-9 suicide gene system as a “safety switch” to limit on-target, off-tumor toxicities of chimeric antigen receptor T cells.’ Front Pharmacol., 5:235.
Gilbreth, R. N., B. M. Chacko, L. Grinberg, J. S. Swers, and M. Baca. 2014. ‘Stabilization of the third fibronectin type III domain of human tenascin-C through minimal mutation and rational design’, Protein Eng Des Sel, 27: 411-8.
Grakoui A, McCourt D W, Wychowski C, Feinstone S M, Rice C M. 1993 ‘Characterization of the hepatitis C virus-encoded serine proteinase: determination of proteinase-dependent polyprotein cleavage sites.’ J Virol, 67(5):2832-2843)
Hijikata M, Mizushima H, Akagi T, et al. 1993 ‘Two distinct proteinase activities required for the processing of a putative nonstructural precursor protein of hepatitis C virus.’ J Virol.; 67(8):4665-4675.
Hill, Z. B., A. J. Martinko, D. P. Nguyen, and J. A. Wells. 2018. ‘Human antibody-based chemically induced dimerizers for cell therapeutic applications’, Nat Chem Biol, 14: 112-17.
Kotterman M A & Schaffer D V. 2014. ‘Engineering adeno-associated viruses for clinical gene therapy.’ Nat. Rev. Genet. 15(7): 445-51.
Leahy, D. J., W. A. Hendrickson, I. Aukhil, and H. P. Erickson. 1992. ‘Structure of a fibronectin type III domain from tenascin phased by MAD analysis of the selenomethionyl protein’, Science, 258: 987-91.
Li J, Abel R, Zhu K, Cao Y, Zhao S, Friesner R A. The VSGB 2.0 model: a next generation energy model for high resolution protein structure modeling. Proteins. 2011; 79(10):2794-2812.
Li, Kui, Eileen Foy, Josephine C. Ferreon, Mitsuyasu Nakamura, Allan C. M. Ferreon, Masanori Ikeda, Stuart C. Ray, Michael Gale, and Stanley M. Lemon. 2005. ‘Immune evasion by hepatitis C virus NS3/4A protease-mediated cleavage of the Toll-like receptor 3 adaptor protein TRIF’, Proceedings of the National Academy of Sciences of the United States of America, 102: 2992-97.
Li, Xiao-Dong, Lijun Sun, Rashu B. Seth, Gabriel Pineda, and Zhijian J. Chen. 2005. ‘Hepatitis C virus protease NS3/4A cleaves mitochondrial antiviral signaling protein off the mitochondria to evade innate immunity’, Proceedings of the National Academy of Sciences of the United States of America, 102: 17717-22.
Lv Z, Chu Y, Wang Y. 2015. ‘HIV protease inhibitors: a review of molecular selectivity and toxicity.’ HIV AIDS (Auckl)., 7: 95-104
Moraca, F., Negri, A., de Oliveira, C. & Abel, R. Application of Free Energy Perturbation (FEP+) to Understanding Ligand Selectivity: A Case Study to Assess Selectivity Between Pairs of Phosphodiesterases (PDE's). J Chem Inf Model 59, 2729-2740 (2019).
Naso M F, Tomkowicz B, Perry W L 3rd, Strohl W R. 2017 ‘Adeno-Associated Virus (AAV) as a Vector for Gene Therapy.’ BioDrugs, 31(4): 317-334
Oganesyan, V., A. Ferguson, L. Grinberg, L. Wang, S. Phipps, B. Chacko, S. Drabic, T. Thisted, and M. Baca. 2013. ‘Fibronectin type III domains engineered to bind CD40L: cloning, expression, purification, crystallization and preliminary X-ray diffraction analysis of two complexes’, Acta Crystallogr Sect F Struct Biol Cryst Commun, 69: 1045-8.
Osbourn, J. K., A. Field, J. Wilton, E. Derbyshire, J. C. Earnshaw, P. T. Jones, D. Allen, and J. McCafferty. 1996. ‘Generation of a panel of related human scFv antibodies with high affinities for human CEA’, Immunotechnology, 2: 181-96.
Patick A K, Potts K E. 1998. ‘Protease inhibitors as antiviral agents.’ Clin. Microbiol. Rev., 11(4): 614-27
Pomerantz J L, Sharp P A, Pabo C O. 1995. ‘Structure-based design of transcription factors.’ Science. 267(S194): 93-6
Sabariegos, Rosario, Fernando Picazo, Beatriz Domingo, Sandra Franco, Miguel-Angel Martinez, and Juan Llopis. 2009. ‘Fluorescence Resonance Energy Transfer-Based Assay for Characterization of Hepatitis C Virus NS3-4A Protease Activity in Live Cells’, Antimicrobial Agents and Chemotherapy, 53: 728-34.
Sabers, C. J., M. M. Martin, G. J. Brunn, J. M. Williams, F. J. Dumont, G. Wiederrecht, and R. T. Abraham. 1995. ‘Isolation of a protein target of the FKBP12-rapamycin complex in mammalian cells’, J Biol Chem, 270: 815-22.
Sadelain M, Brentjens R, Rivière I. 2013 ‘The basic principles of chimeric antigen receptor design.’ Cancer Discov., 3(4): 388-98
Sastry, G. M., Adzhigirey, M., Day, T., Annabhimoju, R. & Sherman, W. Protein and ligand preparation: parameters, protocols, and influence on virtual screening enrichments. J Comput Aided Mol Des 27, 221-234 (2013).
Smith-Garvin, J. E., G. A. Koretzky, and M. S. Jordan. 2009.‘T cell activation’, Annu Rev Immunol, 27: 591-619.
Srivastava A. 2016. ‘In vivo tissue-tropism of adeno-associated viral vectors.’ Curr. Opin. Virol. 21: 75-80
Stanton, B. Z., E. J. Chory, and G. R. Crabtree. 2018. ‘Chemically induced proximity in biology and medicine’, Science, 359.
Stempniak M, Hostomska Z, Nodes BR, Hostomsky Z. 1997 ‘The NS3 proteinase domain of hepatitis C virus is a zinc-containing enzyme.’ J Virol., 71(4):2881-2886.
Swers, J. S., L. Grinberg, L. Wang, H. Feng, K. Lekstrom, R. Carrasco, Z. Xiao, I. Inigo, C. C. Leow, H. Wu, D. A. Tice, and M. Baca. 2013. ‘Multivalent scaffold proteins as superagonists of TRAIL receptor 2-induced apoptosis’, Mol Cancer Ther, 12: 1235-44.
Vaughan, T. J., A. J. Williams, K. Pritchard, J. K. Osbourn, A. R. Pope, J. C. Earnshaw, J. McCafferty, R. A. Hodits, J. Wilton, and K. S. Johnson. 1996. ‘Human antibodies with sub-nanomolar affinities isolated from a large non-immunized phage display library’, Nat Biotechnol, 14: 309-14.
Wu, C. Y., K. T. Roybal, E. M. Puchner, J. Onuffer, and W. A. Lim. 2015. ‘Remote control of therapeutic T cells through a small molecule-gated chimeric receptor’, Science, 350: aab4077.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

Sequences

| SEQ | | Protein/ | |
|---|---|---|---|
| ID NO: | Description | DNA | Sequence |
| 1 | Wild-type | Protein | MKKKGSVVIVGRINLSGDTAYAQQTRGEEGCQETSQTGRDKNQVEGEVQIVSTATQ |
| | HCV NS3/4APR | | TFLATSINGVLWTVYHGAGTRTIASPKGPVTQMYTNVDKDLVGWQAPQGSRSLTPC |
| | | | TCGSSDLYLVTRHADVIPVRRRGDSRGSLLSPRPISYLKGSSGGPLLCPAGHAVGI |
| | | | FRAAVSTRGVAKAVDFIPVESLETTMRSP |
| 2 | HCV NS3/4APR | Protein | MKKKGSVVIVGRINLSGDTAYAQQTRGEEGCQETSQTGRDKNQVEGEVQIVSTATQ |
| | (S139A) | | TFLATSINGVLWTVYHGAGTRTIASPKGPVTQMYTNVDKDLVGWQAPQGSRSLTPC |
| | | | TCGSSDLYLVTRHADVIPVRRRGDSRGSLLSPRPISYLKGSAGGPLLCPAGHAVGI |
| | | | FRAAVSTRGVAKAVDFIPVESLETTMRSP |
| 3 | Wild-type | Protein | MGSSHHHHHHGSGLNDIFEAQKIEWHEGGGGSMKKKGSVVIVGRINLSGDTAYAQQ |
| | HCV NS3/4APR | | TRGEEGCQETSQTGRDKNQVEGEVQIVSTATQTFLATSINGVLWTVYHGAGTRTIA |
| | with N- | | SPKGPVTQMYTNVDKDLVGWQAPQGSRSLTPCTCGSSDLYLVTRHADVIPVRRRGD |
| | terminal 6His | | SRGSLLSPRPISYLKGSSGGPLLCPAGHAVGIFRAAVSTRGVAKAVDFIPVESLET |
| | and AviTag | | TMRSP |
| 4 | HCV NS3/4APR | Protein | MGSSHHHHHHGSGLNDIFEAQKIEWHEGGGGSMKKKGSVVIVGRINLSGDTAYAQQ |
| | (S139A) | | TRGEEGCQETSQTGRDKNQVEGEVQIVSTATQTFLATSINGVLWTVYHGAGTRTIA |
| | with N- | | SPKGPVTQMYTNVDKDLVGWQAPQGSRSLTPCTCGSSDLYLVTRHADVIPVRRRGD |
| | terminal 6His | | SRGSLLSPRPISYLKGSAGGPLLCPAGHAVGIFRAAVSTRGVAKAVDFIPVESLET |
| | and AviTag | | TMRSP |
| 5 | PRSIM_23 | Protein | RLDAPSQIEVKDVTDTTALITWVDPRYDDIWWFELTYGIKDVPGDRTTIKLYLNDP |
| | | | YYSIGNLKPDTEYEVSLISYTGDSYSRSGSNPAKITFKTGL |
| 6 | PRSIM_32 | Protein | RLDAPSQIEVKDVTDTTALITWWSPRYYYASISGFELTYGIKDVPGDRTTIKLDYA |
| | | | SNDYSIGNLKPDTEYEVSLISWNYGDWRYSSSNPAKITFKTGL |
| 7 | PRSIM_33 | Protein | RLDAPSQIEVKDVTDTTALITWYPPGRWYDDIWYFELTYGIKDVPGDRTTIKLARG |
| | | | DDVYSIGNLKPDTEYEVSLISWGPDRGDRAGSNPAKITFKTGL |
| 8 | PRSIM_36 | Protein | RLDAPSQIEVKDVTDTTALITWSWPRDDDYDIWYFELTYGIKDVPGDRTTIKLLNY |
| | | | ASPYSIGNLKPDTEYEVSLISVVPDTYGRGTSNPAKITFKTGL |
| 9 | PRSIM_47 | Protein | RLDAPSQIEVKDVTDTTALITWSRPGVSIWYFELTYGIKDVPGDRTTIKLDYRSYY |
| | | | YSIGNLKPDTEYEVSLISGSYGLVGVRASNPAKITFKTGL |
| 10 | PRSIM_01 | Protein | QVQLVQSGAEVKKPGSSVKVSCKASGGTFSSYAISWVRQAPGQGLEWMGGIIPIFG |
| | | | TANYAQKFQGRVTITADESTSTAYMELSSLRSEDTAVYYCARGQGYITVFDYWGQG |
| | | | TLVTVSSGGGGSGGGGSGGGGSAQSVLTQPPSASGTPGQRVTISCSGSSSNIGSNT |
| | | | VNWYQQLPGTAPKLLIYSNNQRPSGVPDRFSGSKSGTSASLAISGLQSEDEADYYC |
| | | | AAWDHHWEQVVFGGGTKLTVL |
| 11 | PRSIM_04 | Protein | QVQLVQSGAEVKKPGSSVKVSCKASGGTFSSYAISWVRQAPGQGLEWMGGIIPIFG |
| | | | TANYAQKFQGRVTITADESTSTAYMELSSLRSEDTAVYYCARGMAHFYQFDLWGQG |
| | | | TLVTVSSGGGGSGGGGSGGGGSAQSVLTQPPSASGTPGQRVTISCSGSSSNIGSNT |
| | | | VNWYQQLPGTAPKLLIYSNNQRPSGVPDRFSGSKSGTSASLAISGLQSEDEADYYC |
| | | | AAGDHDHEHVVFGGGTKLTVL |
| 12 | PRSIM_57 | Protein | QVQLVQSGAEVKKPGSSVKVSCKASGGTFSSYAISWVRQAPGQGLEWMGGIIPIFG |
| | | | TANYAQKFQGRVTITADESTSTAYMELSSLRSEDTAVYYCARHTNYITVFDYWGQG |
| | | | TLVTVSSGGGGSGGGGSGGGGSAQSVLTQPPSASGTPGQRVTISCSGSSSNIGSNT |
| | | | VNWYQQLPGTAPKLLIYSNNQRPSGVPDRFSGSKSGTSASLAISGLQSEDEADYYC |
| | | | AAWDHHWEQVVFGGGTKLTVL |
| 13 | PRSIM_67 | Protein | EVQLVQSGAEVKKPGAAVRISCKTSGYVFTSYYVHWVRQAPGQGLEWMGVINPSGG |
| | | | NTNYAQKFQDRVTMTRDTSTTTVYMELSSLMFDDTAVYYCAKRDYGGPLANWGRGT |
| | | | LVTVSSGGGGSGGGGSGGGGSALSYELTQPPSVSEAPRQRVTISCSGSSSNIGNNA |
| | | | VNWYQQLPGKAPKLLIFYDDLLPSGVSDRFSGSKSGTSASLAISGLQSEDEADYYC |
| | | | AAWDDSLNGLVFGTGTKLTVL |
| 14 | PRSIM_72 | Protein | QVQLVQSGAEVKKPGSSVKVSCKASGGTFSSYAISWVRQAPGQGLEWMGGIIPIFG |
| | | | TANYAQKFQGRVTITADESTSTAYMELSSLRSEDTAVYYCARGMAHFYQFDLWGQG |
| | | | TLVTVSSGGGGSGGGGSGGGGSAQSVLTQPPSASGTPGQRVTISCSGSSSNIGSNT |
| | | | VNWYQQLPGTAPKLLIYSNNQRPSGVPDRFSGSKSGTSASLAISGLQSEDEADYYC |
| | | | AAGDHDHEHVVFGGGTKLTVL |
| 15 | PRSIM_75 | Protein | EVQLVQSGAEVKKPGSSVKVSCKASGGSFNSYTLDWVRQAPGQGLEWMGGIIPVFG |
| | | | SPNYGQKFQGRVTITADESTSTAYMELSSLKSDDTAVYYCARGLVYQPLDSWGRGT |
| | | | LVTVSSGGGGSGGGGSGGGGSAQAVLTQPSSASGTPGQRVTISCSGSSSNIGSYTV |
| | | | NWYQQFPGTAPKLLIYSNTQRPSGVPDRFSGSKSGTSASLAISGLQSEDEADYYCA |
| | | | AWDDSLNGVWFGGGTKVTVL |
| 16 | LgBiT | Protein | MVFTLEDFVGDWEQTAAYNLDQVLEQGGVSSLLQNLAVSVTPIQRIVRSGENALKI |
| | | | DIHVIIPYEGLSADQMAQIEEVFKVVYPVDDHHFKVILPYGTLVIDGVTPNMLNYF |
| | | | GRPYEGIAVFDGKKITVTGTLWNGNKIIDERLITPDGSMLFRVTIN |
| 17 | SmBiT | Protein | VTGYRLFEEIL |
| 18 | HCV_NS4A_ | Protein | MKKKGSVVIVGRINLSGDTAYAQQTRGEEGCQETSQTGRDKNQVEGEVQIVSTATQ |
| | NS3_S139A_ | | TFLATSINGVLWTVYHGAGTRTIASPKGPVTQMYTNVDKDLVGWQAPQGSRSLTPC |
| | SmBiT | | TCGSSDLYLVTRHADVIPVRRRGDSRGSLLSPRPISYLKGSAGGPLLCPAGHAVGI |
| | | | FRAAVSTRGVAKAVDFIPVESLETTMRSPGSSGGGGSGGGGSSGVTGYRLFEEIL |
| 19 | SmBiT_HCV_ | Protein | MVTGYRLFEEILGSSGGGGSGGGGSSGKKKGSVVIVGRINLSGDTAYAQQTRGEEG |
| | NS4A_NS3_S139A | | CQETSQTGRDKNQVEGEVQIVSTATQTFLATSINGVLWTVYHGAGTRTIASPKGPV |
| | | | TQMYTNVDKDLVGWQAPQGSRSLTPCTCGSSDLYLVTRHADVIPVRRRGDSRGSLL |
| | | | SPRPISYLKGSAGGPLLCPAGHAVGIFRAAVSTRGVAKAVDFIPVESLETTMRSP |
| 20 | PRSIM_23_LgBiT | Protein | MGSRLDAPSQIEVKDVTDTTALITWVDPRYDDIWWFELTYGIKDVPGDRTTIKLYL |
| | | | NDPYYSIGNLKPDTEYEVSLISYTGDSYSRSGSNPAKITFKTGLSGSSGGGGSGGG |
| | | | GSSGVFTLEDFVGDWEQTAAYNLDQVLEQGGVSSLLQNLAVSVTPIQRIVRSGENA |
| | | | LKIDIHVIIPYEGLSADQMAQIEEVFKVVYPVDDHHFKVILPYGTLVIDGVTPNML |
| | | | NYFGRPYEGIAVFDGKKITVTGTLWNGNKIIDERLITPDGSMLFRVTIN |
| 21 | PRSIM_32_LgBiT | Protein | MGSRLDAPSQIEVKDVTDTTALITWWSPRYYYASISGFELTYGIKDVPGDRTTIKL |
| | | | DYASNDYSIGNLKPDTEYEVSLISWNYGDWRYSSSNPAKITFKTGLSGSSGGGGSG |
| | | | GGGSSGVFTLEDFVGDWEQTAAYNLDQVLEQGGVSSLLQNLAVSVTPIQRIVRSGE |
| | | | NALKIDIHVIIPYEGLSADQMAQIEEVFKVVYPVDDHHFKVILPYGTLVIDGVTPN |
| | | | MLNYFGRPYEGIAVFDGKKITVTGTLWNGNKIIDERLITPDGSMLFRVTIN |
| 22 | PRSIM_33_LgBiT | Protein | MGSRLDAPSQIEVKDVTDTTALITWYPPGRWYDDIWYFELTYGIKDVPGDRTTIKL |
| | | | ARGDDVYSIGNLKPDTEYEVSLISWGPDRGDRAGSNPAKITFKTGLSGSSGGGGSG |
| | | | GGGSSGVFTLEDFVGDWEQTAAYNLDQVLEQGGVSSLLQNLAVSVTPIQRIVRSGE |
| | | | NALKIDIHVIIPYEGLSADQMAQIEEVFKVVYPVDDHHFKVILPYGTLVIDGVTPN |
| | | | MLNYFGRPYEGIAVFDGKKITVTGTLWNGNKIIDERLITPDGSMLFRVTIN |
| 23 | PRSIM_36_LgBiT | Protein | MGSRLDAPSQIEVKDVTDTTALITWSWPRDDDYDIWYFELTYGIKDVPGDRTTIKL |
| | | | LNYASPYSIGNLKPDTEYEVSLISVVPDTYGRGTSNPAKITFKTGLSGSSGGGGSG |
| | | | GGGSSGVFTLEDFVGDWEQTAAYNLDQVLEQGGVSSLLQNLAVSVTPIQRIVRSGE |
| | | | NALKIDIHVIIPYEGLSADQMAQIEEVFKVVYPVDDHHFKVILPYGTLVIDGVTPN |
| | | | MLNYFGRPYEGIAVFDGKKITVTGTLWNGNKIIDERLITPDGSMLFRVTIN |
| 24 | PRSIM_47_LgBiT | Protein | MGSRLDAPSQIEVKDVTDTTALITWSRPGVSIWYFELTYGIKDVPGDRTTIKLDYR |
| | | | SYYYSIGNLKPDTEYEVSLISGSYGLVGVRASNPAKITFKTGLSGSSGGGGSGGGG |
| | | | SSGVFTLEDFVGDWEQTAAYNLDQVLEQGGVSSLLQNLAVSVTPIQRIVRSGENAL |
| | | | KIDIHVIIPYEGLSADQMAQIEEVFKVVYPVDDHHFKVILPYGTLVIDGVTPNMLN |
| | | | YFGRPYEGIAVFDGKKITVTGTLWNGNKIIDERLITPDGSMLFRVTIN |
| 25 | PRSIM_01_LgBiT | Protein | MGSQVQLVQSGAEVKKPGSSVKVSCKASGGTFSSYAISWVRQAPGQGLEWMGGIIP |
| | | | IFGTANYAQKFQGRVTITADESTSTAYMELSSLRSEDTAVYYCARGQGYITVFDYW |
| | | | GQGTLVTVSSGGGGSGGGGSGGGGSAQSVLTQPPSASGTPGQRVTISCSGSSSNIG |
| | | | SNTVNWYQQLPGTAPKLLIYSNNQRPSGVPDRFSGSKSGTSASLAISGLQSEDEAD |
| | | | YYCAAWDHHWEQVVFGGGTKLTVLSGSSGGGGSGGGGSSGVFTLEDFVGDWEQTAA |
| | | | YNLDQVLEQGGVSSLLQNLAVSVTPIQRIVRSGENALKIDIHVIIPYEGLSADQMA |
| | | | QIEEVFKVVYPVDDHHFKVILPYGTLVIDGVTPNMLNYFGRPYEGIAVFDGKKITV |
| | | | TGTLWNGNKIIDERLITPDGSMLFRVTIN |
| 26 | PRSIM_06_LgBiT | Protein | MGSQVQLVQSGAEVKKPGSSVKVSCKASGGTFSSYAISWVRQAPGQGLEWMGGIIP |
| | | | IFGTANYAQKFQGRVTITADESTSTAYMELSSLRSEDTAVYYCARGAGYYMRVDYW |
| | | | GQGTLVTVSSGGGGSGGGGSGGGGSAQSVLTQPPSASGTPGQRVTISCSGSSSNIG |
| | | | SNTVNWYQQLPGTAPKLLIYSNNQRPSGVPDRFSGSKSGTSASLAISGLQSEDEAD |
| | | | YYCAAWDHDVEHVVFGGGTKLTVLSGSSGGGGSGGGGSSGVFTLEDFVGDWEQTAA |
| | | | YNLDQVLEQGGVSSLLQNLAVSVTPIQRIVRSGENALKIDIHVIIPYEGLSADQMA |
| | | | QIEEVFKVVYPVDDHHFKVILPYGTLVIDGVTPNMLNYFGRPYEGIAVFDGKKITV |
| | | | TGTLWNGNKIIDERLITPDGSMLFRVTIN |
| 27 | PRSIM_57_LgBiT | Protein | MGSQVQLVQSGAEVKKPGSSVKVSCKASGGTFSSYAISWVRQAPGQGLEWMGGIIP |
| | | | IFGTANYAQKFQGRVTITADESTSTAYMELSSLRSEDTAVYYCARHTNYITVFDYW |
| | | | GQGTLVTVSSGGGGSGGGGSGGGGSAQSVLTQPPSASGTPGQRVTISCSGSSSNIG |
| | | | SNTVNWYQQLPGTAPKLLIYSNNQRPSGVPDRFSGSKSGTSASLAISGLQSEDEAD |
| | | | YYCAAWDHHWEQVVFGGGTKLTVLSGSSGGGGSGGGGSSGVFTLEDFVGDWEQTAA |
| | | | YNLDQVLEQGGVSSLLQNLAVSVTPIQRIVRSGENALKIDIHVIIPYEGLSADQMA |
| | | | QIEEVFKVVYPVDDHHFKVILPYGTLVIDGVTPNMLNYFGRPYEGIAVFDGKKITV |
| | | | TGTLWNGNKIIDERLITPDGSMLFRVTIN |
| 28 | PRSIM_67_LgBiT | Protein | MGSEVQLVQSGAEVKKPGAAVRISCKTSGYVFTSYYVHWVRQAPGQGLEWMGVINP |
| | | | SGGNTNYAQKFQDRVTMTRDTSTTTVYMELSSLMFDDTAVYYCAKRDYGGPLANWG |
| | | | RGTLVTVSSGGGGSGGGGSGGGGSALSYELTQPPSVSEAPRQRVTISCSGSSSNIG |
| | | | NNAVNWYQQLPGKAPKLLIFYDDLLPSGVSDRFSGSKSGTSASLAISGLQSEDEAD |
| | | | YYCAAWDDSLNGLVFGTGTKLTVLSGSSGGGGSGGGGSSGVFTLEDFVGDWEQTAA |
| | | | YNLDQVLEQGGVSSLLQNLAVSVTPIQRIVRSGENALKIDIHVIIPYEGLSADQMA |
| | | | QIEEVFKVVYPVDDHHFKVILPYGTLVIDGVTPNMLNYFGRPYEGIAVFDGKKITV |
| | | | TGTLWNGNKIIDERLITPDGSMLFRVTIN |
| 29 | PRSIM_72_LgBiT | Protein | MGSQVQLVQSGAEVKKPGSSVKVSCKASGGTFSSYAISWVRQAPGQGLEWMGGIIP |
| | | | IFGTANYAQKFQGRVTITADESTSTAYMELSSLRSEDTAVYYCARGMAHFYQFDLW |
| | | | GQGTLVTVSSGGGGSGGGGSGGGGSAQSVLTQPPSASGTPGQRVTISCSGSSSNIG |
| | | | SNTVNWYQQLPGTAPKLLIYSNNQRPSGVPDRFSGSKSGTSASLAISGLQSEDEAD |
| | | | YYCAAGDHDHEHVVFGGGTKLTVLSGSSGGGGSGGGGSSGVFTLEDFVGDWEQTAA |
| | | | YNLDQVLEQGGVSSLLQNLAVSVTPIQRIVRSGENALKIDIHVIIPYEGLSADQMA |
| | | | QIEEVFKVVYPVDDHHFKVILPYGTLVIDGVTPNMLNYFGRPYEGIAVFDGKKITV |
| | | | TGTLWNGNKIIDERLITPDGSMLFRVTIN |
| 30 | PRSIM_75_LgBiT | Protein | MGSEVQLVQSGAEVKKPGSSVKVSCKASGGSFNSYTLDWVRQAPGQGLEWMGGIIP |
| | | | VFGSPNYGQKFQGRVTITADESTSTAYMELSSLKSDDTAVYYCARGLVYQPLDSWG |
| | | | RGTLVTVSSGGGGSGGGGSGGGGSAQAVLTQPSSASGTPGQRVTISCSGSSSNIGS |
| | | | YTVNWYQQFPGTAPKLLIYSNTQRPSGVPDRFSGSKSGTSASLAISGLQSEDEADY |
| | | | YCAAWDDSLNGWVFGGGTKVTVLSGSSGGGGSGGGGSSGVFTLEDFVGDWEQTAAY |
| | | | NLDQVLEQGGVSSLLQNLAVSVTPIQRIVRSGENALKIDIHVIIPYEGLSADQMAQ |
| | | | IEEVFKVVYPVDDHHFKVILPYGTLVIDGVTPNMLNYFGRPYEGIAVFDGKKITVT |
| | | | GTLWNGNKIIDERLITPDGSMLFRVTIN |
| 31 | LgBiT_PRSIM_23 | Protein | MVFTLEDFVGDWEQTAAYNLDQVLEQGGVSSLLQNLAVSVTPIQRIVRSGENALKI |
| | | | DIHVIIPYEGLSADQMAQIEEVFKVVYPVDDHHFKVILPYGTLVIDGVTPNMLNYF |
| | | | GRPYEGIAVFDGKKITVTGTLWNGNKIIDERLITPDGSMLFRVTINSGSSGGGGSG |
| | | | GGGSSGRLDAPSQIEVKDVTDTTALITWVDPRYDDIWWFELTYGIKDVPGDRTTIK |
| | | | LYLNDPYYSIGNLKPDTEYEVSLISYTGDSYSRSGSNPAKITFKTGL |
| 32 | LgBiT_PRSIM_32 | Protein | MVFTLEDFVGDWEQTAAYNLDQVLEQGGVSSLLQNLAVSVTPIQRIVRSGENALKI |
| | | | DIHVIIPYEGLSADQMAQIEEVFKVVYPVDDHHFKVILPYGTLVIDGVTPNMLNYF |
| | | | GRPYEGIAVFDGKKITVTGTLWNGNKIIDERLITPDGSMLFRVTINSGSSGGGGSG |
| | | | GGGSSGRLDAPSQIEVKDVTDTTALITWWSPRYYYASISGFELTYGIKDVPGDRTT |
| | | | IKLDYASNDYSIGNLKPDTEYEVSLISWNYGDWRYSSSNPAKITFKTGL |
| 33 | LgBiT_PRSIM_33 | Protein | MVFTLEDFVGDWEQTAAYNLDQVLEQGGVSSLLQNLAVSVTPIQRIVRSGENALKI |
| | | | DIHVIIPYEGLSADQMAQIEEVFKVVYPVDDHHFKVILPYGTLVIDGVTPNMLNYF |
| | | | GRPYEGIAVFDGKKITVTGTLWNGNKIIDERLITPDGSMLFRVTINSGSSGGGGSG |
| | | | GGGSSGRLDAPSQIEVKDVTDTTALITWYPPGRWYDDIWYFELTYGIKDVPGDRTT |
| | | | IKLARGDDVYSIGNLKPDTEYEVSLISWGPDRGDRAGSNPAKITFKTGL |
| 34 | LgBiT_PRSIM_36 | Protein | MVFTLEDFVGDWEQTAAYNLDQVLEQGGVSSLLQNLAVSVTPIQRIVRSGENALKI |
| | | | DIHVIIPYEGLSADQMAQIEEVFKVVYPVDDHHFKVILPYGTLVIDGVTPNMLNYF |
| | | | GRPYEGIAVFDGKKITVTGTLWNGNKIIDERLITPDGSMLFRVTINSGSSGGGGSG |
| | | | GGGSSGRLDAPSQIEVKDVTDTTALITWSWPRDDDYDIWYFELTYGIKDVPGDRTT |
| | | | IKLLNYASPYSIGNLKPDTEYEVSLISVVPDTYGRGTSNPAKITFKTGL |
| 35 | LgBiT_PRSIM_47 | Protein | MVFTLEDFVGDWEQTAAYNLDQVLEQGGVSSLLQNLAVSVTPIQRIVRSGENALKI |
| | | | DIHVIIPYEGLSADQMAQIEEVFKVVYPVDDHHFKVILPYGTLVIDGVTPNMLNYF |
| | | | GRPYEGIAVFDGKKITVTGTLWNGNKIIDERLITPDGSMLFRVTINSGSSGGGGSG |
| | | | GGGSSGRLDAPSQIEVKDVTDTTALITWSRPGVSIWYFELTYGIKDVPGDRTTIKL |
| | | | DYRSYYYSIGNLKPDTEYEVSLISGSYGLVGVRASNPAKITFKTGL |
| 36 | LgBiT_PRSIM_01 | Protein | MVFTLEDFVGDWEQTAAYNLDQVLEQGGVSSLLQNLAVSVTPIQRIVRSGENALKI |
| | | | DIHVIIPYEGLSADQMAQIEEVFKVVYPVDDHHFKVILPYGTLVIDGVTPNMLNYF |
| | | | GRPYEGIAVFDGKKITVTGTLWNGNKIIDERLITPDGSMLFRVTINSGSSGGGGSG |
| | | | GGGSSGQVQLVQSGAEVKKPGSSVKVSCKASGGTFSSYAISWVRQAPGQGLEWMGG |
| | | | IIPIFGTANYAQKFQGRVTITADESTSTAYMELSSLRSEDTAVYYCARGQGYITVF |
| | | | DYWGQGTLVTVSSGGGGSGGGGSGGGGSAQSVLTQPPSASGTPGQRVTISCSGSSS |
| | | | NIGSNTVNWYQQLPGTAPKLLIYSNNQRPSGVPDRFSGSKSGTSASLAISGLQSED |
| | | | EADYYCAAWDHHWEQVVFGGGTKLTVL |
| 37 | LgBiT_PRSIM_06 | Protein | MVFTLEDFVGDWEQTAAYNLDQVLEQGGVSSLLQNLAVSVTPIQRIVRSGENALKI |
| | | | DIHVIIPYEGLSADQMAQIEEVFKVVYPVDDHHFKVILPYGTLVIDGVTPNMLNYF |
| | | | GRPYEGIAVFDGKKITVTGTLWNGNKIIDERLITPDGSMLFRVTINSGSSGGGGSG |
| | | | GGGSSGQVQLVQSGAEVKKPGSSVKVSCKASGGTFSSYAISWVRQAPGQGLEWMGG |
| | | | IIPIFGTANYAQKFQGRVTITADESTSTAYMELSSLRSEDTAVYYCARGAGYYMRV |
| | | | DYWGQGTLVTVSSGGGGSGGGGSGGGGSAQSVLTQPPSASGTPGQRVTISCSGSSS |
| | | | NIGSNTVNWYQQLPGTAPKLLIYSNNQRPSGVPDRFSGSKSGTSASLAISGLQSED |
| | | | EADYYCAAWDHDVEHVVFGGGTKLTVL |
| 38 | LgBiT_PRSIM_57 | Protein | MVFTLEDFVGDWEQTAAYNLDQVLEQGGVSSLLQNLAVSVTPIQRIVRSGENALKI |
| | | | DIHVIIPYEGLSADQMAQIEEVFKVVYPVDDHHFKVILPYGTLVIDGVTPNMLNYF |
| | | | GRPYEGIAVFDGKKITVTGTLWNGNKIIDERLITPDGSMLFRVTINSGSSGGGGSG |
| | | | GGGSSGQVQLVQSGAEVKKPGSSVKVSCKASGGTFSSYAISWVRQAPGQGLEWMGG |
| | | | IIPIFGTANYAQKFQGRVTITADESTSTAYMELSSLRSEDTAVYYCARHTNYITVF |
| | | | DYWGQGTLVTVSSGGGGSGGGGSGGGGSAQSVLTQPPSASGTPGQRVTISCSGSSS |
| | | | NIGSNTVNWYQQLPGTAPKLLIYSNNQRPSGVPDRFSGSKSGTSASLAISGLQSED |
| | | | EADYYCAAWDHHWEQVVFGGGTKLTVL |
| 39 | LgBiT_PRSIM_67 | Protein | MVFTLEDFVGDWEQTAAYNLDQVLEQGGVSSLLQNLAVSVTPIQRIVRSGENALKI |
| | | | DIHVIIPYEGLSADQMAQIEEVFKVVYPVDDHHFKVILPYGTLVIDGVTPNMLNYF |
| | | | GRPYEGIAVFDGKKITVTGTLWNGNKIIDERLITPDGSMLFRVTINSGSSGGGGSG |
| | | | GGGSSGEVQLVQSGAEVKKPGAAVRISCKTSGYVFTSYYVHWVRQAPGQGLEWMGV |
| | | | INPSGGNTNYAQKFQDRVTMTRDTSTTTVYMELSSLMFDDTAVYYCAKRDYGGPLA |
| | | | NWGRGTLVTVSSGGGGSGGGGSGGGGSALSYELTQPPSVSEAPRQRVTISCSGSSS |
| | | | NIGNNAVNWYQQLPGKAPKLLIFYDDLLPSGVSDRFSGSKSGTSASLAISGLQSED |
| | | | EADYYCAAWDDSLNGLVFGTGTKLTVL |
| 40 | LgBiT_PRSIM_72 | Protein | MVFTLEDFVGDWEQTAAYNLDQVLEQGGVSSLLQNLAVSVTPIQRIVRSGENALKI |
| | | | DIHVIIPYEGLSADQMAQIEEVFKVVYPVDDHHFKVILPYGTLVIDGVTPNMLNYF |
| | | | GRPYEGIAVFDGKKITVTGTLWNGNKIIDERLITPDGSMLFRVTINSGSSGGGGSG |
| | | | GGGSSGQVQLVQSGAEVKKPGSSVKVSCKASGGTFSSYAISWVRQAPGQGLEWMGG |
| | | | IIPIFGTANYAQKFQGRVTITADESTSTAYMELSSLRSEDTAVYYCARGMAHFYQF |
| | | | DLWGQGTLVTVSSGGGGSGGGGSGGGGSAQSVLTQPPSASGTPGQRVTISCSGSSS |
| | | | NIGSNTVNWYQQLPGTAPKLLIYSNNQRPSGVPDRFSGSKSGTSASLAISGLQSED |
| | | | EADYYCAAGDHDHEHVVFGGGTKLTVL |
| 41 | LgBiT_PRSIM_75 | Protein | MVFTLEDFVGDWEQTAAYNLDQVLEQGGVSSLLQNLAVSVTPIQRIVRSGENALKI |
| | | | DIHVIIPYEGLSADQMAQIEEVFKVVYPVDDHHFKVILPYGTLVIDGVTPNMLNYF |
| | | | GRPYEGIAVFDGKKITVTGTLWNGNKIIDERLITPDGSMLFRVTINSGSSGGGGSG |
| | | | GGGSSGEVQLVQSGAEVKKPGSSVKVSCKASGGSFNSYTLDWVRQAPGQGLEWMGG |
| | | | IIPVFGSPNYGQKFQGRVTITADESTSTAYMELSSLKSDDTAVYYCARGLVYQPLD |
| | | | SWGRGTLVTVSSGGGGSGGGGSGGGGSAQAVLTQPSSASGTPGQRVTISCSGSSSN |
| | | | IGSYTVNWYQQFPGTAPKLLIYSNTQRPSGVPDRFSGSKSGTSASLAISGLQSEDE |
| | | | ADYYCAAWDDSLNGWVFGGGTKVTVL |
| 42 | p65 AD | Protein | DEFPTMVFPSGQISQASALAPAPPQVLPQAPAPAPAPAMVSALAQAPAPVPVLAPG |
| | | | PPQAVAPPAPKPTQAGEGTLSEALLQLQFDDEDLGALLGNSTDPAVFTDLASVDNS |
| | | | EFQQLLNQGIPVAPHTTEPMLMEYPEAITRLVTGAQRPPDPAPAPLGAPGLPNGLL |
| | | | SGDEDFSSIADMDFSALLSQISSTSY |
| 43 | ZFHD1 DBD | Protein | MDYPAAKRVKLDSRERPYACPVESCDRRFSRSDELTRHIRIHTGQKPFQCRICMRN |
| | (+ leader) | | FSRSDHLTTHIRTHTGGGRRRKKRTSIETNIRVALEKSFLENQKPTSEEITMIADQ |
| | | | LNMEKEVIRVWFCNRRQKEKRINT |
| 44 | HCV-Pro-AD | Protein | MGKKKGSVVIVGRINLSGDTAYAQQTRGEEGCQETSQTGRDKNQVEGEVQIVSTAT |
| | fusion | | QTFLATSINGVLWTVYHGAGTRTIASPKGPVTQMYTNVDKDLVGWQAPQGSRSLTP |
| | | | CTCGSSDLYLVTRHADVIPVRRRGDSRGSLLSPRPISYLKGSAGGPLLCPAGHAVG |
| | | | IFRAAVSTRGVAKAVDFIPVESLETTMRSP |
| 45 | DBD-HCV | Protein | MDYPAAKRVKLDSRERPYACPVESCDRRFSRSDELTRHIRIHTGQKPFQCRICMRN |
| | Pro fusion | | FSRSDHLTTHIRTHTGGGRRRKKRTSIETNIRVALEKSFLENQKPTSEEITMIADQ |
| | | | LNMEKEVIRVWFCNRRQKEKRINTSAGSRLDAPSQIEVKDVTDTTALITWVDPRYD |
| | | | DIWWFELTYGIKDVPGDRTTIKLYLNDPYYSIGNLKPDTEYEVSLISYTGDSYSRS |
| | | | GSNPAKITFKTGL |
| 46 | PRSIM_23_DBD_ | Protein | MDYPAAKRVKLDSRERPYACPVESCDRRFSRSDELTRHIRIHTGQKPFQCRICMRN |
| | fusion | | FSRSDHLTTHIRTHTGGGRRRKKRTSIETNIRVALEKSFLENQKPTSEEITMIADQ |
| | | | LNMEKEVIRVWFCNRRQKEKRINTSAGSRLDAPSQIEVKDVTDTTALITWVDPRYD |
| | | | DIWWFELTYGIKDVPGDRTTIKLYLNDPYYSIGNLKPDTEYEVSLISYTGDSYSRS |
| | | | GSNPAKITFKTGL |
| 47 | PRSIM_32_DBD_ | Protein | MDYPAAKRVKLDSRERPYACPVESCDRRFSRSDELTRHIRIHTGQKPFQCRICMRN |
| | fusion | | FSRSDHLTTHIRTHTGGGRRRKKRTSIETNIRVALEKSFLENQKPTSEEITMIADQ |
| | | | LNMEKEVIRVWFCNRRQKEKRINTSAGSRLDAPSQIEVKDVTDTTALITWWSPRYY |
| | | | YASISGFELTYGIKDVPGDRTTIKLDYASNDYSIGNLKPDTEYEVSLISWNYGDWR |
| | | | YSSSNPAKITFKTGL |
| 48 | PRSIM_33_DBD_ | Protein | MDYPAAKRVKLDSRERPYACPVESCDRRFSRSDELTRHIRIHTGQKPFQCRICMRN |
| | fusion | | FSRSDHLTTHIRTHTGGGRRRKKRTSIETNIRVALEKSFLENQKPTSEEITMIADQ |
| | | | LNMEKEVIRVWFCNRRQKEKRINTSAGSRLDAPSQIEVKDVTDTTALITWYPPGRW |
| | | | YDDIWYFELTYGIKDVPGDRTTIKLARGDDVYSIGNLKPDTEYEVSLISWGPDRGD |
| | | | RAGSNPAKITFKTGL |
| 49 | PRSIM_36_DBD_ | Protein | MDYPAAKRVKLDSRERPYACPVESCDRRFSRSDELTRHIRIHTGQKPFQCRICMRN |
| | fusion | | FSRSDHLTTHIRTHTGGGRRRKKRTSIETNIRVALEKSFLENQKPTSEEITMIADQ |
| | | | LNMEKEVIRVWFCNRRQKEKRINTSAGSRLDAPSQIEVKDVTDTTALITWSWPRDD |
| | | | DYDIWYFELTYGIKDVPGDRTTIKLLNYASPYSIGNLKPDTEYEVSLISVVPDTYG |
| | | | RGTSNPAKITFKTGL |
| 50 | PRSIM_47_DBD_ | Protein | MDYPAAKRVKLDSRERPYACPVESCDRRFSRSDELTRHIRIHTGQKPFQCRICMRN |
| | fusion | | FSRSDHLTTHIRTHTGGGRRRKKRTSIETNIRVALEKSFLENQKPTSEEITMIADQ |
| | | | LNMEKEVIRVWFCNRRQKEKRINTSAGSRLDAPSQIEVKDVTDTTALITWSRPGVS |
| | | | IWYFELTYGIKDVPGDRTTIKLDYRSYYYSIGNLKPDTEYEVSLISGSYGLVGVRA |
| | | | SNPAKITFKTGL |
| 51 | PRSIM_01_DBD_ | Protein | MDYPAAKRVKLDSRERPYACPVESCDRRFSRSDELTRHIRIHTGQKPFQCRICMRN |
| | fusion | | FSRSDHLTTHIRTHTGGGRRRKKRTSIETNIRVALEKSFLENQKPTSEEITMIADQ |
| | | | LNMEKEVIRVWFCNRRQKEKRINTSAGSQVQLVQSGAEVKKPGSSVKVSCKASGGT |
| | | | FSSYAISWVRQAPGQGLEWMGGIIPIFGTANYAQKFQGRVTITADESTSTAYMELS |
| | | | SLRSEDTAVYYCARGQGYITVFDYWGQGTLVTVSSGGGGSGGGGSGGGGSAQSVLT |
| | | | QPPSASGTPGQRVTISCSGSSSNIGSNTVNWYQQLPGTAPKLLIYSNNQRPSGVPD |
| | | | RFSGSKSGTSASLAISGLQSEDEADYYCAAWDHHWEQVVFGGGTKLTVL |
| 52 | PRSIM_04_DBD_ | Protein | MDYPAAKRVKLDSRERPYACPVESCDRRFSRSDELTRHIRIHTGQKPFQCRICMRN |
| | fusion | | FSRSDHLTTHIRTHTGGGRRRKKRTSIETNIRVALEKSFLENQKPTSEEITMIADQ |
| | | | LNMEKEVIRVWFCNRRQKEKRINTSAGSQVQLVQSGAEVKKPGSSVKVSCKASGGT |
| | | | FSSYAISWVRQAPGQGLEWMGGIIPIFGTANYAQKFQGRVTITADESTSTAYMELS |
| | | | SLRSEDTAVYYCARGMAHFYQFDLWGQGTLVTVSSGGGGSGGGGSGGGGSAQSVLT |
| | | | QPPSASGTPGQRVTISCSGSSSNIGSNTVNWYQQLPGTAPKLLIYSNNQRPSGVPD |
| | | | RFSGSKSGTSASLAISGLQSEDEADYYCAAGDHDHEHVVFGGGTKLTVL |
| 53 | PRSIM_57_DBD_ | Protein | MDYPAAKRVKLDSRERPYACPVESCDRRFSRSDELTRHIRIHTGQKPFQCRICMRN |
| | fusion | | FSRSDHLTTHIRTHTGGGRRRKKRTSIETNIRVALEKSFLENQKPTSEEITMIADQ |
| | | | LNMEKEVIRVWFCNRRQKEKRINTSAGSQVQLVQSGAEVKKPGSSVKVSCKASGGT |
| | | | FSSYAISWVRQAPGQGLEWMGGIIPIFGTANYAQKFQGRVTITADESTSTAYMELS |
| | | | SLRSEDTAVYYCARHTNYITVFDYWGQGTLVTVSSGGGGSGGGGSGGGGSAQSVLT |
| | | | QPPSASGTPGQRVTISCSGSSSNIGSNTVNWYQQLPGTAPKLLIYSNNQRPSGVPD |
| | | | RFSGSKSGTSASLAISGLQSEDEADYYCAAWDHHWEQVVFGGGTKLTVL |
| 54 | PRSIM_67_DBD_ | Protein | MDYPAAKRVKLDSRERPYACPVESCDRRFSRSDELTRHIRIHTGQKPFQCRICMRN |
| | fusion | | FSRSDHLTTHIRTHTGGGRRRKKRTSIETNIRVALEKSFLENQKPTSEEITMIADQ |
| | | | LNMEKEVIRVWFCNRRQKEKRINTSAGSEVQLVQSGAEVKKPGAAVRISCKTSGYV |
| | | | FTSYYVHWVRQAPGQGLEWMGVINPSGGNTNYAQKFQDRVTMTRDTSTTTVYMELS |
| | | | SLMFDDTAVYYCAKRDYGGPLANWGRGTLVTVSSGGGGSGGGGSGGGGSALSYELT |
| | | | QPPSVSEAPRQRVTISCSGSSSNIGNNAVNWYQQLPGKAPKLLIFYDDLLPSGVSD |
| | | | RFSGSKSGTSASLAISGLQSEDEADYYCAAWDDSLNGLVFGTGTKLTVL |
| 55 | PRSIM_72_DBD_ | Protein | MDYPAAKRVKLDSRERPYACPVESCDRRFSRSDELTRHIRIHTGQKPFQCRICMRN |
| | fusion | | FSRSDHLTTHIRTHTGGGRRRKKRTSIETNIRVALEKSFLENQKPTSEEITMIADQ |
| | | | LNMEKEVIRVWFCNRRQKEKRINTSAGSQVQLVQSGAEVKKPGSSVKVSCKVSGGS |
| | | | FNNYGVSWVRQAPGQGLEWMGRHPIRDTANYAQKFQGRVTITADTSTNIAYMELSG |
| | | | LRSDDTAVYYCARVLEDDFWGGYYDFYFYVMDVWGQGTLVTVSSGGGGSGGGGSGG |
| | | | GGSALSSELTQDPWSVPLGQTARITCQGDSLTTYYATWYQQKPGQAPVLVLYNEHK |
| | | | RPSGISDRFSGSSAGDAASLTITDTQAEDEADYYCSSRDTGGKHVLFGGGTKLTVL |
| 56 | PRSIM_75_DBD_ | Protein | MDYPAAKRVKLDSRERPYACPVESCDRRFSRSDELTRHIRIHTGQKPFQCRICMRN |
| | fusion | | FSRSDHLTTHIRTHTGGGRRRKKRTSIETNIRVALEKSFLENQKPTSEEITMIADQ |
| | | | LNMEKEVIRVWFCNRRQKEKRINTSAGSEVQLVQSGAEVKKPGSSVKVSCKASGGS |
| | | | FNSYTLDWVRQAPGQGLEWMGGIIPVFGSPNYGQKFQGRVTITADESTSTAYMELS |
| | | | SLKSDDTAVYYCARGLVYQPLDSWGRGTLVTVSSGGGGSGGGGSGGGGSAQAVLTQ |
| | | | PSSASGTPGQRVTISCSGSSSNIGSYTVNWYQQFPGTAPKLLIYSNTQRPSGVPDR |
| | | | FSGSKSGTSASLAISGLQSEDEADYYCAAWDDSLNGVWFGGGTKVTVL |
| 57 | PRSIM_23_AD_ | Protein | MGSRLDAPSQIEVKDVTDTTALITWVDPRYDDIWWFELTYGIKDVPGDRTTIKLYL |
| | fusion | | NDPYYSIGNLKPDTEYEVSLISYTGDSYSRSGSNPAKITFKTGLTGGGGSGGGGSD |
| | | | EFPTMVFPSGQISQASALAPAPPQVLPQAPAPAPAPAMVSALAQAPAPVPVLAPGP |
| | | | PQAVAPPAPKPTQAGEGTLSEALLQLQFDDEDLGALLGNSTDPAVFTDLASVDNSE |
| | | | FQQLLNQGIPVAPHTTEPMLMEYPEAITRLVTGAQRPPDPAPAPLGAPGLPNGLLS |
| | | | GDEDFSSIADMDFSALLSQISSTSY |
| 58 | PRSIM_32_AD_ | Protein | MGSRLDAPSQIEVKDVTDTTALITWWSPRYYYASISGFELTYGIKDVPGDRTTIKL |
| | fusion | | DYASNDYSIGNLKPDTEYEVSLISWNYGDWRYSSSNPAKITFKTGLTGGGGSGGGG |
| | | | SDEFPTMVFPSGQISQASALAPAPPQVLPQAPAPAPAPAMVSALAQAPAPVPVLAP |
| | | | GPPQAVAPPAPKPTQAGEGTLSEALLQLQFDDEDLGALLGNSTDPAVFTDLASVDN |
| | | | SEFQQLLNQGIPVAPHTTEPMLMEYPEAITRLVTGAQRPPDPAPAPLGAPGLPNGL |
| | | | LSGDEDFSSIADMDFSALLSQISSTSY |
| 59 | PRSIM_33_AD_ | Protein | MGSRLDAPSQIEVKDVTDTTALITWYPPGRWYDDIWYFELTYGIKDVPGDRTTIKL |
| | fusion | | ARGDDVYSIGNLKPDTEYEVSLISWGPDRGDRAGSNPAKITFKTGLTGGGGSGGGG |
| | | | SDEFPTMVFPSGQISQASALAPAPPQVLPQAPAPAPAPAMVSALAQAPAPVPVLAP |
| | | | GPPQAVAPPAPKPTQAGEGTLSEALLQLQFDDEDLGALLGNSTDPAVFTDLASVDN |
| | | | SEFQQLLNQGIPVAPHTTEPMLMEYPEAITRLVTGAQRPPDPAPAPLGAPGLPNGL |
| | | | LSGDEDFSSIADMDFSALLSQISSTSY |
| 60 | PRSIM_36_AD_ | Protein | MGSRLDAPSQIEVKDVTDTTALITWSWPRDDDYDIWYFELTYGIKDVPGDRTTIKL |
| | fusion | | LNYASPYSIGNLKPDTEYEVSLISVVPDTYGRGTSNPAKITFKTGLTGGGGSGGGG |
| | | | SDEFPTMVFPSGQISQASALAPAPPQVLPQAPAPAPAPAMVSALAQAPAPVPVLAP |
| | | | GPPQAVAPPAPKPTQAGEGTLSEALLQLQFDDEDLGALLGNSTDPAVFTDLASVDN |
| | | | SEFQQLLNQGIPVAPHTTEPMLMEYPEAITRLVTGAQRPPDPAPAPLGAPGLPNGL |
| | | | LSGDEDFSSIADMDFSALLSQISSTSY |
| 61 | PRSIM_47_AD_ | Protein | MGSRLDAPSQIEVKDVTDTTALITWSRPGVSIWYFELTYGIKDVPGDRTTIKLDYR |
| | fusion | | SYYYSIGNLKPDTEYEVSLISGSYGLVGVRASNPAKITFKTGLTGGGGSGGGGSDE |
| | | | FPTMVFPSGQISQASALAPAPPQVLPQAPAPAPAPAMVSALAQAPAPVPVLAPGPP |
| | | | QAVAPPAPKPTQAGEGTLSEALLQLQFDDEDLGALLGNSTDPAVFTDLASVDNSEF |
| | | | QQLLNQGIPVAPHTTEPMLMEYPEAITRLVTGAQRPPDPAPAPLGAPGLPNGLLSG |
| | | | DEDFSSIADMDFSALLSQISSTSY |
| 62 | PRSIM_01_AD_ | Protein | MGSQVQLVQSGAEVKKPGSSVKVSCKASGGTFSSYAISWVRQAPGQGLEWMGGIIP |
| | fusion | | IFGTANYAQKFQGRVTITADESTSTAYMELSSLRSEDTAVYYCARGQGYYGYFDYW |
| | | | GQGTLVTVSSGGGGSGGGGSGGGGSAQSVLTQPPSASGTPGQRVTISCSGSSSNIG |
| | | | SNTVNWYQQLPGTAPKLLIYSNNQRPSGVPDRFSVSKSGTSASLAISGLQSEDEAD |
| | | | YYCAAWDHGHEHVVFGGGTKLTVLTGGGGSGGGGSDEFPTMVFPSGQISQASALAP |
| | | | APPQVLPQAPAPAPAPAMVSALAQAPAPVPVLAPGPPQAVAPPAPKPTQAGEGTLS |
| | | | EALLQLQFDDEDLGALLGNSTDPAVFTDLASVDNSEFQQLLNQGIPVAPHTTEPML |
| | | | MEYPEAITRLVTGAQRPPDPAPAPLGAPGLPNGLLSGDEDFSSIADMDFSALLSQI |
| | | | SSTSY |
| 63 | PRSIM_04_AD_ | Protein | MGSQVQLVQSGAEVKKPGSSVKVSCKASGGTFSSYAISWVRQAPGQGLEWMGGIIP |
| | fusion | | IFGTANYAQKFQGRVTITADESTSTAYMELSSLRSEDTAVYYCARGMAHFYQFDLW |
| | | | GQGTLVTVSSGGGGSGGGGSGGGGSAQSVLTQPPSASGTPGQRVTISCSGSSSNIG |
| | | | SNTVNWYQQLPGTAPKLLIYSNNQRPSGVPDRFSGSKSGTSASLAISGLQSEDEAD |
| | | | YYCAAGDHDHEHVVFGGGTKLTVLTGGGGSGGGGSDEFPTMVFPSGQISQASALAP |
| | | | APPQVLPQAPAPAPAPAMVSALAQAPAPVPVLAPGPPQAVAPPAPKPTQAGEGTLS |
| | | | EALLQLQFDDEDLGALLGNSTDPAVFTDLASVDNSEFQQLLNQGIPVAPHTTEPML |
| | | | MEYPEAITRLVTGAQRPPDPAPAPLGAPGLPNGLLSGDEDFSSIADMDFSALLSQI |
| | | | SSTSY |
| 64 | PRSIM_57_AD_ | Protein | MGSQVQLVQSGAEVKKPGSSVKVSCKASGGTFSSYAISWVRQAPGQGLEWMGGIIP |
| | fusion | | IFGTANYAQKFQGRVTITADESTSTAYMELSSLRSEDTAVYYCARHTNYITVFDYW |
| | | | GQGTLVTVSSGGGGSGGGGSGGGGSAQSVLTQPPSASGTPGQRVTISCSGSSSNIG |
| | | | SNTVNWYQQLPGTAPKLLIYSNNQRPSGVPDRFSGSKSGTSASLAISGLQSEDEAD |
| | | | YYCAAWDHHWEQVVFGGGTKLTVLTGGGGSGGGGSDEFPTMVFPSGQISQASALAP |
| | | | APPQVLPQAPAPAPAPAMVSALAQAPAPVPVLAPGPPQAVAPPAPKPTQAGEGTLS |
| | | | EALLQLQFDDEDLGALLGNSTDPAVFTDLASVDNSEFQQLLNQGIPVAPHTTEPML |
| | | | MEYPEAITRLVTGAQRPPDPAPAPLGAPGLPNGLLSGDEDFSSIADMDFSALLSQI |
| | | | SSTSY |
| 65 | PRSIM_67_AD_ | Protein | MGSEVQLVQSGAEVKKPGAAVRISCKTSGYVFTSYYVHWVRQAPGQGLEWMGVINP |
| | fusion | | SGGNTNYAQKFQDRVTMTRDTSTTTVYMELSSLMFDDTAVYYCAKRDYGGPLANWG |
| | | | RGTLVTVSSGGGGSGGGGSGGGGSALSYELTQPPSVSEAPRQRVTISCSGSSSNIG |
| | | | NNAVNWYQQLPGKAPKLLIFYDDLLPSGVSDRFSGSKSGTSASLAISGLQSEDEAD |
| | | | YYCAAWDDSLNGLVFGTGTKLTVLTGGGGSGGGGSDEFPTMVFPSGQISQASALAP |
| | | | APPQVLPQAPAPAPAPAMVSALAQAPAPVPVLAPGPPQAVAPPAPKPTQAGEGTLS |
| | | | EALLQLQFDDEDLGALLGNSTDPAVFTDLASVDNSEFQQLLNQGIPVAPHTTEPML |
| | | | MEYPEAITRLVTGAQRPPDPAPAPLGAPGLPNGLLSGDEDFSSIADMDFSALLSQI |
| | | | SSTSY |
| 66 | PRSIM_72_AD_ | Protein | MGSQVQLVQSGAEVKKPGSSVKVSCKVSGGSFNNYGVSWVRQAPGQGLEWMGRIIP |
| | fusion | | IRDTANYAQKFQGRVTITADTSTNIAYMELSGLRSDDTAVYYCARVLEDDFWGGYY |
| | | | DFYFYVMDVWGQGTLVTVSSGGGGSGGGGSGGGGSALSSELTQDPVVSVPLGQTAR |
| | | | ITCQGDSLTTYYATWYQQKPGQAPVLVLYNEHKRPSGISDRFSGSSAGDAASLTIT |
| | | | DTQAEDEADYYCSSRDTGGKHVLFGGGTKLTVLTGGGGSGGGGSDEFPTMVFPSGQ |
| | | | ISQASALAPAPPQVLPQAPAPAPAPAMVSALAQAPAPVPVLAPGPPQAVAPPAPKP |
| | | | TQAGEGTLSEALLQLQFDDEDLGALLGNSTDPAVFTDLASVDNSEFQQLLNQGIPV |
| | | | APHTTEPMLMEYPEAITRLVTGAQRPPDPAPAPLGAPGLPNGLLSGDEDFSSIADM |
| | | | DFSALLSQISSTSY |
| 67 | PRSIM_75_AD_ | Protein | MGSEVQLVQSGAEVKKPGSSVKVSCKASGGSFNSYTLDWVRQAPGQGLEWMGGIIP |
| | fusion | | VFGSPNYGQKFQGRVTITADESTSTAYMELSSLKSDDTAVYYCARGLVYQPLDSWG |
| | | | RGTLVTVSSGGGGSGGGGSGGGGSAQAVLTQPSSASGTPGQRVTISCSGSSSNIGS |
| | | | YTVNWYQQFPGTAPKLLIYSNTQRPSGVPDRFSGSKSGTSASLAISGLQSEDEADY |
| | | | YCAAWDDSLNGWVFGGGTKVTVLTGGGGSGGGGSDEFPTMVFPSGQISQASALAPA |
| | | | PPQVLPQAPAPAPAPAMVSALAQAPAPVPVLAPGPPQAVAPPAPKPTQAGEGTLSE |
| | | | ALLQLQFDDEDLGALLGNSTDPAVFTDLASVDNSEFQQLLNQGIPVAPHTTEPMLM |
| | | | EYPEAITRLVTGAQRPPDPAPAPLGAPGLPNGLLSGDEDFSSIADMDFSALLSQIS |
| | | | STSY |
| 68 | NanoLuc-Pest | Protein | MVFTLEDFVGDWRQTAGYNLDQVLEQGGVSSLFQNLGVSVTPIQRIVLSGENGLKI |
| | | | DIHVIIPYEGLSGDQMGQIEKIFKVVYPVDDHHFKVILHYGTLVIDGVTPNMIDYF |
| | | | GRPYEGIAVFDGKKITVTGTLWNGNKIIDERLINPDGSLLFRVTINGVTGWRLCER |
| | | | ILANSHGFPPEVEEQAAGTLPMSCAQESGMDRHPAACASARINV |
| 69 | FKBP12: FRB | Protein | MLLLVTSLLLCELPHPAFLLIPESKYGPPCPPCPFWVLVVVGGVLACYSLLVTVAF |
| | CAR | | IIFWVKRGRKKLLYIFKQPFMRPVQTTQEEDGCSCRFPEEEEGGCELSRGSGSGSG |
| | | | SMGVQVETISPGDGRTFPKRGQTCVVHYTGMLEDGKKFDSSRDRNKPFKFMLGKQE |
| | | | VIRGWEEGVAQMSVGQRAKLTISPDYAYGATGHPGIIPPHATLVFDVELLKLEGSG |
| | | | ATNFSLLKQAGDVEENPGPMIHLGHILFLLLLPVAAAQTTPGERSSLPAFYPGTSG |
| | | | SCSGCGSLSLPESKYGPPCPPCPFVWLVVVGGVLACYSLLVTVAFIIFVWSLKRGR |
| | | | KKLLYIFKQPFMRPVQTTQEEDGCSCRFPEEEEGGCELILWHEMWHEGLEEASRLY |
| | | | FGERNVKGMFEVLEPLHAMMERGPQTLKETSFNQAYGRDLMEAQEWCRKYMKSGNV |
| | | | KDLLQAWDLYYHVFRRISKGSGSGSGSSLRVKFSRSADAPAYQQGQNQLYNELNLG |
| | | | RREEYDVLDKRRGRDPEMGGKPRRKNPQEGLYNELQKDKMAEAYSEIGMKGERRRG |
| | | | KGHDGLYQGLSTATKDTYDALHMQALPPRGSGEGRGSLLTCGDVEENPGPSGMESD |
| | | | ESGLPAMEIECRITGTLNGVEFELVGGGEGTPKQGRMTNKMKSTKGALTFSPYLLS |
| | | | HVMGYGFYHFGTYPSGYENPFLHAINNGGYTNTRIEKYEDGGVLHVSFSYRYEAGR |
| | | | VIGDFKVVGTGFPEDSVIFTDKIIRSNATVEHLHPMGDNVLVGSFARTFSLRDGGY |
| | | | YSFVVDSHMHFKSAIHPSILQNGGPMFAFRRVEELHSNTELGIVEYQHAFKTPIAF |
| | | | ARSRAQSSNSAVDGTAGPGSTGSR |
| 70 | PRSIM_23 | Protein | ESKYGPPCPPCPFWVLVVVGGVLACYSLLVTVAFIIFWVKRGRKKLLYIFKQPFMR |
| | CAR 1^{st} | | PVQTTQEEDGCSCRFPEEEEGGCELGGGGSGGGGSMKKKGSVVIVGRINLSGDTAY |
| | polypeptide | | AQQTRGEEGCQETSQTGRDKNQVEGEVQIVSTATQTFLATSINGVLWTVYHGAGTR |
| | | | TIASPKGPVTQMYTNVDKDLVGWQAPQGSRSLTPCTCGSSDLYLVTRHADVIPVRR |
| | | | RGDSRGSLLSPRPISYLKGSAGGPLLCPAGHAVGIFRAAVSTRGVAKAVDFIPVES |
| | | | LETTMRSPGSG |
| 71 | Wild-type | DNA | ATGGGTAGCAGCCATCACCATCATCATCATGGTAGCGGTCTGAACGATATTTTTGA |
| | HCV NS3/4APR | | AGCCCAGAAAATCGAATGGCATGAAGGTGGTGGTGGTAGCATGAAAAAAAAGGGTA |
| | with N- | | GCGTTGTTATTGTGGGTCGCATTAATCTGAGCGGTGATACCGCATATGCACAGCAG |
| | terminal 6His | | ACCCGTGGTGAAGAAGGTTGTCAAGAAACCAGCCAGACCGGTCGTGATAAAAATCA |
| | | | GGTTGAAGGTGAAGTTCAGATTGTTAGCACCGCAACACAGACCTTTCTGGCAACCA |
| | | | GCATTAATGGTGTTCTGTGGACCGTTTATCATGGTGCAGGCACCCGTACCATTGCA |
| | | | AGCCCGAAAGGTCCGGTTACACAGATGTATACCAATGTGGATAAAGATCTGGTTGG |
| | | | TTGGCAGGCACCGCAGGGTAGCCGTAGTCTGACCCCGTGTACCTGTGGTAGCAGCG |
| | | | ATCTGTATCTGGTTACCCGTCATGCAGATGTTATTCCGGTTCGTCGTCGTGGTGAT |
| | | | AGCCGTGGTAGCCTGCTGAGTCCGCGTCCGATTAGCTATCTGAAAGGTAGCAGTGG |
| | | | TGGTCCGCTGCTGTGTCCGGCAGGTCATGCAGTTGGTATTTTTCGTGCAGCAGTTA |
| | | | GCACCCGTGGCGTTGCAAAAGCAGTTGATTTTATCCCGGTTGAAAGCCTGGAAACC |
| | | | ACCATGCGTAGCCCG |
| 72 | HCV NS3/4APR | DNA | ATGGGTAGCAGCCATCACCATCATCATCATGGTAGCGGTCTGAACGATATTTTTGA |
| | (S139A) | | AGCCCAGAAAATCGAATGGCATGAAGGTGGTGGTGGTAGCATGAAAAAAAAGGGTA |
| | with N- | | GCGTTGTTATTGTGGGTCGCATTAATCTGAGCGGTGATACCGCATATGCACAGCAG |
| | terminal 6His | | ACCCGTGGTGAAGAAGGTTGTCAAGAAACCAGCCAGACCGGTCGTGATAAAAATCA |
| | and AviTag | | GGTTGAAGGTGAAGTTCAGATTGTTAGCACCGCAACACAGACCTTTCTGGCAACCA |
| | | | GCATTAATGGTGTTCTGTGGACCGTTTATCATGGTGCAGGCACCCGTACCATTGCA |
| | | | AGCCCGAAAGGTCCGGTTACACAGATGTATACCAATGTGGATAAAGATCTGGTTGG |
| | | | TTGGCAGGCACCGCAGGGTAGCCGTAGTCTGACCCCGTGTACCTGTGGTAGCAGCG |
| | | | ATCTGTATCTGGTTACCCGTCATGCAGATGTTATTCCGGTTCGTCGTCGTGGTGAT |
| | | | AGCCGTGGTAGCCTGCTGAGTCCGCGTCCGATTAGCTATCTGAAAGGTAGTGCCGG |
| | | | TGGTCCGCTGCTGTGTCCGGCAGGTCATGCAGTTGGTATTTTTCGTGCAGCAGTTA |
| | | | GCACCCGTGGCGTTGCAAAAGCAGTTGATTTTATCCCGGTTGAAAGCCTGGAAACC |
| | | | ACCATGCGTAGCCCG |
| 73 | PRSIM_23 | DNA | CGTCTGGATGCACCGAGCCAGATTGAAGTTAAAGATGTTACCGATACCACCGCACT |
| | | | GATTACCTGGGTTGACCCGCGTTACGACGACATTTGGTGGTTTGAACTGACCTATG |
| | | | GCATCAAAGATGTTCCGGGTGATCGTACCACCATTAAACTGTACCTGAACGACCCG |
| | | | TACTATAGCATTGGTAATCTGAAACCGGATACCGAATATGAAGTTAGCCTGATTAG |
| | | | CTACACTGGTGACTCTTACTCTCGTTCTGGTAGCAATCCGGCAAAAATTACCTTTA |
| | | | AAACCGGTCTG |
| 74 | PRSIM_32 | DNA | CGTCTGGATGCACCGAGCCAGATTGAAGTTAAAGATGTTACCGATACCACCGCACT |
| | | | GATTACCTGGTGGTCTCCGCGTTACTACTACGCTTCTATTTCTGGTTTTGAACTGA |
| | | | CCTATGGCATCAAAGATGTTCCGGGTGATCGTACCACCATTAAACTGGACTACGCT |
| | | | TCTAACGACTATAGCATTGGTAATCTGAAACCGGATACCGAATATGAAGTTAGCCT |
| | | | GATTAGCTGGAACTACGGTGACTGGCGTTACTCTTCTAGCAATCCGGCAAAAATTA |
| | | | CCTTTAAAACCGGTCTG |
| 75 | PRSIM_33 | DNA | CGTCTGGATGCACCGAGCCAGATTGAAGTTAAAGATGTTACCGATACCACCGCACT |
| | | | GATTACCTGGTACCCGCCGGGTCGTTGGTACGACGACATTTGGTACTTTGAACTGA |
| | | | CCTATGGCATCAAAGATGTTCCGGGTGATCGTACCACCATTAAACTGGCTCGTGGT |
| | | | GACGACGTTTATAGCATTGGTAATCTGAAACCGGATACCGAATATGAAGTTAGCCT |
| | | | GATTAGCTGGGGTCCGGACCGTGGTGACCGTGCTGGTAGCAATCCGGCAAAAATTA |
| | | | CCTTTAAAACCGGTCTG |
| 76 | PRSIM_36 | DNA | CGTCTGGATGCACCGAGCCAGATTGAAGTTAAAGATGTTACCGATACCACCGCACT |
| | | | GATTACCTGGTCTTGGCCGCGTGACGACGACTACGACATTTGGTACTTTGAACTGA |
| | | | CCTATGGCATCAAAGATGTTCCGGGTGATCGTACCACCATTAAACTGCTGAACTAC |
| | | | GCTTCTCCGTATAGCATTGGTAATCTGAAACCGGATACCGAATATGAAGTTAGCCT |
| | | | GATTAGCGTTGTTCCGGACACTTACGGTCGTGGTACTAGCAATCCGGCAAAAATTA |
| | | | CCTTTAAAACCGGTCTG |
| 77 | PRSIM_47 | DNA | CGTCTGGATGCACCGAGCCAGATTGAAGTTAAAGATGTTACCGATACCACCGCACT |
| | | | GATTACCTGGTCTCGTCCGGGTGTTTCTATTTGGTACTTTGAACTGACCTATGGCA |
| | | | TCAAAGATGTTCCGGGTGATCGTACCACCATTAAACTGGACTACCGTTCTTACTAC |
| | | | TATAGCATTGGTAATCTGAAACCGGATACCGAATATGAAGTTAGCCTGATTAGCGG |
| | | | TTCTTACGGTCTGGTTGGTGTTCGTGCTAGCAATCCGGCAAAAATTACCTTTAAAA |
| | | | CCGGTCTG |
| 78 | PRSIM_01 | DNA | CAGGTTCAGCTGGTTCAGTCTGGCGCCGAAGTGAAGAAACCTGGCAGCAGCGTGAA |
| | | | GGTGTCCTGCAAAGCTTCTGGCGGCACCTTCAGCAGCTACGCCATCTCTTGGGTTC |
| | | | GACAGGCCCCTGGACAAGGCCTGGAATGGATGGGAGGCATCATCCCCATCTTCGGC |
| | | | ACCGCCAATTACGCCCAGAAATTCCAGGGCAGAGTGACCATCACCGCCGACGAGTC |
| | | | TACAAGCACCGCCTACATGGAACTGAGCAGCCTGAGAAGCGAGGACACCGCCGTGT |
| | | | ACTATTGTGCCAGAGGCCAGGGCTACTACGGCTACTTCGATTATTGGGGCCAGGGC |
| | | | ACCCTGGTCACAGTTTCTAGCGGAGGCGGAGGATCTGGTGGCGGAGGAAGTGGCGG |
| | | | AGGCGGTTCTGCTCAATCTGTGCTGACACAGCCTCCTAGCGCCTCTGGAACACCTG |
| | | | GCCAGAGAGTGACAATCAGCTGTAGCGGCAGCAGCAGCAACATCGGCAGCAACACC |
| | | | GTGAACTGGTATCAGCAGCTGCCTGGCACAGCCCCTAAACTGCTGATCTACAGCAA |
| | | | CAACCAGCGGCCTAGCGGCGTGCCCGATAGATTTTCCGTGTCTAAGAGCGGCACCA |
| | | | GCGCCAGCCTGGCTATTTCTGGACTGCAGAGCGAGGACGAGGCCGACTATTATTGT |
| | | | GCCGCCTGGGATCACGGACACGAGCACGTTGTGTTTGGAGGCGGCACCAAGCTGAC |
| | | | AGTGCTT |
| 79 | PRSIM_04 | DNA | CAGGTGCAGCTGGTGCAGTCTGGCGCTGAAGTGAAGAAGCCGGGCTCTTCTGTGAA |
| | | | GGTGTCTTGCAAGGCTTCTGGCGGCACCTTCTCTTCTTACGCTATCTCTTGGGTGC |
| | | | GTCAGGCTCCGGGCCAGGGGCTGGAGTGGATGGGCGGCATCATCCCGATCTTCGGC |
| | | | ACCGCTAACTACGCTCAGAAATTTCAGGGCCGTGTGACCATCACCGCTGATGAATC |
| | | | TACCTCTACCGCTTACATGGAACTGTCATCTCTGCGTTCTGAAGATACCGCTGTAT |
| | | | ACTACTGCGCTCGTGGCATGGCTCACTTCTACCAGTTCGATCTGTGGGGCCAGGGC |
| | | | ACCCTGGTAACCGTCTCGAGTGGTGGTGGCGGCTCTGGTGGCGGTGGCTCTGGCGG |
| | | | TGGTGGCAGTGCACAGTCTGTGCTGACCCAGCCGCCGTCTGCTTCTGGCACCCCGG |
| | | | GCCAGCGTGTGACCATCTCTTGCTCTGGCTCTTCTTCTAACATCGGCTCTAACACC |
| | | | GTGAACTGGTACCAGCAGCTGCCGGGCACCGCTCCGAAGCTGCTGATATACTCTAA |
| | | | CAACCAGCGTCCGTCTGGCGTGCCGGATCGTTTCTCTGGCTCTAAGTCTGGCACCT |
| | | | CTGCTTCTCTGGCTATCTCTGGCCTGCAGTCTGAAGACGAAGCTGATTACTACTGC |
| | | | GCTGCTGGGGATCACGATCACGAACACGTGGTGTTCGGCGGCGGCACCAAGCTGAC |
| | | | CGTGCTG |
| 80 | PRSIM_57 | DNA | CAGGTGCAGCTGGTGCAGTCTGGCGCTGAAGTGAAGAAGCCGGGCTCTTCTGTGAA |
| | | | GGTGTCTTGCAAGGCTTCTGGCGGCACCTTCTCTTCTTACGCTATCTCTTGGGTGC |
| | | | GTCAGGCTCCGGGCCAGGGGCTGGAGTGGATGGGCGGCATCATCCCGATCTTCGGC |
| | | | ACCGCTAACTACGCTCAGAAATTTCAGGGCCGTGTGACCATCACCGCTGATGAATC |
| | | | TACCTCTACCGCTTACATGGAACTGTCATCTCTGCGTTCTGAAGATACCGCTGTAT |
| | | | ACTACTGCGCTCGTCACACGAACTACATCACGGTTTTCGATTACTGGGGCCAGGGC |
| | | | ACCCTGGTAACCGTCTCGAGTGGTGGTGGCGGCTCTGGTGGCGGTGGCTCTGGCGG |
| | | | TGGTGGCAGTGCACAGTCTGTGCTGACCCAGCCGCCGTCTGCTTCTGGCACCCCGG |
| | | | GCCAGCGTGTGACCATCTCTTGCTCTGGCTCTTCTTCTAACATCGGCTCTAACACC |
| | | | GTGAACTGGTACCAGCAGCTGCCGGGCACCGCTCCGAAGCTGCTGATCTACTCTAA |
| | | | CAACCAGCGTCCGTCTGGCGTGCCGGATCGTTTCTCTGGCTCTAAGTCTGGCACCT |
| | | | CTGCTTCTCTGGCTATCTCTGGCCTGCAGTCTGAAGACGAAGCTGATTACTACTGC |
| | | | GCTGCTTGGGATCACCACTGGGAACAGGTGGTGTTCGGCGGCGGCACCAAGCTGAC |
| | | | CGTGCTG |
| 81 | PRSIM_67 | DNA | GAAGTGCAGCTGGTGCAGTCTGGGGCTGAGGTGAAGAAGCCTGGGGCCGCAGTGAG |
| | | | GATTTCCTGCAAGACATCTGGATACGTCTTCACCAGCTACTATGTGCACTGGGTGC |
| | | | GACAGGCCCCTGGACAAGGGCTTGAGTGGATGGGAGTTATCAACCCTAGTGGTGGT |
| | | | AATACGAACTACGCACAGAAGTTCCAGGACAGAGTCACCATGACCAGGGACACGTC |
| | | | CACGACCACAGTCTATATGGAGTTGAGCAGCCTGATGTTTGATGACACGGCCGTGT |
| | | | ATTACTGTGCGAAGCGAGACTACGGGGGACCCTTGGCAAACTGGGGCCGGGGAACC |
| | | | CTGGTCACCGTCTCGAGTGGAGGCGGCGGTTCAGGCGGAGGTGGCTCTGGCGGTGG |
| | | | CGGAAGTGCACTTTCCTATGAGCTGACTCAGCCACCCTCGGTGTCTGAAGCCCCGA |
| | | | GGCAGAGGGTCACCATCTCCTGTTCTGGAAGCAGCTCCAACATCGGAAATAATGCT |
| | | | GTAAACTGGTACCAGCAGCTCCCAGGAAAGGCTCCCAAACTCCTCATTTTTTATGA |
| | | | TGATCTGCTGCCCTCAGGGGTCTCTGACCGATTCTCTGGCTCCAAGTCTGGCACCT |
| | | | CAGCCTCCCTGGCCATCAGTGGGCTCCAGTCCGAGGATGAGGCTGATTATTACTGT |
| | | | GCAGCATGGGATGACAGCCTGAATGGTCTAGTCTTCGGAACTGGGACCAAGCTGAC |
| | | | CGTCCTA |
| 82 | PRSIM_72 | DNA | CAGGTGCAGCTGGTGCAGTCTGGGGCTGAGGTGAAGAAGCCGGGGTCCTCGGTGAA |
| | | | GGTCTCCTGCAAGGTTTCTGGAGGCAGCTTCAATAATTATGGTGTCAGTTGGGTGC |
| | | | GACAGGCCCCTGGACAAGGGCTTGAGTGGATGGGAAGGATCATCCCTATCCGTGAT |
| | | | ACAGCAAACTACGCACAGAAGTTCCAGGGCAGAGTCACGATTACCGCGGACACATC |
| | | | CACGAACATTGCCTACATGGAACTGAGCGGCCTGAGATCTGACGACACGGCCGTGT |
| | | | ATTACTGTGCGAGAGTACTTGAGGACGATTTCTGGGGTGGTTATTATGACTTCTAT |
| | | | TTCTACGTTATGGACGTCTGGGGCCAGGGCACCCTGGTCACCGTCTCGAGTGGAGG |
| | | | CGGCGGTTCAGGCGGAGGTGGCTCTGGCGGTGGCGGAAGTGCACTTTCTTCTGAGC |
| | | | TGACTCAGGACCCTGTTGTGTCTGTGCCCTTGGGACAGACAGCCAGGATCACATGC |
| | | | CAAGGAGACAGCCTCACCACTTATTATGCAACCTGGTACCAGCAGAAGCCAGGACA |
| | | | GGCCCCTGTTCTTGTCCTCTATAATGAACACAAAAGGCCCTCAGGGATCTCAGACC |
| | | | GATTCTCTGGCTCCAGCGCAGGAGACGCAGCTTCCTTGACCATCACTGACACCCAG |
| | | | GCGGAAGATGAGGCCGACTATTATTGTAGCTCCCGGGACACCGGTGGGAAGCATGT |
| | | | GCTTTTCGGCGGAGGGACCAAGCTGACCGTCCTA |
| 83 | PRSIM_75 | DNA | GAGGTGCAGCTGGTGCAGTCTGGGGCTGAGGTGAAGAAGCCTGGGTCCTCGGTGAA |
| | | | GGTCTCCTGCAAGGCTTCTGGAGGCTCCTTCAACAGTTATACTCTCGACTGGGTGC |
| | | | GACAGGCCCCTGGACAAGGGCTTGAGTGGATGGGAGGGATCATCCCTGTCTTTGGT |
| | | | TCCCCGAACTACGGACAGAAATTCCAGGGCAGAGTCACCATTACCGCGGACGAATC |
| | | | AACGAGCACAGCCTACATGGAGCTGAGCAGTCTCAAATCTGACGACACGGCCGTGT |
| | | | ATTACTGTGCGCGAGGGTTGGTATACCAGCCCCTTGACTCCTGGGGCCGAGGCACC |
| | | | CTGGTCACCGTCTCGAGTGGAGGCGGCGGTTCAGGCGGAGGTGGCTCTGGCGGTGG |
| | | | CGGAAGTGCACAGGCTGTGCTGACTCAGCCGTCCTCAGCGTCTGGGACCCCCGGGC |
| | | | AGAGGGTCACCATCTCTTGTTCTGGAAGCAGCTCCAACATCGGAAGTTATACTGTA |
| | | | AACTGGTACCAGCAATTCCCAGGAACGGCCCCCAAACTCCTCATCTATAGTAATAC |
| | | | TCAGCGGCCCTCAGGGGTCCCTGACCGATTCTCTGGCTCCAAGTCTGGCACCTCAG |
| | | | CCTCCCTGGCCATCAGTGGGCTCCAGTCTGAGGATGAGGCTGATTATTACTGTGCA |
| | | | GCATGGGATGACAGCCTGAATGGTTGGGTGTTCGGCGGAGGGACCAAGGTCACCGT |
| | | | CCTA |
| 84 | HCV_NS4A_ | DNA | ATGGGCAAGAAAAAGGGCTCTGTGGTCATCGTGGGCAGAATCAACCTGAGCGGCGA |
| | NS3_S139A_ | | TACCGCCTACGCTCAGCAGACAAGAGGCGAGGAAGGCTGCCAAGAGACAAGCCAGA |
| | SmBiT | | CCGGCAGAGACAAGAACCAGGTGGAAGGCGAGGTGCAGATCGTGTCTACAGCTACC |
| | | | CAGACCTTCCTGGCCACCAGCATCAATGGCGTGCTGTGGACAGTGTATCACGGCGC |
| | | | TGGCACCAGAACAATCGCCTCTCCAAAGGGCCCCGTGACACAGATGTACACCAACG |
| | | | TGGACAAGGACCTCGTCGGATGGCAAGCCCCTCAGGGCTCTAGAAGCCTGACACCT |
| | | | TGTACCTGCGGCAGCAGCGATCTGTACCTGGTCACAAGACACGCCGACGTGATCCC |
| | | | CGTCAGAAGAAGAGGCGATAGCAGAGGCAGCCTGCTGAGCCCTAGACCTATCAGCT |
| | | | ACCTGAAGGGATCTGCCGGCGGACCTCTGCTTTGTCCTGCTGGACATGCCGTGGGC |
| | | | ATCTTTAGAGCCGCCGTGTCTACTAGAGGCGTGGCCAAGGCCGTGGACTTCATCCC |
| | | | TGTGGAAAGCCTGGAAACCACCATGCGGAGCCCCTCTGGCTCGAGCGGTGGTGGCG |
| | | | GGAGCGGAGGTGGAGGGTCGTCAGGTGTGACCGGCTACCGGCTGTTCGAGGAGATT |
| | | | CTG |
| 85 | SmBiT_HCV_ | DNA | ATGGTGACCGGCTACCGGCTGTTCGAGGAGATTCTCGGGAGTTCCGGTGGTGGCGG |
| | NS4A_NS3_S139A | | GAGCGGAGGTGGAGGCTCGAGCGGTAAGAAAAAGGGCTCTGTGGTCATCGTGGGCA |
| | | | GAATCAACCTGAGCGGCGATACCGCCTACGCTCAGCAGACAAGAGGCGAGGAAGGC |
| | | | TGCCAAGAGACAAGCCAGACCGGCAGAGACAAGAACCAGGTGGAAGGCGAGGTGCA |
| | | | GATCGTGTCTACAGCTACCCAGACCTTCCTGGCCACCAGCATCAATGGCGTGCTGT |
| | | | GGACAGTGTATCACGGCGCTGGCACCAGAACAATCGCCTCTCCAAAGGGCCCCGTG |
| | | | ACACAGATGTACACCAACGTGGACAAGGACCTCGTCGGATGGCAAGCCCCTCAGGG |
| | | | CTCTAGAAGCCTGACACCTTGTACCTGCGGCAGCAGCGATCTGTACCTGGTCACAA |
| | | | GACACGCCGACGTGATCCCCGTCAGAAGAAGAGGCGATAGCAGAGGCAGCCTGCTG |
| | | | AGCCCTAGACCTATCAGCTACCTGAAGGGATCTGCCGGCGGACCTCTGCTTTGTCC |
| | | | TGCTGGACATGCCGTGGGCATCTTTAGAGCCGCCGTGTCTACTAGAGGCGTGGCCA |
| | | | AGGCCGTGGACTTCATCCCTGTGGAAAGCCTGGAAACCACCATGCGGAGCCCC |
| 86 | PRSIM_23_LgBiT | DNA | ATGGGCTCTAGACTGGATGCCCCTAGCCAGATCGAAGTGAAGGACGTGACCGACAC |
| | | | CACCGCTCTGATCACCTGGGTTGACCCCAGATACGACGACATCTGGTGGTTCGAGC |
| | | | TGACCTACGGCATCAAGGATGTGCCCGGCGACAGAACCACCATCAAGCTGTACCTG |
| | | | AACGACCCCTACTACAGCATCGGCAACCTGAAGCCTGACACCGAGTACGAGGTGTC |
| | | | CCTGATCAGCTACACCGGCGACTCCTACAGCAGAAGCGGCAGCAATCCTGCCAAGA |
| | | | TCACCTTCAAGACCGGCCTTTCTGGCTCGAGCGGTGGTGGCGGGAGCGGAGGTGGA |
| | | | GGGTCGTCAGGTGTCTTCACACTCGAAGATTTCGTTGGGGACTGGGAACAGACAGC |
| | | | CGCCTACAACCTGGACCAAGTCCTTGAACAGGGAGGTGTGTCCAGTTTGCTGCAGA |
| | | | ATCTCGCCGTGTCCGTAACTCCGATCCAAAGGATTGTCCGGAGCGGTGAAAATGCC |
| | | | CTGAAGATCGACATCCATGTCATCATCCCGTATGAAGGTCTGAGCGCCGACCAAAT |
| | | | GGCCCAGATCGAAGAGGTGTTTAAGGTGGTGTACCCTGTGGATGATCATCACTTTA |
| | | | AGGTGATCCTGCCCTATGGCACACTGGTAATCGACGGGGTTACGCCGAACATGCTG |
| | | | AACTATTTCGGACGGCCGTATGAAGGCATCGCCGTGTTCGACGGCAAAAAGATCAC |
| | | | TGTAACAGGGACCCTGTGGAACGGCAACAAAATTATCGACGAGCGCCTGATCACCC |
| | | | CCGACGGCTCCATGCTGTTCCGAGTAACCATCAACAGC |
| 87 | PRSIM_32_LgBiT | DNA | ATGGGCTCTAGACTGGATGCCCCTAGCCAGATCGAAGTGAAGGACGTGACCGACAC |
| | | | CACCGCTCTGATCACATGGTGGTCCCCACGGTACTACTACGCCAGCATCAGCGGCT |
| | | | TCGAGCTGACCTACGGCATCAAGGATGTGCCCGGCGACAGAACCACCATCAAGCTG |
| | | | GACTACGCCTCCAACGACTACAGCATCGGCAACCTGAAGCCTGACACCGAGTACGA |
| | | | GGTGTCCCTGATCAGCTGGAACTACGGCGATTGGCGGTACAGCAGCAGCAACCCTG |
| | | | CCAAGATCACCTTCAAGACCGGCCTTTCTGGCTCGAGCGGTGGTGGCGGGAGCGGA |
| | | | GGTGGAGGGTCGTCAGGTGTCTTCACACTCGAAGATTTCGTTGGGGACTGGGAACA |
| | | | GACAGCCGCCTACAACCTGGACCAAGTCCTTGAACAGGGAGGTGTGTCCAGTTTGC |
| | | | TGCAGAATCTCGCCGTGTCCGTAACTCCGATCCAAAGGATTGTCCGGAGCGGTGAA |
| | | | AATGCCCTGAAGATCGACATCCATGTCATCATCCCGTATGAAGGTCTGAGCGCCGA |
| | | | CCAAATGGCCCAGATCGAAGAGGTGTTTAAGGTGGTGTACCCTGTGGATGATCATC |
| | | | ACTTTAAGGTGATCCTGCCCTATGGCACACTGGTAATCGACGGGGTTACGCCGAAC |
| | | | ATGCTGAACTATTTCGGACGGCCGTATGAAGGCATCGCCGTGTTCGACGGCAAAAA |
| | | | GATCACTGTAACAGGGACCCTGTGGAACGGCAACAAAATTATCGACGAGCGCCTGA |
| | | | TCACCCCCGACGGCTCCATGCTGTTCCGAGTAACCATCAACAGC |
| 88 | PRSIM_33_LgBiT | DNA | ATGGGCTCTAGACTGGATGCCCCTAGCCAGATCGAAGTGAAGGACGTGACCGACAC |
| | | | CACCGCTCTGATCACCTGGTATCCACCTGGCCGTTGGTACGACGACATCTGGTACT |
| | | | TCGAGCTGACCTACGGCATCAAGGACGTGCCCGGCGATAGAACCACCATCAAACTG |
| | | | GCCAGAGGCGACGACGTGTACAGCATCGGCAACCTGAAGCCTGACACCGAGTACGA |
| | | | GGTGTCCCTGATCTCTTGGGGCCCTGACAGAGGCGATAGAGCCGGATCTAACCCCG |
| | | | CCAAGATCACCTTCAAGACCGGCCTTTCTGGCTCGAGCGGTGGTGGCGGGAGCGGA |
| | | | GGTGGAGGGTCGTCAGGTGTCTTCACACTCGAAGATTTCGTTGGGGACTGGGAACA |
| | | | GACAGCCGCCTACAACCTGGACCAAGTCCTTGAACAGGGAGGTGTGTCCAGTTTGC |
| | | | TGCAGAATCTCGCCGTGTCCGTAACTCCGATCCAAAGGATTGTCCGGAGCGGTGAA |
| | | | AATGCCCTGAAGATCGACATCCATGTCATCATCCCGTATGAAGGTCTGAGCGCCGA |
| | | | CCAAATGGCCCAGATCGAAGAGGTGTTTAAGGTGGTGTACCCTGTGGATGATCATC |
| | | | ACTTTAAGGTGATCCTGCCCTATGGCACACTGGTAATCGACGGGGTTACGCCGAAC |
| | | | ATGCTGAACTATTTCGGACGGCCGTATGAAGGCATCGCCGTGTTCGACGGCAAAAA |
| | | | GATCACTGTAACAGGGACCCTGTGGAACGGCAACAAAATTATCGACGAGCGCCTGA |
| | | | TCACCCCCGACGGCTCCATGCTGTTCCGAGTAACCATCAACAGC |
| 89 | PRSIM_36_LgBiT | DNA | ATGGGCTCTAGACTGGATGCCCCTAGCCAGATCGAAGTGAAGGACGTGACCGACAC |
| | | | CACCGCTCTGATCACCTGGTCCTGGCCTAGAGATGACGACTACGACATCTGGTACT |
| | | | TCGAGCTGACCTACGGCATCAAGGACGTGCCCGGCGATAGAACCACCATCAAGCTG |
| | | | CTGAACTACGCCTCTCCATACAGCATCGGCAACCTGAAGCCTGACACCGAGTACGA |
| | | | GGTGTCCCTGATCAGCGTGGTGCCCGACACATATGGCAGAGGCACAAGCAACCCCG |
| | | | CCAAGATCACCTTCAAGACCGGACTTTCTGGCTCGAGCGGTGGTGGCGGGAGCGGA |
| | | | GGTGGAGGGTCGTCAGGTGTCTTCACACTCGAAGATTTCGTTGGGGACTGGGAACA |
| | | | GACAGCCGCCTACAACCTGGACCAAGTCCTTGAACAGGGAGGTGTGTCCAGTTTGC |
| | | | TGCAGAATCTCGCCGTGTCCGTAACTCCGATCCAAAGGATTGTCCGGAGCGGTGAA |
| | | | AATGCCCTGAAGATCGACATCCATGTCATCATCCCGTATGAAGGTCTGAGCGCCGA |
| | | | CCAAATGGCCCAGATCGAAGAGGTGTTTAAGGTGGTGTACCCTGTGGATGATCATC |
| | | | ACTTTAAGGTGATCCTGCCCTATGGCACACTGGTAATCGACGGGGTTACGCCGAAC |
| | | | ATGCTGAACTATTTCGGACGGCCGTATGAAGGCATCGCCGTGTTCGACGGCAAAAA |
| | | | GATCACTGTAACAGGGACCCTGTGGAACGGCAACAAAATTATCGACGAGCGCCTGA |
| | | | TCACCCCCGACGGCTCCATGCTGTTCCGAGTAACCATCAACAGC |
| 90 | PRSIM_47_LgBiT | DNA | ATGGGCTCTAGACTGGATGCCCCTAGCCAGATCGAAGTGAAGGACGTGACCGACAC |
| | | | CACCGCTCTGATCACCTGGTCAAGACCTGGCGTGTCCATCTGGTACTTCGAGCTGA |
| | | | CCTACGGCATCAAGGACGTGCCCGGCGATAGAACCACCATCAAGCTGGACTACCGC |
| | | | AGCTACTACTACAGCATCGGCAACCTGAAGCCTGACACCGAGTACGAGGTGTCCCT |
| | | | GATCAGCGGCTCTTATGGCCTCGTGGGCGTCAGAGCCTCTAATCCCGCCAAGATCA |
| | | | CCTTTAAGACCGGCCTTTCTGGCTCGAGCGGTGGTGGCGGGAGCGGAGGTGGAGGG |
| | | | TCGTCAGGTGTCTTCACACTCGAAGATTTCGTTGGGGACTGGGAACAGACAGCCGC |
| | | | CTACAACCTGGACCAAGTCCTTGAACAGGGAGGTGTGTCCAGTTTGCTGCAGAATC |
| | | | TCGCCGTGTCCGTAACTCCGATCCAAAGGATTGTCCGGAGCGGTGAAAATGCCCTG |
| | | | AAGATCGACATCCATGTCATCATCCCGTATGAAGGTCTGAGCGCCGACCAAATGGC |
| | | | CCAGATCGAAGAGGTGTTTAAGGTGGTGTACCCTGTGGATGATCATCACTTTAAGG |
| | | | TGATCCTGCCCTATGGCACACTGGTAATCGACGGGGTTACGCCGAACATGCTGAAC |
| | | | TATTTCGGACGGCCGTATGAAGGCATCGCCGTGTTCGACGGCAAAAAGATCACTGT |
| | | | AACAGGGACCCTGTGGAACGGCAACAAAATTATCGACGAGCGCCTGATCACCCCCG |
| | | | ACGGCTCCATGCTGTTCCGAGTAACCATCAACAGC |
| 91 | PRSIM_01_LgBiT | DNA | ATGGGATCTCAGGTTCAGCTGGTTCAGTCTGGCGCCGAAGTGAAGAAACCTGGCAG |
| | | | CAGCGTGAAGGTGTCCTGCAAAGCTTCTGGCGGCACCTTCAGCAGCTACGCCATCT |
| | | | CTTGGGTTCGACAGGCCCCTGGACAAGGCCTGGAATGGATGGGAGGCATCATCCCC |
| | | | ATCTTCGGCACCGCCAATTACGCCCAGAAATTCCAGGGCAGAGTGACCATCACCGC |
| | | | CGACGAGTCTACAAGCACCGCCTACATGGAACTGAGCAGCCTGAGAAGCGAGGACA |
| | | | CCGCCGTGTACTATTGTGCCAGAGGCCAGGGCTACTACGGCTACTTCGATTATTGG |
| | | | GGCCAGGGCACCCTGGTCACAGTTTCTAGCGGAGGCGGAGGATCTGGTGGCGGAGG |
| | | | AAGTGGCGGAGGCGGTTCTGCTCAATCTGTGCTGACACAGCCTCCTAGCGCCTCTG |
| | | | GAACACCTGGCCAGAGAGTGACAATCAGCTGTAGCGGCAGCAGCAGCAACATCGGC |
| | | | AGCAACACCGTGAACTGGTATCAGCAGCTGCCTGGCACAGCCCCTAAACTGCTGAT |
| | | | CTACAGCAACAACCAGCGGCCTAGCGGCGTGCCCGATAGATTTTCCGTGTCTAAGA |
| | | | GCGGCACCAGCGCCAGCCTGGCTATTTCTGGACTGCAGAGCGAGGACGAGGCCGAC |
| | | | TATTATTGTGCCGCCTGGGATCACGGACACGAGCACGTTGTGTTTGGAGGCGGCAC |
| | | | CAAGCTGACAGTGCTTTCTGGCTCGAGCGGTGGTGGCGGGAGCGGAGGTGGAGGGT |
| | | | CGTCAGGTGTCTTCACACTCGAAGATTTCGTTGGGGACTGGGAACAGACAGCCGCC |
| | | | TACAACCTGGACCAAGTCCTTGAACAGGGAGGTGTGTCCAGTTTGCTGCAGAATCT |
| | | | CGCCGTGTCCGTAACTCCGATCCAAAGGATTGTCCGGAGCGGTGAAAATGCCCTGA |
| | | | AGATCGACATCCATGTCATCATCCCGTATGAAGGTCTGAGCGCCGACCAAATGGCC |
| | | | CAGATCGAAGAGGTGTTTAAGGTGGTGTACCCTGTGGATGATCATCACTTTAAGGT |
| | | | GATCCTGCCCTATGGCACACTGGTAATCGACGGGGTTACGCCGAACATGCTGAACT |
| | | | ATTTCGGACGGCCGTATGAAGGCATCGCCGTGTTCGACGGCAAAAAGATCACTGTA |
| | | | ACAGGGACCCTGTGGAACGGCAACAAAATTATCGACGAGCGCCTGATCACCCCCGA |
| | | | CGGCTCCATGCTGTTCCGAGTAACCATCAACAGC |
| 92 | PRSIM_06_LgBiT | DNA | ATGGGCTCTCAGGTGCAGCTTGTTCAGTCTGGCGCCGAAGTGAAGAAACCCGGCAG |
| | | | CTCTGTGAAGGTGTCCTGCAAAGCTTCCGGCGGCACCTTTAGCAGCTACGCCATCT |
| | | | CTTGGGTCCGACAGGCTCCTGGACAAGGCCTGGAATGGATGGGCGGCATCATCCCT |
| | | | ATCTTCGGCACCGCCAATTACGCCCAGAAATTCCAGGGCAGAGTGACCATCACCGC |
| | | | CGACGAGTCTACAAGCACCGCCTACATGGAACTGAGCAGCCTGAGAAGCGAGGACA |
| | | | CCGCCGTGTACTACTGTGCTAGAGGCGCTGGCTACTACATGAGAGTGGACTATTGG |
| | | | GGCCAGGGCACCCTGGTCACAGTTTCTAGCGGAGGCGGAGGATCTGGCGGCGGAGG |
| | | | TAGTGGTGGTGGCGGATCTGCTCAGTCTGTGCTGACACAGCCTCCTAGCGCCTCTG |
| | | | GAACACCTGGCCAGAGAGTGACAATCAGCTGTAGCGGCAGCAGCAGCAACATCGGC |
| | | | AGCAACACCGTGAACTGGTATCAGCAGCTGCCTGGCACAGCCCCTAAACTGCTGAT |
| | | | CTACAGCAACAACCAGCGGCCTAGCGGCGTGCCCGATAGATTTTCTGGCAGCAAGA |
| | | | GCGGCACAAGCGCCAGCCTGGCTATTTCTGGACTGCAGAGCGAGGACGAGGCCGAC |
| | | | TATTATTGTGCCGCCTGGGACCACGACGTGGAACACGTTGTGTTTGGCGGAGGCAC |
| | | | CAAGCTGACAGTGCTTTCTGGCTCGAGCGGTGGTGGCGGGAGCGGAGGTGGAGGGT |
| | | | CGTCAGGTGTCTTCACACTCGAAGATTTCGTTGGGGACTGGGAACAGACAGCCGCC |
| | | | TACAACCTGGACCAAGTCCTTGAACAGGGAGGTGTGTCCAGTTTGCTGCAGAATCT |
| | | | CGCCGTGTCCGTAACTCCGATCCAAAGGATTGTCCGGAGCGGTGAAAATGCCCTGA |
| | | | AGATCGACATCCATGTCATCATCCCGTATGAAGGTCTGAGCGCCGACCAAATGGCC |
| | | | CAGATCGAAGAGGTGTTTAAGGTGGTGTACCCTGTGGATGATCATCACTTTAAGGT |
| | | | GATCCTGCCCTATGGCACACTGGTAATCGACGGGGTTACGCCGAACATGCTGAACT |
| | | | ATTTCGGACGGCCGTATGAAGGCATCGCCGTGTTCGACGGCAAAAAGATCACTGTA |
| | | | ACAGGGACCCTGTGGAACGGCAACAAAATTATCGACGAGCGCCTGATCACCCCCGA |
| | | | CGGCTCCATGCTGTTCCGAGTAACCATCAACAGC |
| 93 | PRSIM_57_LgBiT | DNA | ATGGGCTCTCAGGTTCAGCTGGTTCAGTCTGGCGCCGAAGTGAAGAAACCTGGCAG |
| | | | CAGCGTGAAGGTGTCCTGCAAAGCTTCTGGCGGCACCTTCAGCAGCTACGCCATCT |
| | | | CTTGGGTTCGACAGGCCCCTGGACAAGGCCTGGAATGGATGGGAGGCATCATCCCC |
| | | | ATCTTCGGCACCGCCAATTACGCCCAGAAATTCCAGGGCAGAGTGACCATCACCGC |
| | | | CGACGAGTCTACAAGCACCGCCTACATGGAACTGAGCAGCCTGAGAAGCGAGGACA |
| | | | CCGCCGTGTACTACTGTGCCAGACACACCAACTACATCACCGTGTTCGACTACTGG |
| | | | GGCCAGGGCACACTGGTCACAGTTTCTAGCGGAGGCGGAGGATCTGGTGGCGGAGG |
| | | | AAGTGGCGGAGGCGGTTCTGCTCAATCTGTGCTGACACAGCCTCCTAGCGCCTCTG |
| | | | GAACACCTGGCCAGAGAGTGACAATCAGCTGTAGCGGCAGCAGCAGCAACATCGGC |
| | | | AGCAACACCGTGAACTGGTATCAGCAGCTGCCTGGCACAGCCCCTAAACTGCTGAT |
| | | | CTACAGCAACAACCAGCGGCCTAGCGGCGTGCCCGATAGATTTTCTGGCAGCAAGA |
| | | | GCGGCACAAGCGCCAGCCTGGCTATTTCTGGACTGCAGAGCGAGGACGAGGCCGAC |
| | | | TATTATTGTGCCGCCTGGGACCACCACTGGGAGCAAGTTGTTTTTGGAGGCGGCAC |
| | | | CAAGCTGACCGTGCTTTCTGGCTCGAGCGGTGGTGGCGGGAGCGGAGGTGGAGGGT |
| | | | CGTCAGGTGTCTTCACACTCGAAGATTTCGTTGGGGACTGGGAACAGACAGCCGCC |
| | | | TACAACCTGGACCAAGTCCTTGAACAGGGAGGTGTGTCCAGTTTGCTGCAGAATCT |
| | | | CGCCGTGTCCGTAACTCCGATCCAAAGGATTGTCCGGAGCGGTGAAAATGCCCTGA |
| | | | AGATCGACATCCATGTCATCATCCCGTATGAAGGTCTGAGCGCCGACCAAATGGCC |
| | | | CAGATCGAAGAGGTGTTTAAGGTGGTGTACCCTGTGGATGATCATCACTTTAAGGT |
| | | | GATCCTGCCCTATGGCACACTGGTAATCGACGGGGTTACGCCGAACATGCTGAACT |
| | | | ATTTCGGACGGCCGTATGAAGGCATCGCCGTGTTCGACGGCAAAAAGATCACTGTA |
| | | | ACAGGGACCCTGTGGAACGGCAACAAAATTATCGACGAGCGCCTGATCACCCCCGA |
| | | | CGGCTCCATGCTGTTCCGAGTAACCATCAACAGC |
| 94 | PRSIM_67_LgBiT | DNA | ATGGGCTCTGAAGTGCAGCTGGTTCAGTCTGGCGCCGAAGTGAAGAAACCTGGCGC |
| | | | CGCTGTCAGAATCAGCTGCAAGACAAGCGGCTACGTGTTCACCAGCTACTACGTGC |
| | | | ACTGGGTCCGACAGGCTCCAGGACAAGGACTGGAATGGATGGGCGTGATCAATCCC |
| | | | AGCGGCGGCAACACCAATTACGCCCAGAAATTCCAGGACCGCGTGACCATGACCAG |
| | | | AGACACCAGCACCACCACCGTGTACATGGAACTGAGCAGCCTGATGTTCGACGACA |
| | | | CCGCCGTGTACTACTGCGCCAAGAGAGATTACGGCGGACCCCTGGCCAATTGGGGC |
| | | | AGAGGAACACTGGTCACAGTGTCTAGCGGAGGCGGAGGATCTGGTGGCGGAGGAAG |
| | | | TGGCGGAGGCGGTTCTGCTCTGAGCTATGAGCTGACACAGCCTCCAAGCGTGTCCG |
| | | | AGGCTCCTAGACAGAGAGTGACCATCAGCTGTAGCGGCAGCAGCAGCAACATCGGC |
| | | | AACAACGCCGTGAACTGGTATCAGCAGCTGCCTGGCAAGGCCCCTAAACTGCTGAT |
| | | | CTTCTACGACGACCTGCTGCCTAGCGGAGTGTCCGATAGATTCAGCGGCTCTAAGA |
| | | | GCGGCACATCTGCCAGCCTGGCCATCTCTGGACTGCAGAGCGAAGATGAGGCCGAC |
| | | | TACTATTGCGCCGCCTGGGACGATTCTCTGAACGGCCTGGTTTTTGGCACCGGCAC |
| | | | CAAGCTGACAGTGCTGTCTGGCTCGAGCGGTGGTGGCGGGAGCGGAGGTGGAGGGT |
| | | | CGTCAGGTGTCTTCACACTCGAAGATTTCGTTGGGGACTGGGAACAGACAGCCGCC |
| | | | TACAACCTGGACCAAGTCCTTGAACAGGGAGGTGTGTCCAGTTTGCTGCAGAATCT |
| | | | CGCCGTGTCCGTAACTCCGATCCAAAGGATTGTCCGGAGCGGTGAAAATGCCCTGA |
| | | | AGATCGACATCCATGTCATCATCCCGTATGAAGGTCTGAGCGCCGACCAAATGGCC |
| | | | CAGATCGAAGAGGTGTTTAAGGTGGTGTACCCTGTGGATGATCATCACTTTAAGGT |
| | | | GATCCTGCCCTATGGCACACTGGTAATCGACGGGGTTACGCCGAACATGCTGAACT |
| | | | ATTTCGGACGGCCGTATGAAGGCATCGCCGTGTTCGACGGCAAAAAGATCACTGTA |
| | | | ACAGGGACCCTGTGGAACGGCAACAAAATTATCGACGAGCGCCTGATCACCCCCGA |
| | | | CGGCTCCATGCTGTTCCGAGTAACCATCAACAGC |
| 95 | PRSIM_72_LgBiT | DNA | ATGGGATCTCAGGTTCAGCTGGTTCAGTCTGGCGCCGAAGTGAAGAAACCTGGCAG |
| | | | CAGCGTGAAGGTGTCCTGCAAAGTGTCTGGCGGCAGCTTCAACAACTACGGCGTGT |
| | | | CCTGGGTTCGACAGGCCCCTGGACAAGGACTGGAATGGATGGGCAGAATCATCCCC |
| | | | ATCCGGGACACCGCCAATTACGCCCAGAAATTCCAGGGCAGAGTGACCATCACCGC |
| | | | CGACACCAGCACCAATATCGCCTACATGGAACTGAGCGGCCTGCGGAGTGATGACA |
| | | | CCGCCGTGTACTATTGCGCCAGAGTGCTGGAAGATGACTTCTGGGGCGGCTACTAC |
| | | | GACTTCTACTTCTACGTGATGGACGTGTGGGGCCAGGGCACACTGGTTACAGTTTC |
| | | | TAGCGGAGGCGGAGGATCTGGTGGCGGAGGAAGTGGCGGAGGCGGTTCTGCTCTTT |
| | | | CTAGCGAGCTGACCCAGGATCCAGTGGTGTCTGTTCCTCTGGGCCAGACCGCCAGA |
| | | | ATTACCTGTCAGGGCGATAGCCTGACCACCTACTACGCCACCTGGTATCAGCAGAA |
| | | | GCCAGGCCAGGCTCCTGTGCTGGTGCTGTACAATGAGCACAAGAGGCCCAGCGGCA |
| | | | TCAGCGACAGATTTTCTGGATCTTCTGCCGGCGACGCCGCCAGCCTGACAATCACA |
| | | | GATACACAGGCCGAGGACGAGGCCGACTACTACTGCAGCTCTAGAGATACCGGCGG |
| | | | CAAACACGTGCTGTTTGGAGGCGGCACAAAGCTGACAGTGCTTTCTGGCTCGAGCG |
| | | | GTGGTGGCGGGAGCGGAGGTGGAGGGTCGTCAGGTGTCTTCACACTCGAAGATTTC |
| | | | GTTGGGGACTGGGAACAGACAGCCGCCTACAACCTGGACCAAGTCCTTGAACAGGG |
| | | | AGGTGTGTCCAGTTTGCTGCAGAATCTCGCCGTGTCCGTAACTCCGATCCAAAGGA |
| | | | TTGTCCGGAGCGGTGAAAATGCCCTGAAGATCGACATCCATGTCATCATCCCGTAT |
| | | | GAAGGTCTGAGCGCCGACCAAATGGCCCAGATCGAAGAGGTGTTTAAGGTGGTGTA |
| | | | CCCTGTGGATGATCATCACTTTAAGGTGATCCTGCCCTATGGCACACTGGTAATCG |
| | | | ACGGGGTTACGCCGAACATGCTGAACTATTTCGGACGGCCGTATGAAGGCATCGCC |
| | | | GTGTTCGACGGCAAAAAGATCACTGTAACAGGGACCCTGTGGAACGGCAACAAAAT |
| | | | TATCGACGAGCGCCTGATCACCCCCGACGGCTCCATGCTGTTCCGAGTAACCATCA |
| | | | ACAGC |
| 96 | PRSIM_75_LgBiT | DNA | ATGGGATCTGAAGTGCAGCTGGTTCAGTCTGGCGCCGAAGTGAAGAAACCTGGCAG |
| | | | CAGCGTGAAGGTGTCCTGCAAAGCTTCTGGCGGCAGCTTCAACAGCTACACCCTGG |
| | | | ACTGGGTTCGACAGGCCCCTGGACAAGGACTGGAATGGATGGGCGGAATCATCCCC |
| | | | GTGTTCGGCAGCCCTAATTACGGCCAGAAATTCCAGGGCAGAGTGACCATCACCGC |
| | | | CGACGAGTCTACAAGCACCGCCTACATGGAACTGAGCAGCCTGAAGTCCGACGACA |
| | | | CCGCCGTGTACTATTGTGCCAGAGGCCTGGTGTACCAGCCACTGGATTCTTGGGGC |
| | | | AGAGGCACCCTGGTCACAGTTTCTAGCGGAGGCGGAGGATCTGGTGGCGGAGGAAG |
| | | | TGGCGGAGGCGGTTCTGCTCAAGCTGTTCTGACACAGCCTAGCAGCGCCTCTGGAA |
| | | | CACCTGGCCAGAGAGTGACAATCAGCTGTAGCGGCAGCAGCAGCAACATCGGCTCC |
| | | | TACACCGTGAACTGGTATCAGCAGTTCCCCGGCACAGCCCCTAAGCTGCTGATCTA |
| | | | CAGCAACACCCAGAGGCCAAGCGGCGTGCCCGATAGATTTTCTGGCAGCAAGAGCG |
| | | | GCACAAGCGCCAGCCTGGCTATTTCTGGACTGCAGAGCGAGGACGAGGCCGACTAT |
| | | | TATTGTGCCGCCTGGGACGACAGCCTGAACGGATGGGTTTTCGGCGGAGGCACCAA |
| | | | AGTGACAGTGCTTTCTGGCTCGAGCGGTGGTGGCGGGAGCGGAGGTGGAGGGTCGT |
| | | | CAGGTGTCTTCACACTCGAAGATTTCGTTGGGGACTGGGAACAGACAGCCGCCTAC |
| | | | AACCTGGACCAAGTCCTTGAACAGGGAGGTGTGTCCAGTTTGCTGCAGAATCTCGC |
| | | | CGTGTCCGTAACTCCGATCCAAAGGATTGTCCGGAGCGGTGAAAATGCCCTGAAGA |
| | | | TCGACATCCATGTCATCATCCCGTATGAAGGTCTGAGCGCCGACCAAATGGCCCAG |
| | | | ATCGAAGAGGTGTTTAAGGTGGTGTACCCTGTGGATGATCATCACTTTAAGGTGAT |
| | | | CCTGCCCTATGGCACACTGGTAATCGACGGGGTTACGCCGAACATGCTGAACTATT |
| | | | TCGGACGGCCGTATGAAGGCATCGCCGTGTTCGACGGCAAAAAGATCACTGTAACA |
| | | | GGGACCCTGTGGAACGGCAACAAAATTATCGACGAGCGCCTGATCACCCCCGACGG |
| | | | CTCCATGCTGTTCCGAGTAACCATCAACAGC |
| 97 | LgBiT_PRSIM_23 | DNA | ATGGTCTTCACACTCGAAGATTTCGTTGGGGACTGGGAACAGACAGCCGCCTACAA |
| | | | CCTGGACCAAGTCCTTGAACAGGGAGGTGTGTCCAGTTTGCTGCAGAATCTCGCCG |
| | | | TGTCCGTAACTCCGATCCAAAGGATTGTCCGGAGCGGTGAAAATGCCCTGAAGATC |
| | | | GACATCCATGTCATCATCCCGTATGAAGGTCTGAGCGCCGACCAAATGGCCCAGAT |
| | | | CGAAGAGGTGTTTAAGGTGGTGTACCCTGTGGATGATCATCACTTTAAGGTGATCC |
| | | | TGCCCTATGGCACACTGGTAATCGACGGGGTTACGCCGAACATGCTGAACTATTTC |
| | | | GGACGGCCGTATGAAGGCATCGCCGTGTTCGACGGCAAAAAGATCACTGTAACAGG |
| | | | GACCCTGTGGAACGGCAACAAAATTATCGACGAGCGCCTGATCACCCCCGACGGCT |
| | | | CCATGCTGTTCCGAGTAACCATCAACAGTGGGAGTTCCGGTGGTGGCGGGAGCGGA |
| | | | GGTGGAGGCTCGAGCGGTAGACTGGATGCCCCTAGCCAGATCGAAGTGAAGGACGT |
| | | | GACCGACACCACCGCTCTGATCACCTGGGTTGACCCCAGATACGACGACATCTGGT |
| | | | GGTTCGAGCTGACCTACGGCATCAAGGATGTGCCCGGCGACAGAACCACCATCAAG |
| | | | CTGTACCTGAACGACCCCTACTACAGCATCGGCAACCTGAAGCCTGACACCGAGTA |
| | | | CGAGGTGTCCCTGATCAGCTACACCGGCGACTCCTACAGCAGAAGCGGCAGCAATC |
| | | | CTGCCAAGATCACCTTCAAGACCGGCCTT |
| 98 | LgBiT_PRSIM_32 | DNA | ATGGTCTTCACACTCGAAGATTTCGTTGGGGACTGGGAACAGACAGCCGCCTACAA |
| | | | CCTGGACCAAGTCCTTGAACAGGGAGGTGTGTCCAGTTTGCTGCAGAATCTCGCCG |
| | | | TGTCCGTAACTCCGATCCAAAGGATTGTCCGGAGCGGTGAAAATGCCCTGAAGATC |
| | | | GACATCCATGTCATCATCCCGTATGAAGGTCTGAGCGCCGACCAAATGGCCCAGAT |
| | | | CGAAGAGGTGTTTAAGGTGGTGTACCCTGTGGATGATCATCACTTTAAGGTGATCC |
| | | | TGCCCTATGGCACACTGGTAATCGACGGGGTTACGCCGAACATGCTGAACTATTTC |
| | | | GGACGGCCGTATGAAGGCATCGCCGTGTTCGACGGCAAAAAGATCACTGTAACAGG |
| | | | GACCCTGTGGAACGGCAACAAAATTATCGACGAGCGCCTGATCACCCCCGACGGCT |
| | | | CCATGCTGTTCCGAGTAACCATCAACAGTGGGAGTTCCGGTGGTGGCGGGAGCGGA |
| | | | GGTGGAGGCTCGAGCGGTAGACTGGATGCCCCTAGCCAGATCGAAGTGAAGGACGT |
| | | | GACCGACACCACCGCTCTGATCACATGGTGGTCCCCACGGTACTACTACGCCAGCA |
| | | | TCAGCGGCTTCGAGCTGACCTACGGCATCAAGGATGTGCCCGGCGACAGAACCACC |
| | | | ATCAAGCTGGACTACGCCTCCAACGACTACAGCATCGGCAACCTGAAGCCTGACAC |
| | | | CGAGTACGAGGTGTCCCTGATCAGCTGGAACTACGGCGATTGGCGGTACAGCAGCA |
| | | | GCAACCCTGCCAAGATCACCTTCAAGACCGGCCTT |
| 99 | LgBiT_PRSIM_33 | DNA | ATGGTCTTCACACTCGAAGATTTCGTTGGGGACTGGGAACAGACAGCCGCCTACAA |
| | | | CCTGGACCAAGTCCTTGAACAGGGAGGTGTGTCCAGTTTGCTGCAGAATCTCGCCG |
| | | | TGTCCGTAACTCCGATCCAAAGGATTGTCCGGAGCGGTGAAAATGCCCTGAAGATC |
| | | | GACATCCATGTCATCATCCCGTATGAAGGTCTGAGCGCCGACCAAATGGCCCAGAT |
| | | | CGAAGAGGTGTTTAAGGTGGTGTACCCTGTGGATGATCATCACTTTAAGGTGATCC |
| | | | TGCCCTATGGCACACTGGTAATCGACGGGGTTACGCCGAACATGCTGAACTATTTC |
| | | | GGACGGCCGTATGAAGGCATCGCCGTGTTCGACGGCAAAAAGATCACTGTAACAGG |
| | | | GACCCTGTGGAACGGCAACAAAATTATCGACGAGCGCCTGATCACCCCCGACGGCT |
| | | | CCATGCTGTTCCGAGTAACCATCAACAGTGGGAGTTCCGGTGGTGGCGGGAGCGGA |
| | | | GGTGGAGGCTCGAGCGGTAGACTGGATGCCCCTAGCCAGATCGAAGTGAAGGACGT |
| | | | GACCGACACCACCGCTCTGATCACCTGGTATCCACCTGGCCGTTGGTACGACGACA |
| | | | TCTGGTACTTCGAGCTGACCTACGGCATCAAGGACGTGCCCGGCGATAGAACCACC |
| | | | ATCAAACTGGCCAGAGGCGACGACGTGTACAGCATCGGCAACCTGAAGCCTGACAC |
| | | | CGAGTACGAGGTGTCCCTGATCTCTTGGGGCCCTGACAGAGGCGATAGAGCCGGAT |
| | | | CTAACCCCGCCAAGATCACCTTCAAGACCGGCCTT |
| 100 | LgBiT_PRSIM_36 | DNA | ATGGTCTTCACACTCGAAGATTTCGTTGGGGACTGGGAACAGACAGCCGCCTACAA |
| | | | CCTGGACCAAGTCCTTGAACAGGGAGGTGTGTCCAGTTTGCTGCAGAATCTCGCCG |
| | | | TGTCCGTAACTCCGATCCAAAGGATTGTCCGGAGCGGTGAAAATGCCCTGAAGATC |
| | | | GACATCCATGTCATCATCCCGTATGAAGGTCTGAGCGCCGACCAAATGGCCCAGAT |
| | | | CGAAGAGGTGTTTAAGGTGGTGTACCCTGTGGATGATCATCACTTTAAGGTGATCC |
| | | | TGCCCTATGGCACACTGGTAATCGACGGGGTTACGCCGAACATGCTGAACTATTTC |
| | | | GGACGGCCGTATGAAGGCATCGCCGTGTTCGACGGCAAAAAGATCACTGTAACAGG |
| | | | GACCCTGTGGAACGGCAACAAAATTATCGACGAGCGCCTGATCACCCCCGACGGCT |
| | | | CCATGCTGTTCCGAGTAACCATCAACAGTGGGAGTTCCGGTGGTGGCGGGAGCGGA |
| | | | GGTGGAGGCTCGAGCGGTAGACTGGATGCCCCTAGCCAGATCGAAGTGAAGGACGT |
| | | | GACCGACACCACCGCTCTGATCACCTGGTCCTGGCCTAGAGATGACGACTACGACA |
| | | | TCTGGTACTTCGAGCTGACCTACGGCATCAAGGACGTGCCCGGCGATAGAACCACC |
| | | | ATCAAGCTGCTGAACTACGCCTCTCCATACAGCATCGGCAACCTGAAGCCTGACAC |
| | | | CGAGTACGAGGTGTCCCTGATCAGCGTGGTGCCCGACACATATGGCAGAGGCACAA |
| | | | GCAACCCCGCCAAGATCACCTTCAAGACCGGACTT |
| 101 | LgBiT_PRSIM_47 | DNA | ATGGTCTTCACACTCGAAGATTTCGTTGGGGACTGGGAACAGACAGCCGCCTACAA |
| | | | CCTGGACCAAGTCCTTGAACAGGGAGGTGTGTCCAGTTTGCTGCAGAATCTCGCCG |
| | | | TGTCCGTAACTCCGATCCAAAGGATTGTCCGGAGCGGTGAAAATGCCCTGAAGATC |
| | | | GACATCCATGTCATCATCCCGTATGAAGGTCTGAGCGCCGACCAAATGGCCCAGAT |
| | | | CGAAGAGGTGTTTAAGGTGGTGTACCCTGTGGATGATCATCACTTTAAGGTGATCC |
| | | | TGCCCTATGGCACACTGGTAATCGACGGGGTTACGCCGAACATGCTGAACTATTTC |
| | | | GGACGGCCGTATGAAGGCATCGCCGTGTTCGACGGCAAAAAGATCACTGTAACAGG |
| | | | GACCCTGTGGAACGGCAACAAAATTATCGACGAGCGCCTGATCACCCCCGACGGCT |
| | | | CCATGCTGTTCCGAGTAACCATCAACAGTGGGAGTTCCGGTGGTGGCGGGAGCGGA |
| | | | GGTGGAGGCTCGAGCGGTAGACTGGATGCCCCTAGCCAGATCGAAGTGAAGGACGT |
| | | | GACCGACACCACCGCTCTGATCACCTGGTCAAGACCTGGCGTGTCCATCTGGTACT |
| | | | TCGAGCTGACCTACGGCATCAAGGACGTGCCCGGCGATAGAACCACCATCAAGCTG |
| | | | GACTACCGCAGCTACTACTACAGCATCGGCAACCTGAAGCCTGACACCGAGTACGA |
| | | | GGTGTCCCTGATCAGCGGCTCTTATGGCCTCGTGGGCGTCAGAGCCTCTAATCCCG |
| | | | CCAAGATCACCTTTAAGACCGGCCTT |
| 102 | LgBiT_PRSIM_01 | DNA | ATGGTCTTCACACTCGAAGATTTCGTTGGGGACTGGGAACAGACAGCCGCCTACAA |
| | | | CCTGGACCAAGTCCTTGAACAGGGAGGTGTGTCCAGTTTGCTGCAGAATCTCGCCG |
| | | | TGTCCGTAACTCCGATCCAAAGGATTGTCCGGAGCGGTGAAAATGCCCTGAAGATC |
| | | | GACATCCATGTCATCATCCCGTATGAAGGTCTGAGCGCCGACCAAATGGCCCAGAT |
| | | | CGAAGAGGTGTTTAAGGTGGTGTACCCTGTGGATGATCATCACTTTAAGGTGATCC |
| | | | TGCCCTATGGCACACTGGTAATCGACGGGGTTACGCCGAACATGCTGAACTATTTC |
| | | | GGACGGCCGTATGAAGGCATCGCCGTGTTCGACGGCAAAAAGATCACTGTAACAGG |
| | | | GACCCTGTGGAACGGCAACAAAATTATCGACGAGCGCCTGATCACCCCCGACGGCT |
| | | | CCATGCTGTTCCGAGTAACCATCAACAGTGGGAGTTCCGGTGGTGGCGGGAGCGGA |
| | | | GGTGGAGGCTCGAGCGGTCAGGTTCAGCTGGTTCAGTCTGGCGCCGAAGTGAAGAA |
| | | | ACCTGGCAGCAGCGTGAAGGTGTCCTGCAAAGCTTCTGGCGGCACCTTCAGCAGCT |
| | | | ACGCCATCTCTTGGGTTCGACAGGCCCCTGGACAAGGCCTGGAATGGATGGGAGGC |
| | | | ATCATCCCCATCTTCGGCACCGCCAATTACGCCCAGAAATTCCAGGGCAGAGTGAC |
| | | | CATCACCGCCGACGAGTCTACAAGCACCGCCTACATGGAACTGAGCAGCCTGAGAA |
| | | | GCGAGGACACCGCCGTGTACTATTGTGCCAGAGGCCAGGGCTACTACGGCTACTTC |
| | | | GATTATTGGGGCCAGGGCACCCTGGTCACAGTTTCTAGCGGAGGCGGAGGATCTGG |
| | | | TGGCGGAGGAAGTGGCGGAGGCGGTTCTGCTCAATCTGTGCTGACACAGCCTCCTA |
| | | | GCGCCTCTGGAACACCTGGCCAGAGAGTGACAATCAGCTGTAGCGGCAGCAGCAGC |
| | | | AACATCGGCAGCAACACCGTGAACTGGTATCAGCAGCTGCCTGGCACAGCCCCTAA |
| | | | ACTGCTGATCTACAGCAACAACCAGCGGCCTAGCGGCGTGCCCGATAGATTTTCCG |
| | | | TGTCTAAGAGCGGCACCAGCGCCAGCCTGGCTATTTCTGGACTGCAGAGCGAGGAC |
| | | | GAGGCCGACTATTATTGTGCCGCCTGGGATCACGGACACGAGCACGTTGTGTTTGG |
| | | | AGGCGGCACCAAGCTGACAGTGCTT |
| 103 | LgBiT_PRSIM_06 | DNA | ATGGTCTTCACACTCGAAGATTTCGTTGGGGACTGGGAACAGACAGCCGCCTACAA |
| | | | CCTGGACCAAGTCCTTGAACAGGGAGGTGTGTCCAGTTTGCTGCAGAATCTCGCCG |
| | | | TGTCCGTAACTCCGATCCAAAGGATTGTCCGGAGCGGTGAAAATGCCCTGAAGATC |
| | | | GACATCCATGTCATCATCCCGTATGAAGGTCTGAGCGCCGACCAAATGGCCCAGAT |
| | | | CGAAGAGGTGTTTAAGGTGGTGTACCCTGTGGATGATCATCACTTTAAGGTGATCC |
| | | | TGCCCTATGGCACACTGGTAATCGACGGGGTTACGCCGAACATGCTGAACTATTTC |
| | | | GGACGGCCGTATGAAGGCATCGCCGTGTTCGACGGCAAAAAGATCACTGTAACAGG |
| | | | GACCCTGTGGAACGGCAACAAAATTATCGACGAGCGCCTGATCACCCCCGACGGCT |
| | | | CCATGCTGTTCCGAGTAACCATCAACAGTGGGAGTTCCGGTGGTGGCGGGAGCGGA |
| | | | GGTGGAGGCTCGAGCGGTCAGGTGCAGCTTGTTCAGTCTGGCGCCGAAGTGAAGAA |
| | | | ACCCGGCAGCTCTGTGAAGGTGTCCTGCAAAGCTTCCGGCGGCACCTTTAGCAGCT |
| | | | ACGCCATCTCTTGGGTCCGACAGGCTCCTGGACAAGGCCTGGAATGGATGGGCGGC |
| | | | ATCATCCCTATCTTCGGCACCGCCAATTACGCCCAGAAATTCCAGGGCAGAGTGAC |
| | | | CATCACCGCCGACGAGTCTACAAGCACCGCCTACATGGAACTGAGCAGCCTGAGAA |
| | | | GCGAGGACACCGCCGTGTACTACTGTGCTAGAGGCGCTGGCTACTACATGAGAGTG |
| | | | GACTATTGGGGCCAGGGCACCCTGGTCACAGTTTCTAGCGGAGGCGGAGGATCTGG |
| | | | CGGCGGAGGTAGTGGTGGTGGCGGATCTGCTCAGTCTGTGCTGACACAGCCTCCTA |
| | | | GCGCCTCTGGAACACCTGGCCAGAGAGTGACAATCAGCTGTAGCGGCAGCAGCAGC |
| | | | AACATCGGCAGCAACACCGTGAACTGGTATCAGCAGCTGCCTGGCACAGCCCCTAA |
| | | | ACTGCTGATCTACAGCAACAACCAGCGGCCTAGCGGCGTGCCCGATAGATTTTCTG |
| | | | GCAGCAAGAGCGGCACAAGCGCCAGCCTGGCTATTTCTGGACTGCAGAGCGAGGAC |
| | | | GAGGCCGACTATTATTGTGCCGCCTGGGACCACGACGTGGAACACGTTGTGTTTGG |
| | | | CGGAGGCACCAAGCTGACAGTGCTT |
| 104 | LgBiT_PRSIM_57 | DNA | ATGGTCTTCACACTCGAAGATTTCGTTGGGGACTGGGAACAGACAGCCGCCTACAA |
| | | | CCTGGACCAAGTCCTTGAACAGGGAGGTGTGTCCAGTTTGCTGCAGAATCTCGCCG |
| | | | TGTCCGTAACTCCGATCCAAAGGATTGTCCGGAGCGGTGAAAATGCCCTGAAGATC |
| | | | GACATCCATGTCATCATCCCGTATGAAGGTCTGAGCGCCGACCAAATGGCCCAGAT |
| | | | CGAAGAGGTGTTTAAGGTGGTGTACCCTGTGGATGATCATCACTTTAAGGTGATCC |
| | | | TGCCCTATGGCACACTGGTAATCGACGGGGTTACGCCGAACATGCTGAACTATTTC |
| | | | GGACGGCCGTATGAAGGCATCGCCGTGTTCGACGGCAAAAAGATCACTGTAACAGG |
| | | | GACCCTGTGGAACGGCAACAAAATTATCGACGAGCGCCTGATCACCCCCGACGGCT |
| | | | CCATGCTGTTCCGAGTAACCATCAACAGTGGGAGTTCCGGTGGTGGCGGGAGCGGA |
| | | | GGTGGAGGCTCGAGCGGTCAGGTTCAGCTGGTTCAGTCTGGCGCCGAAGTGAAGAA |
| | | | ACCTGGCAGCAGCGTGAAGGTGTCCTGCAAAGCTTCTGGCGGCACCTTCAGCAGCT |
| | | | ACGCCATCTCTTGGGTTCGACAGGCCCCTGGACAAGGCCTGGAATGGATGGGAGGC |
| | | | ATCATCCCCATCTTCGGCACCGCCAATTACGCCCAGAAATTCCAGGGCAGAGTGAC |
| | | | CATCACCGCCGACGAGTCTACAAGCACCGCCTACATGGAACTGAGCAGCCTGAGAA |
| | | | GCGAGGACACCGCCGTGTACTACTGTGCCAGACACACCAACTACATCACCGTGTTC |
| | | | GACTACTGGGGCCAGGGCACACTGGTCACAGTTTCTAGCGGAGGCGGAGGATCTGG |
| | | | TGGCGGAGGAAGTGGCGGAGGCGGTTCTGCTCAATCTGTGCTGACACAGCCTCCTA |
| | | | GCGCCTCTGGAACACCTGGCCAGAGAGTGACAATCAGCTGTAGCGGCAGCAGCAGC |
| | | | AACATCGGCAGCAACACCGTGAACTGGTATCAGCAGCTGCCTGGCACAGCCCCTAA |
| | | | ACTGCTGATCTACAGCAACAACCAGCGGCCTAGCGGCGTGCCCGATAGATTTTCTG |
| | | | GCAGCAAGAGCGGCACAAGCGCCAGCCTGGCTATTTCTGGACTGCAGAGCGAGGAC |
| | | | GAGGCCGACTATTATTGTGCCGCCTGGGACCACCACTGGGAGCAAGTTGTTTTTGG |
| | | | AGGCGGCACCAAGCTGACCGTGCTT |
| 105 | LgBiT_PRSIM_67 | DNA | ATGGTCTTCACACTCGAAGATTTCGTTGGGGACTGGGAACAGACAGCCGCCTACAA |
| | | | CCTGGACCAAGTCCTTGAACAGGGAGGTGTGTCCAGTTTGCTGCAGAATCTCGCCG |
| | | | TGTCCGTAACTCCGATCCAAAGGATTGTCCGGAGCGGTGAAAATGCCCTGAAGATC |
| | | | GACATCCATGTCATCATCCCGTATGAAGGTCTGAGCGCCGACCAAATGGCCCAGAT |
| | | | CGAAGAGGTGTTTAAGGTGGTGTACCCTGTGGATGATCATCACTTTAAGGTGATCC |
| | | | TGCCCTATGGCACACTGGTAATCGACGGGGTTACGCCGAACATGCTGAACTATTTC |
| | | | GGACGGCCGTATGAAGGCATCGCCGTGTTCGACGGCAAAAAGATCACTGTAACAGG |
| | | | GACCCTGTGGAACGGCAACAAAATTATCGACGAGCGCCTGATCACCCCCGACGGCT |
| | | | CCATGCTGTTCCGAGTAACCATCAACAGTGGGAGTTCCGGTGGTGGCGGGAGCGGA |
| | | | GGTGGAGGCTCGAGCGGTGAAGTGCAGCTGGTTCAGTCTGGCGCCGAAGTGAAGAA |
| | | | ACCTGGCGCCGCTGTCAGAATCAGCTGCAAGACAAGCGGCTACGTGTTCACCAGCT |
| | | | ACTACGTGCACTGGGTCCGACAGGCTCCAGGACAAGGACTGGAATGGATGGGCGTG |
| | | | ATCAATCCCAGCGGCGGCAACACCAATTACGCCCAGAAATTCCAGGACCGCGTGAC |
| | | | CATGACCAGAGACACCAGCACCACCACCGTGTACATGGAACTGAGCAGCCTGATGT |
| | | | TCGACGACACCGCCGTGTACTACTGCGCCAAGAGAGATTACGGCGGACCCCTGGCC |
| | | | AATTGGGGCAGAGGAACACTGGTCACAGTGTCTAGCGGAGGCGGAGGATCTGGTGG |
| | | | CGGAGGAAGTGGCGGAGGCGGTTCTGCTCTGAGCTATGAGCTGACACAGCCTCCAA |
| | | | GCGTGTCCGAGGCTCCTAGACAGAGAGTGACCATCAGCTGTAGCGGCAGCAGCAGC |
| | | | AACATCGGCAACAACGCCGTGAACTGGTATCAGCAGCTGCCTGGCAAGGCCCCTAA |
| | | | ACTGCTGATCTTCTACGACGACCTGCTGCCTAGCGGAGTGTCCGATAGATTCAGCG |
| | | | GCTCTAAGAGCGGCACATCTGCCAGCCTGGCCATCTCTGGACTGCAGAGCGAAGAT |
| | | | GAGGCCGACTACTATTGCGCCGCCTGGGACGATTCTCTGAACGGCCTGGTTTTTGG |
| | | | CACCGGCACCAAGCTGACAGTGCTG |
| 106 | LgBiT_PRSIM_72 | DNA | ATGGTCTTCACACTCGAAGATTTCGTTGGGGACTGGGAACAGACAGCCGCCTACAA |
| | | | CCTGGACCAAGTCCTTGAACAGGGAGGTGTGTCCAGTTTGCTGCAGAATCTCGCCG |
| | | | TGTCCGTAACTCCGATCCAAAGGATTGTCCGGAGCGGTGAAAATGCCCTGAAGATC |
| | | | GACATCCATGTCATCATCCCGTATGAAGGTCTGAGCGCCGACCAAATGGCCCAGAT |
| | | | CGAAGAGGTGTTTAAGGTGGTGTACCCTGTGGATGATCATCACTTTAAGGTGATCC |
| | | | TGCCCTATGGCACACTGGTAATCGACGGGGTTACGCCGAACATGCTGAACTATTTC |
| | | | GGACGGCCGTATGAAGGCATCGCCGTGTTCGACGGCAAAAAGATCACTGTAACAGG |
| | | | GACCCTGTGGAACGGCAACAAAATTATCGACGAGCGCCTGATCACCCCCGACGGCT |
| | | | CCATGCTGTTCCGAGTAACCATCAACAGTGGGAGTTCCGGTGGTGGCGGGAGCGGA |
| | | | GGTGGAGGCTCGAGCGGTCAGGTTCAGCTGGTTCAGTCTGGCGCCGAAGTGAAGAA |
| | | | ACCTGGCAGCAGCGTGAAGGTGTCCTGCAAAGTGTCTGGCGGCAGCTTCAACAACT |
| | | | ACGGCGTGTCCTGGGTTCGACAGGCCCCTGGACAAGGACTGGAATGGATGGGCAGA |
| | | | ATCATCCCCATCCGGGACACCGCCAATTACGCCCAGAAATTCCAGGGCAGAGTGAC |
| | | | CATCACCGCCGACACCAGCACCAATATCGCCTACATGGAACTGAGCGGCCTGCGGA |
| | | | GTGATGACACCGCCGTGTACTATTGCGCCAGAGTGCTGGAAGATGACTTCTGGGGC |
| | | | GGCTACTACGACTTCTACTTCTACGTGATGGACGTGTGGGGCCAGGGCACACTGGT |
| | | | TACAGTTTCTAGCGGAGGCGGAGGATCTGGTGGCGGAGGAAGTGGCGGAGGCGGTT |
| | | | CTGCTCTTTCTAGCGAGCTGACCCAGGATCCAGTGGTGTCTGTTCCTCTGGGCCAG |
| | | | ACCGCCAGAATTACCTGTCAGGGCGATAGCCTGACCACCTACTACGCCACCTGGTA |
| | | | TCAGCAGAAGCCAGGCCAGGCTCCTGTGCTGGTGCTGTACAATGAGCACAAGAGGC |
| | | | CCAGCGGCATCAGCGACAGATTTTCTGGATCTTCTGCCGGCGACGCCGCCAGCCTG |
| | | | ACAATCACAGATACACAGGCCGAGGACGAGGCCGACTACTACTGCAGCTCTAGAGA |
| | | | TACCGGCGGCAAACACGTGCTGTTTGGAGGCGGCACAAAGCTGACAGTGCT |
| 107 | LgBiT_PRSIM_75 | DNA | ATGGTCTTCACACTCGAAGATTTCGTTGGGGACTGGGAACAGACAGCCGCCTACAA |
| | | | CCTGGACCAAGTCCTTGAACAGGGAGGTGTGTCCAGTTTGCTGCAGAATCTCGCCG |
| | | | TGTCCGTAACTCCGATCCAAAGGATTGTCCGGAGCGGTGAAAATGCCCTGAAGATC |
| | | | GACATCCATGTCATCATCCCGTATGAAGGTCTGAGCGCCGACCAAATGGCCCAGAT |
| | | | CGAAGAGGTGTTTAAGGTGGTGTACCCTGTGGATGATCATCACTTTAAGGTGATCC |
| | | | TGCCCTATGGCACACTGGTAATCGACGGGGTTACGCCGAACATGCTGAACTATTTC |
| | | | GGACGGCCGTATGAAGGCATCGCCGTGTTCGACGGCAAAAAGATCACTGTAACAGG |
| | | | GACCCTGTGGAACGGCAACAAAATTATCGACGAGCGCCTGATCACCCCCGACGGCT |
| | | | CCATGCTGTTCCGAGTAACCATCAACAGTGGGAGTTCCGGTGGTGGCGGGAGCGGA |
| | | | GGTGGAGGCTCGAGCGGTGAAGTGCAGCTGGTTCAGTCTGGCGCCGAAGTGAAGAA |
| | | | ACCTGGCAGCAGCGTGAAGGTGTCCTGCAAAGCTTCTGGCGGCAGCTTCAACAGCT |
| | | | ACACCCTGGACTGGGTTCGACAGGCCCCTGGACAAGGACTGGAATGGATGGGCGGA |
| | | | ATCATCCCCGTGTTCGGCAGCCCTAATTACGGCCAGAAATTCCAGGGCAGAGTGAC |
| | | | CATCACCGCCGACGAGTCTACAAGCACCGCCTACATGGAACTGAGCAGCCTGAAGT |
| | | | CCGACGACACCGCCGTGTACTATTGTGCCAGAGGCCTGGTGTACCAGCCACTGGAT |
| | | | TCTTGGGGCAGAGGCACCCTGGTCACAGTTTCTAGCGGAGGCGGAGGATCTGGTGG |
| | | | CGGAGGAAGTGGCGGAGGCGGTTCTGCTCAAGCTGTTCTGACACAGCCTAGCAGCG |
| | | | CCTCTGGAACACCTGGCCAGAGAGTGACAATCAGCTGTAGCGGCAGCAGCAGCAAC |
| | | | ATCGGCTCCTACACCGTGAACTGGTATCAGCAGTTCCCCGGCACAGCCCCTAAGCT |
| | | | GCTGATCTACAGCAACACCCAGAGGCCAAGCGGCGTGCCCGATAGATTTTCTGGCA |
| | | | GCAAGAGCGGCACAAGCGCCAGCCTGGCTATTTCTGGACTGCAGAGCGAGGACGAG |
| | | | GCCGACTATTATTGTGCCGCCTGGGACGACAGCCTGAACGGATGGGTTTTCGGCGG |
| | | | AGGCACCAAAGTGACAGTGCTT |
| 108 | HCV-Pro-AD | DNA | ATGGGCAAGAAAAAGGGCAGCGTGGTCATCGTGGGCAGAATCAACCTGAGCGGCGA |
| | fusion | | TACCGCCTACGCTCAGCAGACAAGAGGCGAGGAAGGCTGCCAAGAGACAAGCCAGA |
| | | | CCGGCAGAGACAAGAACCAGGTGGAAGGCGAGGTGCAGATCGTGTCTACAGCTACC |
| | | | CAGACCTTCCTGGCCACCAGCATCAATGGCGTGCTGTGGACAGTGTATCACGGCGC |
| | | | TGGCACCAGAACAATCGCCTCTCCAAAGGGCCCCGTGACACAGATGTACACCAACG |
| | | | TGGACAAGGACCTCGTCGGATGGCAAGCCCCTCAGGGCTCTAGAAGCCTGACACCT |
| | | | TGTACCTGCGGCAGCAGCGATCTGTACCTGGTCACAAGACACGCCGACGTGATCCC |
| | | | CGTCAGAAGAAGAGGCGATAGCAGAGGCAGCCTGCTGAGCCCTAGACCTATCAGCT |
| | | | ACCTGAAGGGATCTGCCGGCGGACCTCTGCTTTGTCCTGCTGGACATGCCGTGGGC |
| | | | ATCTTTAGAGCCGCCGTGTCTACTAGAGGCGTGGCCAAAGCCGTGGACTTCATCCC |
| | | | TGTGGAAAGCCTGGAAACCACCATGAGAAGCCCCACCGGTGGCGGAGGATCTGGCG |
| | | | GAGGCGGATCTGATGAATTTCCCACCATGGTGTTTCCTTCTGGGCAGATCAGCCAG |
| | | | GCCTCGGCCTTGGCCCCGGCCCCTCCCCAAGTCCTGCCCCAGGCTCCAGCCCCTGC |
| | | | CCCTGCTCCAGCCATGGTATCAGCTCTGGCCCAGGCCCCAGCCCCTGTCCCAGTCC |
| | | | TAGCCCCAGGCCCTCCTCAGGCTGTGGCCCCACCTGCCCCCAAGCCCACCCAGGCT |
| | | | GGGGAAGGAACGCTGTCAGAGGCCCTGCTGCAGCTGCAGTTTGATGATGAAGACCT |
| | | | GGGGGCCTTGCTTGGCAACAGCACAGACCCAGCTGTGTTCACAGACCTGGCATCCG |
| | | | TCGACAACTCCGAGTTTCAGCAGCTGCTGAACCAGGGCATACCTGTGGCCCCCCAC |
| | | | ACAACTGAGCCCATGCTGATGGAGTACCCTGAGGCTATAACTCGCCTAGTGACAGG |
| | | | GGCCCAGAGGCCCCCCGACCCAGCTCCTGCTCCACTGGGGGCCCCGGGGCTCCCCA |
| | | | ATGGCCTCCTTTCAGGAGATGAAGACTTCTCCTCCATTGCGGACATGGACTTCTCA |
| | | | GCCCTGCTGAGTCAGATCAGCTCCACTAGTTAT |
| 109 | DBD-HCV | DNA | TCTAGAGAACGCCCATATGCTTGCCCTGTCGAGTCCTGCGATCGCCGCTTTTCTCG |
| | Pro fusion | | CTCGGATGAGCTTACCCGCCATATCCGCATCCACACAGGCCAGAAGCCCTTCCAGT |
| | | | GTCGAATCTGCATGCGTAACTTCAGTCGTAGTGACCACCTTACCACCCACATCCGC |
| | | | ACCCACACAGGCGGCGGCCGCAGGAGGAAGAAACGCACCAGCATAGAGACCAACAT |
| | | | CCGTGTGGCCTTAGAGAAGAGTTTCTTGGAGAATCAAAAGCCTACCTCGGAAGAGA |
| | | | TCACTATGATTGCTGATCAGCTCAATATGGAAAAAGAGGTGATTCGTGTTTGGTTC |
| | | | TGTAACCGCCGCCAGAAAGAAAAAAGAATCAACACTAGCGCTAAGAAAAAGGGCAG |
| | | | CGTGGTCATCGTGGGCAGAATCAACCTGAGCGGCGATACCGCCTACGCTCAGCAGA |
| | | | CAAGAGGCGAGGAAGGCTGCCAAGAGACAAGCCAGACCGGCAGAGACAAGAACCAG |
| | | | GTGGAAGGCGAGGTGCAGATCGTGTCTACAGCTACCCAGACCTTCCTGGCCACCAG |
| | | | CATCAATGGCGTGCTGTGGACAGTGTATCACGGCGCTGGCACCAGAACAATCGCCT |
| | | | CTCCAAAGGGCCCCGTGACACAGATGTACACCAACGTGGACAAGGACCTCGTCGGA |
| | | | TGGCAAGCCCCTCAGGGCTCTAGAAGCCTGACACCTTGTACCTGCGGCAGCAGCGA |
| | | | TCTGTACCTGGTCACAAGACACGCCGACGTGATCCCCGTCAGAAGAAGAGGCGATA |
| | | | GCAGAGGCAGCCTGCTGAGCCCTAGACCTATCAGCTACCTGAAGGGATCTGCCGGC |
| | | | GGACCTCTGCTTTGTCCTGCTGGACATGCCGTGGGCATCTTTAGAGCCGCCGTGTC |
| | | | TACTAGAGGCGTGGCCAAAGCCGTGGACTTCATCCCTGTGGAAAGCCTGGAAACCA |
| | | | CCATGAGAAGCCCT |
| 110 | PRSIM_23_DBD_ | DNA | TCTAGAGAACGCCCATATGCTTGCCCTGTCGAGTCCTGCGATCGCCGCTTTTCTCG |
| | fusion | | CTCGGATGAGCTTACCCGCCATATCCGCATCCACACAGGCCAGAAGCCCTTCCAGT |
| | | | GTCGAATCTGCATGCGTAACTTCAGTCGTAGTGACCACCTTACCACCCACATCCGC |
| | | | ACCCACACAGGCGGCGGCCGCAGGAGGAAGAAACGCACCAGCATAGAGACCAACAT |
| | | | CCGTGTGGCCTTAGAGAAGAGTTTCTTGGAGAATCAAAAGCCTACCTCGGAAGAGA |
| | | | TCACTATGATTGCTGATCAGCTCAATATGGAAAAAGAGGTGATTCGTGTTTGGTTC |
| | | | TGTAACCGCCGCCAGAAAGAAAAAAGAATCAACACTAGCGCTGGCTCTAGACTGGA |
| | | | TGCCCCTAGCCAGATCGAAGTGAAGGACGTGACCGACACCACCGCTCTGATCACCT |
| | | | GGGTTGACCCCAGATACGACGACATCTGGTGGTTCGAGCTGACCTACGGCATCAAG |
| | | | GATGTGCCCGGCGACAGAACCACCATCAAGCTGTACCTGAACGACCCCTACTACAG |
| | | | CATCGGCAACCTGAAGCCTGACACCGAGTACGAGGTGTCCCTGATCAGCTACACCG |
| | | | GCGACTCCTACAGCAGAAGCGGCAGCAATCCTGCCAAGATCACCTTCAAGACCGGC |
| | | | CTT |
| 111 | PRSIM_32_DBD_ | DNA | TCTAGAGAACGCCCATATGCTTGCCCTGTCGAGTCCTGCGATCGCCGCTTTTCTCG |
| | fusion | | CTCGGATGAGCTTACCCGCCATATCCGCATCCACACAGGCCAGAAGCCCTTCCAGT |
| | | | GTCGAATCTGCATGCGTAACTTCAGTCGTAGTGACCACCTTACCACCCACATCCGC |
| | | | ACCCACACAGGCGGCGGCCGCAGGAGGAAGAAACGCACCAGCATAGAGACCAACAT |
| | | | CCGTGTGGCCTTAGAGAAGAGTTTCTTGGAGAATCAAAAGCCTACCTCGGAAGAGA |
| | | | TCACTATGATTGCTGATCAGCTCAATATGGAAAAAGAGGTGATTCGTGTTTGGTTC |
| | | | TGTAACCGCCGCCAGAAAGAAAAAAGAATCAACACTAGCGCTGGCTCTAGACTGGA |
| | | | TGCCCCTAGCCAGATCGAAGTGAAGGACGTGACCGACACCACCGCTCTGATCACAT |
| | | | GGTGGTCCCCACGGTACTACTACGCCAGCATCAGCGGCTTCGAGCTGACCTACGGC |
| | | | ATCAAGGATGTGCCCGGCGACAGAACCACCATCAAGCTGGACTACGCCTCCAACGA |
| | | | CTACAGCATCGGCAACCTGAAGCCTGACACCGAGTACGAGGTGTCCCTGATCAGCT |
| | | | GGAACTACGGCGATTGGCGGTACAGCAGCAGCAACCCTGCCAAGATCACCTTCAAG |
| | | | ACCGGCCTT |
| 112 | PRSIM_33_DBD_ | DNA | TCTAGAGAACGCCCATATGCTTGCCCTGTCGAGTCCTGCGATCGCCGCTTTTCTCG |
| | fusion | | CTCGGATGAGCTTACCCGCCATATCCGCATCCACACAGGCCAGAAGCCCTTCCAGT |
| | | | GTCGAATCTGCATGCGTAACTTCAGTCGTAGTGACCACCTTACCACCCACATCCGC |
| | | | ACCCACACAGGCGGCGGCCGCAGGAGGAAGAAACGCACCAGCATAGAGACCAACAT |
| | | | CCGTGTGGCCTTAGAGAAGAGTTTCTTGGAGAATCAAAAGCCTACCTCGGAAGAGA |
| | | | TCACTATGATTGCTGATCAGCTCAATATGGAAAAAGAGGTGATTCGTGTTTGGTTC |
| | | | TGTAACCGCCGCCAGAAAGAAAAAAGAATCAACACTAGCGCTGGCTCTAGACTGGA |
| | | | TGCCCCTAGCCAGATCGAAGTGAAGGACGTGACCGACACCACCGCTCTGATCACCT |
| | | | GGTATCCACCTGGCCGTTGGTACGACGACATCTGGTACTTCGAGCTGACCTACGGC |
| | | | ATCAAGGACGTGCCCGGCGATAGAACCACCATCAAACTGGCCAGAGGCGACGACGT |
| | | | GTACAGCATCGGCAACCTGAAGCCTGACACCGAGTACGAGGTGTCCCTGATCTCTT |
| | | | GGGGCCCTGACAGAGGCGATAGAGCCGGATCTAACCCCGCCAAGATCACCTTCAAG |
| | | | ACCGGCCTT |
| 113 | PRSIM_36_DBD_ | DNA | TCTAGAGAACGCCCATATGCTTGCCCTGTCGAGTCCTGCGATCGCCGCTTTTCTCG |
| | fusion | | CTCGGATGAGCTTACCCGCCATATCCGCATCCACACAGGCCAGAAGCCCTTCCAGT |
| | | | GTCGAATCTGCATGCGTAACTTCAGTCGTAGTGACCACCTTACCACCCACATCCGC |
| | | | ACCCACACAGGCGGCGGCCGCAGGAGGAAGAAACGCACCAGCATAGAGACCAACAT |
| | | | CCGTGTGGCCTTAGAGAAGAGTTTCTTGGAGAATCAAAAGCCTACCTCGGAAGAGA |
| | | | TCACTATGATTGCTGATCAGCTCAATATGGAAAAAGAGGTGATTCGTGTTTGGTTC |
| | | | TGTAACCGCCGCCAGAAAGAAAAAAGAATCAACACTAGCGCTGGCTCTAGACTGGA |
| | | | TGCCCCTAGCCAGATCGAAGTGAAGGACGTGACCGACACCACCGCTCTGATCACCT |
| | | | GGTCCTGGCCTAGAGATGACGACTACGACATCTGGTACTTCGAGCTGACCTACGGC |
| | | | ATCAAGGACGTGCCCGGCGATAGAACCACCATCAAGCTGCTGAACTACGCCTCTCC |
| | | | ATACAGCATCGGCAACCTGAAGCCTGACACCGAGTACGAGGTGTCCCTGATCAGCG |
| | | | TGGTGCCCGACACATATGGCAGAGGCACAAGCAACCCCGCCAAGATCACCTTCAAG |
| | | | ACCGGACTT |
| 114 | PRSIM_47_DBD_ | DNA | TCTAGAGAACGCCCATATGCTTGCCCTGTCGAGTCCTGCGATCGCCGCTTTTCTCG |
| | fusion | | CTCGGATGAGCTTACCCGCCATATCCGCATCCACACAGGCCAGAAGCCCTTCCAGT |
| | | | GTCGAATCTGCATGCGTAACTTCAGTCGTAGTGACCACCTTACCACCCACATCCGC |
| | | | ACCCACACAGGCGGCGGCCGCAGGAGGAAGAAACGCACCAGCATAGAGACCAACAT |
| | | | CCGTGTGGCCTTAGAGAAGAGTTTCTTGGAGAATCAAAAGCCTACCTCGGAAGAGA |
| | | | TCACTATGATTGCTGATCAGCTCAATATGGAAAAAGAGGTGATTCGTGTTTGGTTC |
| | | | TGTAACCGCCGCCAGAAAGAAAAAAGAATCAACACTAGCGCTGGCTCTAGACTGGA |
| | | | TGCCCCTAGCCAGATCGAAGTGAAGGACGTGACCGACACCACCGCTCTGATCACCT |
| | | | GGTCAAGACCTGGCGTGTCCATCTGGTACTTCGAGCTGACCTACGGCATCAAGGAC |
| | | | GTGCCCGGCGATAGAACCACCATCAAGCTGGACTACCGCAGCTACTACTACAGCAT |
| | | | CGGCAACCTGAAGCCTGACACCGAGTACGAGGTGTCCCTGATCAGCGGCTCTTATG |
| | | | GCCTCGTGGGCGTCAGAGCCTCTAATCCCGCCAAGATCACCTTTAAGACCGGCCTT |
| 115 | PRSIM_01_DBD_ | DNA | TCTAGAGAACGCCCATATGCTTGCCCTGTCGAGTCCTGCGATCGCCGCTTTTCTCG |
| | fusion | | CTCGGATGAGCTTACCCGCCATATCCGCATCCACACAGGCCAGAAGCCCTTCCAGT |
| | | | GTCGAATCTGCATGCGTAACTTCAGTCGTAGTGACCACCTTACCACCCACATCCGC |
| | | | ACCCACACAGGCGGCGGCCGCAGGAGGAAGAAACGCACCAGCATAGAGACCAACAT |
| | | | CCGTGTGGCCTTAGAGAAGAGTTTCTTGGAGAATCAAAAGCCTACCTCGGAAGAGA |
| | | | TCACTATGATTGCTGATCAGCTCAATATGGAAAAAGAGGTGATTCGTGTTTGGTTC |
| | | | TGTAACCGCCGCCAGAAAGAAAAAAGAATCAACACTAGCGCTGGCTCTCAGCTGGT |
| | | | TCAGTCTGGCGCCGAAGTGAAGAAACCTGGCAGCAGCGTGAAGGTGTCCTGCAAAG |
| | | | CTTCTGGCGGCACCTTCAGCAGCTACGCCATCTCTTGGGTTCGACAGGCCCCTGGA |
| | | | CAAGGCCTGGAATGGATGGGAGGCATCATCCCCATCTTCGGCACCGCCAATTACGC |
| | | | CCAGAAATTCCAGGGCAGAGTGACCATCACCGCCGACGAGTCTACAAGCACCGCCT |
| | | | ACATGGAACTGAGCAGCCTGAGAAGCGAGGACACCGCCGTGTACTATTGTGCCAGA |
| | | | GGCCAGGGCTACTACGGCTACTTCGATTATTGGGGCCAGGGCACCCTGGTCACAGT |
| | | | TTCTAGCGGAGGCGGAGGATCTGGTGGCGGAGGAAGTGGCGGAGGCGGTTCTGCTC |
| | | | AATCTGTGCTGACACAGCCTCCTAGCGCCTCTGGAACACCTGGCCAGAGAGTGACA |
| | | | ATCAGCTGTAGCGGCAGCAGCAGCAACATCGGCAGCAACACCGTGAACTGGTATCA |
| | | | GCAGCTGCCTGGCACAGCCCCTAAACTGCTGATCTACAGCAACAACCAGCGGCCTA |
| | | | GCGGCGTGCCCGATAGATTTTCCGTGTCTAAGAGCGGCACCAGCGCCAGCCTGGCT |
| | | | ATTTCTGGACTGCAGAGCGAGGACGAGGCCGACTATTATTGTGCCGCCTGGGATCA |
| | | | CGGACACGAGCACGTTGTGTTTGGAGGCGGCACCAAGCTGACAGTGCTT |
| 116 | PRSIM_04_DBD_ | DNA | TCTAGAGAACGCCCATATGCTTGCCCTGTCGAGTCCTGCGATCGCCGCTTTTCTCG |
| | fusion | | CTCGGATGAGCTTACCCGCCATATCCGCATCCACACAGGCCAGAAGCCCTTCCAGT |
| | | | GTCGAATCTGCATGCGTAACTTCAGTCGTAGTGACCACCTTACCACCCACATCCGC |
| | | | ACCCACACAGGCGGCGGCCGCAGGAGGAAGAAACGCACCAGCATAGAGACCAACAT |
| | | | CCGTGTGGCCTTAGAGAAGAGTTTCTTGGAGAATCAAAAGCCTACCTCGGAAGAGA |
| | | | TCACTATGATTGCTGATCAGCTCAATATGGAAAAAGAGGTGATTCGTGTTTGGTTC |
| | | | TGTAACCGCCGCCAGAAAGAAAAAAGAATCAACACTAGCGCTGGCTCTCAGGTGCA |
| | | | GCTTGTTCAGTCTGGCGCCGAAGTGAAGAAACCCGGCAGCTCTGTGAAGGTGTCCT |
| | | | GCAAAGCTTCCGGCGGCACCTTTAGCAGCTACGCCATCTCTTGGGTCCGACAGGCT |
| | | | CCTGGACAAGGCCTGGAATGGATGGGCGGCATCATCCCTATCTTCGGCACCGCCAA |
| | | | TTACGCCCAGAAATTCCAGGGCAGAGTGACCATCACCGCCGACGAGTCTACAAGCA |
| | | | CCGCCTACATGGAACTGAGCAGCCTGAGAAGCGAGGACACCGCCGTGTACTATTGC |
| | | | GCCAGAGGCATGGCCCACTTCTACCAGTTTGATCTGTGGGGCCAGGGCACCCTGGT |
| | | | CACAGTTTCTAGCGGAGGCGGAGGATCTGGCGGCGGAGGTAGTGGTGGTGGCGGAT |
| | | | CTGCTCAGTCTGTGCTGACACAGCCTCCTAGCGCCTCTGGAACACCTGGCCAGAGA |
| | | | GTGACAATCAGCTGTAGCGGCAGCAGCAGCAACATCGGCAGCAACACCGTGAACTG |
| | | | GTATCAGCAGCTGCCTGGCACAGCCCCTAAACTGCTGATCTACAGCAACAACCAGC |
| | | | GGCCTAGCGGCGTGCCCGATAGATTTTCTGGCAGCAAGAGCGGCACAAGCGCCAGC |
| | | | CTGGCTATTTCTGGACTGCAGAGCGAGGACGAGGCCGACTACTATTGTGCTGCCGG |
| | | | CGATCACGACCACGAGCACGTTGTGTTTGGCGGAGGCACCAAGCTGACAGTGCTT |
| 117 | PRSIM_57_DBD_ | DNA | TCTAGAGAACGCCCATATGCTTGCCCTGTCGAGTCCTGCGATCGCCGCTTTTCTCG |
| | fusion | | CTCGGATGAGCTTACCCGCCATATCCGCATCCACACAGGCCAGAAGCCCTTCCAGT |
| | | | GTCGAATCTGCATGCGTAACTTCAGTCGTAGTGACCACCTTACCACCCACATCCGC |
| | | | ACCCACACAGGCGGCGGCCGCAGGAGGAAGAAACGCACCAGCATAGAGACCAACAT |
| | | | CCGTGTGGCCTTAGAGAAGAGTTTCTTGGAGAATCAAAAGCCTACCTCGGAAGAGA |
| | | | TCACTATGATTGCTGATCAGCTCAATATGGAAAAAGAGGTGATTCGTGTTTGGTTC |
| | | | TGTAACCGCCGCCAGAAAGAAAAAAGAATCAACACTAGCGCTGGCTCTCAGGTTCA |
| | | | GCTGGTTCAGTCTGGCGCCGAAGTGAAGAAACCTGGCAGCAGCGTGAAGGTGTCCT |
| | | | GCAAAGCTTCTGGCGGCACCTTCAGCAGCTACGCCATCTCTTGGGTTCGACAGGCC |
| | | | CCTGGACAAGGCCTGGAATGGATGGGAGGCATCATCCCCATCTTCGGCACCGCCAA |
| | | | TTACGCCCAGAAATTCCAGGGCAGAGTGACCATCACCGCCGACGAGTCTACAAGCA |
| | | | CCGCCTACATGGAACTGAGCAGCCTGAGAAGCGAGGACACCGCCGTGTACTACTGT |
| | | | GCCAGACACACCAACTACATCACCGTGTTCGACTACTGGGGCCAGGGCACACTGGT |
| | | | CACAGTTTCTAGCGGAGGCGGAGGATCTGGTGGCGGAGGAAGTGGCGGAGGCGGTT |
| | | | CTGCTCAATCTGTGCTGACACAGCCTCCTAGCGCCTCTGGAACACCTGGCCAGAGA |
| | | | GTGACAATCAGCTGTAGCGGCAGCAGCAGCAACATCGGCAGCAACACCGTGAACTG |
| | | | GTATCAGCAGCTGCCTGGCACAGCCCCTAAACTGCTGATCTACAGCAACAACCAGC |
| | | | GGCCTAGCGGCGTGCCCGATAGATTTTCTGGCAGCAAGAGCGGCACAAGCGCCAGC |
| | | | CTGGCTATTTCTGGACTGCAGAGCGAGGACGAGGCCGACTATTATTGTGCCGCCTG |
| | | | GGACCACCACTGGGAGCAAGTTGTTTTTGGAGGCGGCACCAAGCTGACCGTGCTT |
| 118 | PRSIM_67_DBD_ | DNA | TCTAGAGAACGCCCATATGCTTGCCCTGTCGAGTCCTGCGATCGCCGCTTTTCTCG |
| | fusion | | CTCGGATGAGCTTACCCGCCATATCCGCATCCACACAGGCCAGAAGCCCTTCCAGT |
| | | | GTCGAATCTGCATGCGTAACTTCAGTCGTAGTGACCACCTTACCACCCACATCCGC |
| | | | ACCCACACAGGCGGCGGCCGCAGGAGGAAGAAACGCACCAGCATAGAGACCAACAT |
| | | | CCGTGTGGCCTTAGAGAAGAGTTTCTTGGAGAATCAAAAGCCTACCTCGGAAGAGA |
| | | | TCACTATGATTGCTGATCAGCTCAATATGGAAAAAGAGGTGATTCGTGTTTGGTTC |
| | | | TGTAACCGCCGCCAGAAAGAAAAAAGAATCAACACTAGCGCTGGCTCTGAAGTGCA |
| | | | GCTGGTTCAGTCTGGCGCCGAAGTGAAGAAACCTGGCGCCGCTGTCAGAATCAGCT |
| | | | GCAAGACAAGCGGCTACGTGTTCACCAGCTACTACGTGCACTGGGTCCGACAGGCT |
| | | | CCAGGACAAGGACTGGAATGGATGGGCGTGATCAATCCCAGCGGCGGCAACACCAA |
| | | | TTACGCCCAGAAATTCCAGGACCGCGTGACCATGACCAGAGACACCAGCACCACCA |
| | | | CCGTGTACATGGAACTGAGCAGCCTGATGTTCGACGACACCGCCGTGTACTACTGC |
| | | | GCCAAGAGAGATTACGGCGGACCCCTGGCCAATTGGGGCAGAGGAACACTGGTCAC |
| | | | AGTGTCTAGCGGAGGCGGAGGATCTGGTGGCGGAGGAAGTGGCGGAGGCGGTTCTG |
| | | | CTCTGAGCTATGAGCTGACACAGCCTCCAAGCGTGTCCGAGGCTCCTAGACAGAGA |
| | | | GTGACCATCAGCTGTAGCGGCAGCAGCAGCAACATCGGCAACAACGCCGTGAACTG |
| | | | GTATCAGCAGCTGCCTGGCAAGGCCCCTAAACTGCTGATCTTCTACGACGACCTGC |
| | | | TGCCTAGCGGAGTGTCCGATAGATTCAGCGGCTCTAAGAGCGGCACATCTGCCAGC |
| | | | CTGGCCATCTCTGGACTGCAGAGCGAAGATGAGGCCGACTACTATTGCGCCGCCTG |
| | | | GGACGATTCTCTGAACGGCCTGGTTTTTGGCACCGGCACCAAGCTGACAGTGCTT |
| 119 | PRSIM_72_DBD_ | DNA | TCTAGAGAACGCCCATATGCTTGCCCTGTCGAGTCCTGCGATCGCCGCTTTTCTCG |
| | fusion | | CTCGGATGAGCTTACCCGCCATATCCGCATCCACACAGGCCAGAAGCCCTTCCAGT |
| | | | GTCGAATCTGCATGCGTAACTTCAGTCGTAGTGACCACCTTACCACCCACATCCGC |
| | | | ACCCACACAGGCGGCGGCCGCAGGAGGAAGAAACGCACCAGCATAGAGACCAACAT |
| | | | CCGTGTGGCCTTAGAGAAGAGTTTCTTGGAGAATCAAAAGCCTACCTCGGAAGAGA |
| | | | TCACTATGATTGCTGATCAGCTCAATATGGAAAAAGAGGTGATTCGTGTTTGGTTC |
| | | | TGTAACCGCCGCCAGAAAGAAAAAAGAATCAACACTAGCGCTGGCTCTCAGGTTCA |
| | | | GCTGGTTCAGTCTGGCGCCGAAGTGAAGAAACCTGGCAGCAGCGTGAAGGTGTCCT |
| | | | GCAAAGTGTCTGGCGGCAGCTTCAACAACTACGGCGTGTCCTGGGTTCGACAGGCC |
| | | | CCTGGACAAGGACTGGAATGGATGGGCAGAATCATCCCCATCCGGGACACCGCCAA |
| | | | TTACGCCCAGAAATTCCAGGGCAGAGTGACCATCACCGCCGACACCAGCACCAATA |
| | | | TCGCCTACATGGAACTGAGCGGCCTGCGGAGTGATGACACCGCCGTGTACTATTGC |
| | | | GCCAGAGTGCTGGAAGATGACTTCTGGGGCGGCTACTACGACTTCTACTTCTACGT |
| | | | GATGGACGTGTGGGGCCAGGGCACACTGGTTACAGTTTCTAGCGGAGGCGGAGGAT |
| | | | CTGGTGGCGGAGGAAGTGGCGGAGGCGGTTCTGCTCTTTCTAGCGAGCTGACCCAG |
| | | | GATCCAGTGGTGTCTGTTCCTCTGGGCCAGACCGCCAGAATTACCTGTCAGGGCGA |
| | | | TAGCCTGACCACCTACTACGCCACCTGGTATCAGCAGAAGCCAGGCCAGGCTCCTG |
| | | | TGCTGGTGCTGTACAATGAGCACAAGAGGCCCAGCGGCATCAGCGACAGATTTTCT |
| | | | GGATCTTCTGCCGGCGACGCCGCCAGCCTGACAATCACAGATACACAGGCCGAGGA |
| | | | CGAGGCCGACTACTACTGCAGCTCTAGAGATACCGGCGGCAAACACGTGCTGTTTG |
| | | | GAGGCGGCACAAAGCTGACAGTGCTT |
| 120 | PRSIM_75_DBD_ | DNA | TCTAGAGAACGCCCATATGCTTGCCCTGTCGAGTCCTGCGATCGCCGCTTTTCTCG |
| | fusion | | CTCGGATGAGCTTACCCGCCATATCCGCATCCACACAGGCCAGAAGCCCTTCCAGT |
| | | | GTCGAATCTGCATGCGTAACTTCAGTCGTAGTGACCACCTTACCACCCACATCCGC |
| | | | ACCCACACAGGCGGCGGCCGCAGGAGGAAGAAACGCACCAGCATAGAGACCAACAT |
| | | | CCGTGTGGCCTTAGAGAAGAGTTTCTTGGAGAATCAAAAGCCTACCTCGGAAGAGA |
| | | | TCACTATGATTGCTGATCAGCTCAATATGGAAAAAGAGGTGATTCGTGTTTGGTTC |
| | | | TGTAACCGCCGCCAGAAAGAAAAAAGAATCAACACTAGCGCTGGCTCTGAAGTGCA |
| | | | GCTGGTTCAGTCTGGCGCCGAAGTGAAGAAACCTGGCAGCAGCGTGAAGGTGTCCT |
| | | | GCAAAGCTTCTGGCGGCAGCTTCAACAGCTACACCCTGGACTGGGTTCGACAGGCC |
| | | | CCTGGACAAGGACTGGAATGGATGGGCGGAATCATCCCCGTGTTCGGCAGCCCTAA |
| | | | TTACGGCCAGAAATTCCAGGGCAGAGTGACCATCACCGCCGACGAGTCTACAAGCA |
| | | | CCGCCTACATGGAACTGAGCAGCCTGAAGTCCGACGACACCGCCGTGTACTATTGT |
| | | | GCCAGAGGCCTGGTGTACCAGCCACTGGATTCTTGGGGCAGAGGCACCCTGGTCAC |
| | | | AGTTTCTAGCGGAGGCGGAGGATCTGGTGGCGGAGGAAGTGGCGGAGGCGGTTCTG |
| | | | CTCAAGCTGTTCTGACACAGCCTAGCAGCGCCTCTGGAACACCTGGCCAGAGAGTG |
| | | | ACAATCAGCTGTAGCGGCAGCAGCAGCAACATCGGCTCCTACACCGTGAACTGGTA |
| | | | TCAGCAGTTCCCCGGCACAGCCCCTAAGCTGCTGATCTACAGCAACACCCAGAGGC |
| | | | CAAGCGGCGTGCCCGATAGATTTTCTGGCAGCAAGAGCGGCACAAGCGCCAGCCTG |
| | | | GCTATTTCTGGACTGCAGAGCGAGGACGAGGCCGACTATTATTGTGCCGCCTGGGA |
| | | | CGACAGCCTGAACGGATGGGTTTTCGGCGGAGGCACCAAAGTGACAGTGCTT |
| 121 | PRSIM_23_AD_ | DNA | ATGGGCTCTAGACTGGATGCCCCTAGCCAGATCGAAGTGAAGGACGTGACCGACAC |
| | fusion | | CACCGCTCTGATCACCTGGGTTGACCCCAGATACGACGACATCTGGTGGTTCGAGC |
| | | | TGACCTACGGCATCAAGGATGTGCCCGGCGACAGAACCACCATCAAGCTGTACCTG |
| | | | AACGACCCCTACTACAGCATCGGCAACCTGAAGCCTGACACCGAGTACGAGGTGTC |
| | | | CCTGATCAGCTACACCGGCGACTCCTACAGCAGAAGCGGCAGCAATCCTGCCAAGA |
| | | | TCACCTTCAAGACCGGCCTTACCGGTGGCGGAGGATCTGGCGGAGGCGGATCTGAT |
| | | | GAATTTCCCACCATGGTGTTTCCTTCTGGGCAGATCAGCCAGGCCTCGGCCTTGGC |
| | | | CCCGGCCCCTCCCCAAGTCCTGCCCCAGGCTCCAGCCCCTGCCCCTGCTCCAGCCA |
| | | | TGGTATCAGCTCTGGCCCAGGCCCCAGCCCCTGTCCCAGTCCTAGCCCCAGGCCCT |
| | | | CCTCAGGCTGTGGCCCCACCTGCCCCCAAGCCCACCCAGGCTGGGGAAGGAACGCT |
| | | | GTCAGAGGCCCTGCTGCAGCTGCAGTTTGATGATGAAGACCTGGGGGCCTTGCTTG |
| | | | GCAACAGCACAGACCCAGCTGTGTTCACAGACCTGGCATCCGTCGACAACTCCGAG |
| | | | TTTCAGCAGCTGCTGAACCAGGGCATACCTGTGGCCCCCCACACAACTGAGCCCAT |
| | | | GCTGATGGAGTACCCTGAGGCTATAACTCGCCTAGTGACAGGGGCCCAGAGGCCCC |
| | | | CCGACCCAGCTCCTGCTCCACTGGGGGCCCCGGGGCTCCCCAATGGCCTCCTTTCA |
| | | | GGAGATGAAGACTTCTCCTCCATTGCGGACATGGACTTCTCAGCCCTGCTGAGTCA |
| | | | GATCAGCTCCACTAGTTAT |
| 122 | PRSIM_32_AD_ | DNA | ATGGGCTCTAGACTGGATGCCCCTAGCCAGATCGAAGTGAAGGACGTGACCGACAC |
| | fusion | | CACCGCTCTGATCACATGGTGGTCCCCACGGTACTACTACGCCAGCATCAGCGGCT |
| | | | TCGAGCTGACCTACGGCATCAAGGATGTGCCCGGCGACAGAACCACCATCAAGCTG |
| | | | GACTACGCCTCCAACGACTACAGCATCGGCAACCTGAAGCCTGACACCGAGTACGA |
| | | | GGTGTCCCTGATCAGCTGGAACTACGGCGATTGGCGGTACAGCAGCAGCAACCCTG |
| | | | CCAAGATCACCTTCAAGACCGGCCTGACCGGTGGCGGAGGATCTGGCGGAGGCGGA |
| | | | TCTGATGAATTTCCCACCATGGTGTTTCCTTCTGGGCAGATCAGCCAGGCCTCGGC |
| | | | CTTGGCCCCGGCCCCTCCCCAAGTCCTGCCCCAGGCTCCAGCCCCTGCCCCTGCTC |
| | | | CAGCCATGGTATCAGCTCTGGCCCAGGCCCCAGCCCCTGTCCCAGTCCTAGCCCCA |
| | | | GGCCCTCCTCAGGCTGTGGCCCCACCTGCCCCCAAGCCCACCCAGGCTGGGGAAGG |
| | | | AACGCTGTCAGAGGCCCTGCTGCAGCTGCAGTTTGATGATGAAGACCTGGGGGCCT |
| | | | TGCTTGGCAACAGCACAGACCCAGCTGTGTTCACAGACCTGGCATCCGTCGACAAC |
| | | | TCCGAGTTTCAGCAGCTGCTGAACCAGGGCATACCTGTGGCCCCCCACACAACTGA |
| | | | GCCCATGCTGATGGAGTACCCTGAGGCTATAACTCGCCTAGTGACAGGGGCCCAGA |
| | | | GGCCCCCCGACCCAGCTCCTGCTCCACTGGGGGCCCCGGGGCTCCCCAATGGCCTC |
| | | | CTTTCAGGAGATGAAGACTTCTCCTCCATTGCGGACATGGACTTCTCAGCCCTGCT |
| | | | GAGTCAGATCAGCTCCACTAGTTAT |
| 123 | PRSIM_33_AD_ | DNA | ATGGGCTCTAGACTGGATGCCCCTAGCCAGATCGAAGTGAAGGACGTGACCGACAC |
| | fusion | | CACCGCTCTGATCACCTGGTATCCACCTGGCCGTTGGTACGACGACATCTGGTACT |
| | | | TCGAGCTGACCTACGGCATCAAGGACGTGCCCGGCGATAGAACCACCATCAAACTG |
| | | | GCCAGAGGCGACGACGTGTACAGCATCGGCAACCTGAAGCCTGACACCGAGTACGA |
| | | | GGTGTCCCTGATCTCTTGGGGCCCTGACAGAGGCGATAGAGCCGGATCTAACCCCG |
| | | | CCAAGATCACCTTCAAGACCGGCCTTACCGGTGGCGGAGGATCTGGCGGAGGCGGA |
| | | | TCTGATGAATTTCCCACCATGGTGTTTCCTTCTGGGCAGATCAGCCAGGCCTCGGC |
| | | | CTTGGCCCCGGCCCCTCCCCAAGTCCTGCCCCAGGCTCCAGCCCCTGCCCCTGCTC |
| | | | CAGCCATGGTATCAGCTCTGGCCCAGGCCCCAGCCCCTGTCCCAGTCCTAGCCCCA |
| | | | GGCCCTCCTCAGGCTGTGGCCCCACCTGCCCCCAAGCCCACCCAGGCTGGGGAAGG |
| | | | AACGCTGTCAGAGGCCCTGCTGCAGCTGCAGTTTGATGATGAAGACCTGGGGGCCT |
| | | | TGCTTGGCAACAGCACAGACCCAGCTGTGTTCACAGACCTGGCATCCGTCGACAAC |
| | | | TCCGAGTTTCAGCAGCTGCTGAACCAGGGCATACCTGTGGCCCCCCACACAACTGA |
| | | | GCCCATGCTGATGGAGTACCCTGAGGCTATAACTCGCCTAGTGACAGGGGCCCAGA |
| | | | GGCCCCCCGACCCAGCTCCTGCTCCACTGGGGGCCCCGGGGCTCCCCAATGGCCTC |
| | | | CTTTCAGGAGATGAAGACTTCTCCTCCATTGCGGACATGGACTTCTCAGCCCTGCT |
| | | | GAGTCAGATCAGCTCCACTAGTTAT |
| 124 | PRSIM_36_AD_ | DNA | ATGGGCTCTAGACTGGATGCCCCTAGCCAGATCGAAGTGAAGGACGTGACCGACAC |
| | fusion | | CACCGCTCTGATCACCTGGTCCTGGCCTAGAGATGACGACTACGACATCTGGTACT |
| | | | TCGAGCTGACCTACGGCATCAAGGACGTGCCCGGCGATAGAACCACCATCAAGCTG |
| | | | CTGAACTACGCCTCTCCATACAGCATCGGCAACCTGAAGCCTGACACCGAGTACGA |
| | | | GGTGTCCCTGATCAGCGTGGTGCCCGACACATATGGCAGAGGCACAAGCAACCCCG |
| | | | CCAAGATCACCTTCAAGACCGGACTTACCGGTGGCGGAGGATCTGGCGGAGGCGGA |
| | | | TCTGATGAATTTCCCACCATGGTGTTTCCTTCTGGGCAGATCAGCCAGGCCTCGGC |
| | | | CTTGGCCCCGGCCCCTCCCCAAGTCCTGCCCCAGGCTCCAGCCCCTGCCCCTGCTC |
| | | | CAGCCATGGTATCAGCTCTGGCCCAGGCCCCAGCCCCTGTCCCAGTCCTAGCCCCA |
| | | | GGCCCTCCTCAGGCTGTGGCCCCACCTGCCCCCAAGCCCACCCAGGCTGGGGAAGG |
| | | | AACGCTGTCAGAGGCCCTGCTGCAGCTGCAGTTTGATGATGAAGACCTGGGGGCCT |
| | | | TGCTTGGCAACAGCACAGACCCAGCTGTGTTCACAGACCTGGCATCCGTCGACAAC |
| | | | TCCGAGTTTCAGCAGCTGCTGAACCAGGGCATACCTGTGGCCCCCCACACAACTGA |
| | | | GCCCATGCTGATGGAGTACCCTGAGGCTATAACTCGCCTAGTGACAGGGGCCCAGA |
| | | | GGCCCCCCGACCCAGCTCCTGCTCCACTGGGGGCCCCGGGGCTCCCCAATGGCCTC |
| | | | CTTTCAGGAGATGAAGACTTCTCCTCCATTGCGGACATGGACTTCTCAGCCCTGCT |
| | | | GAGTCAGATCAGCTCCACTAGTTAT |
| 125 | PRSIM_47_AD_ | DNA | ATGGGCTCTAGACTGGATGCCCCTAGCCAGATCGAAGTGAAGGACGTGACCGACAC |
| | fusion | | CACCGCTCTGATCACCTGGTCAAGACCTGGCGTGTCCATCTGGTACTTCGAGCTGA |
| | | | CCTACGGCATCAAGGACGTGCCCGGCGATAGAACCACCATCAAGCTGGACTACCGC |
| | | | AGCTACTACTACAGCATCGGCAACCTGAAGCCTGACACCGAGTACGAGGTGTCCCT |
| | | | GATCAGCGGCTCTTATGGCCTCGTGGGCGTCAGAGCCTCTAATCCCGCCAAGATCA |
| | | | CCTTTAAGACCGGCCTTACCGGTGGCGGAGGATCTGGCGGAGGCGGATCTGATGAA |
| | | | TTTCCCACCATGGTGTTTCCTTCTGGGCAGATCAGCCAGGCCTCGGCCTTGGCCCC |
| | | | GGCCCCTCCCCAAGTCCTGCCCCAGGCTCCAGCCCCTGCCCCTGCTCCAGCCATGG |
| | | | TATCAGCTCTGGCCCAGGCCCCAGCCCCTGTCCCAGTCCTAGCCCCAGGCCCTCCT |
| | | | CAGGCTGTGGCCCCACCTGCCCCCAAGCCCACCCAGGCTGGGGAAGGAACGCTGTC |
| | | | AGAGGCCCTGCTGCAGCTGCAGTTTGATGATGAAGACCTGGGGGCCTTGCTTGGCA |
| | | | ACAGCACAGACCCAGCTGTGTTCACAGACCTGGCATCCGTCGACAACTCCGAGTTT |
| | | | CAGCAGCTGCTGAACCAGGGCATACCTGTGGCCCCCCACACAACTGAGCCCATGCT |
| | | | GATGGAGTACCCTGAGGCTATAACTCGCCTAGTGACAGGGGCCCAGAGGCCCCCCG |
| | | | ACCCAGCTCCTGCTCCACTGGGGGCCCCGGGGCTCCCCAATGGCCTCCTTTCAGGA |
| | | | GATGAAGACTTCTCCTCCATTGCGGACATGGACTTCTCAGCCCTGCTGAGTCAGAT |
| | | | CAGCTCCACTAGTTAT |
| 126 | PRSIM_01_AD_ | DNA | ATGGGCTCTCAGGTTCAGCTGGTTCAGTCTGGCGCCGAAGTGAAGAAACCTGGCAG |
| | fusion | | CAGCGTGAAGGTGTCCTGCAAAGCTTCTGGCGGCACCTTCAGCAGCTACGCCATCT |
| | | | CTTGGGTTCGACAGGCCCCTGGACAAGGCCTGGAATGGATGGGAGGCATCATCCCC |
| | | | ATCTTCGGCACCGCCAATTACGCCCAGAAATTCCAGGGCAGAGTGACCATCACCGC |
| | | | CGACGAGTCTACAAGCACCGCCTACATGGAACTGAGCAGCCTGAGAAGCGAGGACA |
| | | | CCGCCGTGTACTATTGTGCCAGAGGCCAGGGCTACTACGGCTACTTCGATTATTGG |
| | | | GGCCAGGGCACCCTGGTCACAGTTTCTAGCGGAGGCGGAGGATCTGGTGGCGGAGG |
| | | | AAGTGGCGGAGGCGGTTCTGCTCAATCTGTGCTGACACAGCCTCCTAGCGCCTCTG |
| | | | GAACACCTGGCCAGAGAGTGACAATCAGCTGTAGCGGCAGCAGCAGCAACATCGGC |
| | | | AGCAACACCGTGAACTGGTATCAGCAGCTGCCTGGCACAGCCCCTAAACTGCTGAT |
| | | | CTACAGCAACAACCAGCGGCCTAGCGGCGTGCCCGATAGATTTTCCGTGTCTAAGA |
| | | | GCGGCACCAGCGCCAGCCTGGCTATTTCTGGACTGCAGAGCGAGGACGAGGCCGAC |
| | | | TATTATTGTGCCGCCTGGGATCACGGACACGAGCACGTTGTGTTTGGAGGCGGCAC |
| | | | CAAGCTGACAGTGCTTACCGGTGGCGGAGGATCTGGCGGAGGCGGATCTGATGAAT |
| | | | TTCCCACCATGGTGTTTCCTTCTGGGCAGATCAGCCAGGCCTCGGCCTTGGCCCCG |
| | | | GCCCCTCCCCAAGTCCTGCCCCAGGCTCCAGCCCCTGCCCCTGCTCCAGCCATGGT |
| | | | ATCAGCTCTGGCCCAGGCCCCAGCCCCTGTCCCAGTCCTAGCCCCAGGCCCTCCTC |
| | | | AGGCTGTGGCCCCACCTGCCCCCAAGCCCACCCAGGCTGGGGAAGGAACGCTGTCA |
| | | | GAGGCCCTGCTGCAGCTGCAGTTTGATGATGAAGACCTGGGGGCCTTGCTTGGCAA |
| | | | CAGCACAGACCCAGCTGTGTTCACAGACCTGGCATCCGTCGACAACTCCGAGTTTC |
| | | | AGCAGCTGCTGAACCAGGGCATACCTGTGGCCCCCCACACAACTGAGCCCATGCTG |
| | | | ATGGAGTACCCTGAGGCTATAACTCGCCTAGTGACAGGGGCCCAGAGGCCCCCCGA |
| | | | CCCAGCTCCTGCTCCACTGGGGGCCCCGGGGCTCCCCAATGGCCTCCTTTCAGGAG |
| | | | ATGAAGACTTCTCCTCCATTGCGGACATGGACTTCTCAGCCCTGCTGAGTCAGATC |
| | | | AGCTCCACTAGTTAT |
| 127 | PRSIM_04_AD_ | DNA | ATGGGCTCTCAGGTGCAGCTTGTTCAGTCTGGCGCCGAAGTGAAGAAACCCGGCAG |
| | fusion | | CTCTGTGAAGGTGTCCTGCAAAGCTTCCGGCGGCACCTTTAGCAGCTACGCCATCT |
| | | | CTTGGGTCCGACAGGCTCCTGGACAAGGCCTGGAATGGATGGGCGGCATCATCCCT |
| | | | ATCTTCGGCACCGCCAATTACGCCCAGAAATTCCAGGGCAGAGTGACCATCACCGC |
| | | | CGACGAGTCTACAAGCACCGCCTACATGGAACTGAGCAGCCTGAGAAGCGAGGACA |
| | | | CCGCCGTGTACTATTGCGCCAGAGGCATGGCCCACTTCTACCAGTTTGATCTGTGG |
| | | | GGCCAGGGCACCCTGGTCACAGTTTCTAGCGGAGGCGGAGGATCTGGCGGCGGAGG |
| | | | TAGTGGTGGTGGCGGATCTGCTCAGTCTGTGCTGACACAGCCTCCTAGCGCCTCTG |
| | | | GAACACCTGGCCAGAGAGTGACAATCAGCTGTAGCGGCAGCAGCAGCAACATCGGC |
| | | | AGCAACACCGTGAACTGGTATCAGCAGCTGCCTGGCACAGCCCCTAAACTGCTGAT |
| | | | CTACAGCAACAACCAGCGGCCTAGCGGCGTGCCCGATAGATTTTCTGGCAGCAAGA |
| | | | GCGGCACAAGCGCCAGCCTGGCTATTTCTGGACTGCAGAGCGAGGACGAGGCCGAC |
| | | | TACTATTGTGCTGCCGGCGATCACGACCACGAGCACGTTGTGTTTGGCGGAGGCAC |
| | | | CAAGCTGACAGTGCTTACCGGTGGCGGAGGATCTGGCGGAGGCGGATCTGATGAAT |
| | | | TTCCCACCATGGTGTTTCCTTCTGGGCAGATCAGCCAGGCCTCGGCCTTGGCCCCG |
| | | | GCCCCTCCCCAAGTCCTGCCCCAGGCTCCAGCCCCTGCCCCTGCTCCAGCCATGGT |
| | | | ATCAGCTCTGGCCCAGGCCCCAGCCCCTGTCCCAGTCCTAGCCCCAGGCCCTCCTC |
| | | | AGGCTGTGGCCCCACCTGCCCCCAAGCCCACCCAGGCTGGGGAAGGAACGCTGTCA |
| | | | GAGGCCCTGCTGCAGCTGCAGTTTGATGATGAAGACCTGGGGGCCTTGCTTGGCAA |
| | | | CAGCACAGACCCAGCTGTGTTCACAGACCTGGCATCCGTCGACAACTCCGAGTTTC |
| | | | AGCAGCTGCTGAACCAGGGCATACCTGTGGCCCCCCACACAACTGAGCCCATGCTG |
| | | | ATGGAGTACCCTGAGGCTATAACTCGCCTAGTGACAGGGGCCCAGAGGCCCCCCGA |
| | | | CCCAGCTCCTGCTCCACTGGGGGCCCCGGGGCTCCCCAATGGCCTCCTTTCAGGAG |
| | | | ATGAAGACTTCTCCTCCATTGCGGACATGGACTTCTCAGCCCTGCTGAGTCAGATC |
| | | | AGCTCCACTAGTTAT |
| 128 | PRSIM_57_AD_ | DNA | ATGGGCTCTCAGGTTCAGCTGGTTCAGTCTGGCGCCGAAGTGAAGAAACCTGGCAG |
| | fusion | | CAGCGTGAAGGTGTCCTGCAAAGCTTCTGGCGGCACCTTCAGCAGCTACGCCATCT |
| | | | CTTGGGTTCGACAGGCCCCTGGACAAGGCCTGGAATGGATGGGAGGCATCATCCCC |
| | | | ATCTTCGGCACCGCCAATTACGCCCAGAAATTCCAGGGCAGAGTGACCATCACCGC |
| | | | CGACGAGTCTACAAGCACCGCCTACATGGAACTGAGCAGCCTGAGAAGCGAGGACA |
| | | | CCGCCGTGTACTACTGTGCCAGACACACCAACTACATCACCGTGTTCGACTACTGG |
| | | | GGCCAGGGCACACTGGTCACAGTTTCTAGCGGAGGCGGAGGATCTGGTGGCGGAGG |
| | | | AAGTGGCGGAGGCGGTTCTGCTCAATCTGTGCTGACACAGCCTCCTAGCGCCTCTG |
| | | | GAACACCTGGCCAGAGAGTGACAATCAGCTGTAGCGGCAGCAGCAGCAACATCGGC |
| | | | AGCAACACCGTGAACTGGTATCAGCAGCTGCCTGGCACAGCCCCTAAACTGCTGAT |
| | | | CTACAGCAACAACCAGCGGCCTAGCGGCGTGCCCGATAGATTTTCTGGCAGCAAGA |
| | | | GCGGCACAAGCGCCAGCCTGGCTATTTCTGGACTGCAGAGCGAGGACGAGGCCGAC |
| | | | TATTATTGTGCCGCCTGGGACCACCACTGGGAGCAAGTTGTTTTTGGAGGCGGCAC |
| | | | CAAGCTGACCGTGCTTACCGGTGGCGGAGGATCTGGCGGAGGCGGATCTGATGAAT |
| | | | TTCCCACCATGGTGTTTCCTTCTGGGCAGATCAGCCAGGCCTCGGCCTTGGCCCCG |
| | | | GCCCCTCCCCAAGTCCTGCCCCAGGCTCCAGCCCCTGCCCCTGCTCCAGCCATGGT |
| | | | ATCAGCTCTGGCCCAGGCCCCAGCCCCTGTCCCAGTCCTAGCCCCAGGCCCTCCTC |
| | | | AGGCTGTGGCCCCACCTGCCCCCAAGCCCACCCAGGCTGGGGAAGGAACGCTGTCA |
| | | | GAGGCCCTGCTGCAGCTGCAGTTTGATGATGAAGACCTGGGGGCCTTGCTTGGCAA |
| | | | CAGCACAGACCCAGCTGTGTTCACAGACCTGGCATCCGTCGACAACTCCGAGTTTC |
| | | | AGCAGCTGCTGAACCAGGGCATACCTGTGGCCCCCCACACAACTGAGCCCATGCTG |
| | | | ATGGAGTACCCTGAGGCTATAACTCGCCTAGTGACAGGGGCCCAGAGGCCCCCCGA |
| | | | CCCAGCTCCTGCTCCACTGGGGGCCCCGGGGCTCCCCAATGGCCTCCTTTCAGGAG |
| | | | ATGAAGACTTCTCCTCCATTGCGGACATGGACTTCTCAGCCCTGCTGAGTCAGATC |
| | | | AGCTCCACTAGTTAT |
| 129 | PRSIM_67_AD_ | DNA | ATGGGCTCTGAAGTGCAGCTGGTTCAGTCTGGCGCCGAAGTGAAGAAACCTGGCGC |
| | fusion | | CGCTGTCAGAATCAGCTGCAAGACAAGCGGCTACGTGTTCACCAGCTACTACGTGC |
| | | | ACTGGGTCCGACAGGCTCCAGGACAAGGACTGGAATGGATGGGCGTGATCAATCCC |
| | | | AGCGGCGGCAACACCAATTACGCCCAGAAATTCCAGGACCGCGTGACCATGACCAG |
| | | | AGACACCAGCACCACCACCGTGTACATGGAACTGAGCAGCCTGATGTTCGACGACA |
| | | | CCGCCGTGTACTACTGCGCCAAGAGAGATTACGGCGGACCCCTGGCCAATTGGGGC |
| | | | AGAGGAACACTGGTCACAGTGTCTAGCGGAGGCGGAGGATCTGGTGGCGGAGGAAG |
| | | | TGGCGGAGGCGGTTCTGCTCTGAGCTATGAGCTGACACAGCCTCCAAGCGTGTCCG |
| | | | AGGCTCCTAGACAGAGAGTGACCATCAGCTGTAGCGGCAGCAGCAGCAACATCGGC |
| | | | AACAACGCCGTGAACTGGTATCAGCAGCTGCCTGGCAAGGCCCCTAAACTGCTGAT |
| | | | CTTCTACGACGACCTGCTGCCTAGCGGAGTGTCCGATAGATTCAGCGGCTCTAAGA |
| | | | GCGGCACATCTGCCAGCCTGGCCATCTCTGGACTGCAGAGCGAAGATGAGGCCGAC |
| | | | TACTATTGCGCCGCCTGGGACGATTCTCTGAACGGCCTGGTTTTTGGCACCGGCAC |
| | | | CAAGCTGACAGTGCTGACCGGTGGCGGAGGATCTGGCGGAGGCGGATCTGATGAAT |
| | | | TTCCCACCATGGTGTTTCCTTCTGGGCAGATCAGCCAGGCCTCGGCCTTGGCCCCG |
| | | | GCCCCTCCCCAAGTCCTGCCCCAGGCTCCAGCCCCTGCCCCTGCTCCAGCCATGGT |
| | | | ATCAGCTCTGGCCCAGGCCCCAGCCCCTGTCCCAGTCCTAGCCCCAGGCCCTCCTC |
| | | | AGGCTGTGGCCCCACCTGCCCCCAAGCCCACCCAGGCTGGGGAAGGAACGCTGTCA |
| | | | GAGGCCCTGCTGCAGCTGCAGTTTGATGATGAAGACCTGGGGGCCTTGCTTGGCAA |
| | | | CAGCACAGACCCAGCTGTGTTCACAGACCTGGCATCCGTCGACAACTCCGAGTTTC |
| | | | AGCAGCTGCTGAACCAGGGCATACCTGTGGCCCCCCACACAACTGAGCCCATGCTG |
| | | | ATGGAGTACCCTGAGGCTATAACTCGCCTAGTGACAGGGGCCCAGAGGCCCCCCGA |
| | | | CCCAGCTCCTGCTCCACTGGGGGCCCCGGGGCTCCCCAATGGCCTCCTTTCAGGAG |
| | | | ATGAAGACTTCTCCTCCATTGCGGACATGGACTTCTCAGCCCTGCTGAGTCAGATC |
| | | | AGCTCCACTAGTTAT |
| 130 | PRSIM_72_AD_ | DNA | ATGGGCTCTCAGGTTCAGCTGGTTCAGTCTGGCGCCGAAGTGAAGAAACCTGGCAG |
| | fusion | | CAGCGTGAAGGTGTCCTGCAAAGTGTCTGGCGGCAGCTTCAACAACTACGGCGTGT |
| | | | CCTGGGTTCGACAGGCCCCTGGACAAGGACTGGAATGGATGGGCAGAATCATCCCC |
| | | | ATCCGGGACACCGCCAATTACGCCCAGAAATTCCAGGGCAGAGTGACCATCACCGC |
| | | | CGACACCAGCACCAATATCGCCTACATGGAACTGAGCGGCCTGCGGAGTGATGACA |
| | | | CCGCCGTGTACTATTGCGCCAGAGTGCTGGAAGATGACTTCTGGGGCGGCTACTAC |
| | | | GACTTCTACTTCTACGTGATGGACGTGTGGGGCCAGGGCACACTGGTTACAGTTTC |
| | | | TAGCGGAGGCGGAGGATCTGGTGGCGGAGGAAGTGGCGGAGGCGGTTCTGCTCTTT |
| | | | CTAGCGAGCTGACCCAGGATCCAGTGGTGTCTGTTCCTCTGGGCCAGACCGCCAGA |
| | | | ATTACCTGTCAGGGCGATAGCCTGACCACCTACTACGCCACCTGGTATCAGCAGAA |
| | | | GCCAGGCCAGGCTCCTGTGCTGGTGCTGTACAATGAGCACAAGAGGCCCAGCGGCA |
| | | | TCAGCGACAGATTTTCTGGATCTTCTGCCGGCGACGCCGCCAGCCTGACAATCACA |
| | | | GATACACAGGCCGAGGACGAGGCCGACTACTACTGCAGCTCTAGAGATACCGGCGG |
| | | | CAAACACGTGCTGTTTGGAGGCGGCACAAAGCTGACAGTGCTGACCGGTGGCGGAG |
| | | | GATCTGGCGGAGGCGGATCTGATGAATTTCCCACCATGGTGTTTCCTTCTGGGCAG |
| | | | ATCAGCCAGGCCTCGGCCTTGGCCCCGGCCCCTCCCCAAGTCCTGCCCCAGGCTCC |
| | | | AGCCCCTGCCCCTGCTCCAGCCATGGTATCAGCTCTGGCCCAGGCCCCAGCCCCTG |
| | | | TCCCAGTCCTAGCCCCAGGCCCTCCTCAGGCTGTGGCCCCACCTGCCCCCAAGCCC |
| | | | ACCCAGGCTGGGGAAGGAACGCTGTCAGAGGCCCTGCTGCAGCTGCAGTTTGATGA |
| | | | TGAAGACCTGGGGGCCTTGCTTGGCAACAGCACAGACCCAGCTGTGTTCACAGACC |
| | | | TGGCATCCGTCGACAACTCCGAGTTTCAGCAGCTGCTGAACCAGGGCATACCTGTG |
| | | | GCCCCCCACACAACTGAGCCCATGCTGATGGAGTACCCTGAGGCTATAACTCGCCT |
| | | | AGTGACAGGGGCCCAGAGGCCCCCCGACCCAGCTCCTGCTCCACTGGGGGCCCCGG |
| | | | GGCTCCCCAATGGCCTCCTTTCAGGAGATGAAGACTTCTCCTCCATTGCGGACATG |
| | | | GACTTCTCAGCCCTGCTGAGTCAGATCAGCTCCACTAGTTAT |
| 131 | PRSIM_75_AD_ | DNA | ATGGGCTCTGAAGTGCAGCTGGTTCAGTCTGGCGCCGAAGTGAAGAAACCTGGCAG |
| | fusion | | CAGCGTGAAGGTGTCCTGCAAAGCTTCTGGCGGCAGCTTCAACAGCTACACCCTGG |
| | | | ACTGGGTTCGACAGGCCCCTGGACAAGGACTGGAATGGATGGGCGGAATCATCCCC |
| | | | GTGTTCGGCAGCCCTAATTACGGCCAGAAATTCCAGGGCAGAGTGACCATCACCGC |
| | | | CGACGAGTCTACAAGCACCGCCTACATGGAACTGAGCAGCCTGAAGTCCGACGACA |
| | | | CCGCCGTGTACTATTGTGCCAGAGGCCTGGTGTACCAGCCACTGGATTCTTGGGGC |
| | | | AGAGGCACCCTGGTCACAGTTTCTAGCGGAGGCGGAGGATCTGGTGGCGGAGGAAG |
| | | | TGGCGGAGGCGGTTCTGCTCAAGCTGTTCTGACACAGCCTAGCAGCGCCTCTGGAA |
| | | | CACCTGGCCAGAGAGTGACAATCAGCTGTAGCGGCAGCAGCAGCAACATCGGCTCC |
| | | | TACACCGTGAACTGGTATCAGCAGTTCCCCGGCACAGCCCCTAAGCTGCTGATCTA |
| | | | CAGCAACACCCAGAGGCCAAGCGGCGTGCCCGATAGATTTTCTGGCAGCAAGAGCG |
| | | | GCACAAGCGCCAGCCTGGCTATTTCTGGACTGCAGAGCGAGGACGAGGCCGACTAT |
| | | | TATTGTGCCGCCTGGGACGACAGCCTGAACGGATGGGTTTTCGGCGGAGGCACCAA |
| | | | AGTGACAGTGCTGACCGGTGGCGGAGGATCTGGCGGAGGCGGATCTGATGAATTTC |
| | | | CCACCATGGTGTTTCCTTCTGGGCAGATCAGCCAGGCCTCGGCCTTGGCCCCGGCC |
| | | | CCTCCCCAAGTCCTGCCCCAGGCTCCAGCCCCTGCCCCTGCTCCAGCCATGGTATC |
| | | | AGCTCTGGCCCAGGCCCCAGCCCCTGTCCCAGTCCTAGCCCCAGGCCCTCCTCAGG |
| | | | CTGTGGCCCCACCTGCCCCCAAGCCCACCCAGGCTGGGGAAGGAACGCTGTCAGAG |
| | | | GCCCTGCTGCAGCTGCAGTTTGATGATGAAGACCTGGGGGCCTTGCTTGGCAACAG |
| | | | CACAGACCCAGCTGTGTTCACAGACCTGGCATCCGTCGACAACTCCGAGTTTCAGC |
| | | | AGCTGCTGAACCAGGGCATACCTGTGGCCCCCCACACAACTGAGCCCATGCTGATG |
| | | | GAGTACCCTGAGGCTATAACTCGCCTAGTGACAGGGGCCCAGAGGCCCCCCGACCC |
| | | | AGCTCCTGCTCCACTGGGGGCCCCGGGGCTCCCCAATGGCCTCCTTTCAGGAGATG |
| | | | AAGACTTCTCCTCCATTGCGGACATGGACTTCTCAGCCCTGCTGAGTCAGATCAGC |
| | | | TCCACTAGTTAT |
| 132 | FKBP12: FRB | DNA | ATGCTGCTGCTGGTTACATCTCTGCTGCTGTGCGAGCTGCCCCATCCTGCCTTTCT |
| | CAR | | GCTGATTCCTGAAAGCAAATACGGCCCTCCGTGTCCTCCTTGTCCTTTCTGGGTGC |
| | | | TCGTGGTTGTTGGCGGAGTGCTGGCCTGTTATAGCCTGCTTGTGACCGTGGCCTTC |
| | | | ATCATCTTTTGGGTCAAGCGGGGCAGAAAGAAGCTGCTGTACATCTTCAAGCAGCC |
| | | | CTTCATGCGGCCCGTGCAGACCACACAAGAGGAAGATGGCTGCTCCTGCAGATTCC |
| | | | CCGAGGAAGAAGAAGGCGGCTGCGAGCTGTCTAGAGGCAGCGGATCAGGCTCTGGA |
| | | | TCTATGGGCGTGCAGGTCGAGACAATCTCTCCTGGCGACGGCAGAACATTCCCCAA |
| | | | GAGGGGCCAGACATGCGTGGTGCACTATACCGGCATGCTCGAGGACGGCAAGAAGT |
| | | | TCGACAGCTCCCGGGACAGAAACAAGCCCTTCAAGTTCATGCTGGGCAAGCAAGAA |
| | | | GTGATCAGAGGCTGGGAAGAGGGCGTCGCCCAGATGTCTGTTGGCCAGAGAGCCAA |
| | | | ACTGACAATCAGCCCCGATTACGCCTACGGCGCCACAGGACACCCTGGAATCATTC |
| | | | CTCCACACGCCACACTGGTGTTCGACGTGGAACTGCTGAAGCTGGAAGGCAGCGGC |
| | | | GCCACCAATTTCAGCCTGCTGAAACAGGCCGGCGACGTCGAAGAGAACCCCGGACC |
| | | | TATGATCCACCTGGGCCACATTCTGTTTCTGTTGCTGCTGCCTGTGGCCGCTGCTC |
| | | | AGACAACACCTGGCGAGAGATCTAGCCTGCCTGCCTTCTATCCTGGCACCAGCGGC |
| | | | TCTTGTTCTGGCTGTGGATCTCTGAGCCTGCCAGAGTCTAAGTACGGCCCTCCGTG |
| | | | TCCACCATGTCCATTTTGGGTCCTCGTTGTCGTCGGAGGCGTGCTGGCTTGCTATT |
| | | | CTCTGCTCGTGACAGTCGCCTTTATTATCTTCTGGGTGTCCCTGAAGAGAGGCCGG |
| | | | AAAAAACTGCTCTATATCTTTAAACAGCCGTTTATGCGCCCGGTCCAGACAACCCA |
| | | | AGAAGAGGACGGCTGTAGCTGCCGGTTTCCTGAAGAAGAGGAAGGCGGTTGCGAAC |
| | | | TGATCCTGTGGCACGAGATGTGGCATGAAGGCCTGGAAGAGGCCAGCAGACTGTAC |
| | | | TTCGGCGAGAGAAACGTGAAAGGCATGTTCGAGGTGCTGGAACCTCTGCACGCCAT |
| | | | GATGGAAAGAGGCCCTCAGACACTGAAAGAGACAAGCTTCAACCAGGCCTACGGCC |
| | | | GGGATCTGATGGAAGCCCAAGAGTGGTGCCGGAAGTACATGAAGTCCGGCAACGTG |
| | | | AAGGACCTGCTGCAGGCCTGGGATCTGTACTACCACGTGTTCCGGCGGATCAGCAA |
| | | | AGGCTCCGGAAGCGGATCTGGAAGCTCCCTGAGAGTGAAGTTCAGCAGAAGCGCCG |
| | | | ACGCTCCTGCCTATCAGCAGGGACAGAACCAGCTGTACAACGAGCTGAACCTGGGG |
| | | | AGAAGAGAAGAGTACGACGTGCTGGACAAGCGGAGAGGCAGAGATCCTGAGATGGG |
| | | | CGGCAAGCCCAGACGGAAGAATCCTCAAGAGGGCCTGTATAATGAGCTGCAGAAAG |
| | | | ACAAGATGGCCGAGGCCTACAGCGAGATCGGAATGAAGGGCGAGCGCAGAAGAGGC |
| | | | AAGGGACACGATGGACTGTACCAGGGCCTGAGCACCGCCACCAAGGATACCTATGA |
| | | | TGCCCTGCACATGCAGGCCCTGCCTCCAAGAGGTAGTGGCGAAGGCAGAGGCTCTC |
| | | | TGCTGACATGCGGAGATGTGGAAGAGAATCCTGGGCCAAGCGGCATGGAAAGCGAC |
| | | | GAATCTGGACTCCCCGCCATGGAAATCGAGTGCAGAATCACCGGCACACTGAACGG |
| | | | CGTGGAATTCGAACTCGTTGGAGGCGGCGAGGGCACACCTAAGCAGGGCAGAATGA |
| | | | CCAACAAGATGAAGTCCACCAAAGGCGCCCTGACTTTCAGCCCCTACCTGCTGTCT |
| | | | CACGTGATGGGCTACGGCTTCTACCACTTCGGCACATACCCTAGCGGCTACGAGAA |
| | | | CCCCTTCCTGCATGCCATCAACAACGGCGGCTACACCAACACCAGAATCGAGAAGT |
| | | | ACGAGGATGGCGGCGTGCTGCACGTGTCCTTCAGCTACAGATATGAGGCCGGCAGA |
| | | | GTGATCGGCGACTTCAAGGTTGTCGGCACCGGCTTTCCAGAGGACAGCGTGATCTT |
| | | | CACCGACAAGATCATCCGGTCCAACGCCACCGTCGAGCATCTGCACCCTATGGGCG |
| | | | ATAATGTGCTTGTGGGCAGCTTCGCCAGAACCTTCAGTCTGCGTGATGGCGGCTAC |
| | | | TACAGCTTCGTGGTGGACAGCCACATGCACTTCAAGAGCGCCATCCATCCTAGCAT |
| | | | CCTGCAGAACGGCGGACCCATGTTCGCCTTCAGAAGAGTGGAAGAACTGCACTCCA |
| | | | ACACCGAGCTGGGCATCGTGGAATACCAGCACGCTTTCAAGACCCCTATCGCCTTC |
| | | | GCAAGAAGCAGAGCCCAGAGCAGCAATAGCGCCGTGGATGGAACAGCCGGACCTGG |
| | | | CTCTACAGGCTCCAGA |
| 133 | PRSIM_23 | DNA | ATGCTGCTGCTGGTTACATCTCTGCTGCTGTGCGAGCTGCCCCATCCTGCCTTTCT |
| | CAR | | GCTGATTCCTGAAAGCAAATACGGCCCTCCGTGTCCTCCTTGTCCTTTCTGGGTGC |
| | | | TCGTGGTTGTTGGCGGAGTGCTGGCCTGTTATAGCCTGCTTGTGACCGTGGCCTTC |
| | | | ATCATCTTTTGGGTCAAGCGGGGCAGAAAGAAGCTGCTGTACATCTTCAAGCAGCC |
| | | | CTTCATGCGGCCCGTGCAGACCACACAAGAGGAAGATGGCTGCTCCTGCAGATTCC |
| | | | CCGAGGAAGAAGAAGGCGGCTGCGAGCTGGGTGGCGGAGGATCTGGCGGAGGCGGA |
| | | | TCTATGAAGAAAAAGGGCTCTGTGGTCATCGTGGGCAGAATCAACCTGAGCGGCGA |
| | | | TACCGCCTACGCTCAGCAGACAAGAGGCGAGGAAGGCTGCCAAGAGACAAGCCAGA |
| | | | CCGGCAGAGACAAGAACCAGGTGGAAGGCGAGGTGCAGATCGTGTCTACAGCTACC |
| | | | CAGACCTTCCTGGCCACCAGCATCAATGGCGTGCTGTGGACAGTGTATCACGGCGC |
| | | | TGGCACCAGAACAATCGCCTCTCCAAAGGGCCCCGTGACACAGATGTACACCAACG |
| | | | TGGACAAGGACCTCGTCGGATGGCAAGCCCCTCAGGGCTCTAGAAGCCTGACACCT |
| | | | TGTACCTGCGGCAGCAGCGATCTGTACCTGGTCACAAGACACGCCGACGTGATCCC |
| | | | CGTCAGAAGAAGAGGCGATAGCAGAGGCAGCCTGCTGAGCCCTAGACCTATCAGCT |
| | | | ACCTGAAGGGATCTGCCGGCGGACCTCTGCTTTGTCCTGCTGGACATGCCGTGGGC |
| | | | ATCTTTAGAGCCGCCGTGTCTACTAGAGGCGTGGCCAAGGCCGTGGACTTCATCCC |
| | | | TGTGGAAAGCCTGGAAACCACCATGCGGAGCCCCGGCAGCGGCGCCACCAATTTCA |
| | | | GCCTGCTGAAACAGGCCGGCGACGTCGAAGAGAACCCCGGACCTATGATCCACCTG |
| | | | GGCCACATTCTGTTTCTGTTGCTGCTGCCTGTGGCCGCTGCTCAGACAACACCTGG |
| | | | CGAGAGATCTAGCCTGCCTGCCTTCTATCCTGGCACCAGCGGCTCTTGTTCTGGCT |
| | | | GTGGATCTCTGAGCCTGCCAGAGTCTAAGTACGGCCCTCCGTGTCCACCATGTCCA |
| | | | TTTTGGGTCCTCGTTGTCGTCGGAGGCGTGCTGGCTTGCTATTCTCTGCTCGTGAC |
| | | | AGTCGCCTTTATTATCTTCTGGGTGTCCCTGAAGAGAGGCCGGAAAAAACTGCTCT |
| | | | ATATCTTTAAACAGCCGTTTATGCGCCCGGTCCAGACAACCCAAGAAGAGGACGGC |
| | | | TGTAGCTGCCGGTTTCCTGAAGAAGAGGAAGGCGGTTGCGAACTGGGTGGCGGAGG |
| | | | ATCTGGCGGAGGCGGATCTATGGGCTCTAGACTGGATGCCCCTAGCCAGATCGAAG |
| | | | TGAAGGACGTGACCGACACCACCGCTCTGATCACCTGGGTTGACCCCAGATACGAC |
| | | | GACATCTGGTGGTTCGAGCTGACCTACGGCATCAAGGATGTGCCCGGCGACAGAAC |
| | | | CACCATCAAGCTGTACCTGAACGACCCCTACTACAGCATCGGCAACCTGAAGCCTG |
| | | | ACACCGAGTACGAGGTGTCCCTGATCAGCTACACCGGCGACTCCTACAGCAGAAGC |
| | | | GGCAGCAATCCTGCCAAGATCACCTTCAAGACCGGCCTTGGTGGCGGAGGATCTGG |
| | | | CGGAGGCGGATCTCTGAGAGTGAAGTTCAGCAGAAGCGCCGACGCTCCTGCCTATC |
| | | | AGCAGGGACAGAACCAGCTGTACAACGAGCTGAACCTGGGGAGAAGAGAAGAGTAC |
| | | | GACGTGCTGGACAAGCGGAGAGGCAGAGATCCTGAGATGGGCGGCAAGCCCAGACG |
| | | | GAAGAATCCTCAAGAGGGCCTGTATAATGAGCTGCAGAAAGACAAGATGGCCGAGG |
| | | | CCTACAGCGAGATCGGAATGAAGGGCGAGCGCAGAAGAGGCAAGGGACACGATGGA |
| | | | CTGTACCAGGGCCTGAGCACCGCCACCAAGGATACCTATGATGCCCTGCACATGCA |
| | | | GGCCCTGCCTCCAAGAGGTAGTGGCGAAGGCAGAGGCTCTCTGCTGACATGCGGAG |
| | | | ATGTGGAAGAGAATCCTGGGCCAAGCGGCATGGAAAGCGACGAATCTGGACTCCCC |
| | | | GCCATGGAAATCGAGTGCAGAATCACCGGCACACTGAACGGCGTGGAATTCGAACT |
| | | | CGTTGGAGGCGGCGAGGGCACACCTAAGCAGGGCAGAATGACCAACAAGATGAAGT |
| | | | CCACCAAAGGCGCCCTGACTTTCAGCCCCTACCTGCTGTCTCACGTGATGGGCTAC |
| | | | GGCTTCTACCACTTCGGCACATACCCTAGCGGCTACGAGAACCCCTTCCTGCATGC |
| | | | CATCAACAACGGCGGCTACACCAACACCAGAATCGAGAAGTACGAGGATGGCGGCG |
| | | | TGCTGCACGTGTCCTTCAGCTACAGATATGAGGCCGGCAGAGTGATCGGCGACTTC |
| | | | AAGGTTGTCGGCACCGGCTTTCCAGAGGACAGCGTGATCTTCACCGACAAGATCAT |
| | | | CCGGTCCAACGCCACCGTCGAGCATCTGCACCCTATGGGCGATAATGTGCTTGTGG |
| | | | GCAGCTTCGCCAGAACCTTCAGTCTGCGTGATGGCGGCTACTACAGCTTCGTGGTG |
| | | | GACAGCCACATGCACTTCAAGAGCGCCATCCATCCTAGCATCCTGCAGAACGGCGG |
| | | | ACCCATGTTCGCCTTCAGAAGAGTGGAAGAACTGCACTCCAACACCGAGCTGGGCA |
| | | | TCGTGGAATACCAGCACGCTTTCAAGACCCCTATCGCCTTCGCAAGAAGCAGAGCC |
| | | | CAGAGCAGCAATAGCGCCGTGGATGGAACAGCCGGACCTGGCTCTACAGGCTCCAG |
| | | | A |
| 134 | Wild type TN3 | Protein | RLDAPSQIEVKDVTDTTALITWFKPLAEIDGIELTYGIKDVPGDRTTIDL |
| | | | TEDENQYSIGNLKPDTEYEVSLISRRGDMSSNPAKETFTTGL |
| 135 | Wild type TN3 | Protein | RLDAPSQIEVKDVTDTTALITWFKPLAEIDGFELTYGIKDVPGDRTTIK |
| | with stabilising | | LTEDENQYSIGNLKPDTEYEVSLISRRGDMSSNPAKITFKTGL |
| | mutations | | |
| 136 | PRSIM_23 | Protein | VDPRYDDIWW |
| | BC loop | | |
| 137 | PRSIM_23 | Protein | YLNDPY |
| | DE loop | | |
| 138 | PRSIM_23 | Protein | YTGDSYSRSGSNPA |
| | FG loop | | |
| 139 | PRSIM_32 | Protein | WSPRYYYASISG |
| | BC loop | | |
| 140 | PRSIM_32 | Protein | DYASND |
| | DE loop | | |
| 141 | PRSIM_32 | Protein | WNYGDWRYSSSNPA |
| | FG loop | | |
| 142 | PRSIM_33 | Protein | YPPGRWYDDIWY |
| | BC loop | | |
| 143 | PRSIM_33 | Protein | ARGDDV |
| | DE loop | | |
| 144 | PRSIM_33 | Protein | WGPDRGDRAGSNPA |
| | FG loop | | |
| 145 | PRSIM_36 | Protein | SWPRDDDYDIWY |
| | BC loop | | |
| 146 | PRSIM_36 | Protein | LNYASP |
| | DE loop | | |
| 147 | PRSIM_36 | Protein | VPDTYGRGTSNPA |
| | FG loop | | |
| 148 | PRSIM_47 | Protein | SRPGVSIWY |
| | BC loop | | |
| 149 | PRSIM_47 | Protein | DYRSYY |
| | DE loop | | |
| 150 | PRSIM_47 | Protein | GSYGLVGVRASNPA |
| | FG loop | | |
| 151 | PRSIM_57 | Protein | SYAIS |
| | HCDR1 | | |
| 152 | PRSIM_57 | Protein | GIIPIFGTANYAQKFQG |
| | HCDR2 | | |
| 153 | PRSIM_57 | Protein | HTNYITVFDY |
| | HCDR3 | | |
| 154 | PRSIM_57 | Protein | SGSSSNIGSNTVN |
| | LCDR1 | | |
| 155 | PRSIM_57 | Protein | SNNQRPS |
| | LCDR2 | | |
| 156 | PRSIM_57 | Protein | AAWDHHWEQVV |
| | LCDR3 | | |
| 151 | PRSIM_01 | Protein | SYAIS |
| | HCDR1 | | |
| 152 | PRSIM_01 | Protein | GIIPIFGTANYAQKFQG |
| | HCDR2 | | |
| 198 | PRSIM_01 | Protein | GQGYITVFDY |
| | HCDR3 | | |
| 154 | PRSIM_01 | Protein | SGSSSNIGSNTVN |
| | LCDR1 | | |
| 155 | PRSIM_01 | Protein | SNNQRPS |
| | LCDR2 | | |
| 156 | PRSIM_01 | Protein | AAWDHHWEQVV |
| | LCDR3 | | |
| 151 | PRSIM_04 | Protein | SYAIS |
| | HCDR1 | | |
| 152 | PRSIM_04 | Protein | GIIPIFGTANYAQKFQG |
| | HCDR2 | | |
| 163 | PRSIM_04 | Protein | GMAHFYQFDL |
| | HCDR3 | | |
| 154 | PRSIM_04 | Protein | SGSSSNIGSNTVN |
| | LCDR1 | | |
| 155 | PRSIM_04 | Protein | SNNQRPS |
| | LCDR2 | | |
| 164 | PRSIM_04 | Protein | AAGDHDHEHVV |
| | LCDR3 | | |
| 165 | PRSIM_67 | Protein | SYYVH |
| | HCDR1 | | |
| 166 | PRSIM_67 | Protein | VINPSGGNTNYAQKFQD |
| | HCDR2 | | |
| 167 | PRSIM_67 | Protein | RDYGGPLAN |
| | HCDR3 | | |
| 168 | PRSIM_67 | Protein | SGSSSNIGNNAVN |
| | LCDR1 | | |
| 169 | PRSIM_67 | Protein | YDDLLPS |
| | LCDR2 | | |
| 170 | PRSIM_67 | Protein | AAWDDSLNGLV |
| | LCDR3 | | |
| 171 | PRSIM_72 | Protein | NYGVS |
| | HCDR1 | | |
| 172 | PRSIM_72 | Protein | RIIPIRDTANYAQKFQG |
| | HCDR2 | | |
| 173 | PRSIM_72 | Protein | VLEDDFWGGYYDFYFYVMDV |
| | HCDR3 | | |
| 174 | PRSIM_72 | Protein | QGDSLTTYYAT |
| | LCDR1 | | |
| 175 | PRSIM_72 | Protein | NEHKRPS |
| | LCDR2 | | |
| 176 | PRSIM_72 | Protein | SSRDTGGKHVL |
| | LCDR3 | | |
| 177 | PRSIM_75 | Protein | SYTLD |
| | HCDR1 | | |
| 178 | PRSIM_75 | Protein | GIIPVFGSPNYGQKFQG |
| | HCDR2 | | |
| 179 | PRSIM_75 | Protein | GLVYQPLDS |
| | HCDR3 | | |
| 180 | PRSIM_75 | Protein | SGSSSNIGSYTVN |
| | LCDR1 | | |
| 181 | PRSIM_75 | Protein | SNTQRPS |
| | LCDR2 | | |
| 182 | PRSIM_75 | Protein | AAWDDSLNGW |
| | LCDR3 | | |
| 183 | Tn3_pETFwd2 | DNA | CGATCATATGGACTACAAGGACGACGATGACAAGGGCAGCCGTCTGGATGCACCGA |
| | | | GCCAG |
| 184 | Tn3_pETRev2 | DNA | ATCGGGATCCCTACAGACCGGTTTTAAAGGTAATTTTTGCCGG |
| 185 | Linker | Protein | TGGGGSGGGGS |
| 186 | PRSIM_57 VH | Protein | QVQLVQSGAEVKKPGSSVKVSCKASGGTFSSYAISWVRQAPGQGLEWMGGIIPIFG |
| | | | TANYAQKFQGRVTITADESTSTAYMELSSLRSEDTAVYYCARHTNYITVFDYWGQG |
| | | | TLVTVSS |
| 187 | PRSIM_57 VL | Protein | QSVLTQPPSASGTPGQRVTISCSGSSSNIGSNTVNWYQQLPGTAPKLLIYSNNQRP |
| | | | SGVPDRFSGSKSGTSASLAISGLQSEDEADYYCAAWDHHWEQVVFGGGTKLTVL |
| 188 | PRSIM_01 VH | Protein | QVQLVQSGAEVKKPGSSVKVSCKASGGTFSSYAISWVRQAPGQGLEWMGGIIPIFG |
| | | | TANYAQKFQGRVTITADESTSTAYMELSSLRSEDTAVYYCARGQGYITVFDYWGQG |
| | | | TLVTVSS |
| 189 | PRSIM_01 VL | Protein | QSVLTQPPSASGTPGQRVTISCSGSSSNIGSNTVNWYQQLPGTAPKLLIYSNNQRP |
| | | | SGVPDRFSGSKSGTSASLAISGLQSEDEADYYCAAWDHHWEQVVFGGGTKLTVL |
| 190 | PRSIM_04 VH | Protein | QVQLVQSGAEVKKPGSSVKVSCKASGGTFSSYAISWVRQAPGQGLEWMGGIIPIFG |
| | | | TANYAQKFQGRVTITADESTSTAYMELSSLRSEDTAVYYCARGMAHFYQFDLWGQG |
| | | | TLVTVSS |
| 191 | PRSIM_04 VL | Protein | QSVLTQPPSASGTPGQRVTISCSGSSSNIGSNTVNWYQQLPGTAPKLLIYSNNQRP |
| | | | SGVPDRFSGSKSGTSASLAISGLQSEDEADYYCAAGDHDHEHVVFGGGTKLTVL |
| 192 | PRSIM_67 VH | Protein | EVQLVQSGAEVKKPGAAVRISCKTSGYVFTSYYVHWVRQAPGQGLEWMGVINPSGG |
| | | | NTNYAQKFQDRVTMTRDTSTTTVYMELSSLMFDDTAVYYCAKRDYGGPLANWGRGT |
| | | | LVTVSS |
| 193 | PRSIM_67 VL | Protein | SYELTQPPSVSEAPRQRVTISCSGSSSNIGNNAVNWYQQLPGKAPKLLIFYDDLLP |
| | | | SGVSDRFSGSKSGTSASLAISGLQSEDEADYYCAAWDDSLNGLVFGTGTKLTVL |
| 194 | PRSIM_72 VH | Protein | QVQLVQSGAEVKKPGSSVKVSCKASGGTFSSYAISWVRQAPGQGLEWMGGIIPIFG |
| | | | TANYAQKFQGRVTITADESTSTAYMELSSLRSEDTAVYYCARGMAHFYQFDLWGQG |
| | | | TLVTVSS |
| 195 | PRSIM_72 VL | Protein | QSVLTQPPSASGTPGQRVTISCSGSSSNIGSNTVNWYQQLPGTAPKLLIYSNNQRP |
| | | | SGVPDRFSGSKSGTSASLAISGLQSEDEADYYCAAGDHDHEHVVFGGGTKLTVL |
| 196 | PRSIM_75 VH | Protein | EVQLVQSGAEVKKPGSSVKVSCKASGGSFNSYTLDWVRQAPGQGLEWMGGIIPVFG |
| | | | SPNYGQKFQGRVTITADESTSTAYMELSSLKSDDTAVYYCARGLVYQPLDSWGRGT |
| | | | LVTVSS |
| 197 | PRSIM_75 VL | Protein | QAVLTQPSSASGTPGQRVTISCSGSSSNIGSYTVNWYQQFPGTAPKLLIYSNTQRP |
| | | | SGVPDRFSGSKSGTSASLAISGLQSEDEADYYCAAWDDSLNGWVFGGGTKVTVL |
| 199 | Full length | Protein | APITAYAQQTRGEEGCQETSLTGRDKNQVEGEVQIVSTAAQTFLATSINGVCWTVY |
| | NS3 protein | | HGAGTRTIASPKGPVIQMYTNVDQDLVGWPAPQGSRSLTPCTCGSSDLYLVTRHAD |
| | | | VIPVRRRGDSRGSLLSPRPISYLKGSSGGPLLCPAGHAVGIFRAAVCTRGVAKAVD |
| | | | FIPVENLETTMRSPVFTDNSSPPVVPQSFQVAHLHAPTGSGKSTKVPAAYAAQGYK |
| | | | VLVLNPSVAATLGFGAYMSKAHGIDPNIRTGVRTITTGSPITYSTYGKFLADGGCS |
| | | | GGAYDIIICDECHSTDATSILGIGTVLDQAETAGARLVVLATATPPGSVTVPHPNI |
| | | | EEVALSTTGEIPFYGKAIPLEVIKGGRHLIFCHSKKKCDELAAKLVALGINAVAYY |
| | | | RGLDVSVIPTSGDVVVVATDALMTGYTGDFDSVIDCNTCVTQTVDFSLDPTFTIET |
| | | | ITLPQDAVSRTQRRGRTGRGKPGIYRFVAPGERPSGMFDSSVLCECYDAGCAWYEL |
| | | | TPAETTVRLRAYMNTPGLPVCQDHLEFWEGVFTGLTHIDAHFLSQTKQSGENLPYL |
| | | | VAYQATVCARAQAPPPSWDQMWKCLIRLKPTLHGPTPLLYRLGAVQNEITLTHPVT |
| | | | KYIMTCMSADLEVVT |
| 200 | PRSIM_23 | Protein | ESKYGPPCPPCPFWVLVVVGGVLACYSLLVTVAFIIFWVSLKRGRKKLLYIFKQPF |
| | CAR 2nd | | MRPVQTTQEEDGCSCRFPEEEEGGCELGGGGSGGGGSMGSRLDAPSQIEVKDVTDT |
| | polypeptide | | TALITWVDPRYDDIWWFELTYGIKDVPGDRTTIKLYLNDPYYSIGNLKPDTEYEVS |
| | | | LISYTGDSYSRSGSNPAKITFKTGLGGGGSGGGGSLRVKFSRSADAPAYQQGQNQL |
| | | | YNELNLGRREEYDVLDKRRGRDPEMGGKPRRKNPQEGLYNELQKDKMAEAYSEIGM |
| | | | KGERRRGKGHDGLYQGLSTATKDTYDALHMQALPPRGSG |
| 201 | PRSIM_23 | Protein | MLLLVTSLLLCELPHPAFLLIP |
| | CAR 1st | | |
| | signal peptide | | |
| 202 | PRSIM_23 | Protein | MIHLGHILFLLLLPVAAAQTTPGERSSLPAFYPGTSGSCSGCGSLSLP |
| | CAR 2nd | | |
| | signal peptide | | |
| 203 | PRSIM_23 | Protein | MIHLGHILFLLLLPVAAAQTTPGERSSLPAFYPGTSGSCSGCGSLSLPESKYGPPC |
| | CAR 2nd | | PPCPFWVLVVVGGVLACYSLLVTVAFIIFWVSLKRGRKKLLYIFKQPFMRPVQTTQ |
| | polypeptide + | | EEDGCSCRFPEEEEGGCELGGGGSGGGGSMGSRLDAPSQIEVKDVTDTTALITWVD |
| | signal peptide | | PRYDDIWWFELTYGIKDVPGDRTTIKLYLNDPYYSIGNLKPDTEYEVSLISYTGDS |
| | | | YSRSGSNPAKITFKTGLGGGGSGGGGSLRVKFSRSADAPAYQQGQNQLYNELNLGR |
| | | | REEYDVLDKRRGRDPEMGGKPRRKNPQEGLYNELQKDKMAEAYSEIGMKGERRRGK |
| | | | GHDGLYQGLSTATKDTYDALHMQALPPRGSG |
| 204 | Linker | Protein | GGGGSGGGGS |
| 205 | MEDI8852 | Protein | QVQLQQSGPGLVKPSQTLSLTCAISGDSVSSYNAVWNWIRQSPSRGLEWLGRTYYR |
| | heavy chain | | SGWYNDYAESVKSRITINPDTSKNQFSLQLNSVTPEDTAVYYCARSGHITVFGVNV |
| | | | DAFDMWGQGTMVTVSSASTKGPSVFPLAPSSKSTSGGTAALGCLVKDYFPEPVTVS |
| | | | WNSGALTSGVHTFPAVLQSSGLYSLSSVVTVPSSSLGTQTYICNVNHKPSNTKVDK |
| | | | RVEPKSCDKTHTCPPCPAPELLGGPSVFLFPPKPKDTLMISRTPEVTCVVVDVSHE |
| | | | DPEVKFNWYVDGVEVHNAKTKPREEQYNSTYRVVSVLTVLHQDWLNGKEYKCKVSN |
| | | | KALPAPIEKTISKAKGQPREPQVYTLPPSREEMTKNQVSLTCLVKGFYPSDIAVEW |
| | | | ESNGQPENNYKTTPPVLDSDGSFFLYSKLTVDKSRWQQGNVFSCSVMHEALHNHYT |
| | | | QKSLSLSPGK |
| 206 | MEDI8852 | Protein | DIQMTQSPSSLSASVGDRVTITCRTSQSLSSYTHWYQQKPGKAPKLLIYAASSRGS |
| | light chain | | GVPSRFSGSGSGTDFTLTISSLQPEDFATYYCQQSRTFGQGTKVEIKRTVAAPSVF |
| | | | IFPPSDEQLKSGTASVVCLLNNFYPREAKVQWKVDNALQSGNSQESVTEQDSKDST |
| | | | YSLSSTLTLSKADYEKHKVYACEVTHQGLSSPVTKSFNRGEC |
| 207 | DBD- | Protein | MDYPAAKRVKLDSRERPYACPVESCDRRFSRSDELTRHIRIHTGQKPFQCRICMRN |
| | PRSIM_23x3- | | FSRSDHLTTHIRTHTGGGRRRKKRTSIETNIRVALEKSFLENQKPTSEEITMIADQ |
| | P2A-NS4A/3 | | LNMEKEVIRVWFCNRRQKEKRINTSAGSRLDAPSQIEVKDVTDTTALITWVDPRYD |
| | PR S139A-AD | | DIWWFELTYGIKDVPGDRTTIKLYLNDPYYSIGNLKPDTEYEVSLISYTGDSYSRS |
| | | | GSNPAKITFKTGLRLDAPSQIEVKDVTDTTALITWVDPRYDDIWWFELTYGIKDVP |
| | | | GDRTTIKLYLNDPYYSIGNLKPDTEYEVSLISYTGDSYSRSGSNPAKITFKTGLRL |
| | | | DAPSQIEVKDVTDTTALITWVDPRYDDIWWFELTYGIKDVPGDRTTIKLYLNDPYY |
| | | | SIGNLKPDTEYEVSLISYTGDSYSRSGSNPAKITFKTGLTSKGSGATNFSLLKQAG |
| | | | DVEENPGPMAKKGSVVIVGRINLSGDTAYAQQTRGEEGCQETSQTGRDKNQVEGEV |
| | | | QIVSTATQTFLATSINGVLWTVYHGAGTRTIASPKGPVTQMYTNVDKDLVGWQAPQ |
| | | | GSRSLTPCTCGSSDLYLVTRHADVIPVRRRGDSRGSLLSPRPISYLKGSAGGPLLC |
| | | | PAGHAVGIFRAAVSTRGVAKAVDFIPVESLETTMRSPTRDEFPTMVFPSGQISQAS |
| | | | ALAPAPPQVLPQAPAPAPAPAMVSALAQAPAPVPVLAPGPPQAVAPPAPKPTQAGE |
| | | | GTLSEALLQLQFDDEDLGALLGNSTDPAVFTDLASVDNSEFQQLLNQGIPVAPHTT |
| | | | EPMLMEYPEAITRLVTGAQRPPDPAPAPLGAPGLPNGLLSGDEDFSSIADMDFSAL |
| | | | LSQISSTSY |
| 208 | DBD- | DNA | ATGGACTATCCTGCTGCCAAGAGGGTCAAGTTGGACTCTAGAGAACGCCCATATGC |
| | PRSIM_23x3- | | TTGCCCTGTCGAGTCCTGCGATCGCCGCTTTTCTCGCTCGGATGAGCTTACCCGCC |
| | P2A-NS4A/3 | | ATATCCGCATCCACACAGGCCAGAAGCCCTTCCAGTGTCGAATCTGCATGCGTAAC |
| | PR S139A-AD | | TTCAGTCGTAGTGACCACCTTACCACCCACATCCGCACCCACACAGGCGGCGGCCG |
| | | | CAGGAGGAAGAAACGCACCAGCATAGAGACCAACATCCGTGTGGCCTTAGAGAAGA |
| | | | GTTTCTTGGAGAATCAAAAGCCTACCTCGGAAGAGATCACTATGATTGCTGATCAG |
| | | | CTCAATATGGAAAAAGAGGTGATTCGTGTTTGGTTCTGTAACCGCCGCCAGAAAGA |
| | | | AAAAAGAATCAACACTAGCGCTGGCTCTAGACTGGATGCCCCTAGCCAGATCGAAG |
| | | | TGAAGGACGTGACCGACACCACCGCTCTGATCACCTGGGTTGACCCCAGATACGAC |
| | | | GACATCTGGTGGTTCGAGCTGACCTACGGCATCAAGGATGTGCCCGGCGACAGAAC |
| | | | CACCATCAAGCTGTACCTGAACGACCCCTACTACAGCATCGGCAACCTGAAGCCTG |
| | | | ACACCGAGTACGAGGTGTCCCTGATCAGCTACACCGGCGACTCCTACAGCAGAAGC |
| | | | GGCAGCAATCCTGCCAAGATCACCTTCAAGACCGGCCTGAGACTGGACGCACCCTC |
| | | | TCAGATTGAAGTCAAAGATGTCACCGACACGACAGCCCTGATTACATGGGTTGACC |
| | | | CTCGCTACGATGATATTTGGTGGTTTGAACTCACGTACGGGATCAAAGACGTGCCA |
| | | | GGGGATCGCACAACAATCAAGCTCTATCTCAATGATCCGTACTACTCCATCGGGAA |
| | | | TCTGAAACCCGATACAGAGTACGAAGTCTCCCTCATCTCTTACACCGGGGACAGCT |
| | | | ACTCCAGATCCGGCTCCAATCCAGCCAAAATTACGTTTAAGACAGGCCTGCGGCTG |
| | | | GATGCTCCATCTCAAATAGAAGTTAAGGATGTGACGGATACGACGGCCCTCATCAC |
| | | | TTGGGTTGACCCTCGATATGACGATATTTGGTGGTTCGAATTGACGTATGGCATTA |
| | | | AGGACGTCCCAGGCGACCGGACAACTATTAAGCTGTATCTTAACGATCCTTATTAT |
| | | | AGCATCGGAAATCTCAAGCCGGATACCGAATATGAGGTTTCCCTCATTTCCTATAC |
| | | | TGGGGACTCCTACTCTCGCTCCGGCTCTAACCCAGCTAAGATCACTTTTAAAACCG |
| | | | GGCTTACTTCGAAAGGAAGCGGCGCCACAAACTTTAGCCTGCTGAAACAGGCCGGC |
| | | | GACGTCGAAGAAAATCCCGGGCCTATGGCTAAAAAGGGCTCTGTGGTCATCGTGGG |
| | | | CAGAATCAACCTGAGCGGCGATACCGCCTACGCTCAGCAGACAAGAGGCGAGGAAG |
| | | | GCTGCCAAGAGACAAGCCAGACCGGCAGAGACAAGAACCAGGTGGAAGGCGAGGTG |
| | | | CAGATCGTGTCTACAGCTACCCAGACCTTCCTGGCCACCAGCATCAATGGCGTGCT |
| | | | GTGGACAGTGTATCACGGCGCTGGCACCAGAACAATCGCCTCTCCAAAGGGCCCCG |
| | | | TGACACAGATGTATACCAACGTGGACAAGGACCTCGTCGGATGGCAAGCCCCTCAG |
| | | | GGCTCTAGAAGCCTGACACCTTGTACCTGCGGCAGCAGCGATCTGTACCTGGTCAC |
| | | | AAGACACGCCGACGTGATCCCCGTCAGAAGAAGAGGCGATAGCAGAGGCAGCCTGC |
| | | | TGAGCCCTAGACCTATCAGCTACCTGAAGGGATCTGCCGGCGGACCTCTGCTTTGT |
| | | | CCTGCTGGACATGCCGTGGGCATCTTTAGAGCCGCCGTGTCTACTAGAGGCGTGGC |
| | | | CAAGGCCGTGGACTTCATCCCTGTGGAAAGCCTGGAAACCACCATGCGGAGCCCCA |
| | | | CTAGAGATGAGTTTCCCACCATGGTGTTTCCTTCTGGGCAGATCAGCCAGGCCTCG |
| | | | GCCTTGGCCCCGGCCCCTCCCCAAGTCCTGCCCCAGGCTCCAGCCCCTGCCCCTGC |
| | | | TCCAGCCATGGTATCAGCTCTGGCCCAGGCCCCAGCCCCTGTCCCAGTCCTAGCCC |
| | | | CAGGCCCTCCTCAGGCTGTGGCCCCACCTGCCCCCAAGCCCACCCAGGCTGGGGAA |
| | | | GGAACGCTGTCAGAGGCCCTGCTGCAGCTGCAGTTTGATGATGAAGACCTGGGGGC |
| | | | CTTGCTTGGCAACAGCACAGACCCAGCTGTGTTCACAGACCTGGCATCCGTCGACA |
| | | | ACTCCGAGTTTCAGCAGCTGCTGAACCAGGGCATACCTGTGGCCCCCCACACAACT |
| | | | GAGCCCATGCTGATGGAGTACCCTGAGGCTATAACTCGCCTAGTGACAGGGGCCCA |
| | | | GAGGCCCCCCGACCCAGCTCCTGCTCCACTGGGGGCCCCGGGGCTCCCCAATGGCC |
| | | | TCCTTTCAGGAGATGAAGACTTCTCCTCCATTGCGGACATGGACTTCTCAGCCCTG |
| | | | CTGAGTCAGATCAGCTCCACTAGTTAT |
| 209 | hulL-2 | Protein | MYRMQLLSCIALSLALVTNSAPTSSSTKKTQLQLEHLLLDLQMILNGINNYKNPKL |
| | | | TRMLTFKFYMPKKATELKHLQCLEEELKPLEEVLNLAQSKNFHLRPRDLISNINVI |
| | | | VLELKGSETTFMCEYADETATIVEFLNRWITFCQSIISTLT |
| 210 | hulL-2 | DNA | ATGTACAGGATGCAACTCCTGTCTTGCATTGCACTAAGTCTTGCACTTGTCACAAA |
| | | | CAGTGCCCCTACCAGCAGCAGCACCAAGAAAACCCAGCTGCAACTGGAACACCTCC |
| | | | TGCTGGACCTGCAGATGATCCTGAACGGCATCAACAACTACAAGAACCCCAAGCTG |
| | | | ACCCGGATGCTGACCTTCAAGTTCTACATGCCCAAGAAGGCCACCGAGCTGAAGCA |
| | | | CCTCCAGTGCCTGGAAGAGGAACTGAAGCCCCTGGAAGAAGTGCTGAATCTGGCCC |
| | | | AGAGCAAGAACTTCCACCTGAGGCCTAGGGACCTGATCAGCAACATCAACGTGATC |
| | | | GTGCTGGAACTGAAAGGCAGCGAGACAACCTTCATGTGCGAGTACGCCGACGAGAC |
| | | | AGCTACCATCGTGGAATTTCTGAACCGGTGGATCACCTTCTGCCAGAGCATCATCA |
| | | | GCACCCTGACC |
| 211 | NS4A/3 PR | Protein | MGKKKGSVVIVGRINLSGDTAYAQQTRGEEGCQETSQTGRDKNQVEGEVQIVSTAT |
| | S139A K136D | | QTFLATSINGVLWTVYHGAGTRTIASPKGPVTQMYTNVDKDLVGWQAPQGSRSLTP |
| | | | CTCGSSDLYLVTRHADVIPVRRRGDSRGSLLSPRPISYLDGSAGGPLLCPAGHAVG |
| | | | IFRAAVSTRGVAKAVDFIPVESLETTMRSPT |
| 212 | NS4A/3 PR | DNA | ATGGGCAAGAAAAAGGGCAGCGTGGTCATCGTGGGCAGAATCAACCTGAGCGGCGA |
| | S139A K136D | | TACCGCCTACGCTCAGCAGACAAGAGGCGAGGAAGGCTGCCAAGAGACAAGCCAGA |
| | | | CCGGCAGAGACAAGAACCAGGTGGAAGGCGAGGTGCAGATCGTGTCTACAGCTACC |
| | | | CAGACCTTCCTGGCCACCAGCATCAATGGCGTGCTGTGGACAGTGTATCACGGCGC |
| | | | TGGCACCAGAACAATCGCCTCTCCAAAGGGCCCCGTGACACAGATGTACACCAACG |
| | | | TGGACAAGGACCTCGTCGGATGGCAAGCCCCTCAGGGAAGCAGAAGCCTGACACCT |
| | | | TGTACCTGCGGCAGCAGCGATCTGTACCTGGTCACAAGACACGCCGACGTGATCCC |
| | | | CGTCAGAAGAAGAGGCGATAGCAGAGGCAGCCTGCTGAGCCCTAGACCTATCAGCT |
| | | | ACCTGGATGGATCTGCCGGCGGACCTCTGCTTTGTCCTGCTGGACATGCCGTGGGC |
| | | | ATCTTTAGAGCCGCCGTGTCTACTAGAGGCGTGGCCAAAGCCGTGGACTTCATCCC |
| | | | CGTGGAAAGCCTGGAAACCACCATGAGATCTCCAACC |
| 213 | NS4A/3 PR | Protein | MGKKKGSVVIVGRINLSGDTAYAQQTRGEEGCQETSQTGRDKNQVEGEVQIVSTAT |
| | S139A D168E | | QTFLATSINGVLWTVYHGAGTRTIASPKGPVTQMYTNVDKDLVGWQAPQGSRSLTP |
| | | | CTCGSSDLYLVTRHADVIPVRRRGDSRGSLLSPRPISYLKGSAGGPLLCPAGHAVG |
| | | | IFRAAVSTRGVAKAVEFIPVESLETTMRSPT |
| 214 | NS4A/3 PR | DNA | ATGGGCAAGAAAAAGGGCAGCGTGGTCATCGTGGGCAGAATCAACCTGAGCGGCGA |
| | S139A D168E | | TACCGCCTACGCTCAGCAGACAAGAGGCGAGGAAGGCTGCCAAGAGACAAGCCAGA |
| | | | CCGGCAGAGACAAGAACCAGGTGGAAGGCGAGGTGCAGATCGTGTCTACAGCTACC |
| | | | CAGACCTTCCTGGCCACCAGCATCAATGGCGTGCTGTGGACAGTGTATCACGGCGC |
| | | | TGGCACCAGAACAATCGCCTCTCCAAAGGGCCCCGTGACACAGATGTACACCAACG |
| | | | TGGACAAGGACCTCGTCGGATGGCAAGCCCCTCAGGGAAGCAGAAGCCTGACACCT |
| | | | TGTACCTGCGGCAGCAGCGATCTGTACCTGGTCACAAGACACGCCGACGTGATCCC |
| | | | CGTCAGAAGAAGAGGCGATAGCAGAGGCAGCCTGCTGAGCCCTAGACCTATCAGCT |
| | | | ACCTGAAGGGATCTGCCGGCGGACCTCTGCTTTGTCCTGCTGGACATGCCGTGGGC |
| | | | ATCTTTAGAGCCGCCGTGTCTACTAGAGGCGTGGCCAAAGCCGTGGAATTCATCCC |
| | | | CGTGGAAAGCCTGGAAACCACCATGAGATCTCCAACC |
| 215 | NS4A/3 PR | Protein | MGKKKGSWIVGRINLSGDTAYAQQTRGEEGCQETSQTGRDKNQVEGEVQIVSTATQ |
| | S139A K136N | | TFLATSINGVLWTVYHGAGTRTIASPKGPVTQMYTNVDKDLVGWQAPQGSRSLTPC |
| | | | TCGSSDLYLVTRHADVIPVRRRGDSRGSLLSPRPISYLNGSAGGPLLCPAGHAVGI |
| | | | FRAAVSTRGVAKAVDFIPVESLETTMRSPT |
| 216 | NS4A/3 PR | DNA | ATGGGCAAGAAAAAGGGCAGCGTGGTCATCGTGGGCAGAATCAACCTGAGCGGCGA |
| | S139A K136N | | TACCGCCTACGCTCAGCAGACAAGAGGCGAGGAAGGCTGCCAAGAGACAAGCCAGA |
| | | | CCGGCAGAGACAAGAACCAGGTGGAAGGCGAGGTGCAGATCGTGTCTACAGCTACC |
| | | | CAGACCTTCCTGGCCACCAGCATCAATGGCGTGCTGTGGACAGTGTATCACGGCGC |
| | | | TGGCACCAGAACAATCGCCTCTCCAAAGGGCCCCGTGACACAGATGTACACCAACG |
| | | | TGGACAAGGACCTCGTCGGATGGCAGGCTCCTCAGGGCTCTAGAAGCCTGACACCT |
| | | | TGTACCTGCGGCAGCAGCGATCTGTACCTGGTCACAAGACACGCCGACGTGATCCC |
| | | | CGTCAGAAGAAGAGGCGATAGCAGAGGCAGCCTGCTGAGCCCTAGACCTATCAGCT |
| | | | ACCTGAACGGATCTGCCGGCGGACCTCTGCTTTGTCCTGCTGGACATGCCGTGGGC |
| | | | ATCTTTAGAGCCGCCGTGTCTACTAGAGGCGTGGCCAAAGCCGTGGACTTCATCCC |
| | | | TGTGGAAAGCCTGGAAACCACCATGAGAAGCCCCACC |
| 217 | His-TEV- | Protein | MGSSHHHHHHGSENLYFQSKKKGSVVIVGRINLSGDTAYAQQTRGEEGCQETSQTG |
| | NS4A/3 PR | | RDKNQVEGEVQIVSTATQTFLATSINGVLWTVYHGAGTRTIASPKGPVTQMYTNVD |
| | S139A | | KDLVGWQAPQGSRSLTPCTCGSSDLYLVTRHADVIPVRRRGDSRGSLLSPRPISYL |
| | | | KGSAGGPLLCPAGHAVGIFRAAVSTRGVAKAVDFIPVESLETTMRSP |
| 218 | His-TEV- | DNA | ATGGGTAGCAGCCATCACCATCATCATCATGGTAGCGAAAATCTGTATTTCCAGAG |
| | NS4A/3 PR | | CAAAAAAAAGGGCAGCGTTGTTATTGTGGGTCGTATTAATCTGAGCGGTGATACCG |
| | S139A | | CATATGCACAGCAGACCCGTGGTGAAGAAGGTTGTCAAGAAACCAGCCAGACCGGT |
| | | | CGTGATAAAAATCAGGTTGAAGGTGAAGTTCAGATTGTTAGCACCGCAACACAGAC |
| | | | CTTTCTGGCAACCAGCATTAATGGTGTTCTGTGGACCGTTTATCATGGTGCAGGCA |
| | | | CCCGTACCATTGCAAGCCCGAAAGGTCCGGTTACACAGATGTATACCAATGTGGAT |
| | | | AAAGATCTGGTTGGTTGGCAGGCACCGCAGGGTAGCCGTAGTCTGACCCCGTGTAC |
| | | | CTGTGGTAGCAGCGATCTGTATCTGGTTACCCGTCATGCAGATGTTATTCCGGTTC |
| | | | GTCGTCGTGGTGATAGCCGTGGTAGCCTGCTGAGTCCGCGTCCGATTAGCTATCTG |
| | | | AAAGGTAGTGCCGGTGGTCCGCTGCTGTGTCCGGCAGGTCATGCAGTTGGTATTTT |
| | | | TCGTGCAGCAGTTAGCACCCGTGGCGTTGCAAAAGCAGTTGATTTTATCCCGGTTG |
| | | | AAAGCCTGGAAACCACCATGCGTAGCCCG |
| 219 | NS4A/3 | Protein | SKKKGSVVIVGRINLSGDTAYAQQTRGEEGCQETSQTGRDKNQVEGEVQIVSTATQ |
| | S139A post- | | TFLATSINGVLWTVYHGAGTRTIASPKGPVTQMYTNVDKDLVGWQAPQGSRSLTPC |
| | TEV cleavage | | TCGSSDLYLVTRHADVIPVRRRGDSRGSLLSPRPISYLKGSAGGPLLCPAGHAVGI |
| | | | FRAAVSTRGVAKAVDFIPVESLETTMRSP |
| 220 | pelB-PRSIM_57- | Protein | MKYLLPTAAAGLLLLAAQPAMAQVQLVQSGAEVKKPGSSVKVSCKASGGTFSSYAI |
| | TEV-His | | SWVRQAPGQGLEWMGGIIPIFGTANYAQKFQGRVTITADESTSTAYMELSSLRSED |
| | | | TAVYYCARHTNYITVFDYWGQGTLVTVSSGGGGSGGGGSGGGGSAQSVLTQPPSAS |
| | | | GTPGQRVTISCSGSSSNIGSNTVNWYQQLPGTAPKLLIYSNNQRPSGVPDRFSGSK |
| | | | SGTSASLAISGLQSEDEADYYCAAWDHHWEQVVFGGGTKLTVLENLYFQSHHHHHH |
| 221 | pelB_PRSIM_57- | DNA | ATGAAATATCTGCTGCCGACCGCAGCAGCGGGTCTGCTGCTGCTGGCAGCACAGCC |
| | TEV-His | | TGCAATGGCACAGGTTCAGCTGGTTCAGAGCGGTGCAGAAGTTAAAAAACCGGGTA |
| | | | GCAGCGTTAAAGTTAGCTGTAAAGCAAGCGGTGGCACCTTTAGCAGCTATGCAATT |
| | | | AGCTGGGTTCGTCAGGCACCTGGTCAAGGTCTGGAATGGATGGGTGGTATTATTCC |
| | | | GATTTTTGGCACCGCAAATTATGCCCAGAAATTTCAGGGTCGTGTTACCATTACCG |
| | | | CAGATGAAAGCACCAGCACCGCATATATGGAACTGAGCAGCCTGCGTAGCGAAGAT |
| | | | ACCGCAGTGTATTATTGTGCACGTCATACCAACTATATCACCGTGTTTGATTATTG |
| | | | GGGTCAGGGCACCCTGGTTACCGTTAGCAGCGGTGGTGGTGGTAGCGGTGGCGGAG |
| | | | GTTCAGGTGGTGGCGGTTCAGCACAGAGCGTTCTGACCCAGCCTCCGAGCGCAAGC |
| | | | GGTACACCGGGTCAGCGTGTGACCATTAGCTGTAGCGGTAGCAGCAGTAATATTGG |
| | | | TAGCAATACCGTTAATTGGTATCAGCAGCTGCCAGGCACCGCACCGAAACTGCTGA |
| | | | TTTATAGCAATAATCAGCGTCCGAGCGGTGTTCCGGATCGTTTTAGCGGTAGTAAA |
| | | | AGCGGCACCAGCGCAAGCCTGGCAATTAGCGGTCTGCAGAGCGAAGATGAAGCAGA |
| | | | TTATTACTGTGCAGCATGGGATCATCATTGGGAACAAGTTGTTTTTGGTGGTGGCA |
| | | | CCAAACTGACCGTTCTGGAAAATCTGTATTTCCAGAGCCATCACCATCATCATCAT |
| 222 | PRSIM_57 | Protein | QVQLVQSGAEVKKPGSSVKVSCKASGGTFSSYAISWVRQAPGQGLEWMGGHPIFGT |
| | post-TEV | | ANYAQKFQGRVTITADESTSTAYMELSSLRSEDTAVYYCARHTNYITVFDYWGQGT |
| | cleavage, | | LVTVSSGGGGSGGGGSGGGGSAQSVLTQPPSASGTPGQRVTISCSGSSSNIGSNTV |
| | pelB removal | | NWYQQLPGTAPKLLIYSNNQRPSGVPDRFSGSKSGTSASLAISGLQSEDEADYYCA |
| | | | AWDHHWEQVVFGGGTKLTVLENLYFQ |
| 223 | PRSIM23_NS | Protein | MGSRLDAPSQIEVKDVTDTTALITWVDPRYDDIWWFELTYGIKDVPGDRTTIKLYL |
| | 4A/3 PR | | NDPYYSIGNLKPDTEYEVSLISYTGDSYSRSGSNPAKITFKTGLGGGSGMKKKGSV |
| | S139A_DCasp9 | | VIVGRINLSGDTAYAQQTRGEEGCQETSQTGRDKNQVEGEVQIVSTATQTFLATSI |
| | | | NGVLWTVYHGAGTRTIASPKGPVTQMYTNVDKDLVGWQAPQGSRSLTPCTCGSSDL |
| | | | YLVTRHADVIPVRRRGDSRGSLLSPRPISYLKGSAGGPLLCPAGHAVGIFRAAVST |
| | | | RGVAKAVDFIPVESLETTMRSPGGGSGVDGFGDVGALESLRGNADLAYILSMEPCG |
| | | | HCLIINNVNFCRESGLRTRTGSNIDCEKLRRRFSSLHFMVEVKGDLTAKKMVLALL |
| | | | ELARQDHGALDCCVVVILSHGCQASHLQFPGAVYGTDGCPVSVEKIVNIFNGTSCP |
| | | | SLGGKPKLFFIQACGGEQKDHGFEVASTSPEDESPGSNPEPDATPFQEGLRTFDQL |
| | | | DAISSLPTPSDIFVSYSTFPGFVSWRDPKSGSWYVETLDDIFEQWAHSEDLQSLLL |
| | | | RVANAVSVKGIYKQMPGCFNFLRKKLFFKTS |
| 224 | PRSIM23_NS | DNA | ATGGGCTCTAGACTGGATGCCCCTAGCCAGATCGAAGTGAAGGACGTGACCGACAC |
| | 4A/3 PR | | CACCGCTCTGATCACCTGGGTTGACCCCAGATACGACGATATCTGGTGGTTCGAGC |
| | S139A_DCasp9 | | TGACCTACGGCATCAAGGATGTGCCCGGCGACAGAACCACCATCAAGCTGTACCTG |
| | | | AACGACCCCTACTACAGCATCGGCAACCTGAAGCCTGACACCGAGTACGAGGTGTC |
| | | | CCTGATCAGCTACACCGGCGACTCCTACAGCAGAAGCGGCAGCAATCCTGCCAAGA |
| | | | TCACCTTCAAGACCGGCCTTGGGGGCGGATCCGGCATGAAGAAAAAGGGCTCTGTG |
| | | | GTCATCGTGGGCAGAATCAACCTGAGCGGCGATACCGCCTACGCTCAGCAGACAAG |
| | | | AGGCGAGGAAGGCTGCCAAGAGACAAGCCAGACCGGCAGAGACAAGAACCAGGTGG |
| | | | AAGGCGAGGTGCAGATCGTGTCTACAGCTACCCAGACCTTCCTGGCCACCAGCATC |
| | | | AATGGCGTGCTGTGGACAGTGTATCACGGCGCTGGCACCAGAACAATCGCCTCTCC |
| | | | AAAGGGCCCCGTGACACAGATGTACACCAACGTGGACAAGGACCTCGTCGGATGGC |
| | | | AAGCCCCTCAGGGCTCTAGAAGCCTGACACCTTGTACCTGCGGCAGCAGCGATCTG |
| | | | TACCTGGTCACAAGACACGCCGACGTGATCCCCGTCAGAAGAAGAGGCGATAGCAG |
| | | | AGGCAGCCTGCTGAGCCCTAGACCTATCAGCTACCTGAAGGGATCTGCCGGCGGAC |
| | | | CTCTGCTTTGTCCTGCTGGACATGCCGTGGGCATCTTTAGAGCCGCCGTGTCTACT |
| | | | AGAGGCGTGGCCAAAGCCGTGGACTTCATCCCTGTGGAAAGCCTGGAAACCACCAT |
| | | | GCGGAGCCCCGGGGGAGGCTCCGGCGTGGATGGCTTTGGAGATGTGGGCGCCCTGG |
| | | | AATCCCTGAGAGGAAATGCCGATCTGGCCTACATCCTGAGCATGGAACCTTGCGGC |
| | | | CACTGCCTGATTATCAACAATGTGAACTTCTGCCGCGAGAGCGGCCTGAGAACAAG |
| | | | AACCGGCAGCAACATCGATTGCGAGAAGCTGCGGAGAAGATTCAGCAGCCTGCACT |
| | | | TCATGGTGGAAGTGAAGGGCGACCTGACCGCCAAGAAAATGGTGCTGGCTCTGCTG |
| | | | GAACTGGCCAGACAGGATCATGGCGCACTGGATTGCTGCGTGGTCGTGATTCTGAG |
| | | | CCACGGCTGTCAGGCCAGCCATCTGCAATTCCCTGGCGCCGTGTATGGCACCGATG |
| | | | GCTGTCCTGTGTCCGTGGAAAAGATCGTGAACATCTTCAACGGCACCAGCTGTCCT |
| | | | AGCCTCGGCGGAAAGCCCAAGCTGTTCTTCATCCAAGCCTGTGGCGGCGAGCAGAA |
| | | | GGATCACGGATTTGAGGTGGCCAGCACAAGCCCCGAGGATGAGAGCCCTGGAAGCA |
| | | | ACCCTGAGCCTGACGCCACACCTTTCCAAGAGGGACTGAGAACCTTCGACCAGCTG |
| | | | GACGCTATCAGCTCCCTGCCTACACCTAGCGACATCTTCGTGTCCTACAGCACATT |
| | | | CCCCGGCTTTGTGTCTTGGCGGGACCCCAAGTCTGGCTCTTGGTACGTGGAAACCC |
| | | | TGGATGACATCTTCGAGCAGTGGGCCCATAGCGAGGACCTGCAATCTCTGCTGCTG |
| | | | AGAGTGGCCAATGCCGTGTCCGTGAAGGGCATCTACAAGCAGATGCCCGGCTGCTT |
| | | | CAACTTCCTGCGGAAGAAGCTGTTTTTCAAGACCAGCTGATAG |
| 225 | NS4A/3 PR | Protein | MGKKKGSWIVGRINLSGDTAYAQQTRGEEGCQETSQTGRDKNQVEGEVQIVSTATQ |
| | S139A-VPR | | TFLATSINGVLWTVYHGAGTRTIASPKGPVTQMYTNVDKDLVGWQAPQGSRSLTPC |
| | | | TCGSSDLYLVTRHADVIPVRRRGDSRGSLLSPRPISYLKGSAGGPLLCPAGHAVGI |
| | | | FRAAVSTRGVAKAVDFIPVESLETTMRSPTGGGGSGGGGSEASGSGRADALDDFDL |
| | | | DMLGSDALDDFDLDMLGSDALDDFDLDMLGSDALDDFDLDMLINSRSSGSPKKKRK |
| | | | VGSQYLPDTDDRHRIEEKRKRTYETFKSIMKKSPFSGPTDPRPPPRRIAVPSRSSA |
| | | | SVPKPAPQPYPFTSSLSTINYDEFPTMVFPSGQISQASALAPAPPQVLPQAPAPAP |
| | | | APAMVSALAQAPAPVPVLAPGPPQAVAPPAPKPTQAGEGTLSEALLQLQFDDEDLG |
| | | | ALLGNSTDPAVFTDLASVDNSEFQQLLNQGIPVAPHTTEPMLMEYPEAITRLVTGA |
| | | | QRPPDPAPAPLGAPGLPNGLLSGDEDFSSIADMDFSALLGSGSGSRDSREGMFLPK |
| | | | PEAGSAISDVFEGREVCQPKRIRPFHPPGSPWANRPLPASLAPTPTGPVHEPVGSL |
| | | | TPAPVPQPLDPAPAVTPEASHLLEDPDEETSQAVKALREMADTVIPQKEEAAICGQ |
| | | | MDLSHPPPRGHLDELTTTLESMTEDLNLDSPLTPELNEILDTFLNDECLLHAMHIS |
| | | | TGLSIFDTSLF |
| 226 | NS4A/3 PR | DNA | ATGGGCAAGAAAAAGGGCAGCGTGGTCATCGTGGGCAGAATCAACCTGAGCGGCGA |
| | S139A-VPR | | TACCGCCTACGCTCAGCAGACAAGAGGCGAGGAAGGCTGCCAAGAGACAAGCCAGA |
| | | | CCGGCAGAGACAAGAACCAGGTGGAAGGCGAGGTGCAGATCGTGTCTACAGCTACC |
| | | | CAGACCTTCCTGGCCACCAGCATCAATGGCGTGCTGTGGACAGTGTATCACGGCGC |
| | | | TGGCACCAGAACAATCGCCTCTCCAAAGGGCCCCGTGACACAGATGTACACCAACG |
| | | | TGGACAAGGACCTCGTCGGATGGCAAGCCCCTCAGGGCTCCAGAAGCCTGACACCT |
| | | | TGTACCTGCGGCAGCAGCGATCTGTACCTGGTCACAAGACACGCCGACGTGATCCC |
| | | | CGTCAGAAGAAGAGGCGATAGCAGAGGCAGCCTGCTGAGCCCTAGACCTATCAGCT |
| | | | ACCTGAAGGGATCTGCCGGCGGACCTCTGCTTTGTCCTGCTGGACATGCCGTGGGC |
| | | | ATCTTTAGAGCCGCCGTGTCTACTAGAGGCGTGGCCAAAGCCGTGGACTTCATCCC |
| | | | TGTGGAAAGCCTGGAAACCACCATGAGAAGCCCCACCGGTGGCGGCGGATCTGGCG |
| | | | GAGGTGGAAGTGAAGCTTCTGGCAGCGGTAGAGCCGACGCTCTGGACGACTTCGAC |
| | | | CTGGATATGCTGGGCTCTGACGCCCTGGATGATTTTGATCTGGACATGCTCGGCAG |
| | | | CGACGCCCTCGACGATTTCGATCTCGATATGTTGGGAAGCGACGCACTTGATGACT |
| | | | TTGACCTCGACATGTTGATCAATAGCAGAAGCAGCGGCAGCCCCAAGAAAAAGCGG |
| | | | AAAGTGGGCAGCCAGTACCTGCCTGACACCGACGACAGACACCGGATCGAAGAGAA |
| | | | GCGGAAGCGGACCTACGAGACATTCAAGAGCATCATGAAGAAGTCCCCATTCAGCG |
| | | | GCCCCACCGATCCTAGACCTCCACCTAGAAGAATCGCCGTGCCTAGCAGATCTAGC |
| | | | GCCAGCGTGCCAAAACCTGCTCCTCAGCCTTATCCTTTCACCAGCAGCCTGAGCAC |
| | | | CATCAACTACGACGAGTTCCCTACCATGGTGTTCCCCAGCGGCCAGATCTCTCAGG |
| | | | CTTCTGCTCTTGCTCCAGCTCCTCCTCAGGTTCTGCCTCAAGCTCCTGCACCAGCA |
| | | | CCGGCTCCAGCTATGGTTTCTGCTTTGGCTCAGGCCCCTGCTCCTGTGCCTGTTCT |
| | | | TGCTCCTGGACCACCTCAGGCTGTTGCTCCTCCTGCTCCAAAACCTACACAGGCCG |
| | | | GCGAGGGAACACTGTCTGAAGCTCTGCTGCAGCTCCAGTTCGACGACGAAGATCTG |
| | | | GGAGCCCTGCTGGGCAATAGCACAGATCCTGCCGTGTTCACCGATCTGGCCAGCGT |
| | | | GGACAATAGCGAGTTCCAGCAGCTCCTGAATCAGGGCATCCCTGTGGCTCCTCACA |
| | | | CCACCGAACCTATGCTGATGGAATACCCCGAGGCCATCACCAGACTGGTCACCGGC |
| | | | GCTCAAAGACCACCTGATCCAGCTCCAGCACCTCTGGGAGCACCAGGACTGCCTAA |
| | | | TGGACTGCTGTCTGGCGACGAGGACTTCAGCTCTATCGCCGACATGGATTTCAGCG |
| | | | CCCTGCTCGGCTCTGGCTCCGGCTCTAGAGATAGCAGAGAAGGCATGTTCCTGCCT |
| | | | AAGCCTGAGGCCGGCTCTGCCATCTCCGATGTGTTTGAGGGCAGAGAAGTGTGCCA |
| | | | GCCTAAGCGGATCCGGCCTTTTCACCCTCCTGGAAGCCCTTGGGCCAACAGACCTC |
| | | | TGCCTGCTTCTCTGGCCCCTACACCAACAGGACCTGTGCACGAACCTGTGGGCAGT |
| | | | CTGACCCCAGCTCCTGTTCCTCAACCTCTGGATCCCGCTCCTGCTGTGACACCTGA |
| | | | AGCCTCTCATCTGCTGGAAGATCCCGACGAAGAGACAAGCCAGGCCGTGAAGGCCC |
| | | | TGAGAGAAATGGCCGACACAGTGATCCCTCAGAAAGAGGAAGCCGCCATCTGCGGA |
| | | | CAGATGGACCTGTCTCATCCTCCACCAAGAGGCCACCTGGACGAGCTGACAACCAC |
| | | | ACTGGAATCCATGACCGAGGACCTGAACCTGGACAGCCCTCTGACACCCGAGCTGA |
| | | | ACGAGATCCTGGACACCTTCCTGAACGACGAGTGTCTGCTGCACGCCATGCACATC |
| | | | TCTACCGGCCTGAGCATCTTCGACACCAGCCTGTTC |
| 227 | spdCas9- | Protein | MDYYPYDVPDYADKKYSIGLAIGTNSVGWAVITDEYKVPSKKFKVLGNTDRHSIKK |
| | PRSIM_23x3 | | NLIGALLFDSGETAEATRLKRTARRRYTRRKNRICYLQEIFSNEMAKVDDSFFHRL |
| | | | EESFLVEEDKKHERHPIFGNIVDEVAYHEKYPTIYHLRKKLVDSTDKADLRLIYLA |
| | | | LAHMIKFRGHFLIEGDLNPDNSDVDKLFIQLVQTYNQLFEENPINASGVDAKAILS |
| | | | ARLSKSRRLENLIAQLPGEKKNGLFGNLIALSLGLTPNFKSNFDLAEDAKLQLSKD |
| | | | TYDDDLDNLLAQIGDQYADLFLAAKNLSDAILLSDILRVNTEITKAPLSASMIKRY |
| | | | DEHHQDLTLLKALVRQQLPEKYKEIFFDQSKNGYAGYIDGGASQEEFYKFIKPILE |
| | | | KMDGTEELLVKLNREDLLRKQRTFDNGSIPHQIHLGELHAILRRQEDFYPFLKDNR |
| | | | EKIEKILTFRIPYYVGPLARGNSRFAWMTRKSEETITPWNFEEVVDKGASAQSFIE |
| | | | RMTNFDKNLPNEKVLPKHSLLYEYFTVYNELTKVKYVTEGMRKPAFLSGEQKKAIV |
| | | | DLLFKTNRKVTVKQLKEDYFKKIECFDSVEISGVEDRFNASLGTYHDLLKIIKDKD |
| | | | FLDNEENEDILEDIVLTLTLFEDREMIEERLKTYAHLFDDKVMKQLKRRRYTGWGR |
| | | | LSRKLINGIRDKQSGKTILDFLKSDGFANRNFMQLIHDDSLTFKEDIQKAQVSGQG |
| | | | DSLHEHIANLAGSPAIKKGILQTVKVVDELVKVMGRHKPENIVIEMARENQTTQKG |
| | | | QKNSRERMKRIEEGIKELGSQILKEHPVENTQLQNEKLYLYYLQNGRDMYVDQELD |
| | | | INRLSDYDVDAIVPQSFLKDDSIDNKVLTRSDKNRGKSDNVPSEEVVKKMKNYWRQ |
| | | | LLNAKLITQRKFDNLTKAERGGLSELDKAGFIKRQLVETRQITKHVAQILDSRMNT |
| | | | KYDENDKLIREVKVITLKSKLVSDFRKDFQFYKVREINNYHHAHDAYLNAVVGTAL |
| | | | IKKYPKLESEFVYGDYKVYDVRKMIAKSEQEIGKATAKYFFYSNIMNFFKTEITLA |
| | | | NGEIRKRPLIETNGETGEIVWDKGRDFATVRKVLSMPQVNIVKKTEVQTGGFSKES |
| | | | ILPKRNSDKLIARKKDWDPKKYGGFDSPTVAYSVLVVAKVEKGKSKKLKSVKELLG |
| | | | ITIMERSSFEKNPIDFLEAKGYKEVKKDLIIKLPKYSLFELENGRKRMLASAGELQ |
| | | | KGNELALPSKYVNFLYLASHYEKLKGSPEDNEQKQLFVEQHKHYLDEIIEQISEFS |
| | | | KRVILADANLDKVLSAYNKHRDKPIREQAENIIHLFTLTNLGAPAAFKYFDTTIDR |
| | | | KRYTSTKEVLDATLIHQSITGLYETRIDLSQLGGDPKKKRKVGSAGSRLDAPSQIE |
| | | | VKDVTDTTALITWVDPRYDDIWWFELTYGIKDVPGDRTTIKLYLNDPYYSIGNLKP |
| | | | DTEYEVSLISYTGDSYSRSGSNPAKITFKTGLRLDAPSQIEVKDVTDTTALITWVD |
| | | | PRYDDIWWFELTYGIKDVPGDRTTIKLYLNDPYYSIGNLKPDTEYEVSLISYTGDS |
| | | | YSRSGSNPAKITFKTGLRLDAPSQIEVKDVTDTTALITWVDPRYDDIWWFELTYGI |
| | | | KDVPGDRTTIKLYLNDPYYSIGNLKPDTEYEVSLISYTGDSYSRSGSNPAKITFKT |
| | | | GL |
| 228 | spdCas9- | DNA | ATGGACTATTACCCCTACGACGTGCCCGATTACGCCGACAAGAAGTATTCTATCGG |
| | PRSIM_23x3 | | ACTGGCCATCGGGACTAATAGCGTCGGGTGGGCCGTGATCACTGACGAGTACAAGG |
| | | | TGCCCTCTAAGAAGTTCAAGGTGCTCGGGAACACCGACCGGCATTCCATCAAGAAA |
| | | | AATCTGATCGGAGCTCTCCTCTTTGATTCAGGGGAAACCGCTGAAGCAACCCGCCT |
| | | | CAAGCGGACTGCTAGACGGCGGTACACCAGGAGGAAGAACCGGATTTGTTACCTTC |
| | | | AAGAGATATTCTCCAACGAAATGGCAAAGGTCGACGACAGCTTCTTCCATAGGCTG |
| | | | GAAGAATCATTCCTCGTGGAAGAGGATAAGAAGCATGAACGGCATCCCATCTTCGG |
| | | | TAATATCGTCGACGAGGTGGCCTATCACGAGAAATACCCAACCATCTACCATCTTC |
| | | | GCAAAAAGCTGGTGGACTCAACCGACAAGGCAGACCTCCGGCTTATCTACCTGGCC |
| | | | CTGGCCCACATGATCAAGTTCAGAGGCCACTTCCTGATCGAGGGCGACCTCAATCC |
| | | | TGACAATAGCGATGTGGATAAACTGTTCATCCAGCTGGTGCAGACTTACAACCAGC |
| | | | TCTTTGAAGAGAACCCCATCAATGCAAGCGGAGTCGATGCCAAGGCCATTCTGTCA |
| | | | GCCCGGCTGTCAAAGAGCCGCAGACTTGAGAATCTTATCGCTCAGCTGCCGGGTGA |
| | | | AAAGAAAAATGGACTGTTCGGGAACCTGATTGCTCTTTCACTTGGGCTGACTCCCA |
| | | | ATTTCAAGTCTAATTTCGACCTGGCAGAGGATGCCAAGCTGCAACTGTCCAAGGAC |
| | | | ACCTATGATGACGATCTCGACAACCTCCTGGCCCAGATCGGTGACCAATACGCCGA |
| | | | CCTTTTCCTTGCTGCTAAGAATCTTTCTGACGCCATCCTGCTGTCTGACATTCTCC |
| | | | GCGTGAACACTGAAATCACCAAGGCCCCTCTTTCAGCTTCAATGATTAAGCGGTAT |
| | | | GATGAGCACCACCAGGACCTGACCCTGCTTAAGGCACTCGTCCGGCAGCAGCTTCC |
| | | | GGAGAAGTACAAGGAAATCTTCTTTGACCAGTCAAAGAATGGATACGCCGGCTACA |
| | | | TCGACGGAGGTGCCTCCCAAGAGGAATTTTATAAGTTTATCAAACCTATCCTTGAG |
| | | | AAGATGGACGGCACCGAAGAGCTCCTCGTGAAACTGAATCGGGAGGATCTGCTGCG |
| | | | GAAGCAGCGCACTTTCGACAATGGGAGCATTCCCCACCAGATCCATCTTGGGGAGC |
| | | | TTCACGCCATCCTTCGGCGCCAAGAGGACTTCTACCCCTTTCTTAAGGACAACAGG |
| | | | GAGAAGATTGAGAAAATTCTCACTTTCCGCATCCCCTACTACGTGGGACCCCTCGC |
| | | | CAGAGGAAATAGCCGGTTTGCTTGGATGACCAGAAAGTCAGAAGAAACTATCACTC |
| | | | CCTGGAACTTCGAAGAGGTGGTGGACAAGGGAGCCAGCGCTCAGTCATTCATCGAA |
| | | | CGGATGACTAACTTCGATAAGAACCTCCCCAATGAGAAGGTCCTGCCGAAACATTC |
| | | | CCTGCTCTACGAGTACTTTACCGTGTACAACGAGCTGACCAAGGTGAAATATGTCA |
| | | | CCGAAGGGATGAGGAAGCCCGCATTCCTGTCAGGCGAACAAAAGAAGGCAATTGTG |
| | | | GACCTTCTGTTCAAGACCAATAGAAAGGTGACCGTGAAGCAGCTGAAGGAGGACTA |
| | | | TTTCAAGAAAATTGAATGCTTCGACTCTGTGGAGATTAGCGGGGTCGAAGATCGGT |
| | | | TCAACGCAAGCCTGGGTACCTACCATGATCTGCTTAAGATCATCAAGGACAAGGAT |
| | | | TTTCTGGACAATGAGGAGAACGAGGACATCCTTGAGGACATTGTCCTGACTCTCAC |
| | | | TCTGTTCGAGGACCGGGAAATGATCGAGGAGAGGCTTAAGACCTACGCCCATCTGT |
| | | | TCGACGATAAAGTGATGAAGCAACTTAAACGGAGAAGATATACCGGATGGGGACGC |
| | | | CTTAGCCGCAAACTCATCAACGGAATCCGGGACAAACAGAGCGGAAAGACCATTCT |
| | | | TGATTTCCTTAAGAGCGACGGATTCGCTAATCGCAACTTCATGCAACTTATCCATG |
| | | | ATGATTCCCTGACCTTTAAGGAGGACATCCAGAAGGCCCAAGTGTCTGGACAAGGT |
| | | | GACTCACTGCACGAGCATATCGCAAATCTGGCTGGTTCACCCGCTATTAAGAAGGG |
| | | | TATTCTCCAGACCGTGAAAGTCGTGGACGAGCTGGTCAAGGTGATGGGTCGCCATA |
| | | | AACCAGAGAACATTGTCATCGAGATGGCCAGGGAAAACCAGACTACCCAGAAGGGA |
| | | | CAGAAGAACAGCAGGGAGCGGATGAAAAGAATTGAGGAAGGGATTAAGGAGCTCGG |
| | | | GTCACAGATCCTTAAAGAGCACCCGGTGGAAAACACCCAGCTTCAGAATGAGAAGC |
| | | | TCTATCTGTACTACCTTCAAAATGGACGCGATATGTATGTGGACCAAGAGCTTGAT |
| | | | ATCAACAGGCTCTCAGACTACGACGTGGACGCCATCGTCCCTCAGAGCTTCCTCAA |
| | | | AGACGACTCAATTGACAATAAGGTGCTGACTCGCTCAGACAAGAACCGGGGAAAGT |
| | | | CAGATAACGTGCCCTCAGAGGAAGTCGTGAAAAAGATGAAGAACTATTGGCGCCAG |
| | | | CTTCTGAACGCAAAGCTGATCACTCAGCGGAAGTTCGACAATCTCACTAAGGCTGA |
| | | | GAGGGGCGGACTGAGCGAACTGGACAAAGCAGGATTCATTAAACGGCAACTTGTGG |
| | | | AGACTCGGCAGATTACTAAACATGTCGCCCAAATCCTTGACTCACGCATGAATACC |
| | | | AAGTACGACGAAAACGACAAACTTATCCGCGAGGTGAAGGTGATTACCCTGAAGTC |
| | | | CAAGCTGGTCAGCGATTTCAGAAAGGACTTTCAATTCTACAAAGTGCGGGAGATCA |
| | | | ATAACTATCATCATGCTCATGACGCATATCTGAATGCCGTGGTGGGAACCGCCCTG |
| | | | ATCAAGAAGTACCCAAAGCTGGAAAGCGAGTTCGTGTACGGAGACTACAAGGTCTA |
| | | | CGACGTGCGCAAGATGATTGCCAAATCTGAGCAGGAGATCGGAAAGGCCACCGCAA |
| | | | AGTACTTCTTCTACAGCAACATCATGAATTTCTTCAAGACCGAAATCACCCTTGCA |
| | | | AACGGTGAGATCCGGAAGAGGCCGCTCATCGAGACTAATGGGGAGACTGGCGAAAT |
| | | | CGTGTGGGACAAGGGCAGAGATTTCGCTACCGTGCGCAAAGTGCTTTCTATGCCTC |
| | | | AAGTGAACATCGTGAAGAAAACCGAGGTGCAAACCGGAGGCTTTTCTAAGGAATCA |
| | | | ATCCTCCCCAAGCGCAACTCCGACAAGCTCATTGCAAGGAAGAAGGATTGGGACCC |
| | | | TAAGAAGTACGGCGGATTCGATTCACCAACTGTGGCTTATTCTGTCCTGGTCGTGG |
| | | | CTAAGGTGGAAAAAGGAAAGTCTAAGAAGCTCAAGAGCGTGAAGGAACTGCTGGGT |
| | | | ATCACCATTATGGAGCGCAGCTCCTTCGAGAAGAACCCAATTGACTTTCTCGAAGC |
| | | | CAAAGGTTACAAGGAAGTCAAGAAGGACCTTATCATCAAGCTCCCAAAGTATAGCC |
| | | | TGTTCGAACTGGAGAATGGGCGGAAGCGGATGCTCGCCTCCGCTGGCGAACTTCAG |
| | | | AAGGGTAATGAGCTGGCTCTCCCCTCCAAGTACGTGAATTTCCTCTACCTTGCAAG |
| | | | CCATTACGAGAAGCTGAAGGGGAGCCCCGAGGACAACGAGCAAAAGCAACTGTTTG |
| | | | TGGAGCAGCATAAGCATTATCTGGACGAGATCATTGAGCAGATTTCCGAGTTTTCT |
| | | | AAACGCGTCATTCTCGCTGATGCCAACCTCGATAAAGTCCTTAGCGCATACAATAA |
| | | | GCACAGAGACAAACCAATTCGGGAGCAGGCTGAGAATATCATCCACCTGTTCACCC |
| | | | TCACCAATCTTGGTGCCCCTGCCGCATTCAAGTACTTCGACACCACCATCGACCGG |
| | | | AAACGCTATACCTCCACCAAAGAAGTGCTGGACGCCACCCTCATCCACCAGAGCAT |
| | | | CACCGGACTTTACGAAACTCGGATTGACCTCTCACAGCTCGGAGGGGATCCCAAGA |
| | | | AGAAGCGGAAAGTCGGCAGCGCTGGCTCTAGACTGGATGCCCCTAGCCAGATCGAA |
| | | | GTGAAGGACGTGACCGACACCACCGCTCTGATCACCTGGGTTGACCCCAGATACGA |
| | | | CGACATCTGGTGGTTCGAGCTGACCTACGGCATCAAGGATGTGCCCGGCGACAGAA |
| | | | CCACCATCAAGCTGTACCTGAACGACCCCTACTACAGCATCGGCAACCTGAAGCCT |
| | | | GACACCGAGTACGAGGTGTCCCTGATCAGCTACACCGGCGACTCCTACAGCAGAAG |
| | | | CGGCAGCAATCCTGCCAAGATCACCTTCAAGACCGGCCTGAGACTGGACGCACCCT |
| | | | CTCAGATTGAAGTCAAAGATGTCACCGACACGACAGCCCTGATTACATGGGTTGAC |
| | | | CCTCGCTACGATGATATTTGGTGGTTTGAACTCACGTACGGGATCAAAGACGTGCC |
| | | | AGGGGATCGCACAACAATCAAGCTCTATCTCAATGATCCGTACTACTCCATCGGGA |
| | | | ATCTGAAACCCGATACAGAGTACGAAGTCTCCCTCATCTCTTACACCGGGGACAGC |
| | | | TACTCCAGATCCGGCTCCAATCCAGCCAAAATTACGTTTAAGACAGGCCTGCGGCT |
| | | | GGATGCTCCATCTCAAATAGAAGTTAAGGATGTGACGGATACGACGGCCCTCATCA |
| | | | CTTGGGTTGACCCTCGATATGACGATATTTGGTGGTTCGAATTGACGTATGGCATT |
| | | | AAGGACGTCCCAGGCGACCGGACAACTATTAAGCTGTATCTTAACGATCCTTATTA |
| | | | TAGCATCGGAAATCTCAAGCCGGATACCGAATATGAGGTTTCCCTCATTTCCTATA |
| | | | CTGGGGACTCCTACTCTCGCTCCGGCTCTAACCCAGCTAAGATCACTTTTAAAACC |
| | | | GGGCTTTAA |
| 229 | gRNA IL-2 | DNA | GTTACATTAGCCCACACTT |
| 230 | PRSIM23_NS | Protein | MGSRLDAPSQIEVKDVTDTTALITWVDPRYDDIWWFELTYGIKDVPGDRTTIKLYL |
| | 4A/3 PR | | NDPYYSIGNLKPDTEYEVSLISYTGDSYSRSGSNPAKITFKTGLGGGSGMKKKGSV |
| | S139A_DCasp9 | | VIVGRINLSGDTAYAQQTRGEEGCQETSQTGRDKNQVEGEVQIVSTATQTFLATSI |
| | (S196A) | | NGVLWTVYHGAGTRTIASPKGPVTQMYTNVDKDLVGWQAPQGSRSLTPCTCGSSDL |
| | | | YLVTRHADVIPVRRRGDSRGSLLSPRPISYLKGSAGGPLLCPAGHAVGIFRAAVST |
| | | | RGVAKAVDFIPVESLETTMRSPGGGSGVDGFGDVGALESLRGNADLAYILSMEPCG |
| | | | HCLIINNVNFCRESGLRTRTGSNIDCEKLRRRFSALHFMVEVKGDLTAKKMVLALL |
| | | | ELARQDHGALDCCVVVILSHGCQASHLQFPGAVYGTDGCPVSVEKIVNIFNGTSCP |
| | | | SLGGKPKLFFIQACGGEQKDHGFEVASTSPEDESPGSNPEPDATPFQEGLRTFDQL |
| | | | DAISSLPTPSDIFVSYSTFPGFVSWRDPKSGSWYVETLDDIFEQWAHSEDLQSLLL |
| | | | RVANAVSVKGIYKQMPGCFNFLRKKLFFKTS |
| 231 | PRSIM23_NS | DNA | ATGGGCTCTAGACTGGATGCCCCTAGCCAGATCGAAGTGAAGGACGTGACCGACAC |
| | 4A/3 PR | | CACCGCTCTGATCACCTGGGTTGACCCCAGATACGACGATATCTGGTGGTTCGAGC |
| | S139A_DCasp9 | | TGACCTACGGCATCAAGGATGTGCCCGGCGACAGAACCACCATCAAGCTGTACCTG |
| | (S196A) | | AACGACCCCTACTACAGCATCGGCAACCTGAAGCCTGACACCGAGTACGAGGTGTC |
| | | | CCTGATCAGCTACACCGGCGACTCCTACAGCAGAAGCGGCAGCAATCCTGCCAAGA |
| | | | TCACCTTCAAGACCGGCCTTGGGGGCGGATCCGGCATGAAGAAAAAGGGCTCTGTG |
| | | | GTCATCGTGGGCAGAATCAACCTGAGCGGCGATACCGCCTACGCTCAGCAGACAAG |
| | | | AGGCGAGGAAGGCTGCCAAGAGACAAGCCAGACCGGCAGAGACAAGAACCAGGTGG |
| | | | AAGGCGAGGTGCAGATCGTGTCTACAGCTACCCAGACCTTCCTGGCCACCAGCATC |
| | | | AATGGCGTGCTGTGGACAGTGTATCACGGCGCTGGCACCAGAACAATCGCCTCTCC |
| | | | AAAGGGCCCCGTGACACAGATGTACACCAACGTGGACAAGGACCTCGTCGGATGGC |
| | | | AAGCCCCTCAGGGCTCTAGAAGCCTGACACCTTGTACCTGCGGCAGCAGCGATCTG |
| | | | TACCTGGTCACAAGACACGCCGACGTGATCCCCGTCAGAAGAAGAGGCGATAGCAG |
| | | | AGGCAGCCTGCTGAGCCCTAGACCTATCAGCTACCTGAAGGGATCTGCCGGCGGAC |
| | | | CTCTGCTTTGTCCTGCTGGACATGCCGTGGGCATCTTTAGAGCCGCCGTGTCTACT |
| | | | AGAGGCGTGGCCAAAGCCGTGGACTTCATCCCTGTGGAAAGCCTGGAAACCACCAT |
| | | | GCGGAGCCCCGGGGGAGGCTCCGGCGTGGATGGCTTTGGAGATGTGGGCGCCCTGG |
| | | | AATCCCTGAGAGGAAATGCCGATCTGGCCTACATCCTGAGCATGGAACCTTGCGGC |
| | | | CACTGCCTGATTATCAACAATGTGAACTTCTGCCGCGAGAGCGGCCTGAGAACAAG |
| | | | AACCGGCAGCAACATCGATTGCGAGAAGCTGCGGAGAAGATTCAGCGCCCTGCACT |
| | | | TCATGGTGGAAGTGAAGGGCGACCTGACCGCCAAGAAAATGGTGCTGGCTCTGCTG |
| | | | GAACTGGCCAGACAGGATCATGGCGCACTGGATTGCTGCGTGGTCGTGATTCTGAG |
| | | | CCACGGCTGTCAGGCCAGCCATCTGCAATTCCCTGGCGCCGTGTATGGCACCGATG |
| | | | GCTGTCCTGTGTCCGTGGAAAAGATCGTGAACATCTTCAACGGCACCAGCTGTCCT |
| | | | AGCCTCGGCGGAAAGCCCAAGCTGTTCTTCATCCAAGCCTGTGGCGGCGAGCAGAA |
| | | | GGATCACGGATTTGAGGTGGCCAGCACAAGCCCCGAGGATGAGAGCCCTGGAAGCA |
| | | | ACCCTGAGCCTGACGCCACACCTTTCCAAGAGGGACTGAGAACCTTCGACCAGCTG |
| | | | GACGCTATCAGCTCCCTGCCTACACCTAGCGACATCTTCGTGTCCTACAGCACATT |
| | | | CCCCGGCTTTGTGTCTTGGCGGGACCCCAAGTCTGGCTCTTGGTACGTGGAAACCC |
| | | | TGGATGACATCTTCGAGCAGTGGGCCCATAGCGAGGACCTGCAATCTCTGCTGCTG |
| | | | AGAGTGGCCAATGCCGTGTCCGTGAAGGGCATCTACAAGCAGATGCCCGGCTGCTT |
| | | | CAACTTCCTGCGGAAGAAGCTGTTTTTCAAGACCAGCTGATAG |
| 232 | GFP-PEST | DNA | ATGGAAAGCGACGAGTCTGGCCTGCCTGCTATGGAAATCGAGTGCCGGATCACCGG |
| | | | CACACTGAACGGCGTGGAATTCGAACTCGTTGGCGGCGGAGAGGGCACACCTGAAC |
| | | | AGGGCAGAATGACCAACAAGATGAAGTCCACCAAAGGCGCCCTGACATTCAGCCCC |
| | | | TACCTGCTGTCTCACGTGATGGGCTACGGCTTCTACCACTTCGGCACATACCCTAG |
| | | | CGGCTACGAGAACCCTTTCCTGCACGCCATCAACAACGGCGGCTACACCAACACCA |
| | | | GAATCGAGAAGTACGAGGACGGCGGCGTGCTGCACGTGTCCTTCAGCTACAGATAT |
| | | | GAGGCCGGCAGAGTGATCGGCGACTTCAAAGTGATGGGCACCGGATTTCCCGAGGA |
| | | | CAGCGTGATCTTCACCGACAAGATCATCCGGTCCAACGCCACCGTGGAACATCTGC |
| | | | ACCCTATGGGCGACAACGACCTGGATGGCAGCTTCACCAGAACCTTCAGCCTGAGA |
| | | | GATGGCGGCTACTACAGCAGCGTGGTGGACAGCCACATGCACTTCAAGAGCGCCAT |
| | | | CCATCCTAGCATCCTGCAGAACGGCGGACCCATGTTCGCCTTCAGAAGAGTGGAAG |
| | | | AGGACCACAGCAACACCGAGCTGGGCATCGTGGAATACCAGCACGCCTTCAAGACC |
| | | | CCTGATGCCGATGCCGGCGAGGAAAGAAGCAGAGATATCAGCCACGGCTTCCCACC |
| | | | AGCTGTGGCCGCTCAAGATGATGGCACACTGCCTATGAGCTGCGCCCAAGAGTCCG |
| | | | GCATGGATAGACATCCTGCCGCCTGTGCCAGCGCCAGAATCAATGTGTAA |
| 233 | GFP-PEST | Protein | MESDESGLPAMEIECRITGTLNGVEFELVGGGEGTPEQGRMTNKMKSTKGALTFSP |
| | | | YLLSHVMGYGFYHFGTYPSGYENPFLHAINNGGYTNTRIEKYEDGGVLHVSFSYRY |
| | | | EAGRVIGDFKVMGTGFPEDSVIFTDKIIRSNATVEHLHPMGDNDLDGSFTRTFSLR |
| | | | DGGYYSSWDSHMHFKSAIHPSILQNGGPMFAFRRVEEDHSNTELGIVEYQHAFKTP |
| | | | DADAGEERSRDISHGFPPAVAAQDDGTLPMSCAQESGMDRHPAACASARINV |
| 234 | hAAV1 left | DNA | GAGCACTTCCTTCTCGGCGCTGCACCACGTGATGTCCTCTGAGCGGATCCTCCCCG |
| | homologous | | TGTCTGGGTCCTCTCCGGGCATCTCTCCTCCCTCACCCAACCCCATGCCGTCTTCA |
| | arm | | CTCGCTGGGTTCCCTTTTCCTTCTCCTTCTGGGGCCTGTGCCATCTCTCGTTTCTT |
| | | | AGGATGGCCTTCTCCGACGGATGTCTCCCTTGCGTCCCGCCTCCCCTTCTTGTAGG |
| | | | CCTGCATCATCACCGTTTTTCTGGACAACCCCAAAGTACCCCGTCTCCCTGGCTTT |
| | | | AGCCACCTCTCCATCCTCTTGCTTTCTTTGCCTGGACACCCCGTTCTCCTGTGGAT |
| | | | TCGGGTCACCTCTCACTCCTTTCATTTGGGCAGCTCCCCTACCCCCCTTACCTCTC |
| | | | TAGTCTGTGCTAGCTCTTCCAGCCCCCTGTCATGGCATCTTCCAGGGGTCCGAGAG |
| | | | CTCAGCTAGTCTTCTTCCTCCAACCCGGGCCCCTATGTCCAC |
| 235 | hAAV1 Right | DNA | GATCCTGGGAGGGAGAGCTTGGCAGGGGGTGGGAGGGAAGGGGGGGATGCGTGACC |
| | Homologous | | TGCCCGGTTCTCAGTGGCCACCCTGCGCTACCCTCTCCCAGAACCTGAGCTGCTCT |
| | arm | | GACGCGGCCGTCTGGTGCGTTTCACTGATCCTGGTGCTGCAGCTTCCTTACACTTC |
| | | | CCAAGAGGAGAAGCAGTTTGGAAAAACAAAATCAGAATAAGTTGGTCCTGAGTTCT |
| | | | AACTTTGGCTCTTCACCTTTCTAGTCCCCAATTTATATTGTTCCTCCGTGCGTCAG |
| | | | TTTTACCTGTGAGATAAGGCCAGTAGCCAGCCCCGTCCTGGCAGGGCTGTGGTGAG |
| | | | GAGGGGGGTGTCCGTGTGGAAAACTCCCTTTGTGAGAATGGTGCGTCCTAGGTGTT |
| | | | CACCAGGTCGTGGCCGCCTCTACTCCCTTTCTCTTTCTCCATCCTTCTTTCCTTAA |
| | | | AGAGTCCCCAGTGCTATCTGGGACATATTCCTCCGCCCAGAGCAGGGTCCCGCTTC |
| | | | CCTAAGGCCCTGCTCTGTCTAGA |
| 236 | gRNA | DNA | GTTAATGTGGCTCTGGTTCT |
| | AAVS1 | | |
| 237 | MEDI8852 | DNA | caggttcagctgcagcagtctggacctggcctggtcaagcctagccagacactgtc |
| | heavy chain | | tctgacctgtgccatcagcggcgatagcgtgtccagctacaacgccgtgtggaact |
| | | | ggatcagacagagccctagcagaggcctggaatggctgggcagaacctactacaga |
| | | | agcggctggtacaacgactacgccgagagcgtgaagtcccggatcaccatcaatcc |
| | | | cgacaccagcaagaaccagttcagcctccagctgaacagcgtgacccctgaggata |
| | | | ccgccgtgtactactgtgccagatccggccacatcaccgtgttcggagtgaacgtg |
| | | | gacgccttcgatatgtggggccagggcacaatggtcaccgtgtctagcgcctctac |
| | | | aaagggccctagcgtgttccctctggctcctagcagcaagtctacaagcggaggaa |
| | | | cagccgctctgggctgcctcgtgaaggattactttcccgagcctgtgaccgtgtcc |
| | | | tggaattctggcgctctgacaagcggcgtgcacacctttccagctgtgctgcaaag |
| | | | cagcggcctgtactctctgagcagcgtggtcacagtgccaagctctagcctgggca |
| | | | cccagacctacatctgcaatgtgaatcacaagcccagcaacaccaaggtggacaag |
| | | | agagtggaacccaagagctgcgacaagacccacacctgtcctccatgtcctgctcc |
| | | | agaactgctcggcggaccttccgtgttcctgtttcctccaaagcctaaggacaccc |
| | | | tgatgatcagcagaacccctgaagtgacctgcgtggtggtggatgtgtctcacgag |
| | | | gaccccgaagtgaagttcaattggtacgtggacggcgtggaagtgcacaacgccaa |
| | | | gaccaagcctagagaggaacagtacaacagcacctacagagtggtgtccgtgctga |
| | | | ccgtgctgcaccaggattggctgaacggcaaagagtacaagtgcaaggtgtccaac |
| | | | aaggccctgcctgctcctatcgagaaaaccatcagcaaggccaagggccagcctag |
| | | | ggaaccccaggtttacacactgcctccaagccgggaagagatgaccaagaatcagg |
| | | | tgtccctgacctgcctggttaagggcttctacccctccgatatcgccgtggaatgg |
| | | | gagagcaatggccagcctgagaacaactacaagacaacccctcctgtgctggacag |
| | | | cgacggctcattcttcctgtacagcaagctgacagtggacaagtccagatggcagc |
| | | | agggcaacgtgttctcctgcagcgtgatgcacgaggccctgcacaaccactacacc |
| | | | cagaagtccctgagcctgtctcctggcaaa |
| 238 | MEDI8852 | DNA | gacatccagatgacacagagccctagcagcctgtctgccagcgtgggagacagagt |
| | light chain | | gaccatcacctgtagaaccagccagagcctgagcagctacacccactggtatcagc |
| | | | agaagcctggcaaggcccctaagctgctgatctatgccgccagctctagaggcagc |
| | | | ggagtgccttctagattttccggcagcggctccggcaccgatttcaccctgaccat |
| | | | atctagcctgcagcctgaggacttcgccacctactactgccagcagagcagaacct |
| | | | ttggccagggcaccaaggtggaaatcaagcggacagtggccgctcctagcgtgttc |
| | | | atctttccacctagcgacgagcagctgaagtctggcacagcctctgtcgtgtgcct |
| | | | gctgaacaacttctaccccagagaagccaaggtgcagtggaaggtggacaacgccc |
| | | | tgcagagcggcaatagccaagagagcgtgaccgagcaggacagcaaggactctacc |
| | | | tactctctgagcagcaccctgacactgagcaaggccgactacgagaagcacaaagt |
| | | | gtacgcctgcgaagtgacccaccagggcctttctagccctgtgaccaagagcttca |
| | | | accggggcgaatgt |

1. One or more expression vectors comprising:
i) a first expression cassette encoding a target protein, wherein the target protein is capable of binding to a small molecule in order to form a complex between the target protein and the small molecule (T-SM complex); and
ii) a second expression cassette encoding a binding member, wherein the binding member specifically binds to the T-SM complex such that the binding member binds the T-SM complex at a higher affinity than it binds both the target protein alone and the small molecule alone,
wherein the target protein is derived from a non-human protein and the small molecule is an inhibitor of the non-human protein, and
wherein the target protein is derived from a viral protease and the small molecule inhibitor is a viral protease inhibitor.

2. (canceled)

3. The one or more expression vectors of claim 1, wherein the viral protease is an HCV NS3/4A protease or HIV protease.

4. (canceled)

5. The one or more expression vectors of claim 1, wherein the small molecule is selected from the group consisting of simeprevir, boceprevir, telaprevir, asunaprevir, vaniprevir, voxilaprevir, glecaprevir, paritaprevir and narlaprevir, optionally wherein the small molecule is selected from the group consisting of simeprevir, boceprevir, and telaprevir.

6. The one or more expression vectors of claim 1, wherein the viral protease is an HCV NS3/4A protease and the small molecule is simeprevir.

7. The one or more expression vectors of claim 1, wherein the target protein has an amino acid sequence having at least 90% identity to SEQ ID NO: 1.

8. (canceled)

9. (canceled)

10. The one or more expression vectors of claim 7, wherein the target protein has an amino acid sequence having at least 90% identity to SEQ ID NO: 1 and the target protein comprises an amino acid mutation at one or more amino acids selected from positions 72, 96, 112, 114, 154, 160 and 164, wherein the amino acid numbering corresponds to SEQ ID NO 1.

11. (canceled)

12. The one or more expression vectors of claim 1, wherein the target protein has the amino acid sequence set forth in SEQ ID NO: 2.

13. (canceled)

14. The one or more expression vectors of claim 1, wherein the binding member binds to the T-SM complex with:
i) at least a 10-fold higher affinity;
ii) at least a 50-fold higher affinity;
iii) at least a 100-fold higher affinity; or
iv) at least a 1000-fold higher affinity
than the binding member binds to either the target protein alone and/or the small molecule alone.

15. (canceled)

16. (canceled)

17. (canceled)

18. (canceled)

19. (canceled)

20. The one or more expression vectors of claim 1, wherein the binding member is a Tn3 protein or an antibody molecule.

21. (canceled)

22. The one or more expression vectors of claim 20, wherein
the Tn3 protein comprises the BC, DE and FG loops of:
i) PRSIM_23, set forth in SEQ ID NOs: 136, 137, and 138, respectively;
ii) PRSIM_32, set forth in SEQ ID NOs: 139, 140, and 141, respectively;
iii) PRSIM_33, set forth in SEQ ID NOs: 142, 143, and 144, respectively;
iv) PRSIM_36, set forth in SEQ ID NOs: 145, 146, and 147, respectively; or
v) PRSIM_47, set forth in SEQ ID NOs: 148, 149, and 150, respectively, and optionally wherein the Tn3 protein comprises 3, 2, or 1 sequence alterations in the BC, DE, and/or EF loop.

23. (canceled)

24. (canceled)

25. The one or more expression vectors of claim 20, wherein the Tn3 protein comprises an amino acid sequence having at least 90% identity with the amino acid sequence of PRSIM_23 set forth in SEQ ID NO: 5.

26. (canceled)

27. (canceled)

28. The one or more expression vectors of claim 1, wherein the binding member is a single-chain variable fragment (scFv) and wherein the scFv comprises heavy chain complementarity determining regions (HCDRs) 1 to 3 and light chain complementarity determining regions (LCDRs) of:
i) PRSIM_57 set forth in SEQ ID NOs: 151, 152, 153, 154, 155, and 156, respectively;
ii) PRSIM_01 set forth in SEQ ID NOs 151, 152, 198, 154, 155, and 156, respectively;
iii) PRSIM_04 set forth in SEQ ID NOs: 151, 152, 163, 154, 155, and 164, respectively;
iv) PRSIM_67 set forth in SEQ ID NOs: 165, 166, 167, 168, 169, and 170, respectively;
v) PRSIM_72 set forth in SEQ ID NOs: 171, 172, 173, 174, 175, and 176, respectively; or
vi) PRSIM_75 set forth in SEQ ID NOs: 177, 178, 179, 180, 181, and 182, respectively,
wherein the CDR sequences are defined according to the Kabat numbering scheme, and
optionally wherein the scFv comprises 3, 2, or 1 sequence alterations in the HCDR1, HCDR2, HCDR3, LCDR1, LCDR2, and/or LCDR3.

29. (canceled)

30. (canceled)

31. The one or more expression vectors of claim 28, wherein the scFv comprises an amino acid sequence having at least 90% identity with the amino acid sequence of PRSIM_57 set forth in SEQ ID NO: 12.

32. (canceled)

33. The one or more expression vectors of claim 1, wherein
the target protein is fused to a first component polypeptide; and
the binding member is fused to a second component polypeptide.

34. (canceled)

35. The one or more expression vectors of claim 33, wherein
(1) the first component polypeptide comprises a DNA binding domain and is fused to the target protein to form a DBD-T fusion protein; and
the second component polypeptide comprises a transcriptional regulatory domain and is fused to the binding member to form a TRD-BM fusion protein, or
(2) the first component polypeptide comprises a transcriptional regulatory domain and is fused to the target protein to form a TRD-T fusion protein; and
the second component polypeptide comprises a DNA binding domain and is fused to the binding member to form a DBD-BM fusion protein,
wherein the first and second component polypeptides form a transcription factor upon dimerization.

36. (canceled)

37. The one or more expression vectors of claim 35, further comprising a third expression cassette, wherein the third expression cassette encodes a therapeutic protein, wherein the DNA binding domain binds to a target sequence in the third expression cassette such that the transcription factor is capable of regulating expression of the therapeutic protein.

38. (canceled)

39. (canceled)

40. The one or more expression vectors of claim 33, wherein
(1) the first component polypeptide comprises a first co-stimulatory domain and is fused to the target protein; and
the second component polypeptide comprises an intracellular signalling domain and is fused to the binding member, or
(2) the first component polypeptide comprises an intracellular signalling domain and is fused to the target protein; and
the second component polypeptide comprises a first co-stimulatory domain and is fused to the binding member.

41. The one or more expression vectors of claim 40(1), wherein
the first component polypeptide further comprises an antigen-specific recognition domain and a transmembrane domain; and
the second component polypeptide further comprises a transmembrane domain and a second co-stimulatory domain,
wherein the first and second component polypeptides form a chimeric antigen receptor (CAR) upon dimerization,
optionally wherein the target protein is fused to the C-terminus of the first co-stimulatory domain; and/or the binding member is fused to the C-terminus of the second co-stimulatory domain.

42. The one or more expression vectors of claim 40(2), wherein
the first component polypeptide further comprises a transmembrane domain and a second co-stimulatory domain; and
the second component polypeptide further comprises an antigen-specific recognition domain and a transmembrane domain,
wherein the first and second component polypeptides form a chimeric antigen receptor (CAR) upon dimerization,
optionally wherein the binding member is fused to the C-terminus of the first co-stimulatory domain; and/or the target protein is fused to the C-terminus of the second co-stimulatory domain.

43. (canceled)

44. The one or more expression vectors of claim 33, wherein
the first component polypeptide comprises a first caspase component; and
the second component polypeptide comprises a second caspase component,
and wherein the first and second component polypeptides form a caspase upon dimerization, optionally wherein the first and second caspase components comprise caspase 9 activation domains.

45. (canceled)

46. The one or more expression vectors of claim 1, wherein each of the one or more expression vectors is a DNA plasmid or a viral vector.

47. (canceled)

48. (canceled)

49. (canceled)

50. (canceled)

51. A binding member that specifically binds to a complex between i) a target protein derived from a non-human protein and ii) a small molecule that is an inhibitor of the non-human protein, wherein the binding member binds the complex at a higher affinity than it binds the target protein alone and/or the small molecule alone,
wherein the non-human protein is selected from the group consisting of a viral protease, an HCV NS3/4A protease, and a viral protease having an amino acid sequence having at least 90% identity to SEQ ID NO: 2.

52. (canceled)

53. (canceled)

54. (canceled)

55. (canceled)

56. (canceled)

57. (canceled)

58. (canceled)

59. A dimerization-inducible protein comprising:
a first component polypeptide fused to a target protein; and
a second component polypeptide fused to a binding member,
wherein the target protein is capable of binding to a small molecule in order to form a complex between the target protein and the small molecule (T-SM complex), wherein the binding member specifically binds to the T-SM complex such that the binding member binds the T-SM complex at a higher affinity than it binds both the target protein alone and/or the small molecule alone, and
wherein the target protein is derived from a viral protease and the small molecule is a viral protease inhibitor.

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. A cell expressing the dimerization-inducible protein of claim 59, wherein the cell is a stem cell or immune cell.

72. (canceled)

73. A method of genetically modifying a cell, the method comprising administering the one or more expression vectors of claim 1 to the cell.

74. One or more viral particles comprising:
i) a first expression cassette encoding a target protein, wherein the target protein is capable of binding to a small molecule in order to form a complex between the target protein and the small molecule (T-SM complex); and
ii) a second expression cassette encoding a binding member, wherein the binding member specifically binds to the T-SM complex such that the binding member binds the T-SM complex at a higher affinity than it binds both the target protein alone and/or the small molecule alone,
wherein the target protein is derived from a viral protease and the small molecule is a viral protease inhibitor,
and wherein the first and second expression cassettes form part of a viral genome in the one or more viral particles.

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. A method of treatment comprising administering the cell of claim 71 to an individual in need thereof, the method comprising:
i) administering the cell to the individual; and
ii) administering the small molecule to the individual.

91. (canceled)

92. (canceled)

93. (canceled)

94. (canceled)

95. (canceled)

96. (canceled)

97. A kit comprising the one or more expression vectors of claim 1 and the small molecule.

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. A target protein derived from a HCV NS3/4A protease, wherein the target protein has an amino acid sequence having at least 90% identity to the sequence set forth in SEQ ID NO: 1, wherein the target protein comprises an amino acid mutation compared to SEQ ID NO: 1 at one or more amino acids selected from positions 151 and 183, wherein the amino acid numbering corresponds to SEQ ID NO: 1, and wherein simeprevir is capable of binding the target protein.

111. (canceled)

112. (canceled)

113. (canceled)


SEQUENCE LISTING (247 sequences)

SEQ ID NO: 1 - protein, 197 aa, Artificial Sequence
  Wild-type HCV NS3/4A PR
MKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSP

SEQ ID NO: 2 - protein, 197 aa, Artificial Sequence
  HCV NS3/4A PR (S139A)
MKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 3 - protein, 229 aa, Artificial Sequence
  Wild-type HCV NS3/4A PR with N-terminal 6His and AviTag
MGSSHHHHHHGSGLNDIFEAQKIEWHEGGGGSMKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSP

SEQ ID NO: 4 - protein, 229 aa, Artificial Sequence
  HCV NS3/4A PR (S139A) with N-terminal 6His and AviTag
MGSSHHHHHHGSGLNDIFEAQKIEWHEGGGGSMKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 5 - protein, 97 aa, Artificial Sequence
  PRSIM_23
RLDAPSQIEVKDVTDTTALITWVDPRYDDIWWFELTYGIKDVPGDRTTIKLYLNDPYYSIGNLKPDTEYEVSLISYTGDSYSRSGSNPAKITFKTGL

SEQ ID NO: 6 - protein, 99 aa, Artificial Sequence
  PRSIM_32
RLDAPSQIEVKDVTDTTALITWWSPRYYYASISGFELTYGIKDVPGDRTTIKLDYASNDYSIGNLKPDTEYEVSLISWNYGDWRYSSSNPAKITFKTGL

SEQ ID NO: 7 - protein, 99 aa, Artificial Sequence
  PRSIM_33
RLDAPSQIEVKDVTDTTALITWYPPGRWYDDIWYFELTYGIKDVPGDRTTIKLARGDDVYSIGNLKPDTEYEVSLISWGPDRGDRAGSNPAKITFKTGL

SEQ ID NO: 8 - protein, 99 aa, Artificial Sequence
  PRSIM_36
RLDAPSQIEVKDVTDTTALITWSWPRDDDYDIWYFELTYGIKDVPGDRTTIKLLNYASPYSIGNLKPDTEYEVSLISVVPDTYGRGTSNPAKITFKTGL

SEQ ID NO: 9 - protein, 96 aa, Artificial Sequence
  PRSIM_47
RLDAPSQIEVKDVTDTTALITWSRPGVSIWYFELTYGIKDVPGDRTTIKLDYRSYYYSIGNLKPDTEYEVSLISGSYGLVGVRASNPAKITFKTGL

SEQ ID NO: 10 - protein, 245 aa, Artificial Sequence
  PRSIM_01
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGQGYITVFDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDHHWEQVVFGGGTKLTVL

SEQ ID NO: 11 - protein, 245 aa, Artificial Sequence
  PRSIM_04
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGMAHFYQFDLWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAGDHDHEHVVFGGGTKLTVL

SEQ ID NO: 12 - protein, 245 aa, Artificial Sequence
  PRSIM_57
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARHTNYITVFDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDHHWEQVVFGGGTKLTVL

SEQ ID NO: 13 - protein, 245 aa, Artificial Sequence
  PRSIM_67
EVQLVQSGAEVKKPGAAVRISCKTSGYVFTSYYVHWVRQAPGQGLEWMGVINPSGGNTNYAQKFQDRVTMTRDTSTTTVYMELSSLMFDDTAVYYCAKRDYGGPLANWGRGTLVTVSSGGGGSGGGGSGGGGSALSYELTQPPSVSEAPRQRVTISCSGSSSNIGNNAVNWYQQLPGKAPKLLIFYDDLLPSGVSDRFSGSKSGTSASLAISGLQSEDEADYYCAAWDDSLNGLVFGTGTKLTVL

SEQ ID NO: 14 - protein, 245 aa, Artificial Sequence; identical to SEQ ID NO: 11
  PRSIM_72

SEQ ID NO: 15 - protein, 244 aa, Artificial Sequence
  PRSIM_75
EVQLVQSGAEVKKPGSSVKVSCKASGGSFNSYTLDWVRQAPGQGLEWMGGIIPVFGSPNYGQKFQGRVTITADESTSTAYMELSSLKSDDTAVYYCARGLVYQPLDSWGRGTLVTVSSGGGGSGGGGSGGGGSAQAVLTQPSSASGTPGQRVTISCSGSSSNIGSYTVNWYQQFPGTAPKLLIYSNTQRPSGVPDRFSGSKSGTSASLAISGLQSEDEADYYCAAWDDSLNGWVFGGGTKVTVL

SEQ ID NO: 16 - protein, 158 aa, Artificial Sequence
  LgBiT
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTIN

SEQ ID NO: 17 - protein, 11 aa, Artificial Sequence
  SmBiT
VTGYRLFEEIL

SEQ ID NO: 18 - protein, 223 aa, Artificial Sequence
  HCV_NS4A_NS3_S139A_SmBiT
MKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPGSSGGGGSGGGGSSGVTGYRLFEEIL

SEQ ID NO: 19 - protein, 223 aa, Artificial Sequence
  SmBiT_HCV_NS4A_NS3_S139A
MVTGYRLFEEILGSSGGGGSGGGGSSGKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 20 - protein, 273 aa, Artificial Sequence
  PRSIM_23_LgBiT
MGSRLDAPSQIEVKDVTDTTALITWVDPRYDDIWWFELTYGIKDVPGDRTTIKLYLNDPYYSIGNLKPDTEYEVSLISYTGDSYSRSGSNPAKITFKTGLSGSSGGGGSGGGGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTIN

SEQ ID NO: 21 - protein, 275 aa, Artificial Sequence
  PRSIM_32_LgBiT
MGSRLDAPSQIEVKDVTDTTALITWWSPRYYYASISGFELTYGIKDVPGDRTTIKLDYASNDYSIGNLKPDTEYEVSLISWNYGDWRYSSSNPAKITFKTGLSGSSGGGGSGGGGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTIN

SEQ ID NO: 22 - protein, 275 aa, Artificial Sequence
  PRSIM_33_LgBiT
MGSRLDAPSQIEVKDVTDTTALITWYPPGRWYDDIWYFELTYGIKDVPGDRTTIKLARGDDVYSIGNLKPDTEYEVSLISWGPDRGDRAGSNPAKITFKTGLSGSSGGGGSGGGGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTIN

SEQ ID NO: 23 - protein, 275 aa, Artificial Sequence
  PRSIM_36_LgBiT
MGSRLDAPSQIEVKDVTDTTALITWSWPRDDDYDIWYFELTYGIKDVPGDRTTIKLLNYASPYSIGNLKPDTEYEVSLISVVPDTYGRGTSNPAKITFKTGLSGSSGGGGSGGGGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTIN

SEQ ID NO: 24 - protein, 272 aa, Artificial Sequence
  PRSIM_47_LgBiT
MGSRLDAPSQIEVKDVTDTTALITWSRPGVSIWYFELTYGIKDVPGDRTTIKLDYRSYYYSIGNLKPDTEYEVSLISGSYGLVGVRASNPAKITFKTGLSGSSGGGGSGGGGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTIN

SEQ ID NO: 25 - skipped

SEQ ID NO: 26 - protein, 421 aa, Artificial Sequence
  PRSIM_06_LgBiT
MGSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGAGYYMRVDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDHDVEHVVFGGGTKLTVLSGSSGGGGSGGGGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTIN

SEQ ID NO: 27 - protein, 421 aa, Artificial Sequence
  PRSIM_57_LgBiT
MGSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARHTNYITVFDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDHHWEQVVFGGGTKLTVLSGSSGGGGSGGGGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTIN

SEQ ID NO: 28 - protein, 421 aa, Artificial Sequence
  PRSIM_67_LgBiT
MGSEVQLVQSGAEVKKPGAAVRISCKTSGYVFTSYYVHWVRQAPGQGLEWMGVINPSGGNTNYAQKFQDRVTMTRDTSTTTVYMELSSLMFDDTAVYYCAKRDYGGPLANWGRGTLVTVSSGGGGSGGGGSGGGGSALSYELTQPPSVSEAPRQRVTISCSGSSSNIGNNAVNWYQQLPGKAPKLLIFYDDLLPSGVSDRFSGSKSGTSASLAISGLQSEDEADYYCAAWDDSLNGLVFGTGTKLTVLSGSSGGGGSGGGGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTIN

SEQ ID NO: 29 - protein, 421 aa, Artificial Sequence
  PRSIM_72_LgBiT
MGSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGMAHFYQFDLWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAGDHDHEHVVFGGGTKLTVLSGSSGGGGSGGGGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTIN

SEQ ID NO: 30 - protein, 420 aa, Artificial Sequence
  PRSIM_75_LgBiT
MGSEVQLVQSGAEVKKPGSSVKVSCKASGGSFNSYTLDWVRQAPGQGLEWMGGIIPVFGSPNYGQKFQGRVTITADESTSTAYMELSSLKSDDTAVYYCARGLVYQPLDSWGRGTLVTVSSGGGGSGGGGSGGGGSAQAVLTQPSSASGTPGQRVTISCSGSSSNIGSYTVNWYQQFPGTAPKLLIYSNTQRPSGVPDRFSGSKSGTSASLAISGLQSEDEADYYCAAWDDSLNGWVFGGGTKVTVLSGSSGGGGSGGGGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTIN

SEQ ID NO: 31 - protein, 271 aa, Artificial Sequence
  LgBiT_PRSIM_23
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGSSGGGGSGGGGSSGRLDAPSQIEVKDVTDTTALITWVDPRYDDIWWFELTYGIKDVPGDRTTIKLYLNDPYYSIGNLKPDTEYEVSLISYTGDSYSRSGSNPAKITFKTGL

SEQ ID NO: 32 - protein, 273 aa, Artificial Sequence
  LgBiT_PRSIM_32
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGSSGGGGSGGGGSSGRLDAPSQIEVKDVTDTTALITWWSPRYYYASISGFELTYGIKDVPGDRTTIKLDYASNDYSIGNLKPDTEYEVSLISWNYGDWRYSSSNPAKITFKTGL

SEQ ID NO: 33 - protein, 273 aa, Artificial Sequence
  LgBiT_PRSIM_33
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGSSGGGGSGGGGSSGRLDAPSQIEVKDVTDTTALITWYPPGRWYDDIWYFELTYGIKDVPGDRTTIKLARGDDVYSIGNLKPDTEYEVSLISWGPDRGDRAGSNPAKITFKTGL

SEQ ID NO: 34 - protein, 273 aa, Artificial Sequence
  LgBiT_PRSIM_36
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGSSGGGGSGGGGSSGRLDAPSQIEVKDVTDTTALITWSWPRDDDYDIWYFELTYGIKDVPGDRTTIKLLNYASPYSIGNLKPDTEYEVSLISVVPDTYGRGTSNPAKITFKTGL

SEQ ID NO: 35 - protein, 270 aa, Artificial Sequence
  LgBiT_PRSIM_47
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGSSGGGGSGGGGSSGRLDAPSQIEVKDVTDTTALITWSRPGVSIWYFELTYGIKDVPGDRTTIKLDYRSYYYSIGNLKPDTEYEVSLISGSYGLVGVRASNPAKITFKTGL

SEQ ID NO: 36 - protein, 419 aa, Artificial Sequence
  LgBiT_PRSIM_01
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGSSGGGGSGGGGSSGQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGQGYITVFDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDHHWEQVVFGGGTKLTVL

SEQ ID NO: 37 - protein, 419 aa, Artificial Sequence
  LgBiT_PRSIM_06
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGSSGGGGSGGGGSSGQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGAGYYMRVDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDHDVEHVVFGGGTKLTVL

SEQ ID NO: 38 - protein, 419 aa, Artificial Sequence
  LgBiT_PRSIM_57
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGSSGGGGSGGGGSSGQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARHTNYITVFDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDHHWEQVVFGGGTKLTVL

SEQ ID NO: 39 - protein, 419 aa, Artificial Sequence
  LgBiT_PRSIM_67
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGSSGGGGSGGGGSSGEVQLVQSGAEVKKPGAAVRISCKTSGYVFTSYYVHWVRQAPGQGLEWMGVINPSGGNTNYAQKFQDRVTMTRDTSTTTVYMELSSLMFDDTAVYYCAKRDYGGPLANWGRGTLVTVSSGGGGSGGGGSGGGGSALSYELTQPPSVSEAPRQRVTISCSGSSSNIGNNAVNWYQQLPGKAPKLLIFYDDLLPSGVSDRFSGSKSGTSASLAISGLQSEDEADYYCAAWDDSLNGLVFGTGTKLTVL

SEQ ID NO: 40 - protein, 419 aa, Artificial Sequence
  LgBiT_PRSIM_72
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGSSGGGGSGGGGSSGQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGMAHFYQFDLWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAGDHDHEHVVFGGGTKLTVL

SEQ ID NO: 41 - protein, 418 aa, Artificial Sequence
  LgBiT_PRSIM_75
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGSSGGGGSGGGGSSGEVQLVQSGAEVKKPGSSVKVSCKASGGSFNSYTLDWVRQAPGQGLEWMGGIIPVFGSPNYGQKFQGRVTITADESTSTAYMELSSLKSDDTAVYYCARGLVYQPLDSWGRGTLVTVSSGGGGSGGGGSGGGGSAQAVLTQPSSASGTPGQRVTISCSGSSSNIGSYTVNWYQQFPGTAPKLLIYSNTQRPSGVPDRFSGSKSGTSASLAISGLQSEDEADYYCAAWDDSLNGWVFGGGTKVTVL

SEQ ID NO: 42 - protein, 194 aa, Artificial Sequence
  p65 AD
DEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSTSY

SEQ ID NO: 43 - protein, 136 aa, Artificial Sequence
  ZFHD1 DBD (+ leader)
MDYPAAKRVKLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINT

SEQ ID NO: 44 - protein, 198 aa, Artificial Sequence
  HCV-Pro - AD fusion
MGKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 45 - protein, 237 aa, Artificial Sequence
  DBD - HCV Pro fusion
MDYPAAKRVKLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTSAGSRLDAPSQIEVKDVTDTTALITWVDPRYDDIWWFELTYGIKDVPGDRTTIKLYLNDPYYSIGNLKPDTEYEVSLISYTGDSYSRSGSNPAKITFKTGL

SEQ ID NO: 46 - protein, 237 aa, Artificial Sequence; identical to SEQ ID NO: 45
  PRSIM_23_DBD_fusion

SEQ ID NO: 47 - protein, 239 aa, Artificial Sequence
  PRSIM_32_DBD_fusion
MDYPAAKRVKLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTSAGSRLDAPSQIEVKDVTDTTALITWWSPRYYYASISGFELTYGIKDVPGDRTTIKLDYASNDYSIGNLKPDTEYEVSLISWNYGDWRYSSSNPAKITFKTGL

SEQ ID NO: 48 - protein, 239 aa, Artificial Sequence
  PRSIM_33_DBD_fusion
MDYPAAKRVKLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTSAGSRLDAPSQIEVKDVTDTTALITWYPPGRWYDDIWYFELTYGIKDVPGDRTTIKLARGDDVYSIGNLKPDTEYEVSLISWGPDRGDRAGSNPAKITFKTGL

SEQ ID NO: 49 - protein, 239 aa, Artificial Sequence
  PRSIM_36_DBD_fusion
MDYPAAKRVKLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTSAGSRLDAPSQIEVKDVTDTTALITWSWPRDDDYDIWYFELTYGIKDVPGDRTTIKLLNYASPYSIGNLKPDTEYEVSLISVVPDTYGRGTSNPAKITFKTGL

SEQ ID NO: 50 - protein, 236 aa, Artificial Sequence
  PRSIM_47_DBD_fusion
MDYPAAKRVKLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTSAGSRLDAPSQIEVKDVTDTTALITWSRPGVSIWYFELTYGIKDVPGDRTTIKLDYRSYYYSIGNLKPDTEYEVSLISGSYGLVGVRASNPAKITFKTGL

SEQ ID NO: 51 - protein, 385 aa, Artificial Sequence
  PRSIM_01_DBD_fusion
MDYPAAKRVKLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTSAGSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGQGYITVFDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDHHWEQVVFGGGTKLTVL

SEQ ID NO: 52 - protein, 385 aa, Artificial Sequence
  PRSIM_04_DBD_fusion
MDYPAAKRVKLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTSAGSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGMAHFYQFDLWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAGDHDHEHVVFGGGTKLTVL

SEQ ID NO: 53 - protein, 385 aa, Artificial Sequence
  PRSIM_57_DBD_fusion
MDYPAAKRVKLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTSAGSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARHTNYITVFDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDHHWEQVVFGGGTKLTVL

SEQ ID NO: 54 - protein, 385 aa, Artificial Sequence
  PRSIM_67_DBD_fusion
MDYPAAKRVKLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTSAGSEVQLVQSGAEVKKPGAAVRISCKTSGYVFTSYYVHWVRQAPGQGLEWMGVINPSGGNTNYAQKFQDRVTMTRDTSTTTVYMELSSLMFDDTAVYYCAKRDYGGPLANWGRGTLVTVSSGGGGSGGGGSGGGGSALSYELTQPPSVSEAPRQRVTISCSGSSSNIGNNAVNWYQQLPGKAPKLLIFYDDLLPSGVSDRFSGSKSGTSASLAISGLQSEDEADYYCAAWDDSLNGLVFGTGTKLTVL

SEQ ID NO: 55 - protein, 394 aa, Artificial Sequence
  PRSIM_72_DBD_fusion
MDYPAAKRVKLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTSAGSQVQLVQSGAEVKKPGSSVKVSCKVSGGSFNNYGVSWVRQAPGQGLEWMGRIIPIRDTANYAQKFQGRVTITADTSTNIAYMELSGLRSDDTAVYYCARVLEDDFWGGYYDFYFYVMDVWGQGTLVTVSSGGGGSGGGGSGGGGSALSSELTQDPVVSVPLGQTARITCQGDSLTTYYATWYQQKPGQAPVLVLYNEHKRPSGISDRFSGSSAGDAASLTITDTQAEDEADYYCSSRDTGGKHVLFGGGTKLTVL

SEQ ID NO: 56 - protein, 384 aa, Artificial Sequence
  PRSIM_75_DBD_fusion
MDYPAAKRVKLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTSAGSEVQLVQSGAEVKKPGSSVKVSCKASGGSFNSYTLDWVRQAPGQGLEWMGGIIPVFGSPNYGQKFQGRVTITADESTSTAYMELSSLKSDDTAVYYCARGLVYQPLDSWGRGTLVTVSSGGGGSGGGGSGGGGSAQAVLTQPSSASGTPGQRVTISCSGSSSNIGSYTVNWYQQFPGTAPKLLIYSNTQRPSGVPDRFSGSKSGTSASLAISGLQSEDEADYYCAAWDDSLNGWVFGGGTKVTVL

SEQ ID NO: 57 - protein, 305 aa, Artificial Sequence
  PRSIM_23_AD_fusion
MGSRLDAPSQIEVKDVTDTTALITWVDPRYDDIWWFELTYGIKDVPGDRTTIKLYLNDPYYSIGNLKPDTEYEVSLISYTGDSYSRSGSNPAKITFKTGLTGGGGSGGGGSDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSTSY

SEQ ID NO: 58 - protein, 307 aa, Artificial Sequence
  PRSIM_32_AD_fusion
MGSRLDAPSQIEVKDVTDTTALITWWSPRYYYASISGFELTYGIKDVPGDRTTIKLDYASNDYSIGNLKPDTEYEVSLISWNYGDWRYSSSNPAKITFKTGLTGGGGSGGGGSDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSTSY

SEQ ID NO: 59 - protein, 307 aa, Artificial sequence
  PRSIM_33_AD_fusion
MGSRLDAPSQIEVKDVTDTTALITWYPPGRWYDDIWYFELTYGIKDVPGDRTTIKLARGDDVYSIGNLKPDTEYEVSLISWGPDRGDRAGSNPAKITFKTGLTGGGGSGGGGSDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSTSY

SEQ ID NO: 60 - protein, 307 aa, Artificial sequence
  PRSIM_36_AD_fusion
MGSRLDAPSQIEVKDVTDTTALITWSWPRDDDYDIWYFELTYGIKDVPGDRTTIKLLNYASPYSIGNLKPDTEYEVSLISVVPDTYGRGTSNPAKITFKTGLTGGGGSGGGGSDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSTSY

SEQ ID NO: 61 - protein, 304 aa, Artificial sequence
  PRSIM_47_AD_fusion
MGSRLDAPSQIEVKDVTDTTALITWSRPGVSIWYFELTYGIKDVPGDRTTIKLDYRSYYYSIGNLKPDTEYEVSLISGSYGLVGVRASNPAKITFKTGLTGGGGSGGGGSDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSTSY

SEQ ID NO: 62 - protein, 453 aa, Artificial sequence
  PRSIM_01_AD_fusion
MGSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGQGYYGYFDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSVSKSGTSASLAISGLQSEDEADYYCAAWDHGHEHVVFGGGTKLTVLTGGGGSGGGGSDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSTSY

SEQ ID NO: 63 - protein, 453 aa, Artificial sequence
  PRSIM_04_AD_fusion
MGSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGMAHFYQFDLWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAGDHDHEHVVFGGGTKLTVLTGGGGSGGGGSDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSTSY

SEQ ID NO: 64 - protein, 453 aa, Artificial sequence
  PRSIM_57_AD_fusion
MGSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARHTNYITVFDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDHHWEQVVFGGGTKLTVLTGGGGSGGGGSDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSTSY

SEQ ID NO: 65 - protein, 453 aa, Artificial sequence
  PRSIM_67_AD_fusion
MGSEVQLVQSGAEVKKPGAAVRISCKTSGYVFTSYYVHWVRQAPGQGLEWMGVINPSGGNTNYAQKFQDRVTMTRDTSTTTVYMELSSLMFDDTAVYYCAKRDYGGPLANWGRGTLVTVSSGGGGSGGGGSGGGGSALSYELTQPPSVSEAPRQRVTISCSGSSSNIGNNAVNWYQQLPGKAPKLLIFYDDLLPSGVSDRFSGSKSGTSASLAISGLQSEDEADYYCAAWDDSLNGLVFGTGTKLTVLTGGGGSGGGGSDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSTSY

SEQ ID NO: 66 - protein, 462 aa, Artificial sequence
  PRSIM_72_AD_fusion
MGSQVQLVQSGAEVKKPGSSVKVSCKVSGGSFNNYGVSWVRQAPGQGLEWMGRIIPIRDTANYAQKFQGRVTITADTSTNIAYMELSGLRSDDTAVYYCARVLEDDFWGGYYDFYFYVMDVWGQGTLVTVSSGGGGSGGGGSGGGGSALSSELTQDPVVSVPLGQTARITCQGDSLTTYYATWYQQKPGQAPVLVLYNEHKRPSGISDRFSGSSAGDAASLTITDTQAEDEADYYCSSRDTGGKHVLFGGGTKLTVLTGGGGSGGGGSDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSTSY

SEQ ID NO: 67 - protein, 452 aa, Artificial sequence
  PRSIM_75_AD_fusion
MGSEVQLVQSGAEVKKPGSSVKVSCKASGGSFNSYTLDWVRQAPGQGLEWMGGIIPVFGSPNYGQKFQGRVTITADESTSTAYMELSSLKSDDTAVYYCARGLVYQPLDSWGRGTLVTVSSGGGGSGGGGSGGGGSAQAVLTQPSSASGTPGQRVTISCSGSSSNIGSYTVNWYQQFPGTAPKLLIYSNTQRPSGVPDRFSGSKSGTSASLAISGLQSEDEADYYCAAWDDSLNGWVFGGGTKVTVLTGGGGSGGGGSDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSTSY

SEQ ID NO: 68 - protein, 212 aa, Artificial sequence
  NanoLuc-Pest
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILANSHGFPPEVEEQAAGTLPMSCAQESGMDRHPAACASARINV

SEQ ID NO: 69 - protein, 864 aa, Artificial sequence
  FKBP12:FRB CAR
MLLLVTSLLLCELPHPAFLLIPESKYGPPCPPCPFWVLVVVGGVLACYSLLVTVAFIIFWVKRGRKKLLYIFKQPFMRPVQTTQEEDGCSCRFPEEEEGGCELSRGSGSGSGSMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGSGATNFSLLKQAGDVEENPGPMIHLGHILFLLLLPVAAAQTTPGERSSLPAFYPGTSGSCSGCGSLSLPESKYGPPCPPCPFWVLVVVGGVLACYSLLVTVAFIIFWVSLKRGRKKLLYIFKQPFMRPVQTTQEEDGCSCRFPEEEEGGCELILWHEMWHEGLEEASRLYFGERNVKGMFEVLEPLHAMMERGPQTLKETSFNQAYGRDLMEAQEWCRKYMKSGNVKDLLQAWDLYYHVFRRISKGSGSGSGSSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPSGMESDESGLPAMEIECRITGTLNGVEFELVGGGEGTPKQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFARSRAQSSNSAVDGTAGPGSTGSR

SEQ ID NO: 70 - protein, 291 aa, Artificial sequence
  PRSIM_23 CAR 1st polypeptide
ESKYGPPCPPCPFWVLVVVGGVLACYSLLVTVAFIIFWVKRGRKKLLYIFKQPFMRPVQTTQEEDGCSCRFPEEEEGGCELGGGGSGGGGSMKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPGSG

SEQ ID NO: 71 - DNA, 687 bp, Artificial sequence
  Wild-type HCV NS3/4A PR with N-terminal 6His and AviTag
atgggtagcagccatcaccatcatcatcatggtagcggtctgaacgatatttttgaagcccagaaaatcgaatggcatgaaggtggtggtggtagcatgaaaaaaaagggtagcgttgttattgtgggtcgcattaatctgagcggtgataccgcatatgcacagcagacccgtggtgaagaaggttgtcaagaaaccagccagaccggtcgtgataaaaatcaggttgaaggtgaagttcagattgttagcaccgcaacacagacctttctggcaaccagcattaatggtgttctgtggaccgtttatcatggtgcaggcacccgtaccattgcaagcccgaaaggtccggttacacagatgtataccaatgtggataaagatctggttggttggcaggcaccgcagggtagccgtagtctgaccccgtgtacctgtggtagcagcgatctgtatctggttacccgtcatgcagatgttattccggttcgtcgtcgtggtgatagccgtggtagcctgctgagtccgcgtccgattagctatctgaaaggtagcagtggtggtccgctgctgtgtccggcaggtcatgcagttggtatttttcgtgcagcagttagcacccgtggcgttgcaaaagcagttgattttatcccggttgaaagcctggaaaccaccatgcgtagcccg

SEQ ID NO: 72 - DNA, 687 bp, Artificial sequence
  HCV NS3/4A PR (S139A) with N-terminal 6His and AviTag
atgggtagcagccatcaccatcatcatcatggtagcggtctgaacgatatttttgaagcccagaaaatcgaatggcatgaaggtggtggtggtagcatgaaaaaaaagggtagcgttgttattgtgggtcgcattaatctgagcggtgataccgcatatgcacagcagacccgtggtgaagaaggttgtcaagaaaccagccagaccggtcgtgataaaaatcaggttgaaggtgaagttcagattgttagcaccgcaacacagacctttctggcaaccagcattaatggtgttctgtggaccgtttatcatggtgcaggcacccgtaccattgcaagcccgaaaggtccggttacacagatgtataccaatgtggataaagatctggttggttggcaggcaccgcagggtagccgtagtctgaccccgtgtacctgtggtagcagcgatctgtatctggttacccgtcatgcagatgttattccggttcgtcgtcgtggtgatagccgtggtagcctgctgagtccgcgtccgattagctatctgaaaggtagtgccggtggtccgctgctgtgtccggcaggtcatgcagttggtatttttcgtgcagcagttagcacccgtggcgttgcaaaagcagttgattttatcccggttgaaagcctggaaaccaccatgcgtagcccg

SEQ ID NO: 73 - DNA, 291 bp, Artificial sequence
  PRSIM_23
cgtctggatgcaccgagccagattgaagttaaagatgttaccgataccaccgcactgattacctgggttgacccgcgttacgacgacatttggtggtttgaactgacctatggcatcaaagatgttccgggtgatcgtaccaccattaaactgtacctgaacgacccgtactatagcattggtaatctgaaaccggataccgaatatgaagttagcctgattagctacactggtgactcttactctcgttctggtagcaatccggcaaaaattacctttaaaaccggtctg

SEQ ID NO: 74 - DNA, 297 bp, Artificial sequence
  PRSIM_32
cgtctggatgcaccgagccagattgaagttaaagatgttaccgataccaccgcactgattacctggtggtctccgcgttactactacgcttctatttctggttttgaactgacctatggcatcaaagatgttccgggtgatcgtaccaccattaaactggactacgcttctaacgactatagcattggtaatctgaaaccggataccgaatatgaagttagcctgattagctggaactacggtgactggcgttactcttctagcaatccggcaaaaattacctttaaaaccggtctg

SEQ ID NO: 75 - DNA, 297 bp, Artificial sequence
  PRSIM_33
cgtctggatgcaccgagccagattgaagttaaagatgttaccgataccaccgcactgattacctggtacccgccgggtcgttggtacgacgacatttggtactttgaactgacctatggcatcaaagatgttccgggtgatcgtaccaccattaaactggctcgtggtgacgacgtttatagcattggtaatctgaaaccggataccgaatatgaagttagcctgattagctggggtccggaccgtggtgaccgtgctggtagcaatccggcaaaaattacctttaaaaccggtctg

SEQ ID NO: 76 - DNA, 297 bp, Artificial sequence
  PRSIM_36
cgtctggatgcaccgagccagattgaagttaaagatgttaccgataccaccgcactgattacctggtcttggccgcgtgacgacgactacgacatttggtactttgaactgacctatggcatcaaagatgttccgggtgatcgtaccaccattaaactgctgaactacgcttctccgtatagcattggtaatctgaaaccggataccgaatatgaagttagcctgattagcgttgttccggacacttacggtcgtggtactagcaatccggcaaaaattacctttaaaaccggtctg

SEQ ID NO: 77 - DNA, 288 bp, Artificial sequence
  PRSIM_47
cgtctggatgcaccgagccagattgaagttaaagatgttaccgataccaccgcactgattacctggtctcgtccgggtgtttctatttggtactttgaactgacctatggcatcaaagatgttccgggtgatcgtaccaccattaaactggactaccgttcttactactatagcattggtaatctgaaaccggataccgaatatgaagttagcctgattagcggttcttacggtctggttggtgttcgtgctagcaatccggcaaaaattacctttaaaaccggtctg

SEQ ID NO: 78 - DNA, 735 bp, Artificial sequence
  PRSIM_01
caggttcagctggttcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacgagtctacaagcaccgcctacatggaactgagcagcctgagaagcgaggacaccgccgtgtactattgtgccagaggccagggctactacggctacttcgattattggggccagggcaccctggtcacagtttctagcggaggcggaggatctggtggcggaggaagtggcggaggcggttctgctcaatctgtgctgacacagcctcctagcgcctctggaacacctggccagagagtgacaatcagctgtagcggcagcagcagcaacatcggcagcaacaccgtgaactggtatcagcagctgcctggcacagcccctaaactgctgatctacagcaacaaccagcggcctagcggcgtgcccgatagattttccgtgtctaagagcggcaccagcgccagcctggctatttctggactgcagagcgaggacgaggccgactattattgtgccgcctgggatcacggacacgagcacgttgtgtttggaggcggcaccaagctgacagtgctt

SEQ ID NO: 79 - DNA, 735 bp, Artificial sequence
  PRSIM_04
caggtgcagctggtgcagtctggcgctgaagtgaagaagccgggctcttctgtgaaggtgtcttgcaaggcttctggcggcaccttctcttcttacgctatctcttgggtgcgtcaggctccgggccaggggctggagtggatgggcggcatcatcccgatcttcggcaccgctaactacgctcagaaatttcagggccgtgtgaccatcaccgctgatgaatctacctctaccgcttacatggaactgtcatctctgcgttctgaagataccgctgtatactactgcgctcgtggcatggctcacttctaccagttcgatctgtggggccagggcaccctggtaaccgtctcgagtggtggtggcggctctggtggcggtggctctggcggtggtggcagtgcacagtctgtgctgacccagccgccgtctgcttctggcaccccgggccagcgtgtgaccatctcttgctctggctcttcttctaacatcggctctaacaccgtgaactggtaccagcagctgccgggcaccgctccgaagctgctgatatactctaacaaccagcgtccgtctggcgtgccggatcgtttctctggctctaagtctggcacctctgcttctctggctatctctggcctgcagtctgaagacgaagctgattactactgcgctgctggggatcacgatcacgaacacgtggtgttcggcggcggcaccaagctgaccgtgctg

SEQ ID NO: 80 - DNA, 735 bp, Artificial sequence
  PRSIM_57
caggtgcagctggtgcagtctggcgctgaagtgaagaagccgggctcttctgtgaaggtgtcttgcaaggcttctggcggcaccttctcttcttacgctatctcttgggtgcgtcaggctccgggccaggggctggagtggatgggcggcatcatcccgatcttcggcaccgctaactacgctcagaaatttcagggccgtgtgaccatcaccgctgatgaatctacctctaccgcttacatggaactgtcatctctgcgttctgaagataccgctgtatactactgcgctcgtcacacgaactacatcacggttttcgattactggggccagggcaccctggtaaccgtctcgagtggtggtggcggctctggtggcggtggctctggcggtggtggcagtgcacagtctgtgctgacccagccgccgtctgcttctggcaccccgggccagcgtgtgaccatctcttgctctggctcttcttctaacatcggctctaacaccgtgaactggtaccagcagctgccgggcaccgctccgaagctgctgatctactctaacaaccagcgtccgtctggcgtgccggatcgtttctctggctctaagtctggcacctctgcttctctggctatctctggcctgcagtctgaagacgaagctgattactactgcgctgcttgggatcaccactgggaacaggtggtgttcggcggcggcaccaagctgaccgtgctg

SEQ ID NO: 81 - DNA, 735 bp, Artificial sequence
  PRSIM_67
gaagtgcagctggtgcagtctggggctgaggtgaagaagcctggggccgcagtgaggatttcctgcaagacatctggatacgtcttcaccagctactatgtgcactgggtgcgacaggcccctggacaagggcttgagtggatgggagttatcaaccctagtggtggtaatacgaactacgcacagaagttccaggacagagtcaccatgaccagggacacgtccacgaccacagtctatatggagttgagcagcctgatgtttgatgacacggccgtgtattactgtgcgaagcgagactacgggggacccttggcaaactggggccggggaaccctggtcaccgtctcgagtggaggcggcggttcaggcggaggtggctctggcggtggcggaagtgcactttcctatgagctgactcagccaccctcggtgtctgaagccccgaggcagagggtcaccatctcctgttctggaagcagctccaacatcggaaataatgctgtaaactggtaccagcagctcccaggaaaggctcccaaactcctcattttttatgatgatctgctgccctcaggggtctctgaccgattctctggctccaagtctggcacctcagcctccctggccatcagtgggctccagtccgaggatgaggctgattattactgtgcagcatgggatgacagcctgaatggtctagtcttcggaactgggaccaagctgaccgtccta

SEQ ID NO: 82 - DNA, 762 bp, Artificial sequence
  PRSIM_72
caggtgcagctggtgcagtctggggctgaggtgaagaagccggggtcctcggtgaaggtctcctgcaaggtttctggaggcagcttcaataattatggtgtcagttgggtgcgacaggcccctggacaagggcttgagtggatgggaaggatcatccctatccgtgatacagcaaactacgcacagaagttccagggcagagtcacgattaccgcggacacatccacgaacattgcctacatggaactgagcggcctgagatctgacgacacggccgtgtattactgtgcgagagtacttgaggacgatttctggggtggttattatgacttctatttctacgttatggacgtctggggccagggcaccctggtcaccgtctcgagtggaggcggcggttcaggcggaggtggctctggcggtggcggaagtgcactttcttctgagctgactcaggaccctgttgtgtctgtgcccttgggacagacagccaggatcacatgccaaggagacagcctcaccacttattatgcaacctggtaccagcagaagccaggacaggcccctgttcttgtcctctataatgaacacaaaaggccctcagggatctcagaccgattctctggctccagcgcaggagacgcagcttccttgaccatcactgacacccaggcggaagatgaggccgactattattgtagctcccgggacaccggtgggaagcatgtgcttttcggcggagggaccaagctgaccgtccta

SEQ ID NO: 83 - DNA, 732 bp, Artificial sequence
  PRSIM_75
gaggtgcagctggtgcagtctggggctgaggtgaagaagcctgggtcctcggtgaaggtctcctgcaaggcttctggaggctccttcaacagttatactctcgactgggtgcgacaggcccctggacaagggcttgagtggatgggagggatcatccctgtctttggttccccgaactacggacagaaattccagggcagagtcaccattaccgcggacgaatcaacgagcacagcctacatggagctgagcagtctcaaatctgacgacacggccgtgtattactgtgcgcgagggttggtataccagccccttgactcctggggccgaggcaccctggtcaccgtctcgagtggaggcggcggttcaggcggaggtggctctggcggtggcggaagtgcacaggctgtgctgactcagccgtcctcagcgtctgggacccccgggcagagggtcaccatctcttgttctggaagcagctccaacatcggaagttatactgtaaactggtaccagcaattcccaggaacggcccccaaactcctcatctatagtaatactcagcggccctcaggggtccctgaccgattctctggctccaagtctggcacctcagcctccctggccatcagtgggctccagtctgaggatgaggctgattattactgtgcagcatgggatgacagcctgaatggttgggtgttcggcggagggaccaaggtcaccgtccta

SEQ ID NO: 84 - DNA, 675 bp, Artificial sequence
  HCV_NS4A_NS3_S139A_SmBiT
atgggcaagaaaaagggctctgtggtcatcgtgggcagaatcaacctgagcggcgataccgcctacgctcagcagacaagaggcgaggaaggctgccaagagacaagccagaccggcagagacaagaaccaggtggaaggcgaggtgcagatcgtgtctacagctacccagaccttcctggccaccagcatcaatggcgtgctgtggacagtgtatcacggcgctggcaccagaacaatcgcctctccaaagggccccgtgacacagatgtacaccaacgtggacaaggacctcgtcggatggcaagcccctcagggctctagaagcctgacaccttgtacctgcggcagcagcgatctgtacctggtcacaagacacgccgacgtgatccccgtcagaagaagaggcgatagcagaggcagcctgctgagccctagacctatcagctacctgaagggatctgccggcggacctctgctttgtcctgctggacatgccgtgggcatctttagagccgccgtgtctactagaggcgtggccaaggccgtggacttcatccctgtggaaagcctggaaaccaccatgcggagcccctctggctcgagcggtggtggcgggagcggaggtggagggtcgtcaggtgtgaccggctaccggctgttcgaggagattctg

SEQ ID NO: 85 - DNA, 669 bp, Artificial sequence
  SmBiT_HCV_NS4A_NS3_S139A
atggtgaccggctaccggctgttcgaggagattctcgggagttccggtggtggcgggagcggaggtggaggctcgagcggtaagaaaaagggctctgtggtcatcgtgggcagaatcaacctgagcggcgataccgcctacgctcagcagacaagaggcgaggaaggctgccaagagacaagccagaccggcagagacaagaaccaggtggaaggcgaggtgcagatcgtgtctacagctacccagaccttcctggccaccagcatcaatggcgtgctgtggacagtgtatcacggcgctggcaccagaacaatcgcctctccaaagggccccgtgacacagatgtacaccaacgtggacaaggacctcgtcggatggcaagcccctcagggctctagaagcctgacaccttgtacctgcggcagcagcgatctgtacctggtcacaagacacgccgacgtgatccccgtcagaagaagaggcgatagcagaggcagcctgctgagccctagacctatcagctacctgaagggatctgccggcggacctctgctttgtcctgctggacatgccgtgggcatctttagagccgccgtgtctactagaggcgtggccaaggccgtggacttcatccctgtggaaagcctggaaaccaccatgcggagcccc

SEQ ID NO: 86 - DNA, 822 bp, Artificial sequence
  PRSIM_23_LgBiT
atgggctctagactggatgcccctagccagatcgaagtgaaggacgtgaccgacaccaccgctctgatcacctgggttgaccccagatacgacgacatctggtggttcgagctgacctacggcatcaaggatgtgcccggcgacagaaccaccatcaagctgtacctgaacgacccctactacagcatcggcaacctgaagcctgacaccgagtacgaggtgtccctgatcagctacaccggcgactcctacagcagaagcggcagcaatcctgccaagatcaccttcaagaccggcctttctggctcgagcggtggtggcgggagcggaggtggagggtcgtcaggtgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagc

SEQ ID NO: 87 - DNA, 828 bp, Artificial sequence
  PRSIM_32_LgBiT
atgggctctagactggatgcccctagccagatcgaagtgaaggacgtgaccgacaccaccgctctgatcacatggtggtccccacggtactactacgccagcatcagcggcttcgagctgacctacggcatcaaggatgtgcccggcgacagaaccaccatcaagctggactacgcctccaacgactacagcatcggcaacctgaagcctgacaccgagtacgaggtgtccctgatcagctggaactacggcgattggcggtacagcagcagcaaccctgccaagatcaccttcaagaccggcctttctggctcgagcggtggtggcgggagcggaggtggagggtcgtcaggtgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagc

SEQ ID NO: 88 - DNA, 828 bp, Artificial sequence
  PRSIM_33_LgBiT
atgggctctagactggatgcccctagccagatcgaagtgaaggacgtgaccgacaccaccgctctgatcacctggtatccacctggccgttggtacgacgacatctggtacttcgagctgacctacggcatcaaggacgtgcccggcgatagaaccaccatcaaactggccagaggcgacgacgtgtacagcatcggcaacctgaagcctgacaccgagtacgaggtgtccctgatctcttggggccctgacagaggcgatagagccggatctaaccccgccaagatcaccttcaagaccggcctttctggctcgagcggtggtggcgggagcggaggtggagggtcgtcaggtgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagc

SEQ ID NO: 89 - DNA, 828 bp, Artificial sequence
  PRSIM_36_LgBiT
atgggctctagactggatgcccctagccagatcgaagtgaaggacgtgaccgacaccaccgctctgatcacctggtcctggcctagagatgacgactacgacatctggtacttcgagctgacctacggcatcaaggacgtgcccggcgatagaaccaccatcaagctgctgaactacgcctctccatacagcatcggcaacctgaagcctgacaccgagtacgaggtgtccctgatcagcgtggtgcccgacacatatggcagaggcacaagcaaccccgccaagatcaccttcaagaccggactttctggctcgagcggtggtggcgggagcggaggtggagggtcgtcaggtgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagc

SEQ ID NO: 90 - DNA, 819 bp, Artificial sequence
  PRSIM_47_LgBiT
atgggctctagactggatgcccctagccagatcgaagtgaaggacgtgaccgacaccaccgctctgatcacctggtcaagacctggcgtgtccatctggtacttcgagctgacctacggcatcaaggacgtgcccggcgatagaaccaccatcaagctggactaccgcagctactactacagcatcggcaacctgaagcctgacaccgagtacgaggtgtccctgatcagcggctcttatggcctcgtgggcgtcagagcctctaatcccgccaagatcacctttaagaccggcctttctggctcgagcggtggtggcgggagcggaggtggagggtcgtcaggtgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagc

SEQ ID NO: 91 - DNA, 1266 bp, Artificial sequence
  PRSIM_01_LgBiT
atgggatctcaggttcagctggttcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacgagtctacaagcaccgcctacatggaactgagcagcctgagaagcgaggacaccgccgtgtactattgtgccagaggccagggctactacggctacttcgattattggggccagggcaccctggtcacagtttctagcggaggcggaggatctggtggcggaggaagtggcggaggcggttctgctcaatctgtgctgacacagcctcctagcgcctctggaacacctggccagagagtgacaatcagctgtagcggcagcagcagcaacatcggcagcaacaccgtgaactggtatcagcagctgcctggcacagcccctaaactgctgatctacagcaacaaccagcggcctagcggcgtgcccgatagattttccgtgtctaagagcggcaccagcgccagcctggctatttctggactgcagagcgaggacgaggccgactattattgtgccgcctgggatcacggacacgagcacgttgtgtttggaggcggcaccaagctgacagtgctttctggctcgagcggtggtggcgggagcggaggtggagggtcgtcaggtgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagc

SEQ ID NO: 92 - DNA, 1266 bp, Artificial sequence
  PRSIM_06_LgBiT
atgggctctcaggtgcagcttgttcagtctggcgccgaagtgaagaaacccggcagctctgtgaaggtgtcctgcaaagcttccggcggcacctttagcagctacgccatctcttgggtccgacaggctcctggacaaggcctggaatggatgggcggcatcatccctatcttcggcaccgccaattacgcccagaaattccagggcagagtgaccatcaccgccgacgagtctacaagcaccgcctacatggaactgagcagcctgagaagcgaggacaccgccgtgtactactgtgctagaggcgctggctactacatgagagtggactattggggccagggcaccctggtcacagtttctagcggaggcggaggatctggcggcggaggtagtggtggtggcggatctgctcagtctgtgctgacacagcctcctagcgcctctggaacacctggccagagagtgacaatcagctgtagcggcagcagcagcaacatcggcagcaacaccgtgaactggtatcagcagctgcctggcacagcccctaaactgctgatctacagcaacaaccagcggcctagcggcgtgcccgatagattttctggcagcaagagcggcacaagcgccagcctggctatttctggactgcagagcgaggacgaggccgactattattgtgccgcctgggaccacgacgtggaacacgttgtgtttggcggaggcaccaagctgacagtgctttctggctcgagcggtggtggcgggagcggaggtggagggtcgtcaggtgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagc

SEQ ID NO: 93 - DNA, 1266 bp, Artificial sequence
  PRSIM_57_LgBiT
atgggctctcaggttcagctggttcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacgagtctacaagcaccgcctacatggaactgagcagcctgagaagcgaggacaccgccgtgtactactgtgccagacacaccaactacatcaccgtgttcgactactggggccagggcacactggtcacagtttctagcggaggcggaggatctggtggcggaggaagtggcggaggcggttctgctcaatctgtgctgacacagcctcctagcgcctctggaacacctggccagagagtgacaatcagctgtagcggcagcagcagcaacatcggcagcaacaccgtgaactggtatcagcagctgcctggcacagcccctaaactgctgatctacagcaacaaccagcggcctagcggcgtgcccgatagattttctggcagcaagagcggcacaagcgccagcctggctatttctggactgcagagcgaggacgaggccgactattattgtgccgcctgggaccaccactgggagcaagttgtttttggaggcggcaccaagctgaccgtgctttctggctcgagcggtggtggcgggagcggaggtggagggtcgtcaggtgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagc

SEQ ID NO: 94 - DNA, 1266 bp, Artificial sequence
  PRSIM_67_LgBiT
atgggctctgaagtgcagctggttcagtctggcgccgaagtgaagaaacctggcgccgctgtcagaatcagctgcaagacaagcggctacgtgttcaccagctactacgtgcactgggtccgacaggctccaggacaaggactggaatggatgggcgtgatcaatcccagcggcggcaacaccaattacgcccagaaattccaggaccgcgtgaccatgaccagagacaccagcaccaccaccgtgtacatggaactgagcagcctgatgttcgacgacaccgccgtgtactactgcgccaagagagattacggcggacccctggccaattggggcagaggaacactggtcacagtgtctagcggaggcggaggatctggtggcggaggaagtggcggaggcggttctgctctgagctatgagctgacacagcctccaagcgtgtccgaggctcctagacagagagtgaccatcagctgtagcggcagcagcagcaacatcggcaacaacgccgtgaactggtatcagcagctgcctggcaaggcccctaaactgctgatcttctacgacgacctgctgcctagcggagtgtccgatagattcagcggctctaagagcggcacatctgccagcctggccatctctggactgcagagcgaagatgaggccgactactattgcgccgcctgggacgattctctgaacggcctggtttttggcaccggcaccaagctgacagtgctgtctggctcgagcggtggtggcgggagcggaggtggagggtcgtcaggtgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagc

SEQ ID NO: 95 - DNA, 1293 bp, Artificial sequence
  PRSIM_72_LgBiT
atgggatctcaggttcagctggttcagtctggcgccgaagtgaagaaacctggcagcagcgtgaaggtgtcctgcaaagtgtctggcggcagcttcaacaactacggcgtgtcctgggttcgacaggcccctggacaaggactggaatggatgggcagaatcatccccatccgggacaccgccaattacgcccagaaattccagggcagagtgaccatcaccgccgacaccagcaccaatatcgcctacatggaactgagcggcctgcggagtgatgacaccgccgtgtactattgcgccagagtgctggaagatgacttctggggcggctactacgacttctacttctacgtgatggacgtgtggggccagggcacactggttacagtttctagcggaggcggaggatctggtggcggaggaagtggcggaggcggttctgctctttctagcgagctgacccaggatccagtggtgtctgttcctctgggccagaccgccagaattacctgtcagggcgatagcctgaccacctactacgccacctggtatcagcagaagccaggccaggctcctgtgctggtgctgtacaatgagcacaagaggcccagcggcatcagcgacagattttctggatcttctgccggcgacgccgccagcctgacaatcacagatacacaggccgaggacgaggccgactactactgcagctctagagataccggcggcaaacacgtgctgtttggaggcggcacaaagctgacagtgctttctggctcgagcggtggtggcgggagcggaggtggagggtcgtcaggtgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagc

SEQ ID NO: 96 - DNA, 1263 bp, Artificial sequence
  PRSIM_75_LgBiT
atgggatctgaagtgcagctggttcagtctggcgccgaagtgaagaaacctggcagcagcgtgaaggtgtcctgcaaagcttctggcggcagcttcaacagctacaccctggactgggttcgacaggcccctggacaaggactggaatggatgggcggaatcatccccgtgttcggcagccctaattacggccagaaattccagggcagagtgaccatcaccgccgacgagtctacaagcaccgcctacatggaactgagcagcctgaagtccgacgacaccgccgtgtactattgtgccagaggcctggtgtaccagccactggattcttggggcagaggcaccctggtcacagtttctagcggaggcggaggatctggtggcggaggaagtggcggaggcggttctgctcaagctgttctgacacagcctagcagcgcctctggaacacctggccagagagtgacaatcagctgtagcggcagcagcagcaacatcggctcctacaccgtgaactggtatcagcagttccccggcacagcccctaagctgctgatctacagcaacacccagaggccaagcggcgtgcccgatagattttctggcagcaagagcggcacaagcgccagcctggctatttctggactgcagagcgaggacgaggccgactattattgtgccgcctgggacgacagcctgaacggatgggttttcggcggaggcaccaaagtgacagtgctttctggctcgagcggtggtggcgggagcggaggtggagggtcgtcaggtgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagc

SEQ ID NO: 97 - DNA, 813 bp, Artificial sequence
  LgBiT_PRSIM_23
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggctcgagcggtagactggatgcccctagccagatcgaagtgaaggacgtgaccgacaccaccgctctgatcacctgggttgaccccagatacgacgacatctggtggttcgagctgacctacggcatcaaggatgtgcccggcgacagaaccaccatcaagctgtacctgaacgacccctactacagcatcggcaacctgaagcctgacaccgagtacgaggtgtccctgatcagctacaccggcgactcctacagcagaagcggcagcaatcctgccaagatcaccttcaagaccggcctt

SEQ ID NO: 98 - DNA, 819 bp, Artificial sequence
  LgBiT_PRSIM_32
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggctcgagcggtagactggatgcccctagccagatcgaagtgaaggacgtgaccgacaccaccgctctgatcacatggtggtccccacggtactactacgccagcatcagcggcttcgagctgacctacggcatcaaggatgtgcccggcgacagaaccaccatcaagctggactacgcctccaacgactacagcatcggcaacctgaagcctgacaccgagtacgaggtgtccctgatcagctggaactacggcgattggcggtacagcagcagcaaccctgccaagatcaccttcaagaccggcctt

SEQ ID NO: 99 - DNA, 819 bp, Artificial sequence
  LgBiT_PRSIM_33
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggctcgagcggtagactggatgcccctagccagatcgaagtgaaggacgtgaccgacaccaccgctctgatcacctggtatccacctggccgttggtacgacgacatctggtacttcgagctgacctacggcatcaaggacgtgcccggcgatagaaccaccatcaaactggccagaggcgacgacgtgtacagcatcggcaacctgaagcctgacaccgagtacgaggtgtccctgatctcttggggccctgacagaggcgatagagccggatctaaccccgccaagatcaccttcaagaccggcctt

SEQ ID NO: 100 - DNA, 819 bp, Artificial sequence
  LgBiT_PRSIM_36
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggctcgagcggtagactggatgcccctagccagatcgaagtgaaggacgtgaccgacaccaccgctctgatcacctggtcctggcctagagatgacgactacgacatctggtacttcgagctgacctacggcatcaaggacgtgcccggcgatagaaccaccatcaagctgctgaactacgcctctccatacagcatcggcaacctgaagcctgacaccgagtacgaggtgtccctgatcagcgtggtgcccgacacatatggcagaggcacaagcaaccccgccaagatcaccttcaagaccggactt

SEQ ID NO: 101 - DNA, 810 bp, Artificial sequence
  LgBiT_PRSIM_47
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggctcgagcggtagactggatgcccctagccagatcgaagtgaaggacgtgaccgacaccaccgctctgatcacctggtcaagacctggcgtgtccatctggtacttcgagctgacctacggcatcaaggacgtgcccggcgatagaaccaccatcaagctggactaccgcagctactactacagcatcggcaacctgaagcctgacaccgagtacgaggtgtccctgatcagcggctcttatggcctcgtgggcgtcagagcctctaatcccgccaagatcacctttaagaccggcctt

SEQ ID NO: 102 - DNA, 1257 bp, Artificial sequence
  LgBiT_PRSIM_01
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggctcgagcggtcaggttcagctggttcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacgagtctacaagcaccgcctacatggaactgagcagcctgagaagcgaggacaccgccgtgtactattgtgccagaggccagggctactacggctacttcgattattggggccagggcaccctggtcacagtttctagcggaggcggaggatctggtggcggaggaagtggcggaggcggttctgctcaatctgtgctgacacagcctcctagcgcctctggaacacctggccagagagtgacaatcagctgtagcggcagcagcagcaacatcggcagcaacaccgtgaactggtatcagcagctgcctggcacagcccctaaactgctgatctacagcaacaaccagcggcctagcggcgtgcccgatagattttccgtgtctaagagcggcaccagcgccagcctggctatttctggactgcagagcgaggacgaggccgactattattgtgccgcctgggatcacggacacgagcacgttgtgtttggaggcggcaccaagctgacagtgctt

SEQ ID NO: 103 - DNA, 1257 bp, Artificial sequence
  LgBiT_PRSIM_06
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggctcgagcggtcaggtgcagcttgttcagtctggcgccgaagtgaagaaacccggcagctctgtgaaggtgtcctgcaaagcttccggcggcacctttagcagctacgccatctcttgggtccgacaggctcctggacaaggcctggaatggatgggcggcatcatccctatcttcggcaccgccaattacgcccagaaattccagggcagagtgaccatcaccgccgacgagtctacaagcaccgcctacatggaactgagcagcctgagaagcgaggacaccgccgtgtactactgtgctagaggcgctggctactacatgagagtggactattggggccagggcaccctggtcacagtttctagcggaggcggaggatctggcggcggaggtagtggtggtggcggatctgctcagtctgtgctgacacagcctcctagcgcctctggaacacctggccagagagtgacaatcagctgtagcggcagcagcagcaacatcggcagcaacaccgtgaactggtatcagcagctgcctggcacagcccctaaactgctgatctacagcaacaaccagcggcctagcggcgtgcccgatagattttctggcagcaagagcggcacaagcgccagcctggctatttctggactgcagagcgaggacgaggccgactattattgtgccgcctgggaccacgacgtggaacacgttgtgtttggcggaggcaccaagctgacagtgctt

SEQ ID NO: 104 - DNA, 1257 bp, Artificial sequence
  LgBiT_PRSIM_57
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggctcgagcggtcaggttcagctggttcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacgagtctacaagcaccgcctacatggaactgagcagcctgagaagcgaggacaccgccgtgtactactgtgccagacacaccaactacatcaccgtgttcgactactggggccagggcacactggtcacagtttctagcggaggcggaggatctggtggcggaggaagtggcggaggcggttctgctcaatctgtgctgacacagcctcctagcgcctctggaacacctggccagagagtgacaatcagctgtagcggcagcagcagcaacatcggcagcaacaccgtgaactggtatcagcagctgcctggcacagcccctaaactgctgatctacagcaacaaccagcggcctagcggcgtgcccgatagattttctggcagcaagagcggcacaagcgccagcctggctatttctggactgcagagcgaggacgaggccgactattattgtgccgcctgggaccaccactgggagcaagttgtttttggaggcggcaccaagctgaccgtgctt

SEQ ID NO: 105 - DNA, 1257 bp, Artificial sequence
  LgBiT_PRSIM_67
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggctcgagcggtgaagtgcagctggttcagtctggcgccgaagtgaagaaacctggcgccgctgtcagaatcagctgcaagacaagcggctacgtgttcaccagctactacgtgcactgggtccgacaggctccaggacaaggactggaatggatgggcgtgatcaatcccagcggcggcaacaccaattacgcccagaaattccaggaccgcgtgaccatgaccagagacaccagcaccaccaccgtgtacatggaactgagcagcctgatgttcgacgacaccgccgtgtactactgcgccaagagagattacggcggacccctggccaattggggcagaggaacactggtcacagtgtctagcggaggcggaggatctggtggcggaggaagtggcggaggcggttctgctctgagctatgagctgacacagcctccaagcgtgtccgaggctcctagacagagagtgaccatcagctgtagcggcagcagcagcaacatcggcaacaacgccgtgaactggtatcagcagctgcctggcaaggcccctaaactgctgatcttctacgacgacctgctgcctagcggagtgtccgatagattcagcggctctaagagcggcacatctgccagcctggccatctctggactgcagagcgaagatgaggccgactactattgcgccgcctgggacgattctctgaacggcctggtttttggcaccggcaccaagctgacagtgctg

SEQ ID NO: 106 - DNA, 1283 bp, Artificial sequence
  LgBiT_PRSIM_72
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggctcgagcggtcaggttcagctggttcagtctggcgccgaagtgaagaaacctggcagcagcgtgaaggtgtcctgcaaagtgtctggcggcagcttcaacaactacggcgtgtcctgggttcgacaggcccctggacaaggactggaatggatgggcagaatcatccccatccgggacaccgccaattacgcccagaaattccagggcagagtgaccatcaccgccgacaccagcaccaatatcgcctacatggaactgagcggcctgcggagtgatgacaccgccgtgtactattgcgccagagtgctggaagatgacttctggggcggctactacgacttctacttctacgtgatggacgtgtggggccagggcacactggttacagtttctagcggaggcggaggatctggtggcggaggaagtggcggaggcggttctgctctttctagcgagctgacccaggatccagtggtgtctgttcctctgggccagaccgccagaattacctgtcagggcgatagcctgaccacctactacgccacctggtatcagcagaagccaggccaggctcctgtgctggtgctgtacaatgagcacaagaggcccagcggcatcagcgacagattttctggatcttctgccggcgacgccgccagcctgacaatcacagatacacaggccgaggacgaggccgactactactgcagctctagagataccggcggcaaacacgtgctgtttggaggcggcacaaagctgacagtgct

SEQ ID NO: 107 - DNA, 1254 bp, Artificial sequence
  LgBiT_PRSIM_75
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggctcgagcggtgaagtgcagctggttcagtctggcgccgaagtgaagaaacctggcagcagcgtgaaggtgtcctgcaaagcttctggcggcagcttcaacagctacaccctggactgggttcgacaggcccctggacaaggactggaatggatgggcggaatcatccccgtgttcggcagccctaattacggccagaaattccagggcagagtgaccatcaccgccgacgagtctacaagcaccgcctacatggaactgagcagcctgaagtccgacgacaccgccgtgtactattgtgccagaggcctggtgtaccagccactggattcttggggcagaggcaccctggtcacagtttctagcggaggcggaggatctggtggcggaggaagtggcggaggcggttctgctcaagctgttctgacacagcctagcagcgcctctggaacacctggccagagagtgacaatcagctgtagcggcagcagcagcaacatcggctcctacaccgtgaactggtatcagcagttccccggcacagcccctaagctgctgatctacagcaacacccagaggccaagcggcgtgcccgatagattttctggcagcaagagcggcacaagcgccagcctggctatttctggactgcagagcgaggacgaggccgactattattgtgccgcctgggacgacagcctgaacggatgggttttcggcggaggcaccaaagtgacagtgctt

SEQ ID NO: 108 - DNA, 1209 bp, Artificial sequence
  HCV-Pro - AD fusion
atgggcaagaaaaagggcagcgtggtcatcgtgggcagaatcaacctgagcggcgataccgcctacgctcagcagacaagaggcgaggaaggctgccaagagacaagccagaccggcagagacaagaaccaggtggaaggcgaggtgcagatcgtgtctacagctacccagaccttcctggccaccagcatcaatggcgtgctgtggacagtgtatcacggcgctggcaccagaacaatcgcctctccaaagggccccgtgacacagatgtacaccaacgtggacaaggacctcgtcggatggcaagcccctcagggctctagaagcctgacaccttgtacctgcggcagcagcgatctgtacctggtcacaagacacgccgacgtgatccccgtcagaagaagaggcgatagcagaggcagcctgctgagccctagacctatcagctacctgaagggatctgccggcggacctctgctttgtcctgctggacatgccgtgggcatctttagagccgccgtgtctactagaggcgtggccaaagccgtggacttcatccctgtggaaagcctggaaaccaccatgagaagccccaccggtggcggaggatctggcggaggcggatctgatgaatttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccactagttat

SEQ ID NO: 109 - DNA, 966 bp, Artificial sequence
  DBD - HCV Pro fusion
tctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagcgctaagaaaaagggcagcgtggtcatcgtgggcagaatcaacctgagcggcgataccgcctacgctcagcagacaagaggcgaggaaggctgccaagagacaagccagaccggcagagacaagaaccaggtggaaggcgaggtgcagatcgtgtctacagctacccagaccttcctggccaccagcatcaatggcgtgctgtggacagtgtatcacggcgctggcaccagaacaatcgcctctccaaagggccccgtgacacagatgtacaccaacgtggacaaggacctcgtcggatggcaagcccctcagggctctagaagcctgacaccttgtacctgcggcagcagcgatctgtacctggtcacaagacacgccgacgtgatccccgtcagaagaagaggcgatagcagaggcagcctgctgagccctagacctatcagctacctgaagggatctgccggcggacctctgctttgtcctgctggacatgccgtgggcatctttagagccgccgtgtctactagaggcgtggccaaagccgtggacttcatccctgtggaaagcctggaaaccaccatgagaagccct

SEQ ID NO: 110 - DNA, 675 bp, Artificial sequence
  PRSIM_23_DBD_fusion
tctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagcgctggctctagactggatgcccctagccagatcgaagtgaaggacgtgaccgacaccaccgctctgatcacctgggttgaccccagatacgacgacatctggtggttcgagctgacctacggcatcaaggatgtgcccggcgacagaaccaccatcaagctgtacctgaacgacccctactacagcatcggcaacctgaagcctgacaccgagtacgaggtgtccctgatcagctacaccggcgactcctacagcagaagcggcagcaatcctgccaagatcaccttcaagaccggcctt

SEQ ID NO: 111 - DNA, 681 bp, Artificial sequence
  PRSIM_32_DBD_fusion
tctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagcgctggctctagactggatgcccctagccagatcgaagtgaaggacgtgaccgacaccaccgctctgatcacatggtggtccccacggtactactacgccagcatcagcggcttcgagctgacctacggcatcaaggatgtgcccggcgacagaaccaccatcaagctggactacgcctccaacgactacagcatcggcaacctgaagcctgacaccgagtacgaggtgtccctgatcagctggaactacggcgattggcggtacagcagcagcaaccctgccaagatcaccttcaagaccggcctt

SEQ ID NO: 112 - DNA, 681 bp, Artificial sequence
  PRSIM_33_DBD_fusion
tctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagcgctggctctagactggatgcccctagccagatcgaagtgaaggacgtgaccgacaccaccgctctgatcacctggtatccacctggccgttggtacgacgacatctggtacttcgagctgacctacggcatcaaggacgtgcccggcgatagaaccaccatcaaactggccagaggcgacgacgtgtacagcatcggcaacctgaagcctgacaccgagtacgaggtgtccctgatctcttggggccctgacagaggcgatagagccggatctaaccccgccaagatcaccttcaagaccggcctt

SEQ ID NO: 113 - DNA, 681 bp, Artificial sequence
  PRSIM_36_DBD_fusion
tctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagcgctggctctagactggatgcccctagccagatcgaagtgaaggacgtgaccgacaccaccgctctgatcacctggtcctggcctagagatgacgactacgacatctggtacttcgagctgacctacggcatcaaggacgtgcccggcgatagaaccaccatcaagctgctgaactacgcctctccatacagcatcggcaacctgaagcctgacaccgagtacgaggtgtccctgatcagcgtggtgcccgacacatatggcagaggcacaagcaaccccgccaagatcaccttcaagaccggactt

SEQ ID NO: 114 - DNA, 672 bp, Artificial sequence
  PRSIM_47_DBD_fusion
tctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagcgctggctctagactggatgcccctagccagatcgaagtgaaggacgtgaccgacaccaccgctctgatcacctggtcaagacctggcgtgtccatctggtacttcgagctgacctacggcatcaaggacgtgcccggcgatagaaccaccatcaagctggactaccgcagctactactacagcatcggcaacctgaagcctgacaccgagtacgaggtgtccctgatcagcggctcttatggcctcgtgggcgtcagagcctctaatcccgccaagatcacctttaagaccggcctt

SEQ ID NO: 115 - DNA, 1113 bp, Artificial sequence
  PRSIM_01_DBD_fusion
tctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagcgctggctctcagctggttcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacgagtctacaagcaccgcctacatggaactgagcagcctgagaagcgaggacaccgccgtgtactattgtgccagaggccagggctactacggctacttcgattattggggccagggcaccctggtcacagtttctagcggaggcggaggatctggtggcggaggaagtggcggaggcggttctgctcaatctgtgctgacacagcctcctagcgcctctggaacacctggccagagagtgacaatcagctgtagcggcagcagcagcaacatcggcagcaacaccgtgaactggtatcagcagctgcctggcacagcccctaaactgctgatctacagcaacaaccagcggcctagcggcgtgcccgatagattttccgtgtctaagagcggcaccagcgccagcctggctatttctggactgcagagcgaggacgaggccgactattattgtgccgcctgggatcacggacacgagcacgttgtgtttggaggcggcaccaagctgacagtgctt

SEQ ID NO: 116 - DNA, 1119 bp, Artificial sequence
  PRSIM_04_DBD_fusion
tctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagcgctggctctcaggtgcagcttgttcagtctggcgccgaagtgaagaaacccggcagctctgtgaaggtgtcctgcaaagcttccggcggcacctttagcagctacgccatctcttgggtccgacaggctcctggacaaggcctggaatggatgggcggcatcatccctatcttcggcaccgccaattacgcccagaaattccagggcagagtgaccatcaccgccgacgagtctacaagcaccgcctacatggaactgagcagcctgagaagcgaggacaccgccgtgtactattgcgccagaggcatggcccacttctaccagtttgatctgtggggccagggcaccctggtcacagtttctagcggaggcggaggatctggcggcggaggtagtggtggtggcggatctgctcagtctgtgctgacacagcctcctagcgcctctggaacacctggccagagagtgacaatcagctgtagcggcagcagcagcaacatcggcagcaacaccgtgaactggtatcagcagctgcctggcacagcccctaaactgctgatctacagcaacaaccagcggcctagcggcgtgcccgatagattttctggcagcaagagcggcacaagcgccagcctggctatttctggactgcagagcgaggacgaggccgactactattgtgctgccggcgatcacgaccacgagcacgttgtgtttggcggaggcaccaagctgacagtgctt

SEQ ID NO: 117 - DNA, 1119 bp, Artificial sequence
  PRSIM_57_DBD_fusion
tctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagcgctggctctcaggttcagctggttcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacgagtctacaagcaccgcctacatggaactgagcagcctgagaagcgaggacaccgccgtgtactactgtgccagacacaccaactacatcaccgtgttcgactactggggccagggcacactggtcacagtttctagcggaggcggaggatctggtggcggaggaagtggcggaggcggttctgctcaatctgtgctgacacagcctcctagcgcctctggaacacctggccagagagtgacaatcagctgtagcggcagcagcagcaacatcggcagcaacaccgtgaactggtatcagcagctgcctggcacagcccctaaactgctgatctacagcaacaaccagcggcctagcggcgtgcccgatagattttctggcagcaagagcggcacaagcgccagcctggctatttctggactgcagagcgaggacgaggccgactattattgtgccgcctgggaccaccactgggagcaagttgtttttggaggcggcaccaagctgaccgtgctt

SEQ ID NO: 118 - DNA, 1119 bp, Artificial sequence
  PRSIM_67_DBD_fusion
tctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagcgctggctctgaagtgcagctggttcagtctggcgccgaagtgaagaaacctggcgccgctgtcagaatcagctgcaagacaagcggctacgtgttcaccagctactacgtgcactgggtccgacaggctccaggacaaggactggaatggatgggcgtgatcaatcccagcggcggcaacaccaattacgcccagaaattccaggaccgcgtgaccatgaccagagacaccagcaccaccaccgtgtacatggaactgagcagcctgatgttcgacgacaccgccgtgtactactgcgccaagagagattacggcggacccctggccaattggggcagaggaacactggtcacagtgtctagcggaggcggaggatctggtggcggaggaagtggcggaggcggttctgctctgagctatgagctgacacagcctccaagcgtgtccgaggctcctagacagagagtgaccatcagctgtagcggcagcagcagcaacatcggcaacaacgccgtgaactggtatcagcagctgcctggcaaggcccctaaactgctgatcttctacgacgacctgctgcctagcggagtgtccgatagattcagcggctctaagagcggcacatctgccagcctggccatctctggactgcagagcgaagatgaggccgactactattgcgccgcctgggacgattctctgaacggcctggtttttggcaccggcaccaagctgacagtgctt

SEQ ID NO: 119 - DNA, 1146 bp, Artificial sequence
  PRSIM_72_DBD_fusion
tctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagcgctggctctcaggttcagctggttcagtctggcgccgaagtgaagaaacctggcagcagcgtgaaggtgtcctgcaaagtgtctggcggcagcttcaacaactacggcgtgtcctgggttcgacaggcccctggacaaggactggaatggatgggcagaatcatccccatccgggacaccgccaattacgcccagaaattccagggcagagtgaccatcaccgccgacaccagcaccaatatcgcctacatggaactgagcggcctgcggagtgatgacaccgccgtgtactattgcgccagagtgctggaagatgacttctggggcggctactacgacttctacttctacgtgatggacgtgtggggccagggcacactggttacagtttctagcggaggcggaggatctggtggcggaggaagtggcggaggcggttctgctctttctagcgagctgacccaggatccagtggtgtctgttcctctgggccagaccgccagaattacctgtcagggcgatagcctgaccacctactacgccacctggtatcagcagaagccaggccaggctcctgtgctggtgctgtacaatgagcacaagaggcccagcggcatcagcgacagattttctggatcttctgccggcgacgccgccagcctgacaatcacagatacacaggccgaggacgaggccgactactactgcagctctagagataccggcggcaaacacgtgctgtttggaggcggcacaaagctgacagtgctt

SEQ ID NO: 120 - DNA, 1116 bp, Artificial sequence
  PRSIM_75_DBD_fusion
tctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagcgctggctctgaagtgcagctggttcagtctggcgccgaagtgaagaaacctggcagcagcgtgaaggtgtcctgcaaagcttctggcggcagcttcaacagctacaccctggactgggttcgacaggcccctggacaaggactggaatggatgggcggaatcatccccgtgttcggcagccctaattacggccagaaattccagggcagagtgaccatcaccgccgacgagtctacaagcaccgcctacatggaactgagcagcctgaagtccgacgacaccgccgtgtactattgtgccagaggcctggtgtaccagccactggattcttggggcagaggcaccctggtcacagtttctagcggaggcggaggatctggtggcggaggaagtggcggaggcggttctgctcaagctgttctgacacagcctagcagcgcctctggaacacctggccagagagtgacaatcagctgtagcggcagcagcagcaacatcggctcctacaccgtgaactggtatcagcagttccccggcacagcccctaagctgctgatctacagcaacacccagaggccaagcggcgtgcccgatagattttctggcagcaagagcggcacaagcgccagcctggctatttctggactgcagagcgaggacgaggccgactattattgtgccgcctgggacgacagcctgaacggatgggttttcggcggaggcaccaaagtgacagtgctt

SEQ ID NO: 121 - DNA, 915 bp, Artificial sequence
  PRSIM_23_AD_fusion
atgggctctagactggatgcccctagccagatcgaagtgaaggacgtgaccgacaccaccgctctgatcacctgggttgaccccagatacgacgacatctggtggttcgagctgacctacggcatcaaggatgtgcccggcgacagaaccaccatcaagctgtacctgaacgacccctactacagcatcggcaacctgaagcctgacaccgagtacgaggtgtccctgatcagctacaccggcgactcctacagcagaagcggcagcaatcctgccaagatcaccttcaagaccggccttaccggtggcggaggatctggcggaggcggatctgatgaatttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccactagttat

SEQ ID NO: 122 - DNA, 921 bp, Artificial sequence
  PRSIM_32_AD_fusion
atgggctctagactggatgcccctagccagatcgaagtgaaggacgtgaccgacaccaccgctctgatcacatggtggtccccacggtactactacgccagcatcagcggcttcgagctgacctacggcatcaaggatgtgcccggcgacagaaccaccatcaagctggactacgcctccaacgactacagcatcggcaacctgaagcctgacaccgagtacgaggtgtccctgatcagctggaactacggcgattggcggtacagcagcagcaaccctgccaagatcaccttcaagaccggcctgaccggtggcggaggatctggcggaggcggatctgatgaatttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccactagttat

SEQ ID NO: 123 - DNA, 921 bp, Artificial sequence
  PRSIM_33_AD_fusion
atgggctctagactggatgcccctagccagatcgaagtgaaggacgtgaccgacaccaccgctctgatcacctggtatccacctggccgttggtacgacgacatctggtacttcgagctgacctacggcatcaaggacgtgcccggcgatagaaccaccatcaaactggccagaggcgacgacgtgtacagcatcggcaacctgaagcctgacaccgagtacgaggtgtccctgatctcttggggccctgacagaggcgatagagccggatctaaccccgccaagatcaccttcaagaccggccttaccggtggcggaggatctggcggaggcggatctgatgaatttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccactagttat

SEQ ID NO: 124 - DNA, 921 bp, Artificial sequence
  PRSIM_36_AD_fusion
atgggctctagactggatgcccctagccagatcgaagtgaaggacgtgaccgacaccaccgctctgatcacctggtcctggcctagagatgacgactacgacatctggtacttcgagctgacctacggcatcaaggacgtgcccggcgatagaaccaccatcaagctgctgaactacgcctctccatacagcatcggcaacctgaagcctgacaccgagtacgaggtgtccctgatcagcgtggtgcccgacacatatggcagaggcacaagcaaccccgccaagatcaccttcaagaccggacttaccggtggcggaggatctggcggaggcggatctgatgaatttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccactagttat

SEQ ID NO: 125 - DNA, 912 bp, Artificial sequence
  PRSIM_47_AD_fusion
atgggctctagactggatgcccctagccagatcgaagtgaaggacgtgaccgacaccaccgctctgatcacctggtcaagacctggcgtgtccatctggtacttcgagctgacctacggcatcaaggacgtgcccggcgatagaaccaccatcaagctggactaccgcagctactactacagcatcggcaacctgaagcctgacaccgagtacgaggtgtccctgatcagcggctcttatggcctcgtgggcgtcagagcctctaatcccgccaagatcacctttaagaccggccttaccggtggcggaggatctggcggaggcggatctgatgaatttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccactagttat

SEQ ID NO: 126 - DNA, 1359 bp, Artificial sequence
  PRSIM_01_AD_fusion
atgggctctcaggttcagctggttcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacgagtctacaagcaccgcctacatggaactgagcagcctgagaagcgaggacaccgccgtgtactattgtgccagaggccagggctactacggctacttcgattattggggccagggcaccctggtcacagtttctagcggaggcggaggatctggtggcggaggaagtggcggaggcggttctgctcaatctgtgctgacacagcctcctagcgcctctggaacacctggccagagagtgacaatcagctgtagcggcagcagcagcaacatcggcagcaacaccgtgaactggtatcagcagctgcctggcacagcccctaaactgctgatctacagcaacaaccagcggcctagcggcgtgcccgatagattttccgtgtctaagagcggcaccagcgccagcctggctatttctggactgcagagcgaggacgaggccgactattattgtgccgcctgggatcacggacacgagcacgttgtgtttggaggcggcaccaagctgacagtgcttaccggtggcggaggatctggcggaggcggatctgatgaatttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccactagttat

SEQ ID NO: 127 - DNA, 1359 bp, Artificial sequence
  PRSIM_04_AD_fusion
atgggctctcaggtgcagcttgttcagtctggcgccgaagtgaagaaacccggcagctctgtgaaggtgtcctgcaaagcttccggcggcacctttagcagctacgccatctcttgggtccgacaggctcctggacaaggcctggaatggatgggcggcatcatccctatcttcggcaccgccaattacgcccagaaattccagggcagagtgaccatcaccgccgacgagtctacaagcaccgcctacatggaactgagcagcctgagaagcgaggacaccgccgtgtactattgcgccagaggcatggcccacttctaccagtttgatctgtggggccagggcaccctggtcacagtttctagcggaggcggaggatctggcggcggaggtagtggtggtggcggatctgctcagtctgtgctgacacagcctcctagcgcctctggaacacctggccagagagtgacaatcagctgtagcggcagcagcagcaacatcggcagcaacaccgtgaactggtatcagcagctgcctggcacagcccctaaactgctgatctacagcaacaaccagcggcctagcggcgtgcccgatagattttctggcagcaagagcggcacaagcgccagcctggctatttctggactgcagagcgaggacgaggccgactactattgtgctgccggcgatcacgaccacgagcacgttgtgtttggcggaggcaccaagctgacagtgcttaccggtggcggaggatctggcggaggcggatctgatgaatttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccactagttat

SEQ ID NO: 128 - DNA, 1359 bp, Artificial sequence
  PRSIM_57_AD_fusion
atgggctctcaggttcagctggttcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacgagtctacaagcaccgcctacatggaactgagcagcctgagaagcgaggacaccgccgtgtactactgtgccagacacaccaactacatcaccgtgttcgactactggggccagggcacactggtcacagtttctagcggaggcggaggatctggtggcggaggaagtggcggaggcggttctgctcaatctgtgctgacacagcctcctagcgcctctggaacacctggccagagagtgacaatcagctgtagcggcagcagcagcaacatcggcagcaacaccgtgaactggtatcagcagctgcctggcacagcccctaaactgctgatctacagcaacaaccagcggcctagcggcgtgcccgatagattttctggcagcaagagcggcacaagcgccagcctggctatttctggactgcagagcgaggacgaggccgactattattgtgccgcctgggaccaccactgggagcaagttgtttttggaggcggcaccaagctgaccgtgcttaccggtggcggaggatctggcggaggcggatctgatgaatttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccactagttat

SEQ ID NO: 129 - DNA, 1359 bp, Artificial sequence
  PRSIM_67_AD_fusion
atgggctctgaagtgcagctggttcagtctggcgccgaagtgaagaaacctggcgccgctgtcagaatcagctgcaagacaagcggctacgtgttcaccagctactacgtgcactgggtccgacaggctccaggacaaggactggaatggatgggcgtgatcaatcccagcggcggcaacaccaattacgcccagaaattccaggaccgcgtgaccatgaccagagacaccagcaccaccaccgtgtacatggaactgagcagcctgatgttcgacgacaccgccgtgtactactgcgccaagagagattacggcggacccctggccaattggggcagaggaacactggtcacagtgtctagcggaggcggaggatctggtggcggaggaagtggcggaggcggttctgctctgagctatgagctgacacagcctccaagcgtgtccgaggctcctagacagagagtgaccatcagctgtagcggcagcagcagcaacatcggcaacaacgccgtgaactggtatcagcagctgcctggcaaggcccctaaactgctgatcttctacgacgacctgctgcctagcggagtgtccgatagattcagcggctctaagagcggcacatctgccagcctggccatctctggactgcagagcgaagatgaggccgactactattgcgccgcctgggacgattctctgaacggcctggtttttggcaccggcaccaagctgacagtgctgaccggtggcggaggatctggcggaggcggatctgatgaatttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccactagttat

SEQ ID NO: 130 - DNA, 1386 bp, Artificial sequence
  PRSIM_72_AD_fusion
atgggctctcaggttcagctggttcagtctggcgccgaagtgaagaaacctggcagcagcgtgaaggtgtcctgcaaagtgtctggcggcagcttcaacaactacggcgtgtcctgggttcgacaggcccctggacaaggactggaatggatgggcagaatcatccccatccgggacaccgccaattacgcccagaaattccagggcagagtgaccatcaccgccgacaccagcaccaatatcgcctacatggaactgagcggcctgcggagtgatgacaccgccgtgtactattgcgccagagtgctggaagatgacttctggggcggctactacgacttctacttctacgtgatggacgtgtggggccagggcacactggttacagtttctagcggaggcggaggatctggtggcggaggaagtggcggaggcggttctgctctttctagcgagctgacccaggatccagtggtgtctgttcctctgggccagaccgccagaattacctgtcagggcgatagcctgaccacctactacgccacctggtatcagcagaagccaggccaggctcctgtgctggtgctgtacaatgagcacaagaggcccagcggcatcagcgacagattttctggatcttctgccggcgacgccgccagcctgacaatcacagatacacaggccgaggacgaggccgactactactgcagctctagagataccggcggcaaacacgtgctgtttggaggcggcacaaagctgacagtgctgaccggtggcggaggatctggcggaggcggatctgatgaatttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccactagttat

SEQ ID NO: 131 - DNA, 1356 bp, Artificial sequence
  PRSIM_75_AD_fusion
atgggctctgaagtgcagctggttcagtctggcgccgaagtgaagaaacctggcagcagcgtgaaggtgtcctgcaaagcttctggcggcagcttcaacagctacaccctggactgggttcgacaggcccctggacaaggactggaatggatgggcggaatcatccccgtgttcggcagccctaattacggccagaaattccagggcagagtgaccatcaccgccgacgagtctacaagcaccgcctacatggaactgagcagcctgaagtccgacgacaccgccgtgtactattgtgccagaggcctggtgtaccagccactggattcttggggcagaggcaccctggtcacagtttctagcggaggcggaggatctggtggcggaggaagtggcggaggcggttctgctcaagctgttctgacacagcctagcagcgcctctggaacacctggccagagagtgacaatcagctgtagcggcagcagcagcaacatcggctcctacaccgtgaactggtatcagcagttccccggcacagcccctaagctgctgatctacagcaacacccagaggccaagcggcgtgcccgatagattttctggcagcaagagcggcacaagcgccagcctggctatttctggactgcagagcgaggacgaggccgactattattgtgccgcctgggacgacagcctgaacggatgggttttcggcggaggcaccaaagtgacagtgctgaccggtggcggaggatctggcggaggcggatctgatgaatttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccactagttat

SEQ ID NO: 132 - DNA, 2592 bp, Artificial sequence
  FKBP12:FRB CAR
atgctgctgctggttacatctctgctgctgtgcgagctgccccatcctgcctttctgctgattcctgaaagcaaatacggccctccgtgtcctccttgtcctttctgggtgctcgtggttgttggcggagtgctggcctgttatagcctgcttgtgaccgtggccttcatcatcttttgggtcaagcggggcagaaagaagctgctgtacatcttcaagcagcccttcatgcggcccgtgcagaccacacaagaggaagatggctgctcctgcagattccccgaggaagaagaaggcggctgcgagctgtctagaggcagcggatcaggctctggatctatgggcgtgcaggtcgagacaatctctcctggcgacggcagaacattccccaagaggggccagacatgcgtggtgcactataccggcatgctcgaggacggcaagaagttcgacagctcccgggacagaaacaagcccttcaagttcatgctgggcaagcaagaagtgatcagaggctgggaagagggcgtcgcccagatgtctgttggccagagagccaaactgacaatcagccccgattacgcctacggcgccacaggacaccctggaatcattcctccacacgccacactggtgttcgacgtggaactgctgaagctggaaggcagcggcgccaccaatttcagcctgctgaaacaggccggcgacgtcgaagagaaccccggacctatgatccacctgggccacattctgtttctgttgctgctgcctgtggccgctgctcagacaacacctggcgagagatctagcctgcctgccttctatcctggcaccagcggctcttgttctggctgtggatctctgagcctgccagagtctaagtacggccctccgtgtccaccatgtccattttgggtcctcgttgtcgtcggaggcgtgctggcttgctattctctgctcgtgacagtcgcctttattatcttctgggtgtccctgaagagaggccggaaaaaactgctctatatctttaaacagccgtttatgcgcccggtccagacaacccaagaagaggacggctgtagctgccggtttcctgaagaagaggaaggcggttgcgaactgatcctgtggcacgagatgtggcatgaaggcctggaagaggccagcagactgtacttcggcgagagaaacgtgaaaggcatgttcgaggtgctggaacctctgcacgccatgatggaaagaggccctcagacactgaaagagacaagcttcaaccaggcctacggccgggatctgatggaagcccaagagtggtgccggaagtacatgaagtccggcaacgtgaaggacctgctgcaggcctgggatctgtactaccacgtgttccggcggatcagcaaaggctccggaagcggatctggaagctccctgagagtgaagttcagcagaagcgccgacgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggtagtggcgaaggcagaggctctctgctgacatgcggagatgtggaagagaatcctgggccaagcggcatggaaagcgacgaatctggactccccgccatggaaatcgagtgcagaatcaccggcacactgaacggcgtggaattcgaactcgttggaggcggcgagggcacacctaagcagggcagaatgaccaacaagatgaagtccaccaaaggcgccctgactttcagcccctacctgctgtctcacgtgatgggctacggcttctaccacttcggcacataccctagcggctacgagaaccccttcctgcatgccatcaacaacggcggctacaccaacaccagaatcgagaagtacgaggatggcggcgtgctgcacgtgtccttcagctacagatatgaggccggcagagtgatcggcgacttcaaggttgtcggcaccggctttccagaggacagcgtgatcttcaccgacaagatcatccggtccaacgccaccgtcgagcatctgcaccctatgggcgataatgtgcttgtgggcagcttcgccagaaccttcagtctgcgtgatggcggctactacagcttcgtggtggacagccacatgcacttcaagagcgccatccatcctagcatcctgcagaacggcggacccatgttcgccttcagaagagtggaagaactgcactccaacaccgagctgggcatcgtggaataccagcacgctttcaagacccctatcgccttcgcaagaagcagagcccagagcagcaatagcgccgtggatggaacagccggacctggctctacaggctccaga

SEQ ID NO: 133 - DNA, 2913 bp, Artificial sequence
  PRSIM_23 CAR
atgctgctgctggttacatctctgctgctgtgcgagctgccccatcctgcctttctgctgattcctgaaagcaaatacggccctccgtgtcctccttgtcctttctgggtgctcgtggttgttggcggagtgctggcctgttatagcctgcttgtgaccgtggccttcatcatcttttgggtcaagcggggcagaaagaagctgctgtacatcttcaagcagcccttcatgcggcccgtgcagaccacacaagaggaagatggctgctcctgcagattccccgaggaagaagaaggcggctgcgagctgggtggcggaggatctggcggaggcggatctatgaagaaaaagggctctgtggtcatcgtgggcagaatcaacctgagcggcgataccgcctacgctcagcagacaagaggcgaggaaggctgccaagagacaagccagaccggcagagacaagaaccaggtggaaggcgaggtgcagatcgtgtctacagctacccagaccttcctggccaccagcatcaatggcgtgctgtggacagtgtatcacggcgctggcaccagaacaatcgcctctccaaagggccccgtgacacagatgtacaccaacgtggacaaggacctcgtcggatggcaagcccctcagggctctagaagcctgacaccttgtacctgcggcagcagcgatctgtacctggtcacaagacacgccgacgtgatccccgtcagaagaagaggcgatagcagaggcagcctgctgagccctagacctatcagctacctgaagggatctgccggcggacctctgctttgtcctgctggacatgccgtgggcatctttagagccgccgtgtctactagaggcgtggccaaggccgtggacttcatccctgtggaaagcctggaaaccaccatgcggagccccggcagcggcgccaccaatttcagcctgctgaaacaggccggcgacgtcgaagagaaccccggacctatgatccacctgggccacattctgtttctgttgctgctgcctgtggccgctgctcagacaacacctggcgagagatctagcctgcctgccttctatcctggcaccagcggctcttgttctggctgtggatctctgagcctgccagagtctaagtacggccctccgtgtccaccatgtccattttgggtcctcgttgtcgtcggaggcgtgctggcttgctattctctgctcgtgacagtcgcctttattatcttctgggtgtccctgaagagaggccggaaaaaactgctctatatctttaaacagccgtttatgcgcccggtccagacaacccaagaagaggacggctgtagctgccggtttcctgaagaagaggaaggcggttgcgaactgggtggcggaggatctggcggaggcggatctatgggctctagactggatgcccctagccagatcgaagtgaaggacgtgaccgacaccaccgctctgatcacctgggttgaccccagatacgacgacatctggtggttcgagctgacctacggcatcaaggatgtgcccggcgacagaaccaccatcaagctgtacctgaacgacccctactacagcatcggcaacctgaagcctgacaccgagtacgaggtgtccctgatcagctacaccggcgactcctacagcagaagcggcagcaatcctgccaagatcaccttcaagaccggccttggtggcggaggatctggcggaggcggatctctgagagtgaagttcagcagaagcgccgacgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggtagtggcgaaggcagaggctctctgctgacatgcggagatgtggaagagaatcctgggccaagcggcatggaaagcgacgaatctggactccccgccatggaaatcgagtgcagaatcaccggcacactgaacggcgtggaattcgaactcgttggaggcggcgagggcacacctaagcagggcagaatgaccaacaagatgaagtccaccaaaggcgccctgactttcagcccctacctgctgtctcacgtgatgggctacggcttctaccacttcggcacataccctagcggctacgagaaccccttcctgcatgccatcaacaacggcggctacaccaacaccagaatcgagaagtacgaggatggcggcgtgctgcacgtgtccttcagctacagatatgaggccggcagagtgatcggcgacttcaaggttgtcggcaccggctttccagaggacagcgtgatcttcaccgacaagatcatccggtccaacgccaccgtcgagcatctgcaccctatgggcgataatgtgcttgtgggcagcttcgccagaaccttcagtctgcgtgatggcggctactacagcttcgtggtggacagccacatgcacttcaagagcgccatccatcctagcatcctgcagaacggcggacccatgttcgccttcagaagagtggaagaactgcactccaacaccgagctgggcatcgtggaataccagcacgctttcaagacccctatcgccttcgcaagaagcagagcccagagcagcaatagcgccgtggatggaacagccggacctggctctacaggctccaga

SEQ ID NO: 134 - protein, 92 aa, Artificial sequence
  Wild type TN3
RLDAPSQIEVKDVTDTTALITWFKPLAEIDGIELTYGIKDVPGDRTTIDLTEDENQYSIGNLKPDTEYEVSLISRRGDMSSNPAKETFTTGL

SEQ ID NO: 135 - protein, 92 aa, Artificial sequence
  Wild type TN3 with stabilising mutations
RLDAPSQIEVKDVTDTTALITWFKPLAEIDGFELTYGIKDVPGDRTTIKLTEDENQYSIGNLKPDTEYEVSLISRRGDMSSNPAKITFKTGL

SEQ ID NO: 136 - protein, 10 aa, Artificial sequence
  PRSIM_23 BC loop
VDPRYDDIWW

SEQ ID NO: 137 - protein, 6 aa, Artificial sequence
  PRSIM_23 DE loop
YLNDPY

SEQ ID NO: 138 - protein, 14 aa, Artificial sequence
  PRSIM_23 FG loop
YTGDSYSRSGSNPA

SEQ ID NO: 139 - protein, 12 aa, Artificial sequence
  PRSIM_32 BC loop
WSPRYYYASISG

SEQ ID NO: 140 - protein, 6 aa, Artificial sequence
  PRSIM_32 DE loop
DYASND

SEQ ID NO: 141 - protein, 14 aa, Artificial sequence
  PRSIM_32 FG loop
WNYGDWRYSSSNPA

SEQ ID NO: 142 - protein, 12 aa, Artificial sequence
  PRSIM_33 BC loop
YPPGRWYDDIWY

SEQ ID NO: 143 - protein, 6 aa, Artificial sequence
  PRSIM_33 DE loop
ARGDDV

SEQ ID NO: 144 - protein, 14 aa, Artificial sequence
  PRSIM_33 FG loop
WGPDRGDRAGSNPA

SEQ ID NO: 145 - protein, 12 aa, Artificial sequence
  PRSIM_36 BC loop
SWPRDDDYDIWY

SEQ ID NO: 146 - protein, 6 aa, Artificial sequence
  PRSIM_36 DE loop
LNYASP

SEQ ID NO: 147 - protein, 14 aa, Artificial sequence
  PRSIM_36 FG loop
VVPDTYGRGTSNPA

SEQ ID NO: 148 - protein, 9 aa, Artificial sequence
  PRSIM_47 BC loop
SRPGVSIWY

SEQ ID NO: 149 - protein, 6 aa, Artificial sequence
  PRSIM_47 DE loop
DYRSYY

SEQ ID NO: 150 - protein, 14 aa, Artificial sequence
  PRSIM_47 FG loop
GSYGLVGVRASNPA

SEQ ID NO: 151 - protein, 5 aa, Artificial sequence
  PRSIM_57 HCDR1
SYAIS

SEQ ID NO: 152 - protein, 17 aa, Artificial sequence
  PRSIM_57 HCDR2
GIIPIFGTANYAQKFQG

SEQ ID NO: 153 - protein, 10 aa, Artificial sequence
  PRSIM_57 HCDR3
HTNYITVFDY

SEQ ID NO: 154 - protein, 13 aa, Artificial sequence
  PRSIM_57 LCDR1
SGSSSNIGSNTVN

SEQ ID NO: 155 - protein, 7 aa, Artificial sequence
  PRSIM_57 LCDR2
SNNQRPS

SEQ ID NO: 156 - protein, 11 aa, Artificial sequence
  PRSIM_57 LCDR3
AAWDHHWEQVV

SEQ ID NO: 157-162 - skipped (6 entries)

SEQ ID NO: 163 - protein, 10 aa, Artificial Sequence
  PRSIM_04 HCDR3
GMAHFYQFDL

SEQ ID NO: 164 - protein, 11 aa, Artificial Sequence
  PRSIM_04 LCDR3
AAGDHDHEHVV

SEQ ID NO: 165 - protein, 5 aa, Artificial sequence
  PRSIM_67 HCDR1
SYYVH

SEQ ID NO: 166 - protein, 17 aa, Artificial sequence
  PRSIM_67 HCDR2
VINPSGGNTNYAQKFQD

SEQ ID NO: 167 - protein, 9 aa, Artificial sequence
  PRSIM_67 HCDR3
RDYGGPLAN

SEQ ID NO: 168 - protein, 13 aa, Artificial sequence
  PRSIM_67 LCDR1
SGSSSNIGNNAVN

SEQ ID NO: 169 - protein, 7 aa, Artificial sequence
  PRSIM_67 LCDR2
YDDLLPS

SEQ ID NO: 170 - protein, 11 aa, Artificial sequence
  PRSIM_67 LCDR3
AAWDDSLNGLV

SEQ ID NO: 171 - protein, 5 aa, Artificial sequence
  PRSIM_72 HCDR1
NYGVS

SEQ ID NO: 172 - protein, 17 aa, Artificial sequence
  PRSIM_72 HCDR2
RIIPIRDTANYAQKFQG

SEQ ID NO: 173 - protein, 20 aa, Artificial sequence
  PRSIM_72 HCDR3
VLEDDFWGGYYDFYFYVMDV

SEQ ID NO: 174 - protein, 11 aa, Artificial sequence
  PRSIM_72 LCDR1
QGDSLTTYYAT

SEQ ID NO: 175 - protein, 7 aa, Artificial sequence
  PRSIM_72 LCDR2
NEHKRPS

SEQ ID NO: 176 - protein, 11 aa, Artificial sequence
  PRSIM_72 LCDR3
SSRDTGGKHVL

SEQ ID NO: 177 - protein, 5 aa, Artificial sequence
  PRSIM_75 HCDR1
SYTLD

SEQ ID NO: 178 - protein, 17 aa, Artificial sequence
  PRSIM_75 HCDR2
GIIPVFGSPNYGQKFQG

SEQ ID NO: 179 - protein, 9 aa, Artificial sequence
  PRSIM_75 HCDR3
GLVYQPLDS

SEQ ID NO: 180 - protein, 13 aa, Artificial sequence
  PRSIM_75 LCDR1
SGSSSNIGSYTVN

SEQ ID NO: 181 - protein, 7 aa, Artificial sequence
  PRSIM_75 LCDR2
SNTQRPS

SEQ ID NO: 182 - protein, 11 aa, Artificial sequence
  PRSIM_75 LCDR3
AAWDDSLNGWV

SEQ ID NO: 183 - DNA, 61 bp, Artificial sequence
  Tn3_pETFwd2
cgatcatatggactacaaggacgacgatgacaagggcagccgtctggatgcaccgagccag

SEQ ID NO: 184 - DNA, 43 bp, Artificial sequence
  Tn3_pETRev2
atcgggatccctacagaccggttttaaaggtaatttttgccgg

SEQ ID NO: 185 - protein, 11 aa, Artificial sequence
  Linker
TGGGGSGGGGS

SEQ ID NO: 186 - protein, 119 aa, Artificial sequence
  PRSIM_57 VH
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARHTNYITVFDYWGQGTLVTVSS

SEQ ID NO: 187 - protein, 110 aa, Artificial sequence
  PRSIM_57 VL
QSVLTQPPSASGTPGQRVTISCSGSSSNIGSNTVNWYQQLPGTAPKLLIYSNNQRPSGVPDRFSGSKSGTSASLAISGLQSEDEADYYCAAWDHHWEQVVFGGGTKLTVL

SEQ ID NO: 188 - protein, 119 aa, Artificial sequence
  PRSIM_01 VH
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGQGYITVFDYWGQGTLVTVSS

SEQ ID NO: 189 - protein, 110 aa, Artificial sequence; identical to SEQ ID NO: 187
  PRSIM_01 VL

SEQ ID NO: 190 - protein, 119 aa, Artificial sequence
  PRSIM_04 VH
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGMAHFYQFDLWGQGTLVTVSS

SEQ ID NO: 191 - protein, 110 aa, Artificial sequence
  PRSIM_04 VL
QSVLTQPPSASGTPGQRVTISCSGSSSNIGSNTVNWYQQLPGTAPKLLIYSNNQRPSGVPDRFSGSKSGTSASLAISGLQSEDEADYYCAAGDHDHEHVVFGGGTKLTVL

SEQ ID NO: 192 - protein, 118 aa, Artificial sequence
  PRSIM_67 VH
EVQLVQSGAEVKKPGAAVRISCKTSGYVFTSYYVHWVRQAPGQGLEWMGVINPSGGNTNYAQKFQDRVTMTRDTSTTTVYMELSSLMFDDTAVYYCAKRDYGGPLANWGRGTLVTVSS

SEQ ID NO: 193 - protein, 110 aa, Artificial sequence
  PRSIM_67 VL
SYELTQPPSVSEAPRQRVTISCSGSSSNIGNNAVNWYQQLPGKAPKLLIFYDDLLPSGVSDRFSGSKSGTSASLAISGLQSEDEADYYCAAWDDSLNGLVFGTGTKLTVL

SEQ ID NO: 194 - protein, 119 aa, Artificial sequence; identical to SEQ ID NO: 190
  PRSIM_72 VH

SEQ ID NO: 195 - protein, 110 aa, Artificial sequence; identical to SEQ ID NO: 191
  PRSIM_72 VL

SEQ ID NO: 196 - protein, 118 aa, Artificial sequence
  PRSIM_75 VH
EVQLVQSGAEVKKPGSSVKVSCKASGGSFNSYTLDWVRQAPGQGLEWMGGIIPVFGSPNYGQKFQGRVTITADESTSTAYMELSSLKSDDTAVYYCARGLVYQPLDSWGRGTLVTVSS

SEQ ID NO: 197 - protein, 110 aa, Artificial sequence
  PRSIM_75 VL
QAVLTQPSSASGTPGQRVTISCSGSSSNIGSYTVNWYQQFPGTAPKLLIYSNTQRPSGVPDRFSGSKSGTSASLAISGLQSEDEADYYCAAWDDSLNGWVFGGGTKVTVL

SEQ ID NO: 198 - protein, 10 aa, Artificial Sequence
  PRSIM_01 HCDR3
GQGYITVFDY

SEQ ID NO: 199 - protein, 631 aa, Artificial sequence
  Full length NS3 protein
APITAYAQQTRGEEGCQETSLTGRDKNQVEGEVQIVSTAAQTFLATSINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGIFRAAVCTRGVAKAVDFIPVENLETTMRSPVFTDNSSPPVVPQSFQVAHLHAPTGSGKSTKVPAAYAAQGYKVLVLNPSVAATLGFGAYMSKAHGIDPNIRTGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGYTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEITLTHPVTKYIMTCMSADLEVVT

SEQ ID NO: 200 - protein, 319 aa, Artificial sequence
  PRSIM_23 CAR 2nd  polypeptide
ESKYGPPCPPCPFWVLVVVGGVLACYSLLVTVAFIIFWVSLKRGRKKLLYIFKQPFMRPVQTTQEEDGCSCRFPEEEEGGCELGGGGSGGGGSMGSRLDAPSQIEVKDVTDTTALITWVDPRYDDIWWFELTYGIKDVPGDRTTIKLYLNDPYYSIGNLKPDTEYEVSLISYTGDSYSRSGSNPAKITFKTGLGGGGSGGGGSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSG

SEQ ID NO: 201 - protein, 22 aa, Artificial sequence
  PRSIM_23 CAR 1st  signal peptide
MLLLVTSLLLCELPHPAFLLIP

SEQ ID NO: 202 - protein, 48 aa, Artificial sequence
  PRSIM_23 CAR 2nd signal peptide
MIHLGHILFLLLLPVAAAQTTPGERSSLPAFYPGTSGSCSGCGSLSLP

SEQ ID NO: 203 - protein, 367 aa, Artificial sequence
  PRSIM_23 CAR 2nd  polypeptide + signal peptide
MIHLGHILFLLLLPVAAAQTTPGERSSLPAFYPGTSGSCSGCGSLSLPESKYGPPCPPCPFWVLVVVGGVLACYSLLVTVAFIIFWVSLKRGRKKLLYIFKQPFMRPVQTTQEEDGCSCRFPEEEEGGCELGGGGSGGGGSMGSRLDAPSQIEVKDVTDTTALITWVDPRYDDIWWFELTYGIKDVPGDRTTIKLYLNDPYYSIGNLKPDTEYEVSLISYTGDSYSRSGSNPAKITFKTGLGGGGSGGGGSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSG

SEQ ID NO: 204 - protein, 10 aa, Artificial sequence
  Linker
GGGGSGGGGS

SEQ ID NO: 205 - protein, 458 aa, Artificial sequence
  MEDI8852 heavy chain
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - protein, 210 aa, Artificial Sequence
  MEDI8852 light chain
DIQMTQSPSSLSASVGDRVTITCRTSQSLSSYTHWYQQKPGKAPKLLIYAASSRGSGVPSRFSGSGSGTDFTLTISSLQPEDFATYYCQQSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - protein, 849 aa, Artificial sequence
  DBD-PRSIM_23x3-P2A-NS4A/3 PR S139A-AD
MDYPAAKRVKLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTSAGSRLDAPSQIEVKDVTDTTALITWVDPRYDDIWWFELTYGIKDVPGDRTTIKLYLNDPYYSIGNLKPDTEYEVSLISYTGDSYSRSGSNPAKITFKTGLRLDAPSQIEVKDVTDTTALITWVDPRYDDIWWFELTYGIKDVPGDRTTIKLYLNDPYYSIGNLKPDTEYEVSLISYTGDSYSRSGSNPAKITFKTGLRLDAPSQIEVKDVTDTTALITWVDPRYDDIWWFELTYGIKDVPGDRTTIKLYLNDPYYSIGNLKPDTEYEVSLISYTGDSYSRSGSNPAKITFKTGLTSKGSGATNFSLLKQAGDVEENPGPMAKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPTRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSTSY

SEQ ID NO: 208 - DNA, 2547 bp, Artificial sequence
  DBD-PRSIM_23x3-P2A-NS4A/3 PR S139A-AD
atggactatcctgctgccaagagggtcaagttggactctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagcgctggctctagactggatgcccctagccagatcgaagtgaaggacgtgaccgacaccaccgctctgatcacctgggttgaccccagatacgacgacatctggtggttcgagctgacctacggcatcaaggatgtgcccggcgacagaaccaccatcaagctgtacctgaacgacccctactacagcatcggcaacctgaagcctgacaccgagtacgaggtgtccctgatcagctacaccggcgactcctacagcagaagcggcagcaatcctgccaagatcaccttcaagaccggcctgagactggacgcaccctctcagattgaagtcaaagatgtcaccgacacgacagccctgattacatgggttgaccctcgctacgatgatatttggtggtttgaactcacgtacgggatcaaagacgtgccaggggatcgcacaacaatcaagctctatctcaatgatccgtactactccatcgggaatctgaaacccgatacagagtacgaagtctccctcatctcttacaccggggacagctactccagatccggctccaatccagccaaaattacgtttaagacaggcctgcggctggatgctccatctcaaatagaagttaaggatgtgacggatacgacggccctcatcacttgggttgaccctcgatatgacgatatttggtggttcgaattgacgtatggcattaaggacgtcccaggcgaccggacaactattaagctgtatcttaacgatccttattatagcatcggaaatctcaagccggataccgaatatgaggtttccctcatttcctatactggggactcctactctcgctccggctctaacccagctaagatcacttttaaaaccgggcttacttcgaaaggaagcggcgccacaaactttagcctgctgaaacaggccggcgacgtcgaagaaaatcccgggcctatggctaaaaagggctctgtggtcatcgtgggcagaatcaacctgagcggcgataccgcctacgctcagcagacaagaggcgaggaaggctgccaagagacaagccagaccggcagagacaagaaccaggtggaaggcgaggtgcagatcgtgtctacagctacccagaccttcctggccaccagcatcaatggcgtgctgtggacagtgtatcacggcgctggcaccagaacaatcgcctctccaaagggccccgtgacacagatgtataccaacgtggacaaggacctcgtcggatggcaagcccctcagggctctagaagcctgacaccttgtacctgcggcagcagcgatctgtacctggtcacaagacacgccgacgtgatccccgtcagaagaagaggcgatagcagaggcagcctgctgagccctagacctatcagctacctgaagggatctgccggcggacctctgctttgtcctgctggacatgccgtgggcatctttagagccgccgtgtctactagaggcgtggccaaggccgtggacttcatccctgtggaaagcctggaaaccaccatgcggagccccactagagatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccactagttat

SEQ ID NO: 209 - protein, 153 aa, Artificial sequence
  huIL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 210 - DNA, 459 bp, Artificial sequence
  huIL-2
atgtacaggatgcaactcctgtcttgcattgcactaagtcttgcacttgtcacaaacagtgcccctaccagcagcagcaccaagaaaacccagctgcaactggaacacctcctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacc

SEQ ID NO: 211 - protein, 199 aa, Artificial sequence
  NS4A/3 PR S139A K136D
MGKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLDGSAGGPLLCPAGHAVGIFRAAVSTRGVAKAVDFIPVESLETTMRSPT

SEQ ID NO: 212 - DNA, 597 bp, Artificial sequence
  NS4A/3 PR S139A K136D
atgggcaagaaaaagggcagcgtggtcatcgtgggcagaatcaacctgagcggcgataccgcctacgctcagcagacaagaggcgaggaaggctgccaagagacaagccagaccggcagagacaagaaccaggtggaaggcgaggtgcagatcgtgtctacagctacccagaccttcctggccaccagcatcaatggcgtgctgtggacagtgtatcacggcgctggcaccagaacaatcgcctctccaaagggccccgtgacacagatgtacaccaacgtggacaaggacctcgtcggatggcaagcccctcagggaagcagaagcctgacaccttgtacctgcggcagcagcgatctgtacctggtcacaagacacgccgacgtgatccccgtcagaagaagaggcgatagcagaggcagcctgctgagccctagacctatcagctacctggatggatctgccggcggacctctgctttgtcctgctggacatgccgtgggcatctttagagccgccgtgtctactagaggcgtggccaaagccgtggacttcatccccgtggaaagcctggaaaccaccatgagatctccaacc

SEQ ID NO: 213 - protein, 199 aa, Artificial sequence
  NS4A/3 PR S139A D168E
MGKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVEFIPVESLETTMRSPT

SEQ ID NO: 214 - DNA, 597 bp, Artificial sequence
  NS4A/3 PR S139A D168E
atgggcaagaaaaagggcagcgtggtcatcgtgggcagaatcaacctgagcggcgataccgcctacgctcagcagacaagaggcgaggaaggctgccaagagacaagccagaccggcagagacaagaaccaggtggaaggcgaggtgcagatcgtgtctacagctacccagaccttcctggccaccagcatcaatggcgtgctgtggacagtgtatcacggcgctggcaccagaacaatcgcctctccaaagggccccgtgacacagatgtacaccaacgtggacaaggacctcgtcggatggcaagcccctcagggaagcagaagcctgacaccttgtacctgcggcagcagcgatctgtacctggtcacaagacacgccgacgtgatccccgtcagaagaagaggcgatagcagaggcagcctgctgagccctagacctatcagctacctgaagggatctgccggcggacctctgctttgtcctgctggacatgccgtgggcatctttagagccgccgtgtctactagaggcgtggccaaagccgtggaattcatccccgtggaaagcctggaaaccaccatgagatctccaacc

SEQ ID NO: 215 - protein, 199 aa, Artificial sequence
  NS4A/3 PR S139A K136N
MGKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLNGSAGGPLLCPAGHAVGIFRAAVSTRGVAKAVDFIPVESLETTMRSPT

SEQ ID NO: 216 - DNA, 597 bp, Artificial sequence
  NS4A/3 PR S139A K136N
atgggcaagaaaaagggcagcgtggtcatcgtgggcagaatcaacctgagcggcgataccgcctacgctcagcagacaagaggcgaggaaggctgccaagagacaagccagaccggcagagacaagaaccaggtggaaggcgaggtgcagatcgtgtctacagctacccagaccttcctggccaccagcatcaatggcgtgctgtggacagtgtatcacggcgctggcaccagaacaatcgcctctccaaagggccccgtgacacagatgtacaccaacgtggacaaggacctcgtcggatggcaggctcctcagggctctagaagcctgacaccttgtacctgcggcagcagcgatctgtacctggtcacaagacacgccgacgtgatccccgtcagaagaagaggcgatagcagaggcagcctgctgagccctagacctatcagctacctgaacggatctgccggcggacctctgctttgtcctgctggacatgccgtgggcatctttagagccgccgtgtctactagaggcgtggccaaagccgtggacttcatccctgtggaaagcctggaaaccaccatgagaagccccacc

SEQ ID NO: 217 - protein, 215 aa, Artificial sequence
  His-TEV-NS4A/3 PR S139A
MGSSHHHHHHGSENLYFQSKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 218 - DNA, 645 bp, Artificial sequence
  His-TEV-NS4A/3 PR S139A
atgggtagcagccatcaccatcatcatcatggtagcgaaaatctgtatttccagagcaaaaaaaagggcagcgttgttattgtgggtcgtattaatctgagcggtgataccgcatatgcacagcagacccgtggtgaagaaggttgtcaagaaaccagccagaccggtcgtgataaaaatcaggttgaaggtgaagttcagattgttagcaccgcaacacagacctttctggcaaccagcattaatggtgttctgtggaccgtttatcatggtgcaggcacccgtaccattgcaagcccgaaaggtccggttacacagatgtataccaatgtggataaagatctggttggttggcaggcaccgcagggtagccgtagtctgaccccgtgtacctgtggtagcagcgatctgtatctggttacccgtcatgcagatgttattccggttcgtcgtcgtggtgatagccgtggtagcctgctgagtccgcgtccgattagctatctgaaaggtagtgccggtggtccgctgctgtgtccggcaggtcatgcagttggtatttttcgtgcagcagttagcacccgtggcgttgcaaaagcagttgattttatcccggttgaaagcctggaaaccaccatgcgtagcccg

SEQ ID NO: 219 - protein, 197 aa, Artificial sequence
  NS4A/3 S139A post-TEV cleavage
SKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 220 - protein, 280 aa, Artificial sequence
  pelB-PRSIM_57-TEV-His
MKYLLPTAAAGLLLLAAQPAMAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARHTNYITVFDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDHHWEQVVFGGGTKLTVLENLYFQSHHHHHH

SEQ ID NO: 221 - DNA, 840 bp, Artificial sequence
  pelB_PRSIM_57-TEV-His
atgaaatatctgctgccgaccgcagcagcgggtctgctgctgctggcagcacagcctgcaatggcacaggttcagctggttcagagcggtgcagaagttaaaaaaccgggtagcagcgttaaagttagctgtaaagcaagcggtggcacctttagcagctatgcaattagctgggttcgtcaggcacctggtcaaggtctggaatggatgggtggtattattccgatttttggcaccgcaaattatgcccagaaatttcagggtcgtgttaccattaccgcagatgaaagcaccagcaccgcatatatggaactgagcagcctgcgtagcgaagataccgcagtgtattattgtgcacgtcataccaactatatcaccgtgtttgattattggggtcagggcaccctggttaccgttagcagcggtggtggtggtagcggtggcggaggttcaggtggtggcggttcagcacagagcgttctgacccagcctccgagcgcaagcggtacaccgggtcagcgtgtgaccattagctgtagcggtagcagcagtaatattggtagcaataccgttaattggtatcagcagctgccaggcaccgcaccgaaactgctgatttatagcaataatcagcgtccgagcggtgttccggatcgttttagcggtagtaaaagcggcaccagcgcaagcctggcaattagcggtctgcagagcgaagatgaagcagattattactgtgcagcatgggatcatcattgggaacaagttgtttttggtggtggcaccaaactgaccgttctggaaaatctgtatttccagagccatcaccatcatcatcat

SEQ ID NO: 222 - protein, 251 aa, Artificial sequence
  PRSIM_57 post-TEV cleavage, pelB removal
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARHTNYITVFDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDHHWEQVVFGGGTKLTVLENLYFQ

SEQ ID NO: 223 - protein, 591 aa, Artificial sequence
  PRSIM23_NS4A/3 PR S139A_DCasp9
MGSRLDAPSQIEVKDVTDTTALITWVDPRYDDIWWFELTYGIKDVPGDRTTIKLYLNDPYYSIGNLKPDTEYEVSLISYTGDSYSRSGSNPAKITFKTGLGGGSGMKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 224 - DNA, 1779 bp, Artificial sequence
  PRSIM23_NS4A/3 PR S139A_DCasp9
atgggctctagactggatgcccctagccagatcgaagtgaaggacgtgaccgacaccaccgctctgatcacctgggttgaccccagatacgacgatatctggtggttcgagctgacctacggcatcaaggatgtgcccggcgacagaaccaccatcaagctgtacctgaacgacccctactacagcatcggcaacctgaagcctgacaccgagtacgaggtgtccctgatcagctacaccggcgactcctacagcagaagcggcagcaatcctgccaagatcaccttcaagaccggccttgggggcggatccggcatgaagaaaaagggctctgtggtcatcgtgggcagaatcaacctgagcggcgataccgcctacgctcagcagacaagaggcgaggaaggctgccaagagacaagccagaccggcagagacaagaaccaggtggaaggcgaggtgcagatcgtgtctacagctacccagaccttcctggccaccagcatcaatggcgtgctgtggacagtgtatcacggcgctggcaccagaacaatcgcctctccaaagggccccgtgacacagatgtacaccaacgtggacaaggacctcgtcggatggcaagcccctcagggctctagaagcctgacaccttgtacctgcggcagcagcgatctgtacctggtcacaagacacgccgacgtgatccccgtcagaagaagaggcgatagcagaggcagcctgctgagccctagacctatcagctacctgaagggatctgccggcggacctctgctttgtcctgctggacatgccgtgggcatctttagagccgccgtgtctactagaggcgtggccaaagccgtggacttcatccctgtggaaagcctggaaaccaccatgcggagccccgggggaggctccggcgtggatggctttggagatgtgggcgccctggaatccctgagaggaaatgccgatctggcctacatcctgagcatggaaccttgcggccactgcctgattatcaacaatgtgaacttctgccgcgagagcggcctgagaacaagaaccggcagcaacatcgattgcgagaagctgcggagaagattcagcagcctgcacttcatggtggaagtgaagggcgacctgaccgccaagaaaatggtgctggctctgctggaactggccagacaggatcatggcgcactggattgctgcgtggtcgtgattctgagccacggctgtcaggccagccatctgcaattccctggcgccgtgtatggcaccgatggctgtcctgtgtccgtggaaaagatcgtgaacatcttcaacggcaccagctgtcctagcctcggcggaaagcccaagctgttcttcatccaagcctgtggcggcgagcagaaggatcacggatttgaggtggccagcacaagccccgaggatgagagccctggaagcaaccctgagcctgacgccacacctttccaagagggactgagaaccttcgaccagctggacgctatcagctccctgcctacacctagcgacatcttcgtgtcctacagcacattccccggctttgtgtcttggcgggaccccaagtctggctcttggtacgtggaaaccctggatgacatcttcgagcagtgggcccatagcgaggacctgcaatctctgctgctgagagtggccaatgccgtgtccgtgaagggcatctacaagcagatgcccggctgcttcaacttcctgcggaagaagctgtttttcaagaccagctgatag

SEQ ID NO: 225 - protein, 740 aa, Artificial sequence
  NS4A/3 PR S139A-VPR
MGKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPTGGGGSGGGGSEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 226 - DNA, 2220 bp, Artificial sequence
  NS4A/3 PR S139A-VPR
atgggcaagaaaaagggcagcgtggtcatcgtgggcagaatcaacctgagcggcgataccgcctacgctcagcagacaagaggcgaggaaggctgccaagagacaagccagaccggcagagacaagaaccaggtggaaggcgaggtgcagatcgtgtctacagctacccagaccttcctggccaccagcatcaatggcgtgctgtggacagtgtatcacggcgctggcaccagaacaatcgcctctccaaagggccccgtgacacagatgtacaccaacgtggacaaggacctcgtcggatggcaagcccctcagggctccagaagcctgacaccttgtacctgcggcagcagcgatctgtacctggtcacaagacacgccgacgtgatccccgtcagaagaagaggcgatagcagaggcagcctgctgagccctagacctatcagctacctgaagggatctgccggcggacctctgctttgtcctgctggacatgccgtgggcatctttagagccgccgtgtctactagaggcgtggccaaagccgtggacttcatccctgtggaaagcctggaaaccaccatgagaagccccaccggtggcggcggatctggcggaggtggaagtgaagcttctggcagcggtagagccgacgctctggacgacttcgacctggatatgctgggctctgacgccctggatgattttgatctggacatgctcggcagcgacgccctcgacgatttcgatctcgatatgttgggaagcgacgcacttgatgactttgacctcgacatgttgatcaatagcagaagcagcggcagccccaagaaaaagcggaaagtgggcagccagtacctgcctgacaccgacgacagacaccggatcgaagagaagcggaagcggacctacgagacattcaagagcatcatgaagaagtccccattcagcggccccaccgatcctagacctccacctagaagaatcgccgtgcctagcagatctagcgccagcgtgccaaaacctgctcctcagccttatcctttcaccagcagcctgagcaccatcaactacgacgagttccctaccatggtgttccccagcggccagatctctcaggcttctgctcttgctccagctcctcctcaggttctgcctcaagctcctgcaccagcaccggctccagctatggtttctgctttggctcaggcccctgctcctgtgcctgttcttgctcctggaccacctcaggctgttgctcctcctgctccaaaacctacacaggccggcgagggaacactgtctgaagctctgctgcagctccagttcgacgacgaagatctgggagccctgctgggcaatagcacagatcctgccgtgttcaccgatctggccagcgtggacaatagcgagttccagcagctcctgaatcagggcatccctgtggctcctcacaccaccgaacctatgctgatggaataccccgaggccatcaccagactggtcaccggcgctcaaagaccacctgatccagctccagcacctctgggagcaccaggactgcctaatggactgctgtctggcgacgaggacttcagctctatcgccgacatggatttcagcgccctgctcggctctggctccggctctagagatagcagagaaggcatgttcctgcctaagcctgaggccggctctgccatctccgatgtgtttgagggcagagaagtgtgccagcctaagcggatccggccttttcaccctcctggaagcccttgggccaacagacctctgcctgcttctctggcccctacaccaacaggacctgtgcacgaacctgtgggcagtctgaccccagctcctgttcctcaacctctggatcccgctcctgctgtgacacctgaagcctctcatctgctggaagatcccgacgaagagacaagccaggccgtgaaggccctgagagaaatggccgacacagtgatccctcagaaagaggaagccgccatctgcggacagatggacctgtctcatcctccaccaagaggccacctggacgagctgacaaccacactggaatccatgaccgaggacctgaacctggacagccctctgacacccgagctgaacgagatcctggacaccttcctgaacgacgagtgtctgctgcacgccatgcacatctctaccggcctgagcatcttcgacaccagcctgttc

SEQ ID NO: 227 - protein, 1682 aa, Artificial sequence
  spdCas9-PRSIM_23x3
MDYYPYDVPDYADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVGSAGSRLDAPSQIEVKDVTDTTALITWVDPRYDDIWWFELTYGIKDVPGDRTTIKLYLNDPYYSIGNLKPDTEYEVSLISYTGDSYSRSGSNPAKITFKTGLRLDAPSQIEVKDVTDTTALITWVDPRYDDIWWFELTYGIKDVPGDRTTIKLYLNDPYYSIGNLKPDTEYEVSLISYTGDSYSRSGSNPAKITFKTGLRLDAPSQIEVKDVTDTTALITWVDPRYDDIWWFELTYGIKDVPGDRTTIKLYLNDPYYSIGNLKPDTEYEVSLISYTGDSYSRSGSNPAKITFKTGL

SEQ ID NO: 228 - DNA, 5049 bp, Artificial sequence
  spdCas9-PRSIM_23x3
atggactattacccctacgacgtgcccgattacgccgacaagaagtattctatcggactggccatcgggactaatagcgtcgggtgggccgtgatcactgacgagtacaaggtgccctctaagaagttcaaggtgctcgggaacaccgaccggcattccatcaagaaaaatctgatcggagctctcctctttgattcaggggaaaccgctgaagcaacccgcctcaagcggactgctagacggcggtacaccaggaggaagaaccggatttgttaccttcaagagatattctccaacgaaatggcaaaggtcgacgacagcttcttccataggctggaagaatcattcctcgtggaagaggataagaagcatgaacggcatcccatcttcggtaatatcgtcgacgaggtggcctatcacgagaaatacccaaccatctaccatcttcgcaaaaagctggtggactcaaccgacaaggcagacctccggcttatctacctggccctggcccacatgatcaagttcagaggccacttcctgatcgagggcgacctcaatcctgacaatagcgatgtggataaactgttcatccagctggtgcagacttacaaccagctctttgaagagaaccccatcaatgcaagcggagtcgatgccaaggccattctgtcagcccggctgtcaaagagccgcagacttgagaatcttatcgctcagctgccgggtgaaaagaaaaatggactgttcgggaacctgattgctctttcacttgggctgactcccaatttcaagtctaatttcgacctggcagaggatgccaagctgcaactgtccaaggacacctatgatgacgatctcgacaacctcctggcccagatcggtgaccaatacgccgaccttttccttgctgctaagaatctttctgacgccatcctgctgtctgacattctccgcgtgaacactgaaatcaccaaggcccctctttcagcttcaatgattaagcggtatgatgagcaccaccaggacctgaccctgcttaaggcactcgtccggcagcagcttccggagaagtacaaggaaatcttctttgaccagtcaaagaatggatacgccggctacatcgacggaggtgcctcccaagaggaattttataagtttatcaaacctatccttgagaagatggacggcaccgaagagctcctcgtgaaactgaatcgggaggatctgctgcggaagcagcgcactttcgacaatgggagcattccccaccagatccatcttggggagcttcacgccatccttcggcgccaagaggacttctacccctttcttaaggacaacagggagaagattgagaaaattctcactttccgcatcccctactacgtgggacccctcgccagaggaaatagccggtttgcttggatgaccagaaagtcagaagaaactatcactccctggaacttcgaagaggtggtggacaagggagccagcgctcagtcattcatcgaacggatgactaacttcgataagaacctccccaatgagaaggtcctgccgaaacattccctgctctacgagtactttaccgtgtacaacgagctgaccaaggtgaaatatgtcaccgaagggatgaggaagcccgcattcctgtcaggcgaacaaaagaaggcaattgtggaccttctgttcaagaccaatagaaaggtgaccgtgaagcagctgaaggaggactatttcaagaaaattgaatgcttcgactctgtggagattagcggggtcgaagatcggttcaacgcaagcctgggtacctaccatgatctgcttaagatcatcaaggacaaggattttctggacaatgaggagaacgaggacatccttgaggacattgtcctgactctcactctgttcgaggaccgggaaatgatcgaggagaggcttaagacctacgcccatctgttcgacgataaagtgatgaagcaacttaaacggagaagatataccggatggggacgccttagccgcaaactcatcaacggaatccgggacaaacagagcggaaagaccattcttgatttccttaagagcgacggattcgctaatcgcaacttcatgcaacttatccatgatgattccctgacctttaaggaggacatccagaaggcccaagtgtctggacaaggtgactcactgcacgagcatatcgcaaatctggctggttcacccgctattaagaagggtattctccagaccgtgaaagtcgtggacgagctggtcaaggtgatgggtcgccataaaccagagaacattgtcatcgagatggccagggaaaaccagactacccagaagggacagaagaacagcagggagcggatgaaaagaattgaggaagggattaaggagctcgggtcacagatccttaaagagcacccggtggaaaacacccagcttcagaatgagaagctctatctgtactaccttcaaaatggacgcgatatgtatgtggaccaagagcttgatatcaacaggctctcagactacgacgtggacgccatcgtccctcagagcttcctcaaagacgactcaattgacaataaggtgctgactcgctcagacaagaaccggggaaagtcagataacgtgccctcagaggaagtcgtgaaaaagatgaagaactattggcgccagcttctgaacgcaaagctgatcactcagcggaagttcgacaatctcactaaggctgagaggggcggactgagcgaactggacaaagcaggattcattaaacggcaacttgtggagactcggcagattactaaacatgtcgcccaaatccttgactcacgcatgaataccaagtacgacgaaaacgacaaacttatccgcgaggtgaaggtgattaccctgaagtccaagctggtcagcgatttcagaaaggactttcaattctacaaagtgcgggagatcaataactatcatcatgctcatgacgcatatctgaatgccgtggtgggaaccgccctgatcaagaagtacccaaagctggaaagcgagttcgtgtacggagactacaaggtctacgacgtgcgcaagatgattgccaaatctgagcaggagatcggaaaggccaccgcaaagtacttcttctacagcaacatcatgaatttcttcaagaccgaaatcacccttgcaaacggtgagatccggaagaggccgctcatcgagactaatggggagactggcgaaatcgtgtgggacaagggcagagatttcgctaccgtgcgcaaagtgctttctatgcctcaagtgaacatcgtgaagaaaaccgaggtgcaaaccggaggcttttctaaggaatcaatcctccccaagcgcaactccgacaagctcattgcaaggaagaaggattgggaccctaagaagtacggcggattcgattcaccaactgtggcttattctgtcctggtcgtggctaaggtggaaaaaggaaagtctaagaagctcaagagcgtgaaggaactgctgggtatcaccattatggagcgcagctccttcgagaagaacccaattgactttctcgaagccaaaggttacaaggaagtcaagaaggaccttatcatcaagctcccaaagtatagcctgttcgaactggagaatgggcggaagcggatgctcgcctccgctggcgaacttcagaagggtaatgagctggctctcccctccaagtacgtgaatttcctctaccttgcaagccattacgagaagctgaaggggagccccgaggacaacgagcaaaagcaactgtttgtggagcagcataagcattatctggacgagatcattgagcagatttccgagttttctaaacgcgtcattctcgctgatgccaacctcgataaagtccttagcgcatacaataagcacagagacaaaccaattcgggagcaggctgagaatatcatccacctgttcaccctcaccaatcttggtgcccctgccgcattcaagtacttcgacaccaccatcgaccggaaacgctatacctccaccaaagaagtgctggacgccaccctcatccaccagagcatcaccggactttacgaaactcggattgacctctcacagctcggaggggatcccaagaagaagcggaaagtcggcagcgctggctctagactggatgcccctagccagatcgaagtgaaggacgtgaccgacaccaccgctctgatcacctgggttgaccccagatacgacgacatctggtggttcgagctgacctacggcatcaaggatgtgcccggcgacagaaccaccatcaagctgtacctgaacgacccctactacagcatcggcaacctgaagcctgacaccgagtacgaggtgtccctgatcagctacaccggcgactcctacagcagaagcggcagcaatcctgccaagatcaccttcaagaccggcctgagactggacgcaccctctcagattgaagtcaaagatgtcaccgacacgacagccctgattacatgggttgaccctcgctacgatgatatttggtggtttgaactcacgtacgggatcaaagacgtgccaggggatcgcacaacaatcaagctctatctcaatgatccgtactactccatcgggaatctgaaacccgatacagagtacgaagtctccctcatctcttacaccggggacagctactccagatccggctccaatccagccaaaattacgtttaagacaggcctgcggctggatgctccatctcaaatagaagttaaggatgtgacggatacgacggccctcatcacttgggttgaccctcgatatgacgatatttggtggttcgaattgacgtatggcattaaggacgtcccaggcgaccggacaactattaagctgtatcttaacgatccttattatagcatcggaaatctcaagccggataccgaatatgaggtttccctcatttcctatactggggactcctactctcgctccggctctaacccagctaagatcacttttaaaaccgggctttaa

SEQ ID NO: 229 - DNA, 19 bp, Artificial sequence
  gRNA IL-2
gttacattagcccacactt

SEQ ID NO: 230 - protein, 591 aa, Artificial sequence
  PRSIM23_NS4A/3 PR S139A_DCasp9 (S196A)
MGSRLDAPSQIEVKDVTDTTALITWVDPRYDDIWWFELTYGIKDVPGDRTTIKLYLNDPYYSIGNLKPDTEYEVSLISYTGDSYSRSGSNPAKITFKTGLGGGSGMKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPGGGSGVDGFGDVGALESLRGNADLAYILSMEPCGHCLIINNVNFCRESGLRTRTGSNIDCEKLRRRFSALHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 231 - DNA, 1779 bp, Artificial sequence
  PRSIM23_NS4A/3 PR S139A_DCasp9 (S196A)
atgggctctagactggatgcccctagccagatcgaagtgaaggacgtgaccgacaccaccgctctgatcacctgggttgaccccagatacgacgatatctggtggttcgagctgacctacggcatcaaggatgtgcccggcgacagaaccaccatcaagctgtacctgaacgacccctactacagcatcggcaacctgaagcctgacaccgagtacgaggtgtccctgatcagctacaccggcgactcctacagcagaagcggcagcaatcctgccaagatcaccttcaagaccggccttgggggcggatccggcatgaagaaaaagggctctgtggtcatcgtgggcagaatcaacctgagcggcgataccgcctacgctcagcagacaagaggcgaggaaggctgccaagagacaagccagaccggcagagacaagaaccaggtggaaggcgaggtgcagatcgtgtctacagctacccagaccttcctggccaccagcatcaatggcgtgctgtggacagtgtatcacggcgctggcaccagaacaatcgcctctccaaagggccccgtgacacagatgtacaccaacgtggacaaggacctcgtcggatggcaagcccctcagggctctagaagcctgacaccttgtacctgcggcagcagcgatctgtacctggtcacaagacacgccgacgtgatccccgtcagaagaagaggcgatagcagaggcagcctgctgagccctagacctatcagctacctgaagggatctgccggcggacctctgctttgtcctgctggacatgccgtgggcatctttagagccgccgtgtctactagaggcgtggccaaagccgtggacttcatccctgtggaaagcctggaaaccaccatgcggagccccgggggaggctccggcgtggatggctttggagatgtgggcgccctggaatccctgagaggaaatgccgatctggcctacatcctgagcatggaaccttgcggccactgcctgattatcaacaatgtgaacttctgccgcgagagcggcctgagaacaagaaccggcagcaacatcgattgcgagaagctgcggagaagattcagcgccctgcacttcatggtggaagtgaagggcgacctgaccgccaagaaaatggtgctggctctgctggaactggccagacaggatcatggcgcactggattgctgcgtggtcgtgattctgagccacggctgtcaggccagccatctgcaattccctggcgccgtgtatggcaccgatggctgtcctgtgtccgtggaaaagatcgtgaacatcttcaacggcaccagctgtcctagcctcggcggaaagcccaagctgttcttcatccaagcctgtggcggcgagcagaaggatcacggatttgaggtggccagcacaagccccgaggatgagagccctggaagcaaccctgagcctgacgccacacctttccaagagggactgagaaccttcgaccagctggacgctatcagctccctgcctacacctagcgacatcttcgtgtcctacagcacattccccggctttgtgtcttggcgggaccccaagtctggctcttggtacgtggaaaccctggatgacatcttcgagcagtgggcccatagcgaggacctgcaatctctgctgctgagagtggccaatgccgtgtccgtgaagggcatctacaagcagatgcccggctgcttcaacttcctgcggaagaagctgtttttcaagaccagctgatag

SEQ ID NO: 232 - DNA, 834 bp, Artificial sequence
  GFP-PEST
atggaaagcgacgagtctggcctgcctgctatggaaatcgagtgccggatcaccggcacactgaacggcgtggaattcgaactcgttggcggcggagagggcacacctgaacagggcagaatgaccaacaagatgaagtccaccaaaggcgccctgacattcagcccctacctgctgtctcacgtgatgggctacggcttctaccacttcggcacataccctagcggctacgagaaccctttcctgcacgccatcaacaacggcggctacaccaacaccagaatcgagaagtacgaggacggcggcgtgctgcacgtgtccttcagctacagatatgaggccggcagagtgatcggcgacttcaaagtgatgggcaccggatttcccgaggacagcgtgatcttcaccgacaagatcatccggtccaacgccaccgtggaacatctgcaccctatgggcgacaacgacctggatggcagcttcaccagaaccttcagcctgagagatggcggctactacagcagcgtggtggacagccacatgcacttcaagagcgccatccatcctagcatcctgcagaacggcggacccatgttcgccttcagaagagtggaagaggaccacagcaacaccgagctgggcatcgtggaataccagcacgccttcaagacccctgatgccgatgccggcgaggaaagaagcagagatatcagccacggcttcccaccagctgtggccgctcaagatgatggcacactgcctatgagctgcgcccaagagtccggcatggatagacatcctgccgcctgtgccagcgccagaatcaatgtgtaa

SEQ ID NO: 233 - protein, 277 aa, Artificial sequence
  GFP-PEST
MESDESGLPAMEIECRITGTLNGVEFELVGGGEGTPEQGRMTNKMKSTKGALTFSPYLLSHVMGYGFYHFGTYPSGYENPFLHAINNGGYTNTRIEKYEDGGVLHVSFSYRYEAGRVIGDFKVMGTGFPEDSVIFTDKIIRSNATVEHLHPMGDNDLDGSFTRTFSLRDGGYYSSVVDSHMHFKSAIHPSILQNGGPMFAFRRVEEDHSNTELGIVEYQHAFKTPDADAGEERSRDISHGFPPAVAAQDDGTLPMSCAQESGMDRHPAACASARINV

SEQ ID NO: 234 - DNA, 490 bp, Artificial sequence
  hAAV1 left homologous arm
gagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccac

SEQ ID NO: 235 - DNA, 527 bp, Artificial sequence
  hAAV1 Right Homologous arm
gatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgtctaga

SEQ ID NO: 236 - DNA, 20 bp, Artificial sequence
  gRNA AAVS1
gttaatgtggctctggttct

SEQ ID NO: 237 - DNA, 1374 bp, Artificial Sequence
  MEDI8852 heavy chain
caggttcagctgcagcagtctggacctggcctggtcaagcctagccagacactgtctctgacctgtgccatcagcggcgatagcgtgtccagctacaacgccgtgtggaactggatcagacagagccctagcagaggcctggaatggctgggcagaacctactacagaagcggctggtacaacgactacgccgagagcgtgaagtcccggatcaccatcaatcccgacaccagcaagaaccagttcagcctccagctgaacagcgtgacccctgaggataccgccgtgtactactgtgccagatccggccacatcaccgtgttcggagtgaacgtggacgccttcgatatgtggggccagggcacaatggtcaccgtgtctagcgcctctacaaagggccctagcgtgttccctctggctcctagcagcaagtctacaagcggaggaacagccgctctgggctgcctcgtgaaggattactttcccgagcctgtgaccgtgtcctggaattctggcgctctgacaagcggcgtgcacacctttccagctgtgctgcaaagcagcggcctgtactctctgagcagcgtggtcacagtgccaagctctagcctgggcacccagacctacatctgcaatgtgaatcacaagcccagcaacaccaaggtggacaagagagtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaatcaggtgtccctgacctgcctggttaagggcttctacccctccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttctcctgcagcgtgatgcacgaggccctgcacaaccactacacccagaagtccctgagcctgtctcctggcaaa

SEQ ID NO: 238 - DNA, 630 bp, Artificial Sequence
  MEDI8852 light chain
gacatccagatgacacagagccctagcagcctgtctgccagcgtgggagacagagtgaccatcacctgtagaaccagccagagcctgagcagctacacccactggtatcagcagaagcctggcaaggcccctaagctgctgatctatgccgccagctctagaggcagcggagtgccttctagattttccggcagcggctccggcaccgatttcaccctgaccatatctagcctgcagcctgaggacttcgccacctactactgccagcagagcagaacctttggccagggcaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctactctctgagcagcaccctgacactgagcaaggccgactacgagaagcacaaagtgtacgcctgcgaagtgacccaccagggcctttctagccctgtgaccaagagcttcaaccggggcgaatgt

SEQ ID NO: 239 - protein, 4 aa, Artificial Sequence
  linker
GGGS

SEQ ID NO: 240 - protein, 5 aa, Artificial Sequence
  Linker
GGGGS

SEQ ID NO: 241 - protein, 4 aa, Artificial Sequence
  linker
GGSG

SEQ ID NO: 242 - protein, 4 aa, Artificial Sequence
  linker
GSGG

SEQ ID NO: 243 - protein, 4 aa, Artificial Sequence
  linker
SGGG

SEQ ID NO: 244 - protein, 4 aa, Artificial Sequence
  linker
SSGG

SEQ ID NO: 245 - protein, 4 aa, Artificial Sequence
  linker
SSSG

SEQ ID NO: 246 - DNA, 20 bp, Artificial Sequence
  B2M_LHA_PF2
gggaggaacttcttggcaca

SEQ ID NO: 247 - DNA, 20 bp, Artificial Sequence
  B2M_RHA_PR2
aggagagactcacgctggat